SEQUENCE LISTING (8 sequences)

SEQ ID NO: 1 - protein, 1068 aa, Homo sapiens
  MISC_FEATURE: Human phosphatidylinositol 3-kinase catalytic subunit alpha polypeptide (PIK3CA) encoded by mRNA polynucleotide sequence ID NCBI Accession Reference No NM_006218; GI54792081 (3724 nucleotides)
MPPRPSSGELWGIHLMPPRILVECLLPNGMIVTLECLREATLITIKHELFKEARKYPLHQLLQDESSYIFVSVTQEAEREEFFDETRRLCDLRLFQPFLKVIEPVGNREEKILNREIGFAIGMPVCEFDMVKDPEVQDFRRNILNVCKEAVDLRDLNSPHSRAMYVYPPNVESSPELPKHIYNKLDKGQIIVVIWVIVSPNNDKQKYTLKINHDCVPEQVIAEAIRKKTRSMLLSSEQLKLCVLEYQGKYILKVCGCDEYFLEKYPLSQYKYIRSCIMLGRMPNLMLMAKESLYSQLPMDCFTMPSYSRRISTATPYMNGETSTKSLWVINSALRIKILCATYVNVNIRDIDKIYVRTGIYHGGEPLCDNVNTQRVPCSNPRWNEWLNYDIYIPDLPRAARLCLSICSVKGRKGAKEEHCPLAWGNINLFDYTDTLVSGKMALNLWPVPHGLEDLLNPIGVTGSNPNKETPCLELEFDWFSSVVKFPDMSVIEEHANWSVSREAGFSYSHAGLSNRLARDNELRENDKEQLKAISTRDPLSEITEQEKDFLWSHRHYCVTIPEILPKLLLSVKWNSRDEVAQMYCLVKDWPPIKPEQAMELLDCNYPDPMVRGFAVRCLEKYLTDDKLSQYLIQLVQVLKYEQYLDNLLVRFLLKKALTNQRIGHFFFWHLKSEMHNKTVSQRFGLLLESYCRACGMYLKHLNRQVEAMEKLINLTDILKQEKKDETQKVQMKFLVEQMRRPDFMDALQGFLSPLNPAHQLGNLRLEECRIMSSAKRPLWLNWENPDIMSELLFQNNEIIFKNGDDLRQDMLTLQIIRIMENIWQNQGLDLRMLPYGCLSIGDCVGLIEVVRNSHTIMQIQCKGGLKGALQFNSHTLHQWLKDKNKGEIYDAAIDLFTRSCAGYCVATFILGIGDRHNSNIMVKDDGQLFHIDFGHFLDHKKKKFGYKRERVPFVLTQDFLIVISKGAQECTKTREFERFQEMCYKAYLAIRQHANLFINLFSMMLGSGMPELQSFDDIAYIRKTLALDKTEQEALEYFMKQMNDAHHGGWTTKMDWIFHTIKQHALN

SEQ ID NO: 2 - DNA, 3724 bp, Homo sapiens
  misc_feature: Human phosphatidylinositol 3-kinase catalytic subunit alpha mRNA polynucleotide sequence ID NCBI Accession Reference No NM_006218; GI54792081 (3724 nucleotides)
tctccctcggcgccgccgccgccgcccgcggggctgggacccgatgcggttagagccgcggagcctggaagagccccgagcgtttctgctttgggacaaccatacatctaattccttaaagtagttttatatgtaaaacttgcaaagaatcagaacaatgcctccacgaccatcatcaggtgaactgtggggcatccacttgatgcccccaagaatcctagtagaatgtttactaccaaatggaatgatagtgactttagaatgcctccgtgaggctacattaataaccataaagcatgaactatttaaagaagcaagaaaataccccctccatcaacttcttcaagatgaatcttcttacattttcgtaagtgttactcaagaagcagaaagggaagaattttttgatgaaacaagacgactttgtgaccttcggctttttcaaccctttttaaaagtaattgaaccagtaggcaaccgtgaagaaaagatcctcaatcgagaaattggttttgctatcggcatgccagtgtgtgaatttgatatggttaaagatccagaagtacaggacttccgaagaaatattctgaacgtttgtaaagaagctgtggatcttagggacctcaattcacctcatagtagagcaatgtatgtctatcctccaaatgtagaatcttcaccagaattgccaaagcacatatataataaattagataaagggcaaataatagtggtgatctgggtaatagtttctccaaataatgacaagcagaagtatactctgaaaatcaaccatgactgtgtaccagaacaagtaattgctgaagcaatcaggaaaaaaactcgaagtatgttgctatcctctgaacaactaaaactctgtgttttagaatatcagggcaagtatattttaaaagtgtgtggatgtgatgaatacttcctagaaaaatatcctctgagtcagtataagtatataagaagctgtataatgcttgggaggatgcccaatttgatgttgatggctaaagaaagcctttattctcaactgccaatggactgttttacaatgccatcttattccagacgcatttccacagctacaccatatatgaatggagaaacatctacaaaatccctttgggttataaatagtgcactcagaataaaaattctttgtgcaacctacgtgaatgtaaatattcgagacattgataagatctatgttcgaacaggtatctaccatggaggagaacccttatgtgacaatgtgaacactcaaagagtaccttgttccaatcccaggtggaatgaatggctgaattatgatatatacattcctgatcttcctcgtgctgctcgactttgcctttccatttgctctgttaaaggccgaaagggtgctaaagaggaacactgtccattggcatggggaaatataaacttgtttgattacacagacactctagtatctggaaaaatggctttgaatctttggccagtacctcatggattagaagatttgctgaaccctattggtgttactggatcaaatccaaataaagaaactccatgcttagagttggagtttgactggttcagcagtgtggtaaagttcccagatatgtcagtgattgaagagcatgccaattggtctgtatcccgagaagcaggatttagctattcccacgcaggactgagtaacagactagctagagacaatgaattaagggaaaatgacaaagaacagctcaaagcaatttctacacgagatcctctctctgaaatcactgagcaggagaaagattttctatggagtcacagacactattgtgtaactatccccgaaattctacccaaattgcttctgtctgttaaatggaattctagagatgaagtagcccagatgtattgcttggtaaaagattggcctccaatcaaacctgaacaggctatggaacttctggactgtaattacccagatcctatggttcgaggttttgctgttcggtgcttggaaaaatatttaacagatgacaaactttctcagtatttaattcagctagtacaggtcctaaaatatgaacaatatttggataacttgcttgtgagatttttactgaagaaagcattgactaatcaaaggattgggcactttttcttttggcatttaaaatctgagatgcacaataaaacagttagccagaggtttggcctgcttttggagtcctattgtcgtgcatgtgggatgtatttgaagcacctgaataggcaagtcgaggcaatggaaaagctcattaacttaactgacattctcaaacaggagaagaaggatgaaacacaaaaggtacagatgaagtttttagttgagcaaatgaggcgaccagatttcatggatgctctacagggctttctgtctcctctaaaccctgctcatcaactaggaaacctcaggcttgaagagtgtcgaattatgtcctctgcaaaaaggccactgtggttgaattgggagaacccagacatcatgtcagagttactgtttcagaacaatgagatcatctttaaaaatggggatgatttacggcaagatatgctaacacttcaaattattcgtattatggaaaatatctggcaaaatcaaggtcttgatcttcgaatgttaccttatggttgtctgtcaatcggtgactgtgtgggacttattgaggtggtgcgaaattctcacactattatgcaaattcagtgcaaaggcggcttgaaaggtgcactgcagttcaacagccacacactacatcagtggctcaaagacaagaacaaaggagaaatatatgatgcagccattgacctgtttacacgttcatgtgctggatactgtgtagctaccttcattttgggaattggagatcgtcacaatagtaacatcatggtgaaagacgatggacaactgtttcatatagattttggacactttttggatcacaagaagaaaaaatttggttataaacgagaacgtgtgccatttgttttgacacaggatttcttaatagtgattagtaaaggagcccaagaatgcacaaagacaagagaatttgagaggtttcaggagatgtgttacaaggcttatctagctattcgacagcatgccaatctcttcataaatcttttctcaatgatgcttggctctggaatgccagaactacaatcttttgatgacattgcatacattcgaaagaccctagccttagataaaactgagcaagaggctttggagtatttcatgaaacaaatgaatgatgcacatcatggtggctggacaacaaaaatggattggatcttccacacaattaaacagcatgcattgaactgaaaagataactgagaaaatgaaagctcactctggattccacactgcactgttaataactctcagcaggcaaagaccgattgcataggaattgcacaatccatgaacagcattagaatttacagcaagaacagaaataaaatactatataatttaaataatgtaaacgcaaacagggtttgatagcacttaaactagttcatttcaaaattaagctttagaataatgcgcaatttcatgttatgccttaagtccaaaaaggtaaactttgaagattgtttgtatctttttttaaaaaacaaaacaaaacaaaaatccccaaaatatatagaaatgatggagaaggaaaaaaaaaaaaaaaaa

SEQ ID NO: 3 - DNA, 23 bp, Artificial Sequence
  human exon 9 forward primer
gggaaaaatatgacaaagaaagc

SEQ ID NO: 4 - DNA, 22 bp, Artificial Sequence
  human exon 9 reverse primer
ctgagatcagccaaattcagtt

SEQ ID NO: 5 - DNA, 27 bp, Artificial Sequence
  human exon 9 sequencing primer
tagctagagacaatgaattaagggaaa

SEQ ID NO: 6 - DNA, 20 bp, Artificial Sequence
  human exon 20 forward primer
ctcaatgatgcttggctctg

SEQ ID NO: 7 - DNA, 21 bp, Artificial Sequence
  human exon 20 reverse primers
tggaatccagagtgagctttc

SEQ ID NO: 8 - DNA, 22 bp, Artificial Sequence
  human exon 20 sequencing primer
ttgatgacattgcatacattcg

(19) United States
(12) Patent Application Publication
(10) Pub. No.: US 2014/0024637 A1
(43) Pub. Date: Jan. 23, 2014

(54) Benzoxazepines as Inhibitors of PI3K/mTOR and Methods of Their use and Manufacture

(71) Applicant: Kenneth D. Rice, San Rafael, CA (US)

(72) Inventor: Kenneth D. Rice, San Rafael, CA (US)

(73) Assignee: Exelixis, Inc., South San Francisco, CA (US)

(21) Appl. No.: 13/989,366

(22) PCT Filed: Nov. 23, 2011
(86) PCT No.: PCT/US2011/062040

§ 371 Date: Oct. 10, 2013

Related U.S. Application Data
(60) Provisional application No. 61/417,070, filed on Nov. 24, 2010.

(51) Int. Cl. C07D 413/14; C07D 471/10; C07D 471/04

(52) U.S. Cl. CPC C07D 413/14; C07D 471/04; C07D 471/10
USPC 514/211.09; 540/552; 540/543; 540/480

(57) ABSTRACT

The invention is directed to Compounds of Formula I: and pharmaceutically acceptable salts or solvates thereof, as well as methods of making and using the compounds.

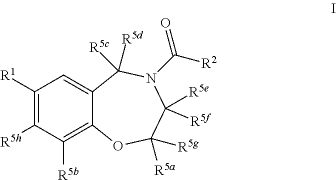

### CROSS-REFERENCE TO RELATED APPLICATIONS

This application claims the benefit of priority to U.S. Provisional Application No. 61/417,070, filed Nov. 24, 2010, which is incorporated herein by reference.

### SEQUENCE LISTING

This application incorporates by reference in its entirety the Sequence Listing entitled “10-035_Sequence.txt” (16.2 KB) which was created Nov. 23, 2011 and filed herewith on Nov. 23, 2011.

### BACKGROUND OF THE INVENTION

1. Field of the Invention

This invention relates to the field of protein kinases and inhibitors thereof. In particular, the invention relates to inhibitors of PI3K and/or the mammalian target of rapamycin (mTOR) signaling pathways, and methods of their use.

2. Background of the Invention

The PI3K pathway regulates cell growth, proliferation and survival, and is dysregulated with high frequency in human tumors. PI3K pathway activation in tumors occurs via multiple mechanisms including prevalent mutation and amplification of the PIK3CA gene (which encodes the p110 subunit of PI3Ka), or downregulation of the lipid phosphatase PTEN. Downstream of PI3K, mTOR controls cell growth and proliferation through its two distinct signaling complexes: mTORC1 and mTORC2. Given the role of PI3K signaling on critical cellular functions, an inhibitor that targets both PI3K and mTOR could provide therapeutic benefit to patient populations with tumors harboring activating mutations in PIK3CA or Ras, PTEN-deletion, or where tumors are upregulated in growth factor signaling.

Phosphatidylinositol 3-kinase (PI3Koc), a dual specificity protein kinase, is composed of an 85 kDa regulatory subunit and a 110 kDa catalytic subunit. The protein encoded by this gene represents the catalytic subunit, which uses ATP to phosphorylate PtdIns, PtdIns4P and PtdIns(4,5)P2. PTEN, a tumor suppressor which inhibits cell growth through multiple mechanisms, can dephosphorylate PIP3, the major product of PIK3CA. PIP3, in turn, is required for translocation of protein kinase B (AKT1, PKB) to the cell membrane, where it is phosphorylated and activated by upstream kinases. The effect of PTEN on cell death is mediated through the PIK3CA/AKT1 pathway.

PI3Kα has been implicated in the control of cytoskeletal reorganization, apoptosis, vesicular trafficking, proliferation and differentiation processes. Increased copy number and expression of PIK3CA is associated with a number of malignancies such as ovarian cancer (Campbell et al., Cancer Res 2004, 64, 7678-7681; Levine et al., Clin Cancer Res 2005, 11, 2875-2878; Wang et al., Hum Mutat 2005, 25, 322; Lee et al., Gynecol Oncol 2005, 97, 26-34), cervical cancer, breast cancer (Bachman, et al. Cancer Biol Ther 2004, 3, 772-775; Levine, et al., supra; Li et al., Breast Cancer Res Treat 2006, 96, 91-95; Saal et al., Cancer Res 2005, 65, 2554-2559; Samuels and Velculescu, Cell Cycle 2004, 3, 1221-1224), colorectal cancer (Samuels, et al. Science 2004, 304, 554; Velho et al. Eur J Cancer 2005, 41, 1649-1654), endometrial cancer (Oda et al. Cancer Res. 2005, 65, 10669-10673), gastric carcinomas (Byun et al., Int J Cancer 2003, 104, 318-327; Li et al., supra; Velho et al., supra; Lee et al., Oncogene 2005, 24, 1477-1480), hepatocellular carcinoma (Lee et al., id.), small and non-small cell lung cancer (Tang et al., Lung Cancer 2006, 51, 181-191; Massion et al., Am J Respir Crit. Care Med 2004, 170, 1088-1094), thyroid carcinoma (Wu et al., J Clin Endocrinol Metab 2005, 90, 4688-4693), acute myelogenous leukemia (AML) (Sujobert et al., Blood 1997, 106, 1063-1066), chronic myelogenous leukemia (CML) (Hickey and Cotter J Biol Chem 2006, 281, 2441-2450), and glioblastomas (Hartmann et al. Acta Neuropathol (Berl) 2005, 109, 639-642; Samuels et al., supra).

The mammalian target, mTOR, is a protein kinase that integrates both extracellular and intracellular signals of cellular growth, proliferation, and survival. Extracellular mitogenic growth factor signaling from cell surface receptors and intracellular pathways that convey hypoxic stress, energy and nutrient status all converge at mTOR. mTOR exists in two distinct complexes: mTOR complex 1 (mTORC1) and mTOR complex 2 (mTORC2). mTORC1 is a key mediator of transcription and cell growth (via its substrates p70S6 kinase and 4E-BP1) and promotes cell survival via the serum and glucocorticoid-activated kinase SGK, whereas mTORC2 promotes activation of the pro-survival kinase AKT. Given its central role in cellular growth, proliferation and survival, it is perhaps not surprising that mTOR signaling is frequently dysregulated in cancer and other diseases (Bjornsti and Houghton Rev Cancer 2004, 4(5), 335-48; Houghton and Huang Microbiol Immunol 2004, 279, 339-59; Inoki, Corradetti et al. Nat Genet. 2005, 37(1), 19-24).

mTOR is a member of the PIKK (PI3K-related Kinase) family of atypical kinases which includes ATM, ATR, and DNAPK, and its catalytic domain is homologous to that of PI3K. Dyregulation of PI3K signaling is a common function of tumor cells. In general, mTOR inhibition may be considered as a strategy in many of the tumor types in which PI3K signaling is implicated such as those discussed below.

Inhibitors of mTOR may be useful in treating a number of cancers, including the following: breast cancer (Nagata, Lan et al., Cancer Cell 2004, 6(2), 117-27; Pandolfi N Engl J Med 2004, 351(22), 2337-8; Nahta, Yu et al. Nat Clin Pract Oncol 2006, 3(5), 269-280); antle cell lymphoma (MCL) (Dal Col, Zancai et al. Blood 2008, 111(10), 5142-51); renal cell carcinoma (Thomas, Tran et al. Nat Med 2006, 12(1), 122-7; Atkins, Hidalgo et al. J Clin Oncol 2004, 22(5), 909-18; Motzer, Hudes et al. J Clin Oncol 2007, 25(25), 3958-64); acute myelogenous leukemia (AML) (Sujobert, Bardet et al. Blood 2005, 106(3), 1063-6; Billottet, Grandage et al. Oncogene 2006, 25(50), 6648-6659; Tamburini, Elie et al. Blood 2007, 110(3), 1025-8); chronic myelogenous leukemia (CML) (Skorski, Bellacosa et al. Embo J 1997, 16(20), 6151-61; Bai, Ouyang et al. Blood 2000, 96(13), 4319-27; Hickey and Cotter Biol Chem 2006, 281(5), 2441-50); diffuse large B cell lymphoma (DLBCL) (Uddin, Hussain et al. Blood 2006, 108(13), 4178-86); several subtypes of sarcoma (Hernando, Charytonowicz et al. Nat Med 2007, 13(6), 748-53; Wan and Helman Oncologist 2007, 12(8), 1007-18); rhabdomyosarcoma (Cao, Yu et al. Cancer Res 2008, 68(19), 8039-8048; Wan, Shen et al. Neoplasia 2006, 8(5), 394-401); ovarian cancer (Shayesteh, Lu et al. Nat Genet, 1999, 21(1), 99-102; (Lee, Choi et al. Gynecol Oncol 2005, 97(1) 26-34); endometrial tumors (Obata, Morland et al. Cancer Res 1998, 58(10), 2095-7; Lu, Wu et al. Clin Cancer Res 2008, 14(9), 2543-50); non small cell lung carcinoma (NSCLC) (Tang, He et al. Lung Cancer 2006, 51(2), 181-91; Marsit, Zheng et al. Hum Pathol 2005, 36(7), 768-76); small cell, squamous, large cell and adenocarcinoma (Massion, Taflan et al. Am J Respir Crit. Care Med 2004, 170(10), 1088-94); lung tumors in general (Kokubo, Gemma et al. Br J Cancer 2005, 92(9), 1711-9; Pao, Wang et al. Pub Library of Science Med 2005, 2(1), e17); colorectal tumors (Velho, Oliveira et al. Eur J Cancer 2005, 41(11), 1649-54; Foukas, Claret et al. Nature, 2006, 441(7091), 366-370), particularly those that display microsatellite instability (Goel, Arnold et al. Cancer Res 2004, 64(9), 3014-21; Nassif, Lobo et al. Oncogene 2004, 23(2), 617-28), KRAS-mutated colorectal tumors (Bos Cancer Res 1989. 49(17), 4682-9; Fearon Ann N Y Acad Sci 1995, 768, 101-10); gastric carcinomas (Byun, Cho et al. Int J Cancer 2003, 104(3), 318-27); hepatocellular tumors (Lee, Soung et al. Oncogene 2005, 24(8), 1477-80); liver tumors (Hu, Huang et al. Cancer 2003, 97(8), 1929-40; Wan, Jiang et al. Cancer Res Clin Oncol 2003, 129(2), 100-6); primary melanomas and associated increased tumor thickness (Guldberg, thor Staten et al. Cancer Res 1997, 57(17), 3660-3; Tsao, Zheng et al. Cancer Res 2000, 60(7), 1800-4; Whiteman, Thou et al. Int J Cancer 2002, 99(1), 63-7; Goel, Lazar et al. J Invest Dermatol 126(1), 2006, 154-60); pancreatic tumors (Asano, Yao et al. Oncogene 2004, 23(53), 8571-80); prostate carcinoma (Cairns, Okami et al. Cancer Res 1997, 57(22), 4997-5000; Gray, Stewart et al. Br J Cancer 1998, 78(10), 1296-300; Wang, Parsons et al. Clin Cancer Res 1998, 4(3), 811-5; Whang, Wu et al. Proc Natl Acad Sci USA 1998, 95(9), 5246-50; Majumder and Sellers Oncogene 2005, 24(50) 7465-74; Wang, Garcia et al. Proc Natl Acad Sci USA 2006, 103(5), 1480-5; (Lu, Ren et al. Int J Oncol 2006, 28(1), 245-51; Mulholland, Dedhar et al. Oncogene 25(3), 2006, 329-37; Xin, Teitell et al. Proc Natl Acad Sci USA 12006, 03(20), 7789-94; Mikhailova, Wang et al. Adv Exp Med Biol 2008, 617, 397-405; Wang, Mikhailova et al. Oncogene 2008, 27(56), 7106-7117); thyroid carcinoma, particularly in the anaplastic subtype (Garcia-Rostan, Costa et al. Cancer Res 2005, 65(22), 10199-207); follicular thyroid carcinoma (Wu, Mambo et al. J Clin Endocrinol Metab 2005, 90(8), 4688-93); anaplastic large cell lymphoma (ALCL); hamaratomas, angiomyelolipomas, TSC-associated and sporadic lymphangioleiomyomatosis: Cowden's disease (multiple hamaratoma syndrome) (Bissler, McCormack et al. N Engl J Med 2008, 358(2), 140-151); sclerosing hemangioma (Randa M. S. Amin Pathology International 2008, 58(1), 38-44); Peutz-Jeghers syndrome (PJS); head and neck cancer (Gupta, McKenna et al. Clin Cancer Res 2002, 8(3), 885-892); neurofibromatosis (Ferner Eur J Hum Genet. 2006, 15(2), 131-138; Sabatini Nat Rev Cancer 2006, 6(9), 729-734; Johannessen, Johnson et al. Current Biology 2008, 18(1), 56-62); macular degeneration; macular edema; myeloid leukemia; systemic lupus; and autoimmune lymphoproliferative syndrome (ALPS).

### SUMMARY OF THE INVENTION

The following only summarizes certain aspects of the invention and is not intended to be limiting in nature. These aspects and other aspects and embodiments are described more fully below. All references cited in this specification are hereby incorporated by reference in their entirety. In the event of a discrepancy between the express disclosure of this specification and the references incorporated by reference, the express disclosure of this specification shall control.

We recognized the important role of PI3K and mTOR in biological processes and disease states and, therefore, realized that inhibitors of these protein kinases would be desirable. Accordingly, the invention provides compounds that inhibit, regulate, and/or modulate PI3K and/or mTOR that are useful in the treatment of hyperproliferative diseases, such as cancer, in mammals. This invention also provides methods of making the compound, methods of using such compounds in the treatment of hyperproliferative diseases in mammals, especially humans, and to pharmaceutical compositions containing such compounds.

A first aspect of the invention provides a compound of Formula I:

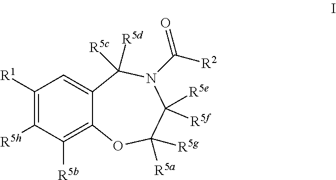

or a single stereoisomer or mixture of isomers thereof and additionally optionally as a pharmaceutically acceptable salt thereof, where
R¹ is phenyl optionally substituted with one, two, or three R⁶ groups; or
R¹ is heteroaryl optionally substituted with one, two, or three R⁷;
R² is —NR³R⁴;
R³ is hydrogen, alkyl, or alkoxycarbonylalkyl; and R⁴ is optionally substituted cycloalkyl, optionally substituted phenyl, optionally substituted phenylalkyl, optionally substituted heteroaryl, or optionally substituted heteroarylalkyl; or
R³ and R⁴ together with the nitrogen to which they are attached form HET optionally substituted on any substitutable atom of the ring with R¹⁰, R^{10a}, R^{10b}, R^{10c}, R^{10d}, R^{10e}, and R^{10f};
BET is
   (a) a saturated or partially unsaturated, but non-aromatic, monocyclic 5- to 8-membered ring optionally containing an additional one or two ring heteroatoms which are independently oxygen, sulfur, or nitrogen where the remaining ring atoms are carbon; or
   (b) a partially unsaturated, but not aromatic, monocyclic 5- to 8-membered ring optionally containing an additional one or two ring heteroatoms which are independently oxygen, sulfur, or nitrogen and the remaining ring atoms are carbon and which ring is fused to a benzo ring; or
   (c) a fused, bridged, or spirocyclic, bicyclic 7- to 11-membered ring optionally containing an additional one or two heteroatoms which are independently oxygen, sulfur, or nitrogen and the remaining ring atoms are carbon and where each ring of the 7- to 11-membered ring is saturated or partially unsaturated but not fully aromatic; or
   (d) a fused, bridged, or spirocyclic, bicyclic 7- to 11-membered ring optionally containing an additional one or two ring heteroatoms which are independently oxygen, sulfur, or nitrogen and the remaining ring atoms are carbon where each ring of the bicyclic 7- to 11-membered ring is saturated or partially unsaturated but not fully aromatic, and where the bicycle 7- to 11-membered ring is fused to a benzo ring;
R^{5a} and R^{5c} are independently hydrogen or alkyl;
R^{5h} is hydrogen or halo;
R^{5b} is (C₁₋₃)alkyl, (C₁₋₃)alkoxy, halo(C₁₋₃)alkyl, (C₁₋₃)haloalkoxy;
R^{5d}, R^{5e}, R^{5f}, and R^{5g} are hydrogen;
each R⁶, when R⁶ is present, is independently nitro; cyano; halo; alkyl; alkenyl; alkynyl; halo; haloalkyl; —OR^{8a}; —NR⁸R^{8a}; —C(O)NR⁸R^{8a}; —NR⁸C(O)OR⁹; —NR⁸C(O)R⁹; —NR⁸S(O)₂R^{8a}; —NR⁸C(O)NR^{8a}R⁹; carboxy, —C(O)OR⁹; alkylcarbonyl; alkyl substituted with one or two —C(O)NR⁸R^{8a}; heteroaryl optionally substituted with 1, 2, or 3 R¹⁴; or optionally substituted heterocycloalkyl;
each R⁷, when R⁷ is present, is independently oxo; nitro; cyano; alkyl; alkenyl; alkynyl; halo; haloalkyl; hydroxyalkyl; alkoxyalkyl; —OR^{8a}; —SR¹³; —S(O)R¹³; —S(O)₂R¹³; —NR⁸R^{8a}; —C(O)NR⁸R^{8a}; —NR⁸C(O)OR⁹; —NR⁸C(O)R⁹; —NR⁸S(O)₂R^{8a}; —NR⁸C(O)NR^{8a}R⁹; carboxy; —C(O)OR⁹; alkylcarbonyl; —S(O)₂NR⁸R⁹; alkyl substituted with one or two —NR⁸R^{8a}; alkyl substituted with one or two —NR⁸C(O)R^{8a}; optionally substituted cycloalkyl; optionally substituted cycloalkylalkyl; optionally substituted heterocycloalkyl; optionally substituted heterocycloalkylalkyl; optionally substituted heteroaryl; or optionally substituted heteroarylalkyl;
R⁸ is hydrogen, alkyl, alkenyl, alkynyl, hydroxyalkyl, or haloalkyl;
R^{8a} is hydrogen, alkyl, alkenyl, alkynyl, haloalkyl, hydroxyalkyl, cyanoalkyl, aminoalkyl, alkylaminoalkyl, dialkylaminoalkyl, alkoxyalkyl, optionally substituted cycloalkyl, optionally substituted cycloalkylalkyl, optionally substituted heterocycloalkyl, optionally substituted heterocycloalkylalkyl, optionally substituted phenyl, optionally substituted phenylalkyl, optionally substituted heteroaryl, or optionally substituted heteroarylalkyl;
R⁹ is alkyl, alkenyl, alkynyl, hydroxyalkyl, alkoxyalkyl, haloalkyl, or optionally substituted heterocycloalkylalkyl;
R¹⁰, R^{10a}, R^{10b}, R^{10c}, R^{10d}, R^{10e}, and R^{10f} are independently hydrogen; halo; alkyl; haloalkyl; haloalkenyl; hydroxyalkyl; alkylthio; alkylsulfonyl; hydroxy; alkoxy; haloalkoxy; cyano; alkoxycarbonyl; carboxy; amino; alkylamino; dialkylamino; —C(O)R¹²; —C(O)NR¹¹R^{11a}; optionally substituted cycloalkyl; optionally substituted cycloalkylalkyl; optionally substituted phenyl; optionally substituted phenylalkyl; optionally substituted phenyloxy; optionally substituted phenyloxyalkyl; optionally substituted heterocycloalkyl; optionally substituted heterocycloalkylalkyl; optionally substituted heteroaryl; or optionally substituted heteroarylalkyl; or two of R¹⁰, R^{10a}, R^{10b}, R^{10c}, R^{10d}, R^{e}, and R^{10f} when attached to the same carbon form oxo, imino, or thiono;
R¹¹ hydrogen, alkyl, or alkenyl;
R^{11a} hydrogen, alkyl, or alkenyl;
R¹² is alkyl, or optionally substituted heteroaryl;
R¹³ is alkyl or haloalkyl; and
   each R¹⁴, when R¹⁴ is present, is independently amino, alkylamino, dialkylamino, acylamino, halo, hydroxy, alkyl, haloalkyl, hydroxyalkyl, aminoalkyl, alkylaminoalkyl, dialkylaminoalkyl, alkoxycarbonyl, aminocarbonyl, alkylaminocarbonyl, dialkylaminocarbonyl, or optionally substituted phenyl.

In a second aspect, the invention is directed to a pharmaceutical composition which comprises 1) a compound of Formula I or a single stereoisomer or mixture of isomers thereof, optionally as a pharmaceutically acceptable salt thereof and 2) a pharmaceutically acceptable carrier, excipient, or diluent.

In a third aspect of the invention is a method of inhibiting the in vivo activity of PI3K and additionally optionally mTOR, the method comprising administering to a patient an effective PI3K-inhibiting and additionally optionally mTOR-inhibiting amount of a Compound of Formula Ia Compound of Formula I or a single stereoisomer or mixture of stereoisomers thereof, optionally as a pharmaceutically acceptable salt or solvate thereof or pharmaceutical composition thereof.

In a fourth aspect, the Invention provides a method for treating a disease, disorder, or syndrome which method comprises administering to a patient a therapeutically effective amount of a compound of Formula I or a single stereoisomer or mixture of isomers thereof, optionally as a pharmaceutically acceptable salt or solvate thereof, or a pharmaceutical composition comprising a therapeutically effective amount of a compound of Formula I or a single stereoisomer or mixture of isomers thereof, optionally as a pharmaceutically acceptable salt or solvate thereof, and a pharmaceutically acceptable carrier, excipient, or diluent.

In a fifth aspect, the Invention provides a method for making a Compound of Formula I(a) which method comprises

(a) reacting the following intermediate, or a salt thereof:

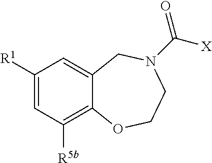

where X is halo and R¹ and R^{5b} are as defined in the Summary of the Invention for a Compound of Formula I; with an intermediate of formula R²H where R² is as defined in in the Summary of the Invention for a Compound of Formula Ito yield a Compound of the Invention of Formula I(a)

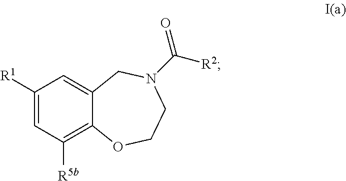

and optionally separating individual isomers; and optionally modifying any of the R¹ and R² groups; and optionally forming a pharmaceutically acceptable salt thereof; or

(b) reacting the following intermediate, or a salt thereof:

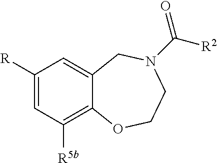

where R is halo or —B(OR′)₂ (where both R¹ are hydrogen or the two R′ together form a boronic ester), and R² is as defined in the Summary of the Invention for a Compound of Formula I; with an intermediate of formula R′Y where Y is halo when R is —B(OR)₂ and Y is —B(OR)₂ when R is halo, and R² is as defined in the Summary of the Invention for a Compound of Formula Ito yield a Compound of the Invention of Formula I(a); and optionally separating individual isomers; and optionally modifying any of the R¹ and R² groups; and optionally forming a pharmaceutically acceptable salt, hydrate, solvate or combination thereof.

In an additional aspect of the invention provides a method for treating a subject having a tumor the method comprising: (a) administering a PI3K-α selective inhibitor, a dual PI3K-α/mTOR selective inhibitor, or a combination of a PI3K-α selective inhibitor and a mTOR selective inhibitor to the subject if said tumor comprises a mutation in a PI3K-α kinase domain; or (b) administering a combination of a PI3K-α selective inhibitor and a

PI3K-β selective inhibitor, a dual PI3K-α/mTOR selective inhibitor, or a PI3K-β selective inhibitor, to said subject if said tumor comprises a mutation in a PI3K-α helical domain.

In an additional aspect, the present invention provides a method for identifying a selective inhibitor of a PI3K isozyme, the method comprising: (a) contacting a first cell bearing a first mutation in a PI3K-α with a candidate inhibitor; (b) contacting a second cell bearing a wild type PI3K-α, a PTEN null mutation, or a second mutation in said PI3K-α with the candidate inhibitor; and (c) measuring AKT phosphorylation in said first and said second cells, wherein decreased AKT phosphorylation in said first cell when compared to said second cell identifies said candidate inhibitor as a selective PI3K-α inhibitor.

In an additional aspect, the present invention provides for a method for determining a treatment regimen for a cancer patient having a tumor comprising a PI3K-α, the method comprising: determining the presence or absence of a mutation in amino acids 1047 and/or 545 of said PI3K-α; wherein if said PI3K-α has a mutation at position 1047, said method comprises administering to the cancer patient a therapeutically effective amount of a PI3K-α selective inhibitor compound, or a dual PI3K-α/mTOR selective inhibitor, or a combination of a PI3K-α selective inhibitor and a mTOR selective inhibitor; or wherein if said PI3K-α has a mutation at position 545, said method comprises administering to the cancer patient a therapeutically effective amount of a combination of a PI3K-α selective inhibitor and a PI3K-β selective inhibitor, or a dual PI3K-α/mTOR selective inhibitor, or a combination of a PI3K-α selective inhibitor and a mTOR selective inhibitor.

In an additional aspect, the cell used to diagnose, treat or screen against includes a cancer or tumor cell obtained from a tumor or cancer derived from: breast cancer, mantle cell lymphoma, renal cell carcinoma, acute myelogenous leukemia, chronic myelogenous leukemia, NPM/ALK-transformed anaplastic large cell lymphoma, diffuse large B cell lymphoma, rhabdomyosarcoma, ovarian cancer, endometrial cancer, cervical cancer, non-small cell lung carcinoma, small cell lung carcinoma, adenocarcinoma, colon cancer, rectal cancer, gastric carcinoma, hepatocellular carcinoma, melanoma, pancreatic cancer, prostate carcinoma, thyroid carcinoma, anaplastic large cell lymphoma, hemangioma, glioblastoma, or head and neck cancer.

### DETAILED DESCRIPTION OF THE INVENTION

### Abbreviations and Definitions

The following abbreviations and terms have the indicated meanings throughout:

| Abbreviation | Meaning |
|---|---|
| AcOH | acetic acid |
| br | broad |
| ° C. | degrees Celsius |
| conc | concentrated |
| d | doublet |
| dd | doublet of doublet |
| dt | doublet of triplet |
| DCM | dichloromethane |
| DIEA or DIPEA | N,N-di-isopropyl-N-ethylamine |
| DMA | N,N-dimethylacetamide |
| DME | 1,2-dimethoxyethane |
| DMF | N,N-dimethylformamide |
| DMSO | dimethyl sulfoxide |
| dppf | 1,1′-bis(diphenylphosphano)ferrocene |
| EI | Electron Impact ionization |
| equiv | equivalents |
| g | gram(s) |
| GC/MS | gas chromatography/mass spectrometry |
| h or hr | hour(s) |
| HATU | 2-(1H-7-azabenzotriazol-1-yl)-1,1,3,3-tetramethyl |
| | uronium hexafluorophosphate |
| HPLC | high pressure liquid chromatography |
| L | liter(s) |
| LC/MS | liquid chromatography/mass spectrometry |
| M | molar or molarity |
| m | Multiplet |
| MeOH | methanol |
| mg | milligram(s) |
| MHz | megahertz (frequency) |
| min | minute(s) |
| mL | milliliter(s) |
| μL | microliter(s) |
| μM | micromolar |
| μmol | micromole(s) |
| mM | Millimolar |
| mmol | millimole(s) |
| mol | mole(s) |
| MS | mass spectral analysis |
| Ms | mesyl |
| N | normal or normality |
| nM | Nanomolar |
| NMR | nuclear magnetic resonance spectroscopy |
| q | Quartet |
| quant | quantitative |
| rt | Room temperature |
| s | Singlet |
| t or tr | Triplet |
| THF | tetrahydrofuran |
| Ts | tosyl |

The symbol “—” means a single bond, “═” means a double bond, “≡” means a triple bond, “ [symbol] ” means a single or double bond. The symbol “ [symbol] ” refers to a group on a double-bond as occupying either position on the terminus of a double bond to which the symbol is attached; that is, the geometry, E- or Z-, of the double bond is ambiguous. When a group is depicted removed from its parent Formula, the “ [symbol] ” symbol will be used at the end of the bond which was theoretically cleaved in order to separate the group from its parent structural Formula.

When chemical structures are depicted or described, unless explicitly stated otherwise, all carbons are assumed to have hydrogen substitution to conform to a valence of four. For example, in the structure on the left-hand side of the schematic below there are nine hydrogens implied. The nine hydrogens are depicted in the right-hand structure. Sometimes a particular atom in a structure is described in textual Formula as having a hydrogen or hydrogens as substitution (expressly defined hydrogen), for example, —CH₂CH₂—. It is understood by one of ordinary skill in the art that the aforementioned descriptive techniques are common in the chemical arts to provide brevity and simplicity to description of otherwise complex structures.

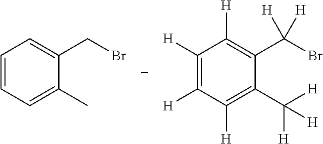

If a group “R” is depicted as “floating” on a ring system, as for example in the Formula:

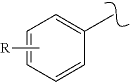

then, unless otherwise defined, a substituent “R” may reside on any atom of the ring system, assuming replacement of a depicted, implied, or expressly defined hydrogen from one of the ring atoms, so long as a stable structure is formed.

If a group “R” is depicted as floating on a fused or bridged ring system, as for example in the Formula e:

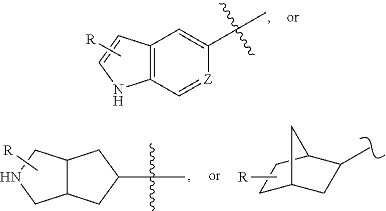

then, unless otherwise defined, a substituent “R” may reside on any atom of the fused or bridged ring system, assuming replacement of a depicted hydrogen (for example the —NH— in the Formula above), implied hydrogen (for example as in the Formula above, where the hydrogens are not shown but understood to be present), or expressly defined hydrogen (for example where in the Formula above, “Z” equals ═CH—) from one of the ring atoms, so long as a stable structure is formed. In the example depicted, the “R” group may reside on either the 5-membered or the 6-membered ring of the fused or bridged ring system.

When a group “R” is depicted as existing on a ring system containing saturated carbons, as for example in the Formula:

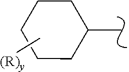

where, in this example, “y” can be more than one, assuming each replaces a currently depicted, implied, or expressly defined hydrogen on the ring; then, unless otherwise defined, where the resulting structure is stable, two “R's” may reside on the same carbon. In another example, two R's on the same carbon, including that carbon, may form a ring, thus creating a spirocyclic ring structure with the depicted ring as for example in the Formula:

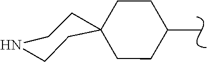

“Acyl” means a —C(O)R radical where R is alkyl, haloalkyl, alkenyl, cycloalkyl, cycloalkylalkyl, aryl, aralkyl, heteroaryl, heteroaralkyl, heterocycloalkyl, or heterocycloalkylalkyl, as defined herein, e.g., acetyl, trifluoromethylcarbonyl, or 2-methoxyethylcarbonyl, and the like.

“Acylamino” means a —NRR′ radical where R is hydrogen, hydroxy, alkyl, or alkoxy and R¹ is acyl, as defined herein.

“Acyloxy” means an —OR radical where R is acyl, as defined herein, e.g. cyanomethylcarbonyloxy, and the like.

“Administration” and variants thereof (e.g., “administering” a compound) in reference to a compound of the invention means introducing the compound of the compound into the system of the animal in need of treatment. When a compound of the invention or prodrug thereof is provided in combination with one or more other active agents (e.g., surgery, radiation, and chemotherapy, etc.), “administration” and its variants are each understood to include concurrent and sequential introduction of the compound or prodrug thereof and other agents.

“Alkenyl” means a means a linear monovalent hydrocarbon radical of two to six carbon atoms or a branched monovalent hydrocarbon radical of three to 6 carbon atoms which radical contains at least one double bond, e.g., ethenyl, propenyl, 1-but-3-enyl, and 1-pent-3-enyl, and the like.

“Alkoxy” means an —OR group where R is alkyl group as defined herein. Examples include methoxy, ethoxy, propoxy, isopropoxy, and the like.

“Alkoxyalkyl” means an alkyl group, as defined herein, substituted with at least one, specifically one, two, or three, alkoxy groups as defined herein. Representative examples include methoxymethyl and the like.

“Alkoxycarbonyl” means a —C(O)R group where R is alkoxy, as defined herein.

“Alkyl” means a linear saturated monovalent hydrocarbon radical of one to six carbon atoms or a branched saturated monovalent hydrocarbon radical of three to 6 carbon atoms, e.g., methyl, ethyl, propyl, 2-propyl, butyl (including all isomeric forms), or pentyl (including all isomeric forms), and the like.

“Alkylamino” means an —NHR group where R is alkyl, as defined herein.

“Alkylaminoalkyl” means an alkyl group substituted with one or two alkylamino groups, as defined herein.

“Alkylaminoalkyloxy” means an —OR group where R is alkylaminoalkyl, as defined herein.

“Alkylcarbonyl” means a —C(O)R group where R is alkyl, as defined herein.

“Alkylsulfonyl” means an —S(O)₂R group where R is alkyl, as defined herein.

“Alkylsulfonylalkyl” means an alkyl group, as defined herein, substituted with at least one, preferably one or two, alkylsulfonyl groups, as defined herein.

“Alkynyl” means a linear monovalent hydrocarbon radical of two to six carbon atoms or a branched monovalent hydrocarbon radical of three to 6 carbon atoms which radical contains at least one triple bond, e.g., ethynyl, propynyl, butynyl, pentyn-2-yl and the like.

“Amino” means —NH₂.

“Aminoalkyl” means an alkyl group substiuted with at least one, specifically one, two or three, amino groups.

“Aminoalkyloxy” means an —OR group where R is aminoalkyl, as defined herein.

“Aminocarbonyl” means a —C(O)NH₂ group.

“Alkylaminocarbonyl” means a —C(O)NHR group where R is alkyl as defined herein.

“Aryl” means a monovalent six- to fourteen-membered, mono- or bi-carbocyclic ring, wherein the monocyclic ring is aromatic and at least one of the rings in the bicyclic ring is aromatic. Unless stated otherwise, the valency of the group may be located on any atom of any ring within the radical, valency rules permitting. Representative examples include phenyl, naphthyl, and indanyl, and the like.

“Arylalkyl” means an alkyl radical, as defined herein, substituted with one or two aryl groups, as defined herein, e.g., benzyl and phenethyl, and the like.

“Arylalkyloxy” means an —OR group where R is arylakyl, as defiend herein.

“Cyanoalkyl” means an alkyl group, as defined herein, substituted with one or two cyano groups.

“Cycloalkyl” means a monocyclic or fused or bridged bicyclic or tricyclic, saturated or partially unsaturated (but not aromatic), monovalent hydrocarbon radical of three to ten carbon ring atoms. Unless stated otherwise, the valency of the group may be located on any atom of any ring within the radical, valency rules permitting. One or two ring carbon atoms may be replaced by a —C(O)—, —C(S)—, or —C(═NH)— group. More specifically, the term cycloalkyl includes, but is not limited to, cyclopropyl, cyclobutyl, cyclopentyl, cyclohexyl, cyclohexyl, cyclohex-3-enyl, or (1r,3r,5R,7R)-tricyclo[3.3.1.1^{3,7}]decan-2-yl, and the like.

“Cycloalkylalkyl” means an alkyl group substituted with at least one, specifically one or two, cycloalkyl group(s) as defined herein.

“Dialkylamino” means a —NRR′ radical where R and R¹ are alkyl as defined herein, or an N-oxide derivative, or a protected derivative thereof, e.g., dimethylamino, diethylamino, N,N-methylpropylamino or N,N-methylethylamino, and the like.

“Dialkylaminoalkyl” means an alkyl group substituted with one or two dialkylamino groups, as defined herein.

“Dialkylaminoalkyloxy” means an —OR group where R is dialkylaminoalkyl, as defined herein. Representative examples include 2-(N,N-diethylamino)-ethyloxy, and the like.

“Dialkylaminocarbonyl” means a —C(O)NRR′ group where R and R¹ are alkyl as defined herein.

“Halogen” or “halo” refers to fluorine, chlorine, bromine and iodine.

“Haloalkoxy” means an —OR¹ group where R¹ is haloalkyl as defined herein, e.g., trifluoromethoxy or 2,2,2-trifluoroethoxy, and the like.

“Haloalkyl” mean an alkyl group substituted with one or more halogens, specifically 1, 2, 3, 4, 5, or 6 halo atoms, e.g., trifluoromethyl, 2-chloroethyl, and 2,2-difluoroethyl, and the like.

“Heteroaryl” means a monocyclic or fused or bridged bicyclic monovalent radical of 5 to 14 ring atoms containing one or more, specifically one, two, three, or four ring heteroatoms where each heteroatom is independently —O—, —S(O)ₙ— (n is 0, 1, or 2), —NH—, —N═, or N-oxide, with the remaining ring atoms being carbon, wherein the ring comprising a monocyclic radical is aromatic and wherein at least one of the fused rings comprising the bicyclic radical is aromatic. One or two ring carbon atoms of any nonaromatic rings comprising a bicyclic radical may be replaced by a —C(O)—, —C(S)—, or —C(═NH)— group. Unless stated otherwise, the valency may be located on any atom of any ring of the heteroaryl group, valency rules permitting. More specifically, the term heteroaryl includes, but is not limited to, 1,2,4-triazolyl, 1,3,5-triazolyl, phthalimidyl, pyridinyl, pyrrolyl, imidazolyl, thienyl, furanyl, indolyl, 2,3-dihydro-1H-indolyl (including, for example, 2,3-dihydro-1H-indol-2-yl or 2,3-dihydro-1H-indol-5-yl, and the like), isoindolyl, indolinyl, isoindolinyl, benzimidazolyl, benzodioxol-4-yl, benzofuranyl, cinnolinyl, indolizinyl, naphthyridin-3-yl, phthalazin-3-yl, phthalazin-4-yl, pteridinyl, purinyl, quinazolinyl, quinoxalinyl, tetrazoyl, pyrazolyl, pyrazinyl, pyrimidinyl, pyridazinyl, oxazolyl, isooxazolyl, oxadiazolyl, benzoxazolyl, quinolinyl, isoquinolinyl, tetrahydroisoquinolinyl (including, for example, tetrahydroisoquinolin-4-yl or tetrahydroisoquinolin-6-yl, and the like), pyrrolo[3,2-c]pyridinyl (including, for example, pyrrolo[3,2-c]pyridin-2-yl or pyrrolo[3,2-c]pyridin-7-yl, and the like), benzopyranyl, 2,3-dihydrobenzofuranyl, benzo[d][1,3]dioxolyl, 2,3-dihydrobenzo[b][1,4]dioxinyl, thiazolyl, isothiazolyl, thiadiazolyl, benzothiazolyl, benzothienyl, and the derivatives thereof, or N-oxide or a protected derivative thereof. The term “5- or 6-membered heteroaryl” describes a subset of the term “heteroaryl.”

“Heteroarylalkyl” means an alkyl group, as defined herein, substituted with at least one, specifically one or two heteroaryl group(s), as defined herein.

“Heterocycloalkyl” means a saturated or partially unsaturated (but not aromatic) monovalent monocyclic group of 3 to 8 ring atoms or a saturated or partially unsaturated (but not aromatic) monovalent fused or bridged, bicyclic or tricyclic group of 5 to 12 ring atoms in which one or more, specifically one, two, three, or four ring heteroatoms where each heteroatom is independently O, S(O)ₙ (n is 0, 1, or 2), —N═, or —NH—, the remaining ring atoms being carbon. One or two ring carbon atoms may be replaced by a —C(O)—, —C(S)—, or —C(═NH)— group. Unless otherwise stated, the valency of the group may be located on any atom of any ring within the radical, valency rules permitting. When the point of valency is located on a nitrogen atom, R^{y} is absent. More specifically the term heterocycloalkyl includes, but is not limited to, azetidinyl, pyrrolidinyl, 2-oxopyrrolidinyl, 2,5-dihydro-1H-pyrrolyl, piperidinyl, 4-piperidonyl, morpholinyl, piperazinyl, 2-oxopiperazinyl, tetrahydropyranyl, 2-oxopiperidinyl, thiomorpholinyl, thiamorpholinyl, perhydroazepinyl, pyrazolidinyl, imidazolinyl, imidazolidinyl, dihydropyridinyl, tetrahydropyridinyl, oxazolinyl, oxazolidinyl, isoxazolidinyl, thiazolinyl, thiazolidinyl, quinuclidinyl, isothiazolidinyl, octahydrocyclopenta[c]pyrrolyl, octahydroindolyl, octahydroisoindolyl, decahydroisoquinolyl, tetrahydrofuryl, tetrahydropyranyl, (3aR,6aS)-5-methyloctahycirocyclopenta[c]pyrrolyl, and (3aS,6aR)-5-methyl-1,2,3,3a,4,6a-hexahydrocyclopenta[c]pyrrolyl, and the derivatives thereof and N-oxide or a protected derivative thereof.

“Heterocycloalkylalkyl” means an alkyl radical, as defined herein, substituted with one or two heterocycloalkyl groups, as defined herein, e.g., morpholinylmethyl, N-pyrrolidinylethyl, and 3-(N-azetidinyl)propyl, and the like.

“Heterocycloalkyloxy” means an —OR group where R is heterocycloalkyl, as defined herein.

“Hydroxyalkyl” means an alkyl group, as defined herein, substituted with at least one, preferably 1, 2, 3, or 4, hydroxy groups.

“Phenylalkyl” means an alkyl group, as defined herein, substituted with one or two phenyl groups.

“Phenylalkyloxy” means an —OR group where R is phenylalkyl, as defined herein.

“Optional” or “optionally” means that the subsequently described event or circumstance may or may not occur, and that the description includes instances where said event or circumstance occurs and instances in which it does not. One of ordinary skill in the art would understand that with respect to any molecule described as containing one or more optional substituents, only sterically practical and/or synthetically feasible compounds are meant to be included. “Optionally substituted” refers to all subsequent modifiers in a term, unless stated otherwise. A list of exemplary optional substitutions is presented below in the definition of “substituted.”

“Optionally substituted aryl” means an aryl group, as defined herein, optionally substituted with one, two, or three substituents independently acyl, acylamino, acyloxy, alkyl, haloalkyl, alkenyl, alkoxy, alkenyloxy, halo, hydroxy, alkoxycarbonyl, alkenyloxycarbonyl, amino, alkylamino, dialkylamino, nitro, aminocarbonyl, alkylaminocarbonyl, dialkylaminocarbonyl, carboxy, cyano, alkylthio, alkylsulfinyl, alkylsulfonyl, aminosulfonyl, alkylaminosulfonyl, dialkylaminosulfonyl, alkylsulfonylamino, or aminoalkoxy; or aryl is pentafluorophenyl. Within the optional substituents on “aryl”, the alkyl and alkenyl, either alone or as part of another group (including, for example, the alkyl in alkoxycarbonyl), are independently optionally substituted with one, two, three, four, or five halo.

“Optionally substituted arylalkyl” means an alkyl group, as defined herein, substituted with optionally substituted aryl, as defined herein.

“Optionally substituted cycloalkyl” means a cycloalkyl group, as defined herein, substituted with one, two, or three groups independently acyl, acyloxy, acylamino, alkyl, haloalkyl, alkenyl, alkoxy, alkenyloxy, alkoxycarbonyl, alkenyloxycarbonyl, alkylthio, alkylsulfinyl, alkylsulfonyl, aminosulfonyl, alkylaminosulfonyl, dialkylaminosulfonyl, alkylsulfonylamino, halo, hydroxy, amino, alkylamino, dialkylamino, aminocarbonyl, alkylaminocarbonyl, dialkylaminocarbonyl, nitro, alkoxyalkyloxy, aminoalkoxy, alkylaminoalkoxy, dialkylaminoalkoxy, carboxy, or cyano. Within the above optional substitutents on “cycloalkyl”, the alkyl and alkenyl, either alone or as part of another substituent on the cycloalkyl ring, are independently optionally substituted with one, two, three, four, or five halo, e.g. haloalkyl, haloalkoxy, haloalkenyloxy, or haloalkylsulfonyl.

“Optionally substituted cycloalkylalkyl” means an alkyl group substituted with at least one, specifically one or two, optionally substituted cycloalkyl groups, as defined herein.

“Optionally substituted heteroaryl” means a heteroaryl group optionally substituted with one, two, or three substituents independently acyl, acylamino, acyloxy, alkyl, haloalkyl, alkenyl, alkoxy, alkenyloxy, halo, hydroxy, alkoxycarbonyl, alkenyloxycarbonyl, amino, alkylamino, dialkylamino, nitro, aminocarbonyl, alkylaminocarbonyl, dialkylaminocarbonyl, carboxy, cyano, alkylthio, alkylsulfinyl, alkylsulfonyl, aminosulfonyl, alkylaminosulfonyl, dialkylaminosulfonyl, alkylsulfonylamino, aminoalkoxy, alkylaminoalkoxy, or dialkylaminoalkoxy. Within the optional substituents on “heteroaryl”, the alkyl and alkenyl, either alone or as part of another group (including, for example, the alkyl in alkoxycarbonyl), are independently optionally substituted with one, two, three, four, or five halo.

“Optionally substituted heteroarylalkyl” means an alkyl group, as defined herein, substituted with at least one, specifically one or two, optionally substituted heteroaryl group(s), as defined herein.

“Optionally substituted heterocycloalkyl” means a heterocycloalkyl group, as defined herein, optionally substituted with one, two, or three substituents independently acyl, acylamino, acyloxy, haloalkyl, alkyl, alkenyl, alkoxy, alkenyloxy, halo, hydroxy, alkoxycarbonyl, alkenyloxycarbonyl, amino, alkylamino, dialkylamino, nitro, aminocarbonyl, alkylaminocarbonyl, dialkylaminocarbonyl, carboxy, cyano, alkylthio, alkylsulfinyl, alkylsulfonyl, aminosulfonyl, alkylaminosulfonyl, dialkylaminosulfonyl, alkylsulfonylamino, aminoalkoxy, or phenylalkyl. Within the optional substituents on “heterocycloalkyl”, the alkyl and alkenyl, either alone or as part of another group (including, for example, the alkyl in alkoxycarbonyl), are independently optionally substituted with one, two, three, four, or five halo.

“Optionally substituted heterocycloalkylalkyl” means an alkyl group, as defined herein, substituted with at least one, specifically one or two, optionally substituted heterocycloalkyl group(s) as defined herein.

“Optionally substituted phenyl” means a phenyl group optionally substituted with one, two, or three substituents independently acyl, acylamino, acyloxy, alkyl, haloalkyl, alkenyl, alkoxy, alkenyloxy, halo, hydroxy, alkoxycarbonyl, alkenyloxycarbonyl, amino, alkylamino, dialkylamino, nitro, aminocarbonyl, alkylaminocarbonyl, dialkylaminocarbonyl, carboxy, cyano, alkylthio, alkylsulfinyl, alkylsulfonyl, aminosulfonyl, alkylaminosulfonyl, dialkylaminosulfonyl, alkylsulfonylamino, or aminoalkoxy, or aryl is pentafluorophenyl. Within the optional substituents on “phenyl”, the alkyl and alkenyl, either alone or as part of another group (including, for example, the alkyl in alkoxycarbonyl), are independently optionally substituted with one, two, three, four, or five halo.

“Optionally substituted phenylalkyl” means an alkyl group, as defined herein, substituted with one or two optionally substituted phenyl groups, as defined herein.

“Optionally substituted phenylsulfonyl” means an —S(O)₂R group where R is optionally substituted phenyl, as defined herein.

“Oxo” means an oxygen which is attached via a double bond.

“Yield” for each of the reactions described herein is expressed as a percentage of the theoretical yield.

“Metabolite” refers to the break-down or end product of a compound or its salt produced by metabolism or biotransformation in the animal or human body; for example, biotransformation to a more polar molecule such as by oxidation, reduction, or hydrolysis, or to a conjugate (see Goodman and Gilman, “The Pharmacological Basis of Therapeutics” 8.sup.th Ed., Pergamon Press, Gilman et al. (eds), 1990 for a discussion of biotransformation). As used herein, the metabolite of a compound of the invention or its salt may be the biologically active form of the compound in the body. In one example, a prodrug may be used such that the biologically active form, a metabolite, is released in vivo. In another example, a biologically active metabolite is discovered serendipitously, that is, no prodrug design per se was undertaken. An assay for activity of a metabolite of a compound of the present invention is known to one of skill in the art in light of the present disclosure.

“Patient” for the purposes of the present invention includes humans and other animals, particularly mammals, and other organisms. Thus the methods are applicable to both human therapy and veterinary applications. In a specific embodiment the patient is a mammal, and in a more specific embodiment the patient is human.

A “pharmaceutically acceptable salt” of a compound means a salt that is pharmaceutically acceptable and that possesses the desired pharmacological activity of the parent compound. It is understood that the pharmaceutically acceptable salts are non-toxic. Additional information on suitable pharmaceutically acceptable salts can be found in Remington's Pharmaceutical Sciences, 17^{th} ed., Mack Publishing Company, Easton, Pa., 1985, which is incorporated herein by reference or S. M. Berge, et al., “Pharmaceutical Salts,” J. Pharm. Sci., 1977; 66:1-19 both of which are incorporated herein by reference.

Examples of pharmaceutically acceptable acid addition salts include those formed with inorganic acids such as hydrochloric acid, hydrobromic acid, sulfuric acid, nitric acid, phosphoric acid, and the like; as well as organic acids such as acetic acid, trifluoroacetic acid, propionic acid, hexanoic acid, cyclopentanepropionic acid, glycolic acid, pyruvic acid, lactic acid, oxalic acid, maleic acid, malonic acid, succinic acid, fumaric acid, tartaric acid, citric acid, benzoic acid, cinnamic acid, 3-(4-hydroxybenzoyl)benzoic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, p-toluenesulfonic acid, and salicylic acid and the like.

Examples of a pharmaceutically acceptable base addition salts include those formed when an acidic proton present in the parent compound is replaced by a metal ion, such as sodium, potassium, lithium, ammonium, calcium, magnesium, iron, zinc, copper, manganese, aluminum salts and the like. Specific salts are the ammonium, potassium, sodium, calcium, and magnesium salts. Salts derived from pharmaceutically acceptable organic non-toxic bases include, but are not limited to, salts of primary, secondary, and tertiary amines, substituted amines including naturally occurring substituted amines, cyclic amines and basic ion exchange resins. Examples of organic bases include isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, tromethamine, N-methylglucamine, polyamine resins, and the like. Exemplary organic bases are isopropylamine, diethylamine, ethanolamine, trimethylamine, dicyclohexylamine, choline, and caffeine. “Platin(s),” and “platin-containing agent(s)” include, for example, cisplatin, carboplatin, and oxaliplatin.

“Therapeutically effective amount” is an amount of a compound of the invention, that when administered to a patient, ameliorates a symptom of the disease. The amount of a compound of the invention which constitutes a “therapeutically effective amount” will vary depending on the compound, the disease state and its severity, the age of the patient to be treated, and the like. The therapeutically effective amount can be determined routinely by one of ordinary skill in the art having regard to their knowledge and to this disclosure.

“Preventing” or “prevention” of a disease, disorder, or syndrome includes inhibiting the disease from occurring in a human, i.e. causing the clinical symptoms of the disease, disorder, or syndrome not to develop in an animal that may be exposed to or predisposed to the disease, disorder, or syndrome but does not yet experience or display symptoms of the disease, disorder, or syndrome.

“Treating” or “treatment” of a disease, disorder, or syndrome, as used herein, includes (i) inhibiting the disease, disorder, or syndrome, i.e., arresting its development; and (ii) relieving the disease, disorder, or syndrome, i.e., causing regression of the disease, disorder, or syndrome. As is known in the art, adjustments for systemic versus localized delivery, age, body weight, general health, sex, diet, time of administration, drug interaction and the severity of the condition may be necessary, and will be ascertainable with routine experimentation by one of ordinary skill in the art.

The compounds disclosed herein also include all pharmaceutically acceptable isotopic variations, in which at least one atom is replaced by an atom having the same atomic number, but an atomic mass different from the atomic mass usually found in nature. Examples of isotopes suitable for inclusion in the disclosed compounds include, without limitation, isotopes of hydrogen, such as ²H and ³H; isotopes of carbon, such as ¹³C and ¹⁴C; isotopes of nitrogen, such as ¹⁵N; isotopes of oxygen, such as ¹⁷O and ¹⁸O; isotopes of phosphorus, such as ³¹P and ³²P; isotopes of sulfur, such as .sup.³⁵S; isotopes of fluorine, such as ¹⁸F; and isotopes of chlorine, such as ³⁶Cl. Use of isotopic variations (e.g., deuterium, ²H) may afford certain therapeutic advantages resulting from greater metabolic stability, for example, increased in vivo half-life or reduced dosage requirements. Additionally, certain isotopic variations of the disclosed compounds may incorporate a radioactive isotope (e.g., tritium, ³H, or ¹⁴C), which may be useful in drug and/or substrate tissue distribution studies.

### Embodiments of the Invention

The following paragraphs present a number of embodiments of compounds of the invention. In each instance the embodiment includes both the recited compounds, as well as a single stereoisomer or mixture of stereoisomers thereof, as well as a pharmaceutically acceptable salt thereof.

### Embodiments (A1)

In another embodiment, the Compound of Formula I is that where R^{5a} is hydrogen or alkyl and R^{5c}, R^{5d}, R^{5e}, R^{5f}, and R^{5g} are hydrogen; and all other groups are as defined in the Summary of the Invention for a Compound of Formula I. In another embodiment, the Compound of Formula I is that where R^{5a} is alkyl and R^{5c}, R^{5d}, R^{5e}, R^{5f}, and R^{5g} are hydrogen; and all other groups are as defined in the Summary of the Invention for a Compound of Formula I.

### Embodiments (A2)

In another embodiment, the Compound of Formula I is that where R^{5b} is (C₁₋₃)alkyl, or halo(C₁₋₃)alkyl and R^{5a}, R^{5c}, R^{5d}, R^{5e}, R^{5f}, R^{5g}, and R^{5h} are hydrogen; and all other groups are as defined in the Summary of the Invention for a Compound of Formula I. In another embodiment, the Compound of Formula I is that where R^{5b} is (C₁₋₃)alkyl and R^{5a}, R^{5c}, R^{5d}, R^{5e}, R^{5f}, R^{5g}, and R^{5h} are are as defined in the Summary of the Invention for a Compound of Formula I. In another embodiment, the Compound of Formula I is that where R^{5b} is (C₁₋₃)alkyl and R^{5a}, R^{5d}, R^{5e}, R^{5f}, R^{5g}, and R^{5h} are hydrogen; and all other groups are as defined in the Summary of the Invention for a Compound of Formula I. In another embodiment, the Compound of Formula I is that where R^{5b} is (C₁₋₃)alkyl; R^{5a}, R^{5c}, R^{5d}, R^{5e}, R^{5f}, R^{5g}, and R^{5h} are hydrogen; and all other groups are as defined in the Summary of the Invention for a Compound of Formula I.

### Embodiments (A3)

In another embodiment, the Compound of Formula I is that where R⁵′ is hydrogen or alkyl and R^{5a}, R^{5d}, R^{5e}, R^{5f}, and R^{5g} are hydrogen; and all other groups are as defined in the Summary of the Invention for a Compound of Formula I. In another embodiment, the Compound of Formula I is that where R^{5c} is alkyl and R^{5a}, R^{5d}, R^{5e}, R^{5f}, and R^{59g} are hydrogen; and all other groups are as defined in the Summary of the Invention for a Compound of Formula I.

### Embodiments (A4)

In another embodiment, the Compound of Formula I is that where R^{5h} is hydrogen or halo and R^{5a}, R^{5c}, R^{5d}, R^{5e}, R^{5f}, R^{5g}, is hydrogen; and all other groups are as defined in the Summary of the Invention for a Compound of Formula I. In another embodiment, the Compound of Formula I is that where R^{5h} is halo and R^{5a}, R^{5c}, R^{5d}, R^{5e}, R^{5f}, and R^{5g} are hydrogen; and all other groups are as defined in the Summary of the Invention for a Compound of Formula I. In another embodiment, the Compound of Formula I is that where R^{5h} is fluoro and R^{5a}, R^{5c}, R^{5d}, R^{5e}, R^{5f}, and R^{5g} are hydrogen; and all other groups are as defined in the Summary of the Invention for a Compound of Formula I.

### Embodiment (B)

Another embodiment of the Invention is directed to a Compound of Formula I(a)

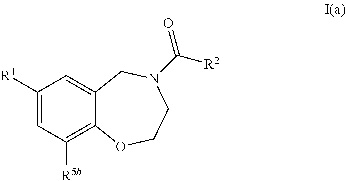

where R¹, R², R^{5b}, and all other groups are as defined in the Summary of the Invention for a Compound of Formula I. In this and other embodiments, R^{5b} is methyl, ethyl propyl, or trifluoromethyl. In this and other embodiments, R^{5b} is methyl or trifluoromethyl.

### Embodiment (B1)

In another embodiment, the Compound is according to Formula I(a) where
R¹ is phenyl substituted with one or two R⁶ groups; or
R¹ is heteroaryl optionally substituted with one, two, or three R⁷;
R² is —NR³R⁴;
R³ is hydrogen, alkyl, or alkoxycarbonylalkyl; and R⁴ is optionally substituted cycloalkyl, optionally substituted phenyl, optionally substituted phenylalkyl, or optionally substituted heteroarylalkyl; or
R³ and R⁴ together with the nitrogen to which they are attached form HET optionally substituted on any substitutable atom of the ring with R¹⁰, R^{10a}, R^{10b}, R^{10c}, R^{10d}, R^{10e}, and R^{10f};
BET is
   (a) a saturated or partially unsaturated, but non-aromatic, monocyclic 5- to 8-membered ring optionally containing an additional one or two ring heteroatoms which are independently oxygen, sulfur, or nitrogen where the remaining ring atoms are carbon; or
   (b) a partially unsaturated, but not aromatic, monocyclic 5- to 8-membered ring optionally containing an additional one or two ring heteroatoms which are independently oxygen, sulfur, or nitrogen and the remaining ring atoms are carbon and which ring is fused to a benzo ring; or
   (c) a fused, bridged, or spirocyclic, bicyclic 7- to 11-membered ring optionally containing an additional one or two heteroatoms which are independently oxygen, sulfur, or nitrogen and the remaining ring atoms are carbon and where each ring of the 7- to 11-membered ring is saturated or partially unsaturated but not fully aromatic; or
   (d) a fused, bridged, or spirocyclic, bicyclic 7- to 11-membered ring optionally containing an additional one or two ring heteroatoms which are independently oxygen, sulfur, or nitrogen and the remaining ring atoms are carbon where each ring of the bicyclic 7- to 11-membered ring is saturated or partially unsaturated but not fully aromatic, and where the bicyclic 7- to 11-membered ring is fused to a benzo ring;
each R⁶, when R⁶ is present, is independently nitro, —NR⁸R^{8a}, —C(O)NR⁸R^{8a}, —NR⁸C(O)OR⁹, or heteroaryl optionally substituted with 1, 2, or 3 R¹⁴;
each R⁷, when present, is independently alkyl, cycloalkyl, halo, —NR⁸R^{8a}, —C(O)NR⁸R^{8a}, —NR⁸C(O)OR⁹, —NR⁸C(O)R⁹, —NR⁸S(O)₂R^{8a}, or —S(O)₂NR⁸R⁹;
R⁸ is hydrogen, alkyl, or alkenyl;
R^{8a} is hydrogen, alkyl, haloalkyl, optionally substituted heterocycloalkyl, or optionally substituted phenylalkyl;
R⁹ is alkyl or haloalkyl; and
R¹⁰, R^{10a}, R^{10b}, R^{10c}, R^{10d}, R^{10e}, and R^{10f} are independently hydrogen, halo, alkyl, haloalkyl, haloalkenyl, hydroxyalkyl, alkylthio, alkylsulfonyl, hydroxy, alkoxy, haloalkoxy, cyano, alkoxycarbonyl, carboxy, amino, alkylamino, dialkylamino, —C(O)R¹², —C(O)NR¹¹R^{11a}, optionally substituted cycloalkyl, optionally substituted cycloalkylalkyl, optionally substituted phenyl, optionally substituted phenylalkyl, optionally substituted phenyloxy, optionally substituted phenyloxyalkyl, optionally substituted heterocycloalkyl, optionally substituted heterocycloalkylalkyl, optionally substituted heteroaryl, or optionally substituted heteroarylalkyl; or two of R¹⁰, R^{10a}, R^{10b}, R^{10c}, R^{10d}, R^{10e}, and when attached to the same carbon form oxo, imino, or thiono;
R¹¹ hydrogen, alkyl, or alkenyl;
R^{11a} hydrogen, alkyl, or alkenyl;
R¹² is alkyl, or optionally substituted heteroaryl; and
each R¹⁴, when present, is halo, alkyl, or alkoxycarbonyl.

### Embodiment (B1a)

In another embodiment, the Compound is according to Formula I(a) where
R¹ is phenyl substituted with one or two R⁶ groups; or
R¹ is heteroaryl optionally substituted with one, two, or three R⁷;
R² is —NR³R⁴;
R³ is hydrogen, alkyl, or alkoxycarbonylalkyl; and R⁴ is cycloalkyl, phenylalkyl, heteroarylalkyl, phenyl, or phenyl substituted with one or two alkyl; or
R³ and R⁴ together with the nitrogen to which they are attached form HET optionally substituted on any substitutable atom of the ring with R¹⁰, R^{10a}, R^{10b}, R^{10c}, R^{d}, R^{10e}, and R^{10f};
HET is
   (a) a saturated or partially unsaturated, but non-aromatic, monocyclic 5- to 8-membered ring optionally containing an additional one or two ring heteroatoms which are independently oxygen, sulfur, or nitrogen where the remaining ring atoms are carbon; or
   (b) a partially unsaturated, but not aromatic, monocyclic 5- to 8-membered ring optionally containing an additional one or two ring heteroatoms which are independently oxygen, sulfur, or nitrogen and the remaining ring atoms are carbon and which ring is fused to a benzo ring; or
   (c) a fused, bridged, or spirocyclic, bicyclic 7- to 11-membered ring optionally containing an additional one or two heteroatoms which are independently oxygen, sulfur, or nitrogen and the remaining ring atoms are carbon and where each ring of the 7- to 11-membered ring is saturated or partially unsaturated but not fully aromatic; or
   (d) a fused, bridged, or spirocyclic, bicyclic 7- to 11-membered ring optionally containing an additional one or two ring heteroatoms which are independently oxygen, sulfur, or nitrogen and the remaining ring atoms are carbon where each ring of the bicyclic 7- to 11-membered ring is saturated or partially unsaturated but not fully aromatic, and where the bicyclic 7- to 11-membered ring is fused to a benzo ring;
R^{5b} is (C₁₋₃)alkyl or halo(C₁₋₃)alkyl;
each R⁶, when R⁶ is present, is independently nitro, —NR⁸R^{8a}, —C(O)NR⁸R^{8a}, —NR⁸C(O)OR⁹, or heteroaryl optionally substituted with 1, 2, or 3 R¹⁴;
each R⁷, when present, is independently alkyl, cycloalkyl, halo, —NR⁸R^{8a}, —C(O)NR⁸R^{8a}, —NR⁸C(O)OR⁹, —NR⁸C(O)R⁹, —NR⁸S(O)₂R^{8a}, or —S(O)₂NR⁸R⁹;
R⁸ is hydrogen, alkyl, or alkenyl;
R^{8a} is hydrogen, alkyl, haloalkyl, heterocycloalkyl, or phenylalkyl;
R⁹ is alkyl or haloalkyl; and
R¹⁰, R^{10a}, R^{10b}, R^{10d}, R^{10e}, and R^{10f} are independently hydrogen, halo, alkyl, haloalkyl, haloalkenyl, hydroxyalkyl, alkylthio, alkylsulfonyl, hydroxy, alkoxy, haloalkoxy, cyano, alkoxycarbonyl, carboxy, amino, alkylamino, dialkylamino, —C(O)R¹², —C(O)NR¹¹R^{11a}, cycloalkyl, cycloalkylalkyl, phenyl, phenylalkyl, phenyloxy, phenyloxyalkyl, heterocycloalkyl, heterocycloalkylalkyl, heteroaryl, or heteroarylalkyl where the ring portion of any R¹⁰, R^{10a}, R^{10b}, R^{10c}, R^{10d}, R^{10e}, and R^{10f} phenyl, phenylalkyl, phenyloxy, phenyloxyalkyl, heteroaryl, or heteroarylalkyl is optionally substituted with one, two, or three groups which are independently halo, hydroxy, nitro, alkyl, haloalkyl, alkylcarbonyl, alkoxy, amino, alkylamino, dialkylamino, or cycloalkyl; or two of R¹⁰, R^{10a}, R^{10b}, R^{10c}, R^{10d}, R^{10e}, and R^{10f} when attached to the same carbon form oxo, imino, or thiono;
R¹¹ hydrogen, alkyl, or alkenyl;
R^{11a} hydrogen, alkyl, or alkenyl;
R¹² is alkyl, or optionally substituted heteroaryl; and
each R¹⁴, when present, is halo, alkyl, or alkoxycarbonyl.

### Embodiment (B2)

In another embodiment, the Compound is according to Formula I(a) where R¹ is as defined in the Summary of the Invention for a Compound of Formula I;
R² is —NR³R⁴ where R³ is hydrogen, alkyl, or alkoxycarbonylalkyl; and R⁴ is optionally substituted cycloalkyl, optionally substituted phenyl, optionally substituted phenylalkyl, or optionally substituted heteroarylalkyl; or
R² is —NR³R⁴ where R³ and R⁴ together with the nitrogen to which they are attached form HET and HET is indolin-1-yl, isoindolin-2-yl, 1,2,3,4-tetrahydroquinolin-1-yl, 1,2,3,4-tetrahydroisoquinolin-2-yl, or 1,2,3,4-tetrahydro-1,4-epiminonaphth-9-yl, where any substitutable atom on BET is optionally substituted with R¹⁰, R^{10a}, and R^{10b}; or
R² is —NR³R⁴ where R³ and R⁴ together with the nitrogen to which they are attached form HET according to formula (a):

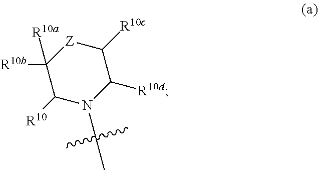

where Z is a bond, —C(O)—, —O—, —S—, —S(O)—, —S(O)₂—, —N(R^{z})—, —C(R^{10e})(R^{10f})—, or C₂₋₃-alkylene; or
R² is —NR³R⁴ where R³ and R⁴ together with the nitrogen to which they are attached form HET according to formula (b):

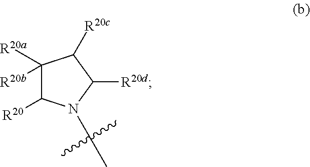

where
(a) R²⁰ and R^{20c} or R²⁰ and R^{20d} together with the carbons to which they are bonded form a cycloalkyl or hetercycloalkyl such that HET is a bridged bicyclic moiety; or
(b) R^{20a} and R^{20b} together with the carbons to which they are bonded form a cycloalkyl or hetercycloalkyl such that HET is a fused bicyclic moiety; or
(c) R^{20a} and R^{wb} together with the carbon to which they are attached form cycloalkyl or heterocycloalkyl such that HET is a spirocyclic bicyclic moiety;
where the cycloalkyl and heterocycloalkyl are optionally substituted with R¹⁰ and R^{10a}; and the remaining of R²⁰, R^{20a}, R^{20b}, R^{20c}, and R^{20d} are hydrogen; or
R² is —NR³R⁴ where R³ and R⁴ together with the nitrogen to which they are attached form HET according to formula (b):

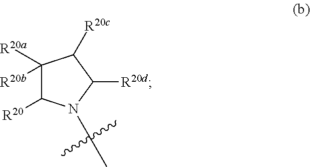

where R²⁰ and R^{20d} together with the carbons to which they are bonded form a cycloalkyl or hetercycloalkyl and R^{20a} and R^{20c} together with the carbons to which they are bonded form a cycloalkyl or hetercycloalkyl such that HET is a tricyclic moiety where the cycloalkyl and heterocycloalkyl are optionally substituted with R¹⁰ and R^{10a}; and and R^{20b} is hydrogen; or
R² is —NR³R⁴ where R³ and R⁴ together with the nitrogen to which they are attached form HET according to formula (c):

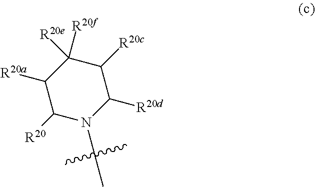

where
(a) R²⁰ and R^{20d} or R²⁰ and R^{2c} together with the carbons to which they are bonded form a cycloalkyl or hetercycloalkyl such that BET is a bridged bicyclic moiety
(b) R^{20e} and R^{20f} together with the carbons to which they are bonded form cycloalkyl or heterocycloalkyl such that HET is a spirocyclic bicyclic moiety,
(c) R²⁰ and R^{20a} or R^{20a} and R^{20e} together with the carbons to which they are bonded form a cycloalkyl or hetercycloalkyl such that HET is a fused bicyclic moiety;
   where the cycloalkyl and heterocycloalkyl are optionally substituted with R¹⁰ and R^{10a}; and where the remaining of R²⁰, R^{20a}, R^{20c}, R^{20d}, R^{20e}, and R^{20f} are R¹⁰, R^{10a}, R^{10c}, R^{10d}, R^{10e}, R^{10f}, respectively; or
R² is —NR³R⁴ where R³ and R⁴ together with the nitrogen to which they are attached form BET according to formula (d), (e), or (f):

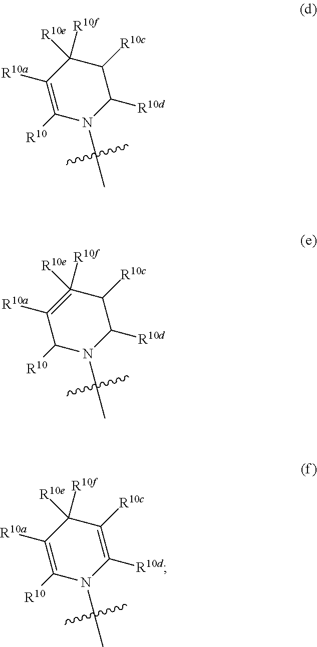

R¹⁰, R^{10a}, R^{20b}, R^{10c}, R^{10d}, R^{10e}, and R^{10f} are independently hydrogen; halo; alkyl; haloalkyl; haloalkenyl; hydroxyalkyl; alkylthio; alkylsulfonyl; hydroxy; alkoxy; haloalkoxy; cyano; alkoxycarbonyl; carboxy; amino; alkylamino; dialkylamino; —C(O)R¹²; —C(O)NR¹¹R^{11a}; optionally substituted cycloalkyl; optionally substituted cycloalkylalkyl; optionally substituted phenyl; optionally substituted phenylalkyl; optionally substituted phenyloxy; optionally substituted phenyloxyalkyl; optionally substituted heterocycloalkyl; optionally substituted heterocycloalkylalkyl; optionally substituted heteroaryl; or optionally substituted heteroarylalkyl; or two of R¹⁰, R^{10a}, R^{10b}, R^{10c}, R^{10d}, R^{10e}, and R^{10f} when attached to the same carbon form oxo, imino, or thiono;
R¹¹ hydrogen, alkyl, or alkenyl;
R^{11a} hydrogen, alkyl, or alkenyl; and
R¹² is alkyl, or optionally substituted heteroaryl.

### Embodiment (B2a)

In another embodiment, the Compound is according to Formula I(a) where
R¹ is phenyl substituted with one or two R⁶ groups; or
R¹ is heteroaryl optionally substituted with one, two, or three R⁷;
R² is —NR³R⁴ where R³ is hydrogen, alkyl, or alkoxycarbonylalkyl; and R⁴ is optionally substituted cycloalkyl, optionally substituted phenyl, optionally substituted phenylalkyl, or optionally substituted heteroarylalkyl; or
R² is —NR³R⁴ where R³ and R⁴ together with the nitrogen to which they are attached form HET and HET is indolin-1-yl, isoindolin-2-yl, 1,2,3,4-tetrahydroquinolin-1-yl, 1,2,3,4-tetrahydroisoquinolin-2-yl, or 1,2,3,4-tetrahydro-1,4-epiminonaphth-9-yl, where any substitutable atom on HET is optionally substituted with R¹⁰, R^{10a}, and R^{10b}; or
R² is —NR³R⁴ where R³ and R⁴ together with the nitrogen to which they are attached form HET according to formula (a):

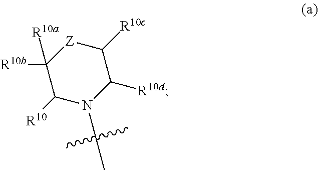

where Z is a bond, —C(O)—, —O—, —S—, —S(O)—, —S(O)₂—, —N(R^{z})—, —C(R^{10e})(R^{10f})—, or C₂₋₃-alkylene; or
R² is —NR³R⁴ where R³ and R⁴ together with the nitrogen to which they are attached form HET according to formula (b):

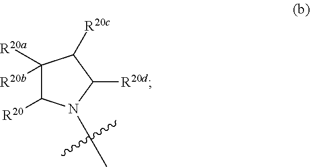

where
(a) R²⁰ and R^{20c} or R²⁰ and R^{20d} together with the carbons to which they are bonded form a cycloalkyl or hetercycloalkyl such that HET is a bridged bicyclic moiety; or
(b) R^{20a} and R^{20c} together with the carbons to which they are bonded form a cycloalkyl or hetercycloalkyl such that HET is a fused bicyclic moiety; or
(c) R^{20a} and R^{20b} together with the carbon to which they are attached form cycloalkyl or heterocycloalkyl such that HET is a spirocyclic bicyclic moiety;
where the cycloalkyl and heterocycloalkyl are optionally substituted with R¹⁰ and R^{10a}; and the remaining of R²⁰, R^{20a}, R^{20b}, R^{20c}, and hydrogen; or
R² is —NR³R⁴ where R³ and R⁴ together with the nitrogen to which they are attached form HET according to formula (b):

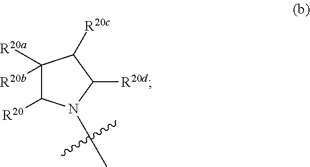

where R²⁰ and R^{20d} together with the carbons to which they are bonded form a cycloalkyl or hetercycloalkyl and R^{20a} and R^{20c} together with the carbons to which they are bonded form a cycloalkyl or hetercycloalkyl such that HET is a tricyclic moiety where the cycloalkyl and heterocycloalkyl are optionally substituted with R¹⁰ and R^{10a}; and and R^{20b} is hydrogen; or
R² is —NR³R⁴ where R³ and R⁴ together with the nitrogen to which they are attached form HET according to formula (c):

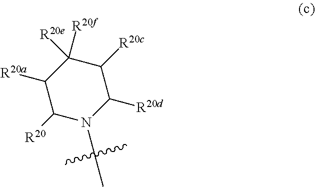

where
(a) R²⁰ and R^{20d} or R²⁰ and R^{20c} together with the carbons to which they are bonded form a cycloalkyl or hetercycloalkyl such that HET is a bridged bicyclic moiety
(b) R^{20e} and R^{20f} together with the carbons to which they are bonded form cycloalkyl or heterocycloalkyl such that BET is a spirocyclic bicyclic moiety,
(c) R²⁰ and R^{20a} or R^{20a} and R^{20e} together with the carbons to which they are bonded form a cycloalkyl or hetercycloalkyl such that HET is a fused bicyclic moiety;
where the cycloalkyl and heterocycloalkyl are optionally substituted with R¹⁰ and R^{10e}; and the remaining of R²⁰, R^{20a}, R^{20c}, R^{20d}, R^{20e}, and R^{20f} are R¹⁰, R^{10a}, R^{10c}, R^{10d}, R^{10e}, and R^{10f}, respectively; or
R² is —NR³R⁴ where R³ and R⁴ together with the nitrogen to which they are attached form HET according to formula (d), (e), or (f):

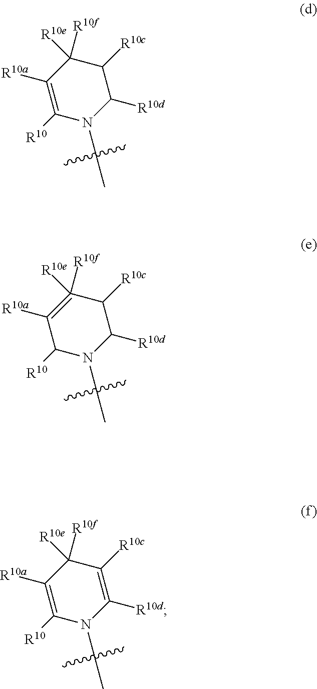

each R⁶, when present, is independently nitro, —NR⁸R^{8a}, —C(O)NR⁸R^{8a}, —NR⁸C(O)OR⁹, or heteroaryl optionally substituted with 1, 2, or 3 R¹⁴;
each R⁷, when present, is independently alkyl, cycloalkyl, —NR⁸R^{8a}, —C(O)NR⁸R^{8a}, —NR⁸C(O)OR⁹, or —NR⁸C(O)R⁹;
R⁸ is hydrogen, alkyl, or alkenyl;
R^{8a} is hydrogen, alkyl, haloalkyl, optionally substituted heterocycloalkyl, or optionally substituted phenylalkyl;
R⁹ is alkyl or haloalkyl; and
R¹⁰, R^{10a}, R^{10b}, R^{10c}, R^{10d}, R^{10e}, and R^{10f} are independently hydrogen, alkyl, halo, haloalkyl, haloalkenyl, hydroxyalkyl, alkylthio, alkylsulfonyl, hydroxy, alkoxy, haloalkoxy, cyano, alkoxycarbonyl, carboxy, amino, alkylamino, dialkylamino, —C(O)R¹², —C(O)NR¹¹R^{11a}, optionally substituted cycloalkyl, optionally substituted cycloalkylalkyl, optionally substituted phenyl, optionally substituted phenylalkyl, optionally substituted phenyloxy, optionally substituted phenyloxyalkyl, optionally substituted heterocycloalkyl, optionally substituted heterocycloalkylalkyl, optionally substituted heteroaryl, or optionally substituted heteroarylalkyl; or R^{10a} and R^{10b} together form oxo; or R^{10e} and R^{10f} together form oxo;
R^{z} is hydrogen, alkyl, haloalkyl, haloalkenyl, hydroxyalkyl, alkylsulfonyl, hydroxy, alkoxy, alkoxycarbonyl, —C(O)R¹², —C(O)NR¹¹R^{11a}, optionally substituted cycloalkyl, optionally substituted cycloalkylalkyl, optionally substituted phenyl, optionally substituted phenylalkyl, optionally substituted heterocycloalkyl, optionally substituted heterocycloalkylalkyl, optionally substituted heteroaryl, or optionally substituted heteroarylalkyl;
R¹¹ hydrogen, alkyl, or alkenyl;
R^{11a} hydrogen, alkyl, or alkenyl;
R¹² is alkyl, or optionally substituted heteroaryl; and
each R¹⁴, when present, is halo, alkyl, or alkoxycarbonyl.

### Embodiment (B3)

In another embodiment, the Compound is according to Formula I(a) where
R¹ is phenyl substituted with one or two R⁶ groups; or
R¹ is heteroaryl optionally substituted with one, two, or three R⁷;
R² is —NR³R⁴ where R³ is hydrogen, alkyl, or alkoxycarbonylalkyl; and R⁴ is optionally substituted cycloalkyl, optionally substituted phenyl, optionally substituted phenylalkyl, or optionally substituted heteroarylalkyl; or
R² is —NR³R⁴ and R³ and R⁴ together with the nitrogen to which they are attached form HET and HET is indolin-1-yl, isoindolin-2-yl, 1,2,3,4-tetrahydroquinolin-1-yl, 1,2,3,4-tetrahydroisoquinolin-2-yl, or 1,2,3,4-tetrahydro-1,4-epiminonaphth-9-yl, where any substitutable atom on HET is optionally substituted with R¹⁰, R^{10a}, and R^{10b}; or
R² is —NR³R⁴ where R³ and R⁴ together with the nitrogen to which they are attached form HET according to formula (a):

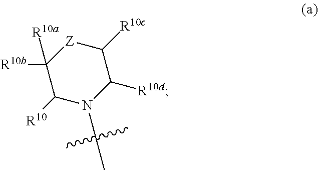

where Z is a bond, —C(O)—, —O—, —S—, —S(O)—, —S(O)₂—, —N(R^{z})—, —C(R^{10e})(R^{10f})—, or C₂₋₃-alkylene; R^{z} is hydrogen, alkyl, haloalkyl, haloalkenyl, hydroxyalkyl, alkylsulfonyl, hydroxy, alkoxy, alkoxycarbonyl, —C(O)R¹², —C(O)NR¹¹R^{11a}, optionally substituted cycloalkyl, optionally substituted cycloalkylalkyl, optionally substituted phenyl, optionally substituted phenylalkyl, optionally substituted heterocycloalkyl, optionally substituted heterocycloalkylalkyl, optionally substituted heteroaryl, or optionally substituted heteroarylalkyl; and R¹⁰, R^{10a}, R^{10b}, R^{10c}, and independently hydrogen, alkyl, haloalkyl, haloalkenyl, hydroxyalkyl, alkylthio, alkylsulfonyl, hydroxy, alkoxy, haloalkoxy, cyano, alkoxycarbonyl, carboxy, amino, alkylamino, dialkylamino, —C(O)R¹², —C(O)NR¹¹R^{11e}, optionally substituted cycloalkyl, optionally substituted cycloalkylalkyl, optionally substituted phenyl, optionally substituted phenylalkyl, optionally substituted phenyloxy, optionally substituted phenyloxyalkyl, optionally substituted heterocycloalkyl, optionally substituted heterocycloalkylalkyl, optionally substituted heteroaryl, or optionally substituted heteroarylalkyl; or R^{10a} and R^{10b} together form oxo; or R^{we} and ee together form oxo; or
R² is —NR³R⁴ where R³ and R⁴ together with the nitrogen to which they are attached form HET according to formula (b):

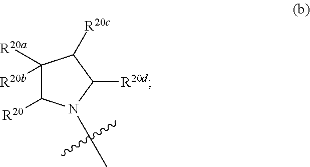

where
(a) R²⁰ and R^{20c} or R²⁰ and R^{20d} together with the carbons to which they are bonded form a cycloalkyl or hetercycloalkyl such that HET is a bridged bicyclic moiety; or
(b) R^{20a} and R^{20c} together with the carbons to which they are bonded form a cycloalkyl or hetercycloalkyl such that HET is a fused bicyclic moiety; or
(c) R^{20a} and R^{20b} together with the carbon to which they are attached form cycloalkyl or heterocycloalkyl such that HET is a spirocyclic bicyclic moiety;
where the cycloalkyl and heterocycloalkyl are optionally substituted with R¹⁰ and R^{10a} where R¹⁰ and R^{10a} are independently hydroxy, alkyl, haloalkyl, or optionally substituted phenyl; and the remaining of R²⁰, R^{20a}, R^{20b}, R^{20c}, and R^{20d} are hydrogen; or
R² is —NR³R⁴ where R³ and R⁴ together with the nitrogen to which they are attached form HET according to formula (b):

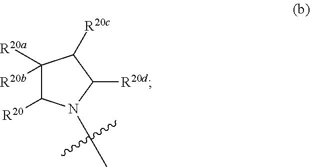

where R²⁰ and R^{20d} together with the carbons to which they are bonded form a cycloalkyl or hetercycloalkyl and R^{20a} and R^{20c} together with the carbons to which they are bonded form a cycloalkyl or hetercycloalkyl such that HET is a tricyclic moiety, and where the cycloalkyl and heterocycloalkyl are optionally substituted with R¹⁰ and R^{10a}; and R^{20b} is hydrogen; or
R² is —NR³R⁴ where R³ and R⁴ together with the nitrogen to which they are attached form HET according to formula (c):

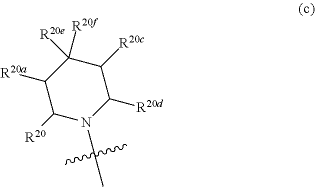

where
(d) R²⁰ and R^{20d} or R²⁰ and R^{20c} together with the carbons to which they are bonded form a cycloalkyl or hetercycloalkyl such that HET is a bridged bicyclic moiety
(e) R^{20e} and R^{20f} together with the carbons to which they are bonded form cycloalkyl or heterocycloalkyl such that HET is a spirocyclic bicyclic moiety,
(f) R²⁰ and R^{20a} or R^{20a} and R^{20e} together with the carbons to which they are bonded form a cycloalkyl or hetercycloalkyl such that HET is a fused bicyclic moiety;
where the cycloalkyl is optionally substituted with R¹⁰ and R^{10a} where R¹⁰ and R^{10a} are independently alkyl or together form oxo; and the remaining of R²⁰, R^{20a}, R^{20c}, R^{20d}, R^{20e}, and R^{20f} are R¹⁰, R^{10a}, R^{10c}, R^{10d}, R^{10e}, and R^{10f}, respectively, and the R¹⁰, R^{10a}, R^{10c}, R^{10d}, R^{10e}, and R^{10f} are independently hydrogen, hydroxy, alkyl, halo, haloalkyl, hydroxyalkyl, optionally substituted phenyl, or amino, or R^{10e} and R^{10f} together form oxo; or
R² is —NR³R⁴ where R³ and R⁴ together with the nitrogen to which they are attached form HET according to formula (d), (e), or (f):

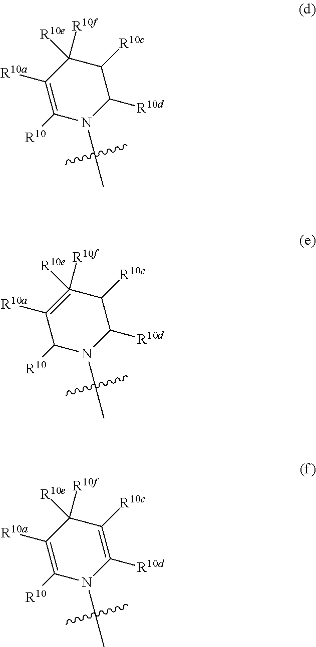

where R¹⁰, R^{10a}, R^{10b}, R^{10c}, R^{10d}, R^{10e}, and R^{10f} are independently hydrogen, hydroxy, alkyl, haloalkyl, or optionally substituted phenyl; or, in formula (d) and (f), R^{10a} and R^{10f} together form oxo;
each R⁶, when present, is independently nitro, —NR⁸R^{8a}, —C(O)NR⁸R^{8a}, —NR⁸C(O)OR⁹, or heteroaryl optionally substituted with 1, 2, or 3 R¹⁴;
each R⁷, when present, is independently alkyl, cycloalkyl, —NR⁸R^{8a}, —C(O)NR⁸R^{8a}, —NR⁸C(O)OR⁹, or —NR⁸C(O)R⁹;
R⁸ is hydrogen, alkyl, or alkenyl;
R^{8a} is hydrogen, alkyl, haloalkyl, optionally substituted heterocycloalkyl, or optionally substituted phenylalkyl;
R⁹ is alkyl or haloalkyl; and
R¹¹ hydrogen, alkyl, or alkenyl;
R^{11a} hydrogen, alkyl, or alkenyl;
R¹² is alkyl, or optionally substituted heteroaryl; and
each R¹⁴, when present, is halo, alkyl, or alkoxycarbonyl.

### Embodiments (C)

In another embodiment, the Compound is according to Formula I(a) where R¹ is heteroaryl optionally substituted with one, two, or three R⁷; and R², R⁷ and all other groups are as defined in the Summary of the Invention for a Compound of Formula I or as defined in any one of embodiments B, B1, B1a, B2, B2a, and B3. In another embodiment, the Compound is according to Formula I(a) where R¹ is heteroaryl optionally substituted with one or two R⁷; and R², R⁷ and all other groups are as defined in the Summary of the Invention for a Compound of Formula I or as defined in any one of embodiments B, B1, B1a, B2, B2a, and B3. In another embodiment, the Compound is according to Formula I(a) where R¹ is heteroaryl substituted with one or two R⁷; and R², R⁷ and all other groups are as defined in the Summary of the Invention for a Compound of Formula I or as defined in any one of embodiments B, B1, B1a, B2, B2a, and B3.

### Embodiments (C1)

In another embodiment, the Compound is according to Formula I(a) where R¹ is a 9-membered heteroaryl optionally substituted with one, two, or three R⁷; and R², R⁷ and all other groups are as defined in the Summary of the Invention for a Compound of Formula I or as defined in any one of embodiments B, B1, B1a, B2, B2a, and B3. In another embodiment, the Compound is according to Formula I(a) where R¹ is a 9-membered heteroaryl optionally substituted with one or two R⁷; and R², R⁷ and all other groups are as defined in the Summary of the Invention for a Compound of Formula I or as defined in any one of embodiments B, B1, B1a, B2, B2a, and B3. In another embodiment, the Compound is according to Formula I(a) where R¹ is a 9-membered heteroaryl substituted with one or two R⁷; and R², R⁷ and all other groups are as defined in the Summary of the Invention for a Compound of Formula I or as defined in any one of embodiments B, B1, B1a, B2, B2a, and B3.

### Embodiments (C2)

In another embodiment, the Compound is according to Formula I(a) where R¹ is benzimidazolyl, 1H-imidazo[4,5-b]pyridinyl, 3H-imidazo[4,5-b]pyridinyl, thiazolo[4,5-b]pyridinyl, or thiazolo[5,4-b]pyridinyl where R¹ is optionally substituted with one or two R⁷; and R², R⁷ and all other groups are as defined in the Summary of the Invention for a Compound of Formula I or as defined in any one of embodiments B, B1, B1a, B2, B2a, and B3. In another embodiment, the Compound is according to Formula I(a) where R¹ is benzimidazolyl, 1H-imidazo[4,5-b]pyridinyl, 3H-imidazo[4,5-b]pyridinyl, thiazolo[4,5-b]pyridinyl, or thiazolo[5,4-b]pyridinyl where R¹ is optionally substituted with one or two R⁷; each R⁷, when present, is alkyl, haloalkyl, cycloalkyl, —NR⁸R^{8a}, or —NR⁸C(O)OR⁹; and R⁸, R^{8a}, R⁹, R² and all other groups are as defined in the Summary of the Invention for a Compound of Formula I or as defined in any one of embodiments B, B1, B1a, B2, B2a, and B3. In another embodiment, the Compound is according to Formula I(a) where R¹ isbenzimidazolyl, 1H-imidazo[4,5-b]pyridinyl, 3H-imidazo[4,5-b]pyridinyl, thiazolo[4,5-b]pyridinyl, or thiazolo[5,4-b]pyridinyl where R¹ is optionally substituted with one or two R⁷; each R⁷, when present, is alkyl, haloalkyl, cycloalkyl, —NR⁸R^{8a}, or —NR⁸C(O)OR⁹; R⁸ is hydrogen; R^{8a} is hydrogen, alkyl, or haloalkyl; R⁹ is alkyl; and R² and all other groups are as defined in the Summary of the Invention for a Compound of Formula I or as defined in any one of embodiments B, B1, B1a, B2, B2a, and B3. In another embodiment, the Compound is according to Formula I(a) where R¹ isbenzimidazolyl, 1H-imidazo[4,5-b]pyridinyl, 3H-imidazo[4,5-b]pyridinyl, thiazolo[4,5-b]pyridinyl, or thiazolo[5,4-b]pyridinyl where R¹ is optionally substituted with one or two R⁷; each R⁷, when present, is alkyl, haloalkyl, cycloalkyl, —NR⁸R^{8a}, or —NR⁸C(O)OR⁹; R⁸ is hydrogen; R^{8a} is hydrogen, C₁₋₃-alkyl, or haloalkyl; R⁹ is C₁₋₃-alkyl; and R² and all other groups are as defined in the Summary of the Invention for a Compound of Formula I or as defined in any one of embodiments B, B1, B1a, B2, B2a, and B3. In another embodiment, the Compound is according to Formula I(a) where R¹ is benzimidazol-6-yl, 2-methyl-benzimidazol-6-yl, 2-cyclopropyl-benzimidazol-6-yl, 2-trifluoromethyl-benzimidazol-6-yl, 2-amino-benzimidazol-6-yl, 2-(2,2,2-trifluoroethylamino)-benzimidazol-6-yl, 2-(2-monofluoroethylamino)-benzimidazol-6-yl, 2-(2,2-difluoroethylamino)-benzimidazol-6-yl, 2-(methoxycarbonylamino)-benzimidazol-6-yl, imidazo[4,5-b]pyridin-6-yl, 2-methyl-imidazo[4,5-b]pyridin-6-yl, 2-amino-imidazo[4,5-b]pyridin-6-yl, 2-cyclopropyl-imidazo[4,5-b]pyridin-6-yl, or 2-trifluoromethyl-imidazo[4,5-b]pyridin-6-yl; and R² and all other groups are as defined in the Summary of the Invention for a Compound of Formula I or as defined in any one of embodiments B, B1, B1a, B2, B2a, and B3.

### Embodiments (C3)

In another embodiment, the Compound is according to Formula I(b)

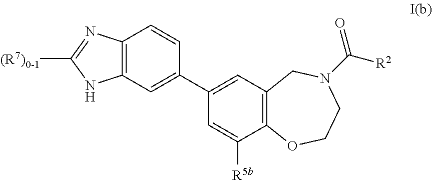

where R² and R⁷, when present, are as defined in the Summary of the Invention for a Compound of Formula I or as defined in any one of embodiments B, B1, B1a, B2, B2a, and B3. In another embodiment, the Compound is according to Formula I(b) where R⁷, when present, is alkyl, haloalkyl, cycloalkyl, —NR⁸R^{8a}, or —NR⁸C(O)OR⁹; R², R⁸, R^{8a}, R⁹, and all other groups are as defined in the Summary of the Invention for a Compound of Formula I or as defined in any one of embodiments B, B1, B1a, B2, B2a, and B3. In another embodiment, the Compound is according to Formula I(b) where R⁷, when present, is alkyl, haloalkyl, cycloalkyl, —NR⁸R^{8a}, or —NR⁸C(O)OR⁹; R⁸ is hydrogen; R^{8a} is hydrogen, alkyl, or haloalkyl; R⁹ is alkyl; and R² is as defined in the Summary of the Invention for a Compound of Formula I or as defined in any one of embodiments B, B1, B1a, B2, B2a, and B3. In another embodiment, the Compound is according to Formula I(b) where R⁷, when present, is C₁₋₃-alkyl, haloalkyl, cycloalkyl, —NR⁸R^{8a}, or —NR⁸C(O)OR⁹; R⁸ is hydrogen; R^{8a} is hydrogen, C₁₋₃-alkyl, or haloalkyl; R⁹ is C₁₋₃-alkyl; and R² is as defined in the Summary of the Invention for a Compound of Formula I or as defined in any one of embodiments B, B1, B1a, B2, B2a, and B3.

### Embodiments (C4)

In another embodiment, the Compound is according to Formula I(c1) or I(c2)

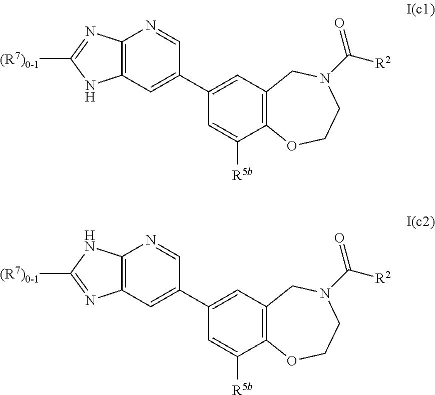

where R², R^{5b}, and R⁷ are as defined in the Summary of the Invention for a Compound of Formula I or as defined in any one of embodiments B, B1, B1a, B2, B2a, and B3. In another embodiment, the Compound is according to Formula I(c1) or I(c2) where R⁷, when present, is alkyl, haloalkyl, cycloalkyl, —NR⁸R^{8a}, or —NR⁸C(O)OR⁹; R², R⁸, R^{8a}, R⁹, and all other groups are as defined in the Summary of the Invention for a Compound of Formula I or as defined in any one of embodiments B, B1, B1a, B2, B2a, and B3. In another embodiment, the Compound is according to Formula I(c1) or I(c2) where R⁷, when present, is alkyl, haloalkyl, cycloalkyl, —NR⁸R^{8a}, or —NR⁸C(O)OR⁹; R⁸ is hydrogen; R^{8a} is hydrogen, alkyl, or haloalkyl; R⁹ is alkyl; and R² is as defined in the Summary of the Invention for a Compound of Formula I or as defined in any one of embodiments B, B1, B1a, B2, B2a, and B3. In another embodiment, the Compound is according to Formula I(c1) or I(c2) where R⁷, when present, is haloalkyl, cycloalkyl, —NR⁸R^{8a}, or —NR⁸C(O)OR⁹; R⁸ is hydrogen; R^{8a} is hydrogen, C₁₋₃-alkyl, or haloalkyl; R⁹ is C₁₋₃-alkyl; and R² is as defined in the Summary of the Invention for a Compound of Formula I or as defined in any one of embodiments B, B1, B1a, B2, B2a, and B3.

### Embodiments (C5)

In another embodiment, the Compound is according to Formula I(d1) or I(d2)

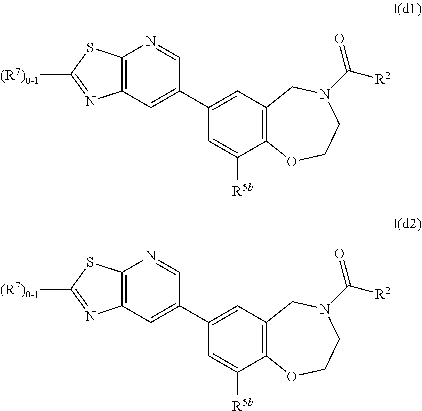

where R² and R⁷ are as defined in the Summary of the Invention for a Compound of Formula I or as defined in any one of embodiments B, B1, B1a, B2, B2a, and B3. In another embodiment, the Compound is according to Formula I(d1) or I(d2) where R⁷, when present, is alkyl, haloalkyl, cycloalkyl, —NR⁸R^{8a}, or —NR⁸C(O)OR⁹; R², R⁸, R^{8a}, R⁹, and all other groups are as defined in the Summary of the Invention for a Compound of Formula I or as defined in any one of embodiments B, B1, B1a, B2, B2a, and B3. In another embodiment, the Compound is according to Formula I(d1) or I(d2) where R⁷, when present, is alkyl, haloalkyl, cycloalkyl, —NR⁸R^{8a}, or —NR⁸C(O)OR⁹; R⁸ is hydrogen; R^{8a} is hydrogen, alkyl, or haloalkyl; R⁹ is alkyl; and R² is as defined in the Summary of the Invention for a Compound of Formula I or as defined in any one of embodiments B, B1, B1a, B2, B2a, and B3. In another embodiment, the Compound is according to Formula I(dl) or I(d2) where R⁷, when present, is C₁₋₃-alkyl, haloalkyl, cycloalkyl, —NR⁸R^{8a}, or —NR⁸C(O)OR⁹; R⁸ is hydrogen; R^{8a} is hydrogen, C₁₋₃-alkyl, or haloalkyl; R⁹ is C₁₋₃-alkyl; and R² is as defined in the Summary of the Invention for a Compound of Formula I or as defined in any one of embodiments B, B1, B1a, B2, B2a, and B3.

### Embodiments (C6)

In another embodiment, the Compound is according to Formula I(a) where R¹ is a 6-membered heteroaryl optionally substituted with one, two, or three R⁷; and R², R⁷ and all other groups are as defined in the Summary of the Invention for a Compound of Formula I or as defined in any one of embodiments B, B1, B1a, B2, B2a, and B3. In another embodiment, the Compound is according to Formula I(a) where R¹ is a 6-membered heteroaryl optionally substituted with one or two R⁷; and R², R⁷ and all other groups are as defined in the Summary of the Invention for a Compound of Formula I or as defined in any one of embodiments B, B1, B1a, B2, B2a, and B3. In another embodiment, the Compound is according to Formula I(a) where R¹ is a 6-membered heteroaryl substituted with one or two R⁷; and R², R⁷ and all other groups are as defined in the Summary of the Invention for a Compound of Formula I or as defined in any one of embodiments B, B1, B1a, B2, B2a, and B3. In another embodiment, the Compound is according to Formula I(a) where R¹ is pyrazinyl, pyridazinyl, pyridinyl, or pyrimidinyl where R¹ is optionally substituted with one or two R⁷; and R², R⁷, and all other groups are as defined in the Summary of the Invention for a Compound of Formula I or as defined in any one of embodiments B, B1, B1a, B2, B2a, and B3. In another embodiment, the Compound is according to Formula I(a) where R¹ is pyrazinyl, pyridazinyl, pyridinyl, or pyrimidinyl where R¹ is substituted with one or two R⁷; and R², R⁷, and all other groups are as defined in the Summary of the Invention for a Compound of Formula I or as defined in any one of embodiments B, B1, B1a, B2, B2a, and B3. In another embodiment, the Compound is according to Formula I(a) where R¹ is pyrazinyl, pyridazinyl, pyridinyl, or pyrimidinyl where R¹ is optionally substituted with one or two R⁷; R⁷ is halo, optionally substituted heteroaryl, —NR⁸S(O)₂R^{8a}, —S(O)₂NR⁸R⁹, —C(O)NR⁸R^{8a}, or —NR⁸R^{8a}; R², R⁸, R^{8a}, and all other groups are as defined in the Summary of the Invention for a Compound of Formula I or as defined in any one of embodiments B, B1, B1a, B2, B2a, and B3. In another embodiment, the Compound is according to Formula I(a) where R¹ is pyrazinyl, pyridazinyl, pyridinyl, or pyrimidinyl where R¹ is optionally substituted with one or two R⁷; R⁷ is halo, optionally substituted heteroaryl, —NR⁸S(O)₂R^{8a}, —S(O)₂NR⁸R⁹, —C(O)NR⁸R^{8a}, or —NR⁸R^{8a}; each R⁸ is hydrogen; each R^{8a} is independently hydrogen or alkyl; R⁹ is hydrogen or alkyl; R² and all other groups are as defined in the Summary of the Invention for a Compound of Formula I or as defined in any one of embodiments B, B1, B1a, B2, B2a, and B3. In another embodiment, the Compound is according to Formula I(a) where R¹ is pyrazinyl, pyridazinyl, pyridinyl, or pyrimidinyl where R¹ is optionally substituted with one or two R⁷; R⁷ is optionally substituted heteroaryl, —C(O)NR⁸R^{8a} or —NR⁸R^{8a}; R², R⁸, R^{8a}, and all other groups are as defined in the Summary of the Invention for a Compound of Formula I or as defined in any one of embodiments B, B1, B1a, B2, B2a, and B3. In another embodiment, the Compound is according to Formula I(a) where R¹ is pyrazinyl, pyridazinyl, pyridinyl, or pyrimidinyl where R¹ is optionally substituted with one or two R⁷; R⁷ is optionally substituted heteroaryl, —C(O)NR⁸R^{8a} or —NR⁸R^{8a}; R⁸ is hydrogen; and R^{8a} is hydrogen or alkyl; and R² and all other groups are as defined in the Summary of the Invention for a Compound of Formula I or as defined in any one of embodiments B, B1, B1a, B2, B2a, and B3. In another embodiment, the Compound is according to Formula I(a) where R¹ is pyrazin-2-yl, 5-amino-pyrazin-2-yl, pyridazin-3-yl, pyridazin-4-yl, pyridazin-5-yl, pyridazin-6-yl, 6-amino-pyridazin-3-yl, pyrimidin-2-yl, pyrimidin-4-yl, pyrimidin-5-yl, pyrimidin-6-yl, 2-amino-pyrimidin-5-yl, pyridin-2-yl, pyridin-3-yl, pyridin-4-yl, pyridin-5-yl, pyridin-6-yl, 5-methylaminocarbonyl-pyridin-2-yl, 4-methylaminocarbonyl-pyridin-3-yl, or 4-(imidazol-2-yl)-pyridin-3-yl; and R² is as defined in the Summary of the Invention for a Compound of Formula I or as defined in any one of embodiments B, B1, B1a, B2, B2a, and B3.

### Embodiments (C6a)

In another embodiment, the Compound is according to Formula I(a) where R¹ is pyridin-3-yl optionally substituted with one, two, or three R⁷; and R², R⁷ and all other groups are as defined in the Summary of the Invention for a Compound of Formula I or as defined in any one of embodiments B, B1, B1a, B2, B2a, and B3. In another embodiment, the Compound is according to Formula I(a) where R¹ is pyridin-3-yl optionally substituted with one or two R⁷; and R², R⁷ and all other groups are as defined in the Summary of the Invention for a Compound of Formula I or as defined in any one of embodiments B, B1, B1a, B2, B2a, and B3. In another embodiment, the Compound is according to Formula I(a) where R¹ is pyridin-3-yl where R¹ is optionally substituted with one or two R⁷; R⁷ is halo, alkoxy, —NR⁸S(O)₂R^{8a}, —S(O)₂NR⁸R⁹, —C(O)NR⁸R^{8a}, or —NR⁸R^{8a}; R², R⁸, R^{8a}, and all other groups are as defined in the Summary of the Invention for a Compound of Formula I or as defined in any one of embodiments B, B1, B1a, B2, B2a, and B3. In another embodiment, the Compound is according to Formula I(a) where R¹ is pyridin-3-yl where R¹ is optionally substituted with one or two R⁷; R⁷ is halo, alkoxy, —NR⁸S(O)₂R^{8a}, —S(O)₂NR⁸R⁹, —C(O)NR⁸R^{8a}, or —NR⁸R^{8a}; each R⁸ is hydrogen; each R^{8a} is independently hydrogen or alkyl; R⁹ is hydrogen or alkyl; R² and all other groups are as defined in the Summary of the Invention for a Compound of Formula I or as defined in any one of embodiments B, B1, B1a, B2, B2a, and B3.

### Embodiments (C7)

In another embodiment, the Compound is according to Formula I(a) where R¹ is a 5-membered heteroaryl optionally substituted with one or two R⁷; and R², R⁷ and all other groups are as defined in the Summary of the Invention for a Compound of Formula I or as defined in any one of embodiments B, B1, B1a, B2, B2a, and B3. In another embodiment, the Compound is according to Formula I(a) where R¹ is a 5-membered heteroaryl substituted with one or two R⁷; and R², R⁷ and all other groups are as defined in the Summary of the Invention for a Compound of Formula I or as defined in any one of embodiments B, B1, B1a, B2, B2a, and B3. In another embodiment, the Compound is according to Formula I(a) where R¹ is pyrazolyl or thiazolyl, where R¹ is optionally substituted with one or two R⁷; and R², R⁷ and all other groups are as defined in the Summary of the Invention for a Compound of Formula I or as defined in any one of embodiments B, B1, B1a, B2, B2a, and B3. In another embodiment, the Compound is according to Formula I(a) where R¹ is pyrazolyl or thiazolyl, where R¹ is optionally substituted with one or two R⁷; each R⁷, when present, is alkyl, —NR⁸R^{8a}, or —NR⁸C(O)R⁹; and R², R⁸, R^{8a}, R⁹, and all other groups are as defined in the Summary of the Invention for a Compound of Formula I or as defined in any one of embodiments B, B1, B1a, B2, B2a, and B3. In another embodiment, the Compound is according to Formula I(a) where R¹ is pyrazolyl or thiazolyl, where R¹ is optionally substituted with one or two R⁷; each R⁷, when present, is alkyl, —NR⁸R^{8a}, or —NR⁸C(O)R⁹; R⁸ is hydrogen; R^{8a} is hydrogen, alkyl, or benzyl; R⁹ is alkyl; and R² and all other groups are as defined in the Summary of the Invention for a Compound of Formula I or as defined in any one of embodiments B, B1, B1a, B2, B2a, and B3. In another embodiment, the Compound is according to Formula I(a) where R¹ is pyrazolyl or thiazolyl, where R¹ is optionally substituted with one or two R⁷; each R⁷, when present, is C₁₋₃-alkyl, —NR⁸R^{8a}, or —NR⁸C(O)R⁹; R⁸ is hydrogen; R^{8a} is hydrogen, C₁₋₃alkyl, or benzyl; R is C₁₋₃-alkyl; and R² and all other groups are as defined in the Summary of the Invention for a Compound of Formula I or as defined in any one of embodiments B, B1, B1a, B2, B2a, and B3. In another embodiment, the Compound is according to Formula I(a) where R¹ is pyrazol-1-yl, pyrazol-3-yl, pyrazol-4-yl, pyrazol 5-phenylmethylamino-pyrazol-3-yl, 5-amino-pyrazol-3-yl, thiazol-5-yl, 2-methylcarbonylamino-thiazol-5-yl, or 2-amino-thiazol-5-yl; and R² and all other groups are as defined, in the Summary of the Invention for a Compound of Formula I or as defined in any one of embodiments B, B1, B1a, B2, B2a, and B3.

### Embodiments (C8)

In another embodiment, the Compound is according to Formula I(a) where R¹ is phenyl substituted with one, two, or three R⁶ groups; each R⁶ is independently nitro; cyano; halo; alkyl: alkenyl; alkynyl; halo; haloalkyl; —NRee, —C(O)NR⁸R^{8a}; —NR⁸C(O)OR⁹; —NR⁸C(O)R⁹; —NR⁸S(O)₂R^{8a}, —NR⁸C(O)NR^{8a}R⁹; carboxy, —C(O)OR; alkylcarbonyl; alkyl substituted with one or two —C(C)NR⁸R^{8a}; heteroaryl optionally substituted with 1, 2, or 3 R¹⁴; or optionally substituted heterocycloalkyl; and all other groups are as defined in the Summary of the Invention for a Compound of Formula I or as defined in any one of embodiments B, B1, B1a, 82. B2a md 133. In another embodiment, the Compound is according m Formula I(a) where R¹ is phenyl substituted with one or two R⁶ groups; each R⁶ is independently nitro; cyano; halo; alkyl; alkenyl; alkynyl; halo; haloalkyl; —OR^{8a}; —NR⁸R^{8a}: —C(O)NR⁸R^{8a}; —NR⁸C(O)OR⁹; —NR⁸O(O)R⁹; —NR⁸S(O)₂R^{8a}; —NR⁸C(O)NR^{8a}R⁹; carboxy, —C(O)OR⁹; alkylcarbonyl; alkyl substituted with one or two —C(O)NR⁸R^{8a}; heteroaryl optionally substituted with 1, 2, or 3 R¹⁴; or optionally substituted heterocycloalkyl; and all other groups are as defined in the Summary of the Invention for a Compound of Formula I or as defined in my one of embodiments B, R1, B1a, B2, B2a, and 133.

### Embodiments (C8a)

In another embodiment, the Compound is according to Formula I(a) where R¹ is phenyl substituted with one or two R⁶ groups; each R⁶ is independently —OR^{8a}; —NR⁸R^{8a}; —C(O)NR⁸R^{8a}; or heteroaryl optionally substituted with 1, 2, or 3 R¹⁴; and all other groups are as defined in the Summary of the Invention for a Compound of Formula I or as defined in any one of embodiments B, B1, B1a, B2, B2a, and B3. In another embodiment, the Compound is according to Formula I(a) where R¹ is phenyl NR⁸R^{8a}; substituted with one or two R⁶ groups; each R⁶ is independently —OR^{8a}; —-C(O)NR⁸R^{8a}; or heteroaryl optionally substituted with 1, 2, or 3 R¹⁴; R⁸ is hydrogen or alkyl; R^{8a} is hydrogen, alkyl, haloalkyl, or optionally substituted heterocycloalkyl; R¹⁴, when present, is halo; and all other groups are as defined in the Summary of the Invention for a Compound of Formula I or as defined in any one of embodiments B, B1, B1a, B2, B2a, and B3. In another embodiment, the Compound is according to Formula I(a) where R¹ is phenyl substituted with one or two R⁶ groups; each R⁶ is independently 2,2-difluoroethylaminocarbonyl, N-pyrrolidin-1-ylaminocarbonyl, N-pyrrolidin-2-ylaminocarbonyl, N-pyrrolidin-3-ylaminocarbonyl, imidazol-2-yl, imidazol-4-yl, imidazol-5-yl, pyrazol-1-yl, pyrazol-3-yl, pyrazol-4-yl, pyrazol-5-yl, benzimidazol-2-yl, 5-fluoro-benzimidazol-2-yl, or benzimidazol-6-yl; and all other groups are as defined in the Summary of the Invention for a Compound of Formula I or as defined in any one of embodiments B, B1, B1a, B2, B2a, and B3.

### Embodiments (D)

In another embodiment, the Compound is according to Formula I(a) where R² is —NR³R⁴ and R³ is hydrogen, alkyl, or alkoxycarbonylalkyl; and R⁴ is optionally substituted cycloalkyl, optionally substituted phenyl, optionally substituted phenylalkyl, or optionally substituted heteroarylalkyl; and R¹ all other groups are as defined in the Summary of the Invention for a Compound of Formula I or as defined in any one of embodiments B, B1, B1a, B2, B2a, B3, (C)—C(8), and (C8a).

Embodiments (D1): In another embodiment, the Compound is according to Formula I(a) where R² is —NR³R⁴ and R³ is alkoxycarbonylalkyl; R⁴ is optionally substituted phenylalkyl; and R¹ and all other groups are as defined in the Summary of the Invention for a Compound of Formula I or as defined in any one of embodiments B, B1, B1a, B2, B2a, B3, (C)-C(8), and (C8a). In another embodiment, the Compound is according to Formula I(a) where R² is —NR³R⁴ and R³ is alkoxycarbonylalkyl; R⁴ is phenylalkyl; and R¹ and all other groups are as defined in the Summary of the Invention for a Compound of Formula I or as defined in any one of embodiments B, B1, B1a, B2, B2a, B3, (C)-C(8), and (C8a). In another embodiment, the Compound is according to Formula I(a) where R² is —NR³R⁴ and R³ is ethoxycarbonylmethyl; R⁴ is benzyl; and R¹ and all other groups are as defined in the Summary of the Invention for a Compound of Formula I or as defined in any one of embodiments B, B1, B1a, B2, B2a, B3, (C)-C(8), and (C8a).

### Embodiments (D2)

In another embodiment, the Compound is according to Formula I(a) where R² is —NR³R⁴ and R³ is hydrogen; and R⁴ is optionally substituted phenyl; and R¹ and all other groups are as defined in the Summary of the Invention for a Compound of Formula I or as defined in any one of embodiments B, B1, B1a, B2, B2a, B3, (C)-C(8), and (C8a). In another embodiment, the Compound is according to Formula I(a) where R² is —NR³R⁴ and R³ is hydrogen; and R⁴ is phenyl optionally substituted with alkyl; and R¹ and all other groups are as defined in the Summary of the Invention for a Compound of Formula I or as defined in any one of embodiments B, B1, B1a, B2, B2a, B3, (C)-C(8), and (C8a). In another embodiment, the Compound is according to Formula I(a) where R² is —NR³R⁴ and R³ is hydrogen; and R⁴ is phenyl or 4-n-pentyl-phenyl; and R¹ and all other groups are as defined in the Summary of the Invention for a Compound of Formula I or as defined in any one of embodiments B, B1, B1a, B2, B2a, B3, (C)-C(8), and (C8a).

### Embodiments (D3)

In another embodiment, the Compound is according to Formula I(a) where R² is —NR³R⁴ and R³ is alkyl; and R⁴ is optionally substituted phenylalkyl; and R¹ and all other groups are as defined in the Summary of the Invention for a Compound of Formula I or as defined in any one of embodiments B, B1, B1a, B2, B2a, B3, (C)-C(8), and (C8a). In another embodiment, the Compound is according to Formula I(a) where R² is —NR³R⁴ and R³ is alkyl; and R⁴ is phenylalkyl optionally substituted with alkyl; and R¹ and all other groups are as defined in the Summary of the Invention for a Compound of Formula I or as defined in any one of embodiments B, B1, B1a, B2, B2a, B3, (C)-C(8), and (C8a). In another embodiment, the Compound is according to Formula I(a) where R² is —NR³R⁴ and R³ is methyl, ethyl, n-propyl, isopropyl, or n-butyl; and R⁴ is 1-phenylethyl, 2-phenylethyl, phenylmethyl, 3-methyl-phenylmethyl; and R¹ and all other groups are as defined in the Summary of the Invention for a Compound of Formula I or as defined in any one of embodiments B, B1, B1a, B2, B2a, B3, (C)-C(8), and (C8a).

### Embodiments (D4)

In another embodiment, the Compound is according to Formula I(a) where R² is —NR³R⁴ and R³ is alkyl; and R⁴ is optionally substituted heteroarylalkyl; and R¹ and all other groups are as defined in the Summary of the Invention for a Compound of Formula I or as defined in any one of embodiments B, B1, B1a, B2, B2a, B3, (C)-C(8), and (C8a). In another embodiment, the Compound is according to Formula I(a) where R² is —NR³R⁴ and R³ is alkyl; and R⁴ is heteroarylalkyl; and R¹ and all other groups are asclefined in the Summary of the Invention for a Compound of Formula I or as defined in any one of embodiments B, B1, B1a, B2, B2a, B3, (C)-C(8), and (C8a). In another embodiment, the Compound is according to Formula I(a) were R² is —NR³R⁴ and R³ is methyl; and R⁴ is pyridinylmethyl; and R¹ and all other groups are as defined in the Summary of the Invention for a Compound of Formula I or as defined in any one of embodiments B, B1, B1a, B2, B2a, B3, (C)-C(8), and (C8a).

### Embodiments (D5)

In another embodiment, the Compound is according to Formula I(a) where R¹ is —NR³R⁴ and R³ is hydrogen; and R⁴ is optionally substituted cycloalkyl: and R¹ and all other groups are as defined in the Summary of the Invention for a Compound of Formula I or as defined in any one of embodiments B, B1, B1a, B2, B2a, B3, (C)-C(8), and (C8a). In another embodiment, the Compound is according to Formula I(a) where R² is —NR³R⁴ and R³ is hydrogen: and R⁴ is cycloalkyl; and R¹ and all other groups are as defined in the Summary of the invention for a Compound of Formula I or as defined in any One of embodiments B, B1, B1a, B2, B2a, B3, (C)-C(8), and (C8a). In another embodiment, the Compound is according to Formula I(a) where R² is —NR³R⁴ and R³ is hydrogen; and R⁴ is (1r,3r,5R,7R)-tricyclo[3.3.1.1^{3.7}]decan-2-yl; and R¹ and all other groups are as defined in the Suimnary of the Invention for a Compound of Formula I or as defined in any one of embodiments B, B1, B1a, B2, B2a, B3, (C)-C(8), and (C8a).

Embodiment (D6): In another embodiment, the Compound is according to Formula I(a) where
R¹ is phenyl substituted with one. or two R⁶ groups independently nitro, —NR^{8e}, —C(O)NR⁸R^{8a}, —NRC(O)OR⁹, or heteroaryl optionally substituted with 1, 2, or 3 R¹⁴; or
R¹ is heteroaryl optionally substituted with one, two, or three R¹;
R² is —NR³R⁴ where R³ is hydrogen, alkyl, or alkoxycarbonylalkyl; and R⁴ is optionally substituted cycloalkyl, optionally substituted phenyl, optionally substituted phenylalkyl, or optionally substituted heteroarylalkyl:
each R⁷, when present, is independently alkyl, —NR⁸R^{8a}, —C(O)NR⁸R^{8a}, —NR⁸C(O)OR⁹, or —NR⁸C(O)R⁹;
R⁸ is hydrogen, alkyl, or alkenyl;
R^{8a} is hydrogen, alkyl, haloalkyl, optionally substituted heteroeycloalkyl, or optionally substituted phenylalkyl:
R⁹ is alkyl or haloalkyl; and
each R¹⁴, when present, is halo, alkyl, or alkoxycarbonyl.

### Embodiments (E)

In another embodiment, the Compound is according to Formula I(a) where R² is —NR³R⁴ and R³ and R⁴ together with the nitrogen to which they are attached form HET optionally substituted on any substitutable atom of the ring with R¹⁰, R^{10a}, R^{10b}, R^{10c}, R^{10d}, R^{10e}, and R^{10f}; and HET, R¹⁰, R^{10a}, R^{10b}, R^{10c}, R^{10d}, R^{10e}, R^{10f} and all other groups are as defined in the Summary of the Invention for a Compound of Formula I or as defined in any one of embodiments B, B1, B1a, B2, B2a, B3, (C)-C(8), and (C8a).

### Embodiments (E1)

In another embodiment, the Compound is according to Formula I(a) where R² is —NR³R⁴ and R³ and R⁴ together with the nitrogen to which they are attached form HET optionally substituted on any substitutable atom of the ring with R¹⁰, R^{10a}, R^{10b}, R^{10c}, R^{10d}, R^{10e}, and R^{10f}; BET is a saturated or partially unsaturated, but non-aromatic, monocyclic 5- to 8-membered ring optionally containing an additional one or two ring heteroatoms which are independently oxygen, sulfur, or nitrogen where the remaining ring atoms are carbon; and R¹⁰, R^{10a}, R^{10b}, R^{10c}, R^{10d}, R^{10e}, R^{10f} and all other groups are as defined in the Summary of the Invention for a Compound of Formula I or as defined in any one of embodiments B, B1, B1a, B2, B2a, B3, (C)-C(8), and (C8a).

### Embodiments (E2)

In another embodiment, the Compound is according to Formula I(a) where R³ and R⁴ together with the nitrogen to which they are attached form HET optionally substituted on any substitutable atom of the ring with R¹⁰, R^{10a}, R^{10b}, R^{10c}, R^{10d}, R^{10e}, and R^{10f}; HET is a partially unsaturated, but not aromatic, monocyclic 5- to 8-membered ring optionally containing an additional one or two ring heteroatoms which are independently oxygen, sulfur, or nitrogen and the remaining ring atoms are carbon and which ring is fused to a benzo ring; and R¹⁰, R^{10a}, R^{10b}, R^{10c}, R^{10d}, R^{10e}, and R^{10f} and all other groups are as defined in the Summary of the Invention for a Compound of Formula I or as defined in any one of embodiments B, B1, B1a, B2, B2a, B3, (C)-C(8), and (C8a). In another embodiment, the Compound is according to Formula I(a) where R³ and R⁴ together with the nitrogen to which they are attached form BET optionally substituted on any substitutable atom of the ring with R¹⁰, R^{10a}, and R^{10b}; R^{10c}, R^{10d}, R^{10e}, and R^{10f} are hydrogen; HET is a partially unsaturated, but not aromatic, monocyclic 5- to 8-membered ring optionally containing an additional one or two ring heteroatoms which are independently oxygen, sulfur, or nitrogen and the remaining ring atoms are carbon and which ring is fused to a benzo ring; and R¹⁰, R^{10a}, R^{10b}, and all other groups are as defined in the Summary of the Invention for a Compound of Formula I or as defined in any one of embodiments B, B1, B1a, B2, B2a, B3, (C)-C(8), and (C8a).

### Embodiments (E3)

In another embodiment, the Compound is according to Formula I(a) where R³ and R⁴ together with the nitrogen to which they are attached form HET optionally substituted on any substitutable atom of the ring with R¹⁰, R^{10a}, R^{10b}, R^{10c}, R^{10d}, R^{10e}, and R^{10f}; HET is a fused, bridged, or spirocyclic, bicyclic 7- to 11-membered ring optionally containing an additional one or two heteroatoms which are independently oxygen, sulfur, or nitrogen and the remaining ring atoms are carbon and where each ring of the 7- to 11-membered ring is saturated or partially unsaturated but not fully aromatic; and R¹⁰, R^{10a}, R^{10b}, R^{10c}, R^{10d}, R^{10e}, and R^{10f} and all other groups are as defined in the Summary of the Invention for a Compound of Formula I or as defined in any one of embodiments B, B1, B1a, B2, B2a, B3, (C)-C(8), and (C8a). In another embodiment, the Compound is according to Formula I(a) where R³ and R⁴ together with the nitrogen to which they are attached form HET optionally substituted on any substitutable atom of the ring with R¹⁰, R^{10a}, and R^{10b}; R^{10c}, R^{10d}, R^{10e}, and R^{10f} are hydrogen; BET is a fused, bridged, or spirocyclic, bicyclic 7- to 11-membered ring optionally containing an additional one or two heteroatoms which are independently oxygen, sulfur, or nitrogen and the remaining ring atoms are carbon and where each ring of the 7- to 11-membered ring is saturated or partially unsaturated but not fully aromatic; and R¹⁰, R^{10a}, and R^{10b} and all other groups are as defined in the Summary of the Invention for a Compound of Formula I or as defined in any one of embodiments B, B1, B1a, B2, B2a, B3, (C)-C(8), and (C8a).

### Embodiments (E4)

In another embodiment, the Compound is according to Formula I(a) where R³ and R⁴ together with the nitrogen to which they are attached form HET optionally substituted on any substitutable atom of the ring with R¹⁰, R^{10a}, R^{10b}, R^{10c}, R^{10d}, R^{10e}, and R^{10f}; HET is a fused, bridged, or spirocyclic, bicyclic 7- to 11-membered ring optionally containing an additional one or two ring heteroatoms which are independently oxygen, sulfur, or nitrogen and the remaining ring atoms are carbon where each ring of the bicyclic 7- to 11-membered ring is saturated or partially unsaturated but not fully aromatic, and where the bicyclic 7- to 11-membered ring is fused to a benzo ring; and R¹⁰, R^{10a}, R^{10b}, R^{10c}, R^{10d}, R^{10e}, and R^{10f} and all other groups are as defined in the Summary of the Invention for a Compound of Formula I or as defined in any one of embodiments B, B1, B1a, B2, B2a, B3, (C)-C(8), and (C8a). In another embodiment, the Compound is according to Formula I(a) where R³ and R⁴ together with the nitrogen to which they are attached form HET optionally substituted on any substitutable atom of the ring with R¹⁰, R^{10a}, R^{10b}, R^{10c}, R^{10d}, R^{10e}, and R^{10f}; HET is a fused, bridged, or spirocyclic, bicyclic 7- to 11-membered ring optionally containing an additional one or two ring heteroatoms which are independently oxygen, sulfur, or nitrogen and the remaining ring atoms are carbon where each ring of the bicyclic 7- to 11-membered ring is saturated or partially unsaturated but not fully aromatic, and where the bicyclic 7- to 11-membered ring is fused to a benzo ring; R¹⁰, R^{10a}, R^{10b}, R^{10c}, R^{10d}, R^{10e}, and R^{10f} are hydrogen; and all other groups are as defined in the Summary of the Invention for a Compound of Formula I or as defined in any one of embodiments B, B1, B1a, B2, B2a, B3, (C)-C(8), and (C8a).

### Embodiments (F)

In another embodiment, the Compound is according to Formula I(a) where R² is —NR³R⁴ and R³ and R⁴ together with the nitrogen to which they are attached form HET and HET is indolin-1-yl, isoindolin-2-yl, 1,2,3,4-tetrahydroquinolin-1-yl, 1,2,3,4-tetrahydroisoquinolin-2-yl, or 1,2,3,4-tetrahydro-1,4-epiminonaphth-9-yl, where any substitutable atom on HET is optionally substituted with R¹⁰, R^{10a}, and R^{10b}; and R¹⁰, R^{10a}, R^{10b} and all other groups are as defined in the Summary of the Invention for a Compound of Formula I or as defined in any one of embodiments B, B1, B1a, B2, B2a, B3, (C)-C(8), and (C8a). In another embodiment, the Compound is according to Formula I(a) where R² is —NR³R⁴ and R³ and R⁴ together with the nitrogen to which they are attached form HET and HET is indolin-1-yl, isoindolin-2-yl, 1,2,3,4-tetrahydroquinolin-1-yl, 1,2,3,4-tetrahydroisoquinolin-2-yl, or 1,2,3,4-tetrahydro-1,4-epiminonaphth-9-yl, where any substitutable atom on HET is optionally substituted with R¹⁰, R^{10a}, and R^{10b}; R¹⁰ is hydrogen or phenyl; R^{10a} and R^{10b} are hydrogen; and all other groups are as defined in the Summary of the Invention for a Compound of Formula I or as defined in any one of embodiments B, B1, B1a, B2, B2a, B3, (C)-C(8), and (C8a). In another embodiment, the Compound is according to Formula I(a) where R² is —NR³R⁴ and R³ and R⁴ together with the nitrogen to which they are attached form HET and HET is indolin-1-yl, isoindolin-2-yl, 1,2,3,4-tetrahydroquinolin-1-yl, 1,2,3,4-tetrahydroisoquinolin-2-yl, or 1,2,3,4-tetrahydro-1,4-epiminonaphth-9-yl, where any substitutable atom on HET is optionally substituted with R¹⁰, R^{10a}, and R^{10b}; R¹⁰, R^{10a} and R^{10b} are hydrogen; and all other groups are as defined in the Summary of the Invention for a Compound of Formula I or as defined in any one of embodiments B, B1, B1a, B2, B2a, B3, (C)-C(8), and (C8a).

### Embodiments (F1)

In another embodiment, the Compound is according to Formula I(a) where
R¹ is phenyl substituted with one or two R⁶ groups independently nitro, —NR⁸R^{8a}, —C(O)NR⁸R^{8a}, —NR⁸C(O)OR⁹, or heteroaryl optionally substituted with 1, 2, or 3 R¹⁴; or
R¹ is heteroaryl optionally substituted with one, two, or three R⁷;
R² is —NR³R⁴ and R³ and R⁴ together with the nitrogen to which they are attached form HET and HET is indolin-1-yl, isoindolin-2-yl, 1,2,3,4-tetrahydroquinolin-1-yl, 1,2,3,4-tetrahydroisoquinolin-2-yl, or 1,2,3,4-tetrahydro-1,4-epiminonaphth-9-yl, where any substitutable atom on HET is optionally substituted with R¹⁰, R^{10a}, and R^{10b};
each R⁷, when present, is independently alkyl, —NR⁸R^{8a}, —C(O)NR⁸R^{8a}, —NR⁸C(O)OR⁹, or —NR⁸C(O)R⁹;
R⁸ is hydrogen, alkyl, or alkenyl;
R^{8a} is hydrogen, alkyl, haloalkyl, optionally substituted heterocycloalkyl, or optionally substituted phenylalkyl;
R⁹ is alkyl or haloalkyl; and
R¹⁰, R^{10a}, and R^{10b} are independently hydrogen; halo; alkyl; haloalkyl; haloalkenyl; hydroxyalkyl; alkylthio; alkylsulfonyl; hydroxy; alkoxy; haloalkoxy; cyano; alkoxycarbonyl; carboxy; amino; alkylamino; dialkylamino; —C(O)R¹²; —C(O)NR¹¹R^{11a}; optionally substituted cycloalkyl; optionally substituted cycloalkylalkyl; optionally substituted phenyl; optionally substituted phenylalkyl; optionally substituted phenyloxy; optionally substituted phenyloxyalkyl; optionally substituted heterocycloalkyl; optionally substituted heterocycloalkylalkyl; optionally substituted heteroaryl; or optionally substituted heteroarylalkyl; or two of R¹⁰, R^{10a}, R^{10b}, R^{10c}, R^{10d}, R^{10e}, and R^{10f} when attached to the same carbon form oxo;
R¹¹ hydrogen, alkyl, or alkenyl;
R^{11a} hydrogen, alkyl, or alkenyl;
R¹² is alkyl, or optionally substituted heteroaryl; and
each R¹⁴, when present, is halo, alkyl, or alkoxycarbonyl.

### Embodiments (F2)

In another embodiment, the Compound is according to Formula I(a) where
R¹ is phenyl substituted with one or two R⁶ groups independently nitro, —NR⁸R^{8a}, —C(O)NR⁸R^{8a}, —NR⁸C(O)OR⁹, or heteroaryl optionally substituted with 1, 2, or 3 R¹⁴; or
R¹ is heteroaryl optionally substituted with one, two, or three R⁷;
R² is —NR³R⁴ and R³ and R⁴ together with the nitrogen to which they are attached form HET and BET is indolin-1-yl, isoindolin-2-yl, 1,2,3,4-tetrahydroquinolin-1-yl, 1,2,3,4-tetrahydroisoquinolin-2-yl, or 1,2,3,4-tetrahydro-1,4-epiminonaphth-9-yl, where any substitutable atom on HET is optionally substituted with R¹⁰, R^{10a}, and R^{10b};
each R⁷, when present, is independently alkyl, —NR⁸R^{8a}, —C(O)NR⁸R^{8a}, —NR⁸C(O)OR⁹, or —NR⁸C(O)R⁹;
R^{5b} is (C₁₋₃)alkyl or halo(C₁₋₃)alkyl;
R⁸ is hydrogen, alkyl, or alkenyl;
R^{8a} is hydrogen, alkyl, haloalkyl, optionally substituted heterocycloalkyl, or optionally substituted phenylalkyl;
R⁹ is alkyl or haloalkyl; and
R¹⁰, R^{10a}, and R^{10b} are independently hydrogen, alkyl, or optionally substituted phenyl;
R¹¹ hydrogen, alkyl, or alkenyl;
R^{11a} hydrogen, alkyl, or alkenyl;
R¹² is alkyl, or optionally substituted heteroaryl; and
each R¹⁴, when present, is halo, alkyl, or alkoxycarbonyl.

### Embodiments (G)

In another embodiment, the Compound is according to Formula I(a) where R² is —NR³R⁴ and R³ and R⁴ together with the nitrogen to which they are attached form HET according to formula (a):

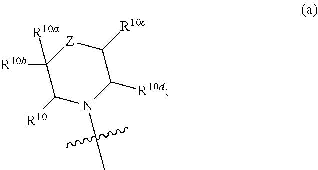

where Z is a bond, —C(O)—, —O—, —S—, —S(O)—, —S(O)₂—, —N(R^{z})—, —C(R^{10e})(R^{10f})—, or C₂₋₃-alkylene; R^{z} is hydrogen, alkyl, haloalkyl, haloalkenyl, hydroxyalkyl, alkylsulfonyl, hydroxy, alkoxy, alkoxycarbonyl, —C(O)R¹², —C(O)NR¹¹R^{11a}, optionally substituted cycloalkyl, optionally substituted cycloalkylalkyl, optionally substituted phenyl, optionally substituted phenylalkyl, optionally substituted heterocycloalkyl, optionally substituted heterocycloalkylalkyl, optionally substituted heteroaryl, or optionally substituted heteroarylalkyl; R¹⁰, R^{10a}, R^{10b}, R^{10c}, R^{10d}, R^{10e}, and R^{10f} are independently hydrogen; halo; alkyl; haloalkyl; haloalkenyl; hydroxyalkyl; alkylthio; alkylsulfonyl; hydroxy; alkoxy; haloalkoxy; cyano; alkoxycarbonyl; carboxy; amino; alkylamino; dialkylamino; —C(O)R¹²; —C(O)NR¹¹R^{11a}; optionally substituted cycloalkyl; optionally substituted cycloalkylalkyl; optionally substituted phenyl; optionally substituted phenylalkyl; optionally substituted phenyloxy; optionally substituted phenyloxyalkyl; optionally substituted heterocycloalkyl; optionally substituted heterocycloalkylalkyl; optionally substituted heteroaryl; or optionally substituted heteroarylalkyl; or R¹⁰ and R^{10b} together form oxo; and all other groups are as defined in the Summary of the Invention for a Compound of Formula I or as defined in any one of embodiments B, B1, B1a, B2, B2a, B3, (C)-C(8), and (C8a).

### Embodiments (G1)

In another embodiment, the Compound is according to Formula I(a) where R² is —NR³R⁴ and R³ and R⁴ together with the nitrogen to which they are attached form HET according to formula (a):

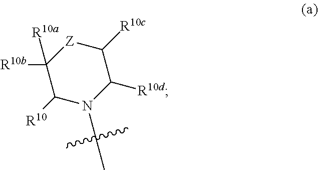

where Z is a bond; R¹⁰, R^{10a}, R^{10b}, R^{10c}m, and R^{10d} are independently hydrogen; halo; alkyl; haloalkyl; haloalkenyl; hydroxyalkyl; alkylthio; alkylsulfonyl; hydroxy; alkoxy; haloalkoxy; cyano; alkoxycarbonyl; carboxy; amino; alkylamino; dialkylamino; —C(O)R¹²; —C(O)NR¹¹R^{11a}; optionally substituted cycloalkyl; optionally substituted cycloalkylalkyl; optionally substituted phenyl; optionally substituted phenylalkyl; optionally substituted phenyloxy; optionally substituted phenyloxyalkyl; optionally substituted heterocycloalkyl; optionally substituted heterocycloalkylalkyl; optionally substituted heteroaryl; or optionally substituted heteroarylalkyl; or R^{10a} and R^{10b} together form oxo; and all other groups are as defined in the Summary of the Invention for a Compound of Formula I or as defined in any one of embodiments B, B1, B1a, B2, B2a, B3, (C)-C(8), and (C8a).

### Embodiments (G1a)

In another embodiment, the Compound is according to Formula I(a) where R² is —NR³R⁴ and R³ and R⁴ together with the nitrogen to which they are attached form HET according to formula (a) where Z is bond; one of R¹⁰, R^{10a}, R^{10b}, R^{10c}, and R^{10d} is alkyl, halo, haloalkyl, haloalkenyl, hydroxyalkyl, alkylthio, alkylsulfonyl, hydroxy, alkoxy, haloalkoxy, cyano, alkoxycarbonyl, carboxy, amino, alkylamino, dialkylamino, —C(O)R¹², —C(O)NR¹¹R^{11a}, optionally substituted cycloalkyl, optionally substituted cycloalkylalkyl, optionally substituted phenyl, optionally substituted phenylalkyl, optionally substituted phenyloxy, optionally substituted phenyloxyalkyl, optionally substituted heterocycloalkyl, optionally substituted heterocycloalkylalkyl, optionally substituted heteroaryl, or optionally substituted heteroarylalkyl; the remaining of R¹⁰, R^{10a}, R^{10b}, R^{10c}, and R^{10d} are hydrogen; and all other groups are as defined in the Summary of the Invention for a Compound of Formula I or as defined in any one of embodiments B, B1, B1a, B2, B2a, B3, (C)-C(8), and (C8a).

### Embodiments (G1b)

In another embodiment, the Compound is according to Formula I(a) where R² is —NR³R⁴ and R³ and R⁴ together with the nitrogen to which they are attached form HET according to formula (a) where Z is bond; R^{10a} is hydrogen, hydroxy, optionally substituted phenyl, or optionally substituted phenylalkyl; R¹⁰, R^{10b}, R^{10c}, and R^{10d} are hydrogen; and all other groups are as defined in the Summary of the Invention for a Compound of Formula I or as defined in any one of embodiments B, B1, B1a, B2, B2a, B3, (C)-C(8), and (C8a). In another embodiment, the Compound is according to Formula I(a) where R² is —NR³R⁴ and R³ and R⁴ together with the nitrogen to which they are attached form HET according to formula (a) where Z is bond; R¹⁰ is alkyl, optionally substituted phenyl, or optionally substituted phenylalkyl; R^{10a}, R^{10b}, R^{10c}, and R^{10d} are hydrogen; and all other groups are as defined in the Summary of the Invention for a Compound of Formula I or as defined in any one of embodiments B, B1, B1a, B2, B2a, B3, (C)-C(8), and (C8a).

### Embodiments (G2)

In another embodiment, the Compound is according to Formula I(a) where R² is —NR³R⁴ and R³ and R⁴ together with the nitrogen to which they are attached form HET according to formula (a):

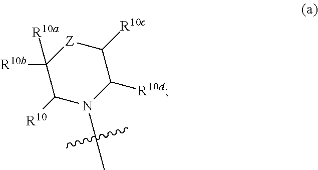

where Z is —O—; R¹⁰, R^{10a}, R^{10b}, R^{10c}, and R^{10d} are independently hydrogen; halo; alkyl; haloalkyl; haloalkenyl; hydroxyalkyl; alkylthio; alkylsulfonyl; hydroxy; alkoxy; haloalkoxy; cyano; alkoxycarbonyl; carboxy; amino; alkylamino; dialkylamino; —C(O)R¹²; —C(O)NR¹¹R^{11a}; optionally substituted cycloalkyl; optionally substituted cycloalkylalkyl; optionally substituted phenyl; optionally substituted phenylalkyl; optionally substituted phenyloxy; optionally substituted phenyloxyalkyl; optionally substituted heterocycloalkyl; optionally substituted heterocycloalkylalkyl; optionally substituted heteroaryl; or optionally substituted heteroarylalkyl; or R^{10a} and R^{10b} together form oxo; and all other groups are as defined in the Summary of the Invention for a Compound of Formula I. In another embodiment, the Compound is according to Formula I(a) where R² is —NR³R⁴ and R³ and R⁴ together with the nitrogen to which they are attached form HET according to formula (a) where Z is —O—; R¹⁰, R^{10a}, R^{10b}, R^{10c}, and R^{10d} are hydrogen; and all other groups are as defined in the Summary of the Invention for a Compound of Formula I or as defined in any one of embodiments B, B1, B1a, B2, B2a, B3, (C)-C(8), and (C8a).

### Embodiments (G2a)

In another embodiment, the Compound is according to Formula I(a) where R² is —NR³R⁴ and R³ and R⁴ together with the nitrogen to which they are attached form HET according to formula (a) where Z is —O—; one of R¹⁰, R^{10a}, R^{10b}, R^{10c}, and R^{10d} is alkyl, halo, haloalkyl, haloalkenyl, hydroxyalkyl, alkylthio, alkylsulfonyl, hydroxy, alkoxy, haloalkoxy, cyano, alkoxycarbonyl, carboxy, amino, alkylamino, dialkylamino, C(O)R¹², —C(O)NR¹¹R^{11a}, optionally substituted cycloalkyl, optionally substituted cycloalkylalkyl, optionally substituted phenyl, optionally substituted phenylalkyl, optionally substituted phenyloxy, optionally substituted phenyloxyalkyl, optionally substituted heterocycloalkyl, optionally substituted heterocycloalkylalkyl, optionally substituted heteroaryl, or optionally substituted heteroarylalkyl; the remaining of R¹⁰, R^{10a}, R^{10b}, R^{10c}, and R^{10d} are hydrogen; and all other groups are as defined in the Summary of the Invention for a Compound of Formula I or as defined in any one of embodiments B, B1, B1a, B2, B2a, B3, (C)-C(8), and (C8a).

### Embodiments (G2b)

In another embodiment, the Compound is according to Formula I(a) where R² is —NR³R⁴ and R³ and R⁴ together with the nitrogen to which they are attached form HET according to formula (a) where Z is —O—; R^{10a} is optionally substituted phenyloxyalkyl; R¹⁰, R^{10a}, R^{10b}, R^{10c}, and R^{10d} are hydrogen; and all other groups are as defined in the Summary of the Invention for a Compound of Formula I or as defined in any one of embodiments B, B1, B1a, B2, B2a, B3, (C)-C(8), and (C8a).

### Embodiments (G3)

In another embodiment, the Compound is according to Formula I(a) where R² is —NR³R⁴ and R³ and R⁴ together with the nitrogen to which they are attached form HET according to formula (a):

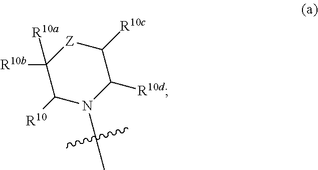

where Z is —S—, —S(O)—, or —S(O)₂—; R¹⁰, R^{10a}, R^{10b}, R^{10c}, and R^{10d} are hydrogen; and all other groups are as defined in the Summary of the Invention for a Compound of Formula I or as defined in any one of embodiments B, B1, B1a, B2, B2a, B3, (C)-C(8), and (C8a).

Embodiments (G4): In another embodiment, the Compound is according to Formula I(a) where R² is —NR³R⁴ and R³ and R⁴ together with the nitrogen to which they are attached form HET according to formula (a):

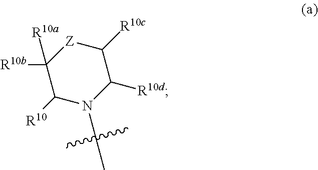

where Z is —N(R^{z})—; R^{z} is hydrogen, alkyl, haloalkyl, haloalkenyl, hydroxyalkyl, alkylsulfonyl, hydroxy, alkoxy, alkoxycarbonyl, —C(O)R¹², —C(O)NR¹¹R^{11a}, optionally substituted cycloalkyl, optionally substituted cycloalkylalkyl, optionally substituted phenyl, optionally substituted phenylalkyl, optionally substituted heterocycloalkyl, optionally substituted heterocycloalkylalkyl, optionally substituted heteroaryl, or optionally substituted heteroarylalkyl; R¹⁰, R^{10a}, R^{10b}, R^{10c}, and R^{10d} are independently hydrogen; halo; alkyl; haloalkyl; haloalkenyl; hydroxyalkyl; alkylthio; alkylsulfonyl; hydroxy; alkoxy; haloalkoxy; cyano; alkoxycarbonyl; carboxy; amino; alkylamino; dialkylamino; —C(O)R¹²; —C(O)NR¹¹R^{11a}; optionally substituted cycloalkyl; optionally substituted cycloalkylalkyl; optionally substituted phenyl; optionally substituted phenylalkyl; optionally substituted phenyloxy; optionally substituted phenyloxyalkyl; optionally substituted heterocycloalkyl; optionally substituted heterocycloalkylalkyl; optionally substituted heteroaryl; or optionally substituted heteroarylalkyl; or R^{10a} and R^{10b} together form oxo; and all other groups are as defined in the Summary of the Invention for a Compound of Formula I or as defined in any one of embodiments B, B1, B1a, B2, B2a, B3, (C)-C(8), and (C8a). In another embodiment, the Compound is according to Formula I(a) where R² is —NR³R⁴ and R³ and R⁴ together with the nitrogen to which they are attached form HET according to formula (a) where Z is —N(R^{z})—; R¹⁰, R^{10a}, R^{10b}, R^{10c}, and R^{10d} are hydrogen; R^{z} is hydrogen, alkyl, haloalkyl, haloalkenyl, hydroxyalkyl, alkylsulfonyl, hydroxy, alkoxy, alkoxycarbonyl, —C(O)R¹², —C(O)NR¹¹R^{11a}, optionally substituted cycloalkyl, optionally substituted cycloalkylalkyl, optionally substituted phenyl, optionally substituted phenylalkyl, optionally substituted heterocycloalkyl, optionally substituted heterocycloalkylalkyl, optionally substituted heteroaryl, or optionally substituted heteroarylalkyl; and all other groups are as defined in the Summary of the Invention for a Compound of Formula I or as defined in any one of embodiments B, B1, B1a, B2, B2a, B3, (C)-C(8), and (C8a).

### Embodiments (G4a)

In another embodiment, the Compound is according to Formula I(a) where R² is —NR³R⁴ and R³ and R⁴ together with the nitrogen to which they are attached form HET according to formula (a) where Z is —N(R^{z})—; one of R^{z}, R¹⁰, R^{10a}, R^{10b}, R^{10c}, and R^{10d} is not hydrogen; the remaining of R^{z}, R¹⁰, R^{10a}, R^{10b}, R^{10c}, and R^{10d} are hydrogen; and all other groups are as defined in the Summary of the Invention for a Compound of Formula I or as defined in any one of embodiments B, B1, B1a, B2, B2a, B3, (C)-C(8), and (C8a).

### Embodiments (G4b)

In another embodiment, the Compound is according to Formula I(a) where R² is —NR³R⁴ and R³ and R⁴ together with the nitrogen to which they are attached form HET according to formula (a) where Z is —N(R^{z})—; R^{z} is alkyl, haloalkyl, haloalkenyl, hydroxyalkyl, alkylsulfonyl, hydroxy, alkoxy, alkoxycarbonyl, —C(O)R¹², C(O)NR¹¹R^{11a}, optionally substituted cycloalkyl, optionally substituted cycloalkylalkyl, optionally substituted phenyl, optionally substituted phenylalkyl, optionally substituted heterocycloalkyl, optionally substituted heterocycloalkylalkyl, optionally substituted heteroaryl, or optionally substituted heteroarylalkyl; R¹⁰, R^{10a}, R^{10b}, R^{10c}, and R^{10d} are hydrogen; and all other groups are as defined in the Summary of the Invention for a Compound of Formula I or as defined in any one of embodiments B, B1, B1a, B2, B2a, B3, (C)-C(8), and (C8a).

### Embodiments (G4c)

In another embodiment, the Compound is according to Formula I(a) where R² is —NR³R⁴ and R³ and R⁴ together with the nitrogen to which they are attached form HET according to formula (a) where Z is —N(R^{z})—; R^{z} is alkyl, optionally substituted phenyl, optionally substituted phenylalkyl, optionally substituted heteroaryl, or —C(O)R¹²; R¹⁰, R^{10a}, R^{10b}, R^{10c}, and R^{10d} and all other groups are as defined in the Summary of the Invention for a Compound of Formula I or as defined in any one of embodiments B, B1, B1a, B2, B2a, B3, (C)-C(8), and (C8a). In another embodiment, the Compound is according to Formula I(a) where R² is —NR³R⁴ and R³ and R⁴ together with the nitrogen to which they are attached form HET according to formula (a) where Z is —N(R^{z})—; R^{z} is alkyl; or R^{z} is phenyl optionally substituted with one, two, or three groups which are independently halo, haloalkyl, hydroxy, alkyl, alkoxy, alkylcarbonyl, and nitro; or R^{z} is phenylmethyl optionally substituted with one, two, or three groups which are independently halo, haloalkyl, hydroxy, alkyl, alkoxy, alkylcarbonyl, or nitro; or R^{z} is heteroaryl optionally substituted with one, two, or three groups which are independently halo, haloalkyl, hydroxy, alkyl, alkoxy, alkylcarbonyl, or nitro; and R¹⁰, R^{10a}, R^{10b}, R^{10c}, and R^{10d} are hydrogen; and all other groups are as defined in the Summary of the Invention for a Compound of Formula I or as defined in any one of embodiments B, B1, B1a, B2, B2a, B3, (C)-C(8), and (C8a).

### Embodiments (G4d)

In another embodiment, the Compound is according to Formula I(a) where R² is —NR³R⁴ and R³ and R⁴ together with the nitrogen to which they are attached form HET according to formula (a) where Z is —N(R^{z})—; R¹⁰ and R^{z} are independently alkyl, haloalkyl, haloalkenyl, hydroxyalkyl, alkylsulfonyl, hydroxy, alkoxy, alkoxycarbonyl, —C(O)R¹², —C(O)NR¹¹R^{11a}, optionally substituted cycloalkyl, optionally substituted cycloalkylalkyl, optionally substituted phenyl, optionally substituted phenylalkyl, optionally substituted heterocycloalkyl, optionally substituted heterocycloalkylalkyl, optionally substituted heteroaryl, or optionally substituted heteroarylalkyl; R^{10a}, R^{10b}, R^{10c}, and R^{10d} are hydrogen; and all other groups are as defined in the Summary of the Invention for a Compound of Formula I or as defined in any one of embodiments B, B1, B1a, B2, B2a, B3, (C)-C(8), and (C8a).

### Embodiments (G4e)

In another embodiment, the Compound is according to Formula I(a) where R² is —NR³R⁴ and R³ and R⁴ together with the nitrogen to which they are attached form HET according to formula (a) where Z is —N(R^{z})—; R¹⁰ is optionally substituted phenyl; R^{z} is alkyl or optionally substituted phenyl; R^{10a}, R^{10b}, R^{10c}, and R^{10d} are hydrogen; and all other groups are as defined in the Summary of the Invention for a Compound of Formula I or as defined in any one of embodiments B, B1, B1a, B2, B2a, B3, (C)-C(8), and (C8a). In another embodiment, the Compound is according to Formula I(a) where R² is —NR³R⁴ and R³ and R⁴ together with the nitrogen to which they are attached form HET according to formula (a) where Z is —N(R²)—; R¹⁰ is phenyl optionally substituted with one, two, or three groups which are independently halo, haloalkyl, hydroxy, alkyl, alkoxy, alkylcarbonyl, or nitro; R^{z} is alkyl, or phenyl optionally substituted with one, two, or three groups which are independently halo, haloalkyl, hydroxy, alkyl, alkoxy, alkylcarbonyl, or nitro; R^{10a}, R^{10b}, R^{10c}, and R^{10d} are hydrogen; and all other groups are as defined in the Summary of the Invention for a Compound of Formula I or as defined in any one of embodiments B, B1, B1a, B2, B2a, B3, (C)-C(8), and (C8a).

### Embodiments (G4f)

In another embodiment, the Compound is according to Formula I(a) where R² is —NR³R⁴ and R³ and R⁴ together with the nitrogen to which they are attached form HET according to formula (a) where Z is —N(R^{z})—; R^{z} is alkyl; R^{10a} and R^{10b} together form oxo; R¹⁰, R^{10c}, and R^{10d} are hydrogen; and all other groups are as defined in the Summary of the Invention for a Compound of Formula I or as defined in any one of embodiments B, B1, B1a, B2, B2a, B3, (C)-C(8), and (C8a).

### Embodiments (G5)

In another embodiment, the Compound is according to Formula I(a) where R² is —NR³R⁴ and R³ and R⁴ together with the nitrogen to which they are attached form HET according to formula (a):

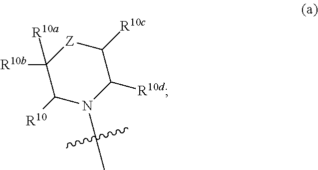

where Z is —C(R^{10e})(R^{10f})—; R¹⁰, R^{10a}, R^{10b}, R^{10c}, R^{10d}, R^{10e}, and R^{10f} are independently hydrogen; halo; alkyl; haloalkyl; haloalkenyl; hydroxyalkyl; alkylthio; alkylsulfonyl; hydroxy; alkoxy; haloalkoxy; cyano; alkoxycarbonyl; carboxy; amino; alkylamino; dialkylamino; —C(O)R¹²; —C(O)NR¹¹R^{11a}; optionally substituted cycloalkyl; optionally substituted cycloalkylalkyl; optionally substituted phenyl; optionally substituted phenylalkyl; optionally substituted phenyloxy; optionally substituted phenyloxyalkyl; optionally substituted heterocycloalkyl; optionally substituted heterocycloalkylalkyl; optionally substituted heteroaryl; or optionally substituted heteroarylalkyl; or R^{10a} and R^{10b} together form oxo; or R^{10e} and R^{10f} together form oxo; and all other groups are as defined in the Summary of the Invention for a Compound of Formula I or as defined in any one of embodiments B, B1, B1a, B2, B2a, B3, (C)-C(8), and (C8a). In another embodiment, the Compound is according to Formula I(a) where R² is —NR³R⁴ and R³ and R⁴ together with the nitrogen to which they are attached form HET according to formula (a) where Z is —C(R^{10e})(R^{10f})—; R¹⁰, R^{10a}, R^{10b}, R^{10c}, R^{10d}, R^{10e}, and R^{10f} are hydrogen; and all other groups are as defined in the Summary of the Invention for a Compound of Formula I or as defined in any one of embodiments B, B1, B1a, B2, B2a, B3, (C)-C(8), and (C8a). In another embodiment, the Compound is according to Formula I(a) where R² is —NR³R⁴ and R³ and R⁴ together with the nitrogen to which they are attached form HET according to formula (a) where Z is —C(R^{10e})(R^{10f})—; R^{10e} and R^{10f} together form oxo; R¹⁰, R^{10a}, R^{10b}, R^{10c}, and R^{10d} are hydrogen; and all other groups are as defined in the Summary of the Invention for a Compound of Frmula I or as defined in any one of embodiments B, B1, B1a, B2, B2a, B3, (C)-C(8), and (C8a).

### Embodiments (G5a)

In another embodiment, the Compound is according to Formula I(a) where R² is —NR³R⁴ and R³ and R⁴ together with the nitrogen to which they are attached form HET according to formula (a) where Z is —C(R^{10e})(R^{10f})—; one of R¹⁰, R^{10a}, R^{10b}, R^{10c}, R^{10d}, R^{10e}, and R^{10f} is alkyl, halo, haloalkyl, haloalkenyl, hydroxyalkyl, alkylthio, alkylsulfonyl, hydroxy, alkoxy, haloalkoxy, cyano, alkoxycarbonyl, carboxy, amino, alkylamino, dialkylamino, —C(O)R¹², —C(O)NR¹¹R^{11a}, optionally substituted cycloalkyl, optionally substituted cycloalkylalkyl, optionally substituted phenyl, optionally substituted phenylalkyl, optionally substituted phenyloxy, optionally substituted phenyloxyalkyl, optionally substituted heterocycloalkyl, optionally substituted heterocycloalkylalkyl, optionally substituted heteroaryl, or optionally substituted heteroarylalkyl; the remaining of R¹⁰, R^{10a}, R^{10b}, R^{10c}, R^{10d}, R^{10e}, and R^{10f} are hydrogen; and all other groups are as defined in the Summary of the Invention for a Compound of Formula I or as defined in any one of embodiments B, B1, B1a, B2, B2a, B3, (C)-C(8), and (C8a).

### Embodiments (G5b)

In another embodiment, the Compound is according to Formula I(a) where R² is —NR³R⁴ and R³ and R⁴ together with the nitrogen to which they are attached form HET according to formula (a) where Z is —C(R^{10e})(R^{10f})—; one of R¹⁰, R^{10a}, R^{10b}, R^{10c}, R^{10d}, R^{10e}, and R^{10f} is alkyl, halo, haloalkyl, haloalkenyl, hydroxyalkyl, alkylthio, alkylsulfonyl, hydroxy, alkoxy, cyano, alkoxycarbonyl, —C(O)NR¹¹R^{11a}, optionally substituted cycloalkyl, optionally substituted phenyl, optionally substituted phenylalkyl, optionally substituted phenyloxy, optionally substituted heterocycloalkyl, or optionally substituted heteroaryl; the remaining of R¹⁰, R^{10a}, R^{10b}, R^{10c}, R^{10d}, R^{10e}, and R^{10f} are hydrogen; and all other groups are as defined in the Summary of the Invention for a Compound of Formula I or as defined in any one of embodiments B, B1, B1a, B2, B2a, B3, (C)-C(8), and (C8a). In another embodiment, the Compound is according to Formula I(a) where R² is —NR³R⁴ and R³ and R⁴ together with the nitrogen to which they are attached form HET according to formula (a) where Z is —C(R^{10e})(R^{10f})—; one of R¹⁰, R^{10a}, R^{10b}, R^{10c}, R^{10d}, R^{10e}, and R^{10f} is alkyl; halo; haloalkyl; haloalkenyl; hydroxyalkyl; alkylthio; alkylsulfonyl; hydroxy; alkoxy; cyano; alkoxycarbonyl; —C(O)NR¹¹R^{11a}; phenyl optionally substituted with one, two, or three groups which are independently alkyl, amino, halo, haloalkyl, alkoxy, or haloalkoxy; phenylalkyl optionally substituted with one, two, or three groups which are independently alkyl, amino, halo, haloalkyl, alkoxy, or haloalkoxy; phenyloxy optionally substituted with one, two, or three groups which are alkyl, amino, alkylamino, dialkylamino, halo, haloalkyl, alkoxy, or haloalkoxy; cycloalkyl; heterocycloalkyl; heteroaryl optionally substituted with one or two groups which are independently alkyl or cycloalkyl; the remaining of R¹⁰, R^{10a}, R^{10b}, R^{10c}, R^{10d}, R^{10e}, and R^{10f} are hydrogen; R¹¹ and R^{11a} are independently hydrogen or alkyl; and all other groups are as defined in the Summary of the Invention for a Compound of Formula I or as defined in any one of embodiments B, B1, B1a, B2, B2a, B3, (C)-C(8), and (C8a).

### Embodiments (G5c)

In another embodiment, the Compound is according to Formula I(a) where R² is —NR³R⁴ and R³ and R⁴ together with the nitrogen to which they are attached form HET according to formula (a) where Z is —C(R^{10e})(R^{10f})—; two of R¹⁰, R^{10a}, R^{10b}, R^{10c}, R^{10d}, R^{10e}, and R^{10f} are independently alkyl, halo, haloalkyl, hydroxyalkyl, hydroxy, cyano, —C(O)NR¹¹R^{11a}, or optionally substituted phenyl; the remaining of R¹⁰, R^{10a}, R^{10b}, R^{10c}, R^{10d}, R^{10e}, and R^{10f} are hydrogen; and all other groups are as defined in the Summary of the Invention for a Compound of Formula I or as defined in any one of embodiments B, B1, B1a, B2, B2a, B3, (C)-C(8), and (C8a). In another embodiment, the Compound is according to Formula I(a) where R² is —NR³R⁴ and R³ and R⁴ together with the nitrogen to which they are attached form HET according to formula (a) where Z is —C(R^{10e})(R^{10f})—; two of R¹⁰, R^{10a}, R^{10b}, R^{10c}, R^{10d}, R^{10e}, and R^{10f} are independently alkyl; halo; haloalkyl; hydroxyalkyl; hydroxy; cyano; —C(O)NR¹¹R^{11a}; or phenyl optionally substituted with one or two halo, alkyl, haloalkyl, or alkoxy; R¹¹ and R^{11a} are independently hydrogen or alkyl; the remaining of R¹⁰, R^{10a}, R^{10b}, R^{10c}, R^{10d}, R^{10e}, and R^{10f} are hydrogen; and all other groups are as defined in the Summary of the Invention for a Compound of Formula I or as defined in any one of embodiments B, B1, B1a, B2, B2a, B3, (C)-C(8), and (C8a).

### Embodiments (G5d)

In another embodiment, the Compound is according to Formula I(a) where R² is —NR³R⁴ and R³ and R⁴ together with the nitrogen to which they are attached form HET according to formula (a) where Z is —C(R^{10e})(R^{10f})—; one of R¹⁰, R^{10a}, R^{10b}, R^{10c}, and R^{10d} is optionally substituted phenyl; R^{10e} and R^{10f} together form oxo; the remaining of R¹⁰, R^{10a}, R^{10b}, R^{10c}, and R^{10d} are hydrogen; and all other groups are as defined in the Summary of the Invention for a Compound of Formula I or as defined in any one of embodiments B, B1, B1a, B2, B2a, B3, (C)-C(8), and (C8a). In another embodiment, the Compound is according to Formula I(a) where R² is —NR³R⁴ and R³ and R⁴ together with the nitrogen to which they are attached form HET according to formula (a) where Z is —C(R^{10a})(R^{10f})—; one of R¹⁰, R^{10a}, R^{10b}, R^{10c}, and R^{10d} phenyl optionally substituted with one or two halo; R^{10e} and R^{10f} together form oxo; the remaining of R¹⁰, R^{10a}, R^{10b}, R^{10c}, and R^{10d} are hydrogen; and all other groups are as defined in the Summary of the Invention for a Compound of Formula I or as defined in any one of embodiments B, B1, B1a, B2, B2a, B3, (C)-C(8), and (C8a).

### Embodiments (G5e)

In another embodiment, the Compound is according to Formula I(a) where R² is —NR³R⁴ and R³ and R⁴ together with the nitrogen to which they are attached form HET according to formula (a) where Z is —C(R^{10e})(R^{10f})—; one of R¹⁰, R^{10a}, R^{10b}, R^{10c}, and R^{10d} is optionally substituted phenyl; R^{10e} and R^{10f} are each halo; the remaining of R¹⁰, R^{10a}, R^{10b}, R^{10c}, and R^{10d} are hydrogen; and all other groups are as defined in the Summary of the Invention for a Compound of Formula I or as defined in any one of embodiments B, B1, B1a, B2, B2a, B3, (C)-C(8), and (C8a).

### Embodiments (G6)

In another embodiment, the Compound is according to Formula I(a) where R² is —NR³R⁴ and R³ and R⁴ together with the nitrogen to which they are attached form HET according to formula (a):

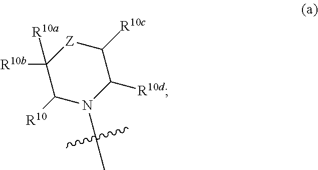

where Z is C₂₋₃-alkylene; R¹⁰, R^{10a}, R^{10b}, R^{10c}, are independently hydrogen; halo; alkyl; haloalkyl; haloalkenyl; hydroxyalkyl; alkylthio; alkylsulfonyl; hydroxy; alkoxy; haloalkoxy; cyano; alkoxycarbonyl; carboxy; amino; alkylamino; dialkylamino; —C(O)R¹²; —C(O)NR¹¹R^{11a}; optionally substituted cycloalkyl; optionally substituted cycloalkylalkyl; optionally substituted phenyl; optionally substituted phenylalkyl; optionally substituted phenyloxy; optionally substituted phenyloxyalkyl; optionally substituted heterocycloalkyl; optionally substituted heterocycloalkylalkyl; optionally substituted heteroaryl; or optionally substituted heteroarylalkyl; or R^{10a} and R^{10b} together form oxo; and all other groups are as defined in the Summary of the Invention for a Compound of Formula I or as defined in any one of embodiments B, B1, B1a, B2, B2a, B3, (C)-C(8), and (C8a).

### Embodiments (G6a)

In another embodiment, the Compound is according to Formula I(a) where R² is —NR³R⁴ and R³ and R⁴ together with the nitrogen to which they are attached form HET according to formula (a) where Z is C₂₋₃-alkylene; one of R¹⁰, R^{10a}, R^{10b}, R^{10c}, and R^{10d} is alkyl, halo, haloalkyl, haloalkenyl, hydroxyalkyl, alkylthio, alkylsulfonyl, hydroxy, alkoxy, haloalkoxy, cyano, alkoxycarbonyl, carboxy, amino, alkylamino, dialkylamino, —C(O)R¹², —C(O)NR¹¹R^{11a}, optionally substituted cycloalkyl, optionally substituted cycloalkylalkyl, optionally substituted phenyl, optionally substituted phenylalkyl, optionally substituted phenyloxy, optionally substituted phenyloxyalkyl, optionally substituted heterocycloalkyl, optionally substituted heterocycloalkylalkyl, optionally substituted heteroaryl, or optionally substituted heteroarylalkyl; or R^{10a} and R^{10b} together form oxo; the remaining of R¹⁰, R^{10a}, R^{10b}, R^{10c}, and R^{10d} are hydrogen; and all other groups are as defined in the Summary of the Invention for a Compound of Formula I or as defined in any one of embodiments B, B1, B1a, B2, B2a, B3, (C)-C(8), and (C8a).

### Embodiments (G6b)

In another embodiment, the Compound is according to Formula I(a) where R² is —NR³R⁴ and R³ and R⁴ together with the nitrogen to which they are attached form HET according to formula (a) where Z is C₂₋₃-alkylene; R¹⁰ is hydrogen or optionally substituted phenyl; and R^{10a}, R^{10b}, and R^{10d} are hydrogen; and all other groups are as defined in the Summary of the Invention for a Compound of Formula I or as defined in any one of embodiments B, B1, B1a, B2, B2a, B3, (C)-C(8), and (C8a). In another embodiment, the Compound is according to Formula I(a) where R² is —NR³R⁴ and R³ and R⁴ together with the nitrogen to which they are attached form HET according to formula (a) where Z is C₂₋₃-alkylene; R¹⁰ is hydrogen or phenyl; and R^{10a}, R^{10b}, R^{10c}, and R^{10d} are hydrogen; and all other groups are as defined in the Summary of the Invention for a Compound of Formula I or as defined in any one of embodiments B, B1, B1a, B2, B2a, B3, (C)-C(8), and (C8a).

### Embodiments (G7)

In another embodiment, the Compound is according to Formula I(a) where
R¹ is phenyl substituted with one or two R⁶ groups which are independently nitro, —NR⁸R^{8a}, —C(O)NR⁸R^{8a}, —NR⁸C(O)OR⁹, or heteroaryl optionally substituted with 1, 2, or 3 R¹⁴; or
R¹ is heteroaryl optionally substituted with one, two, or three R⁷;
R² is —NR³R⁴ and R³ and R⁴ together with the nitrogen to which they are attached form HET according to formula (a):

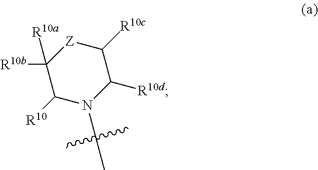

where Z is a bond, —C(O)—, —O—, —S—, —S(O)—, —S(O)₂—, —N(R^{z})—, —C(R^{10e})(R^{10f})—, or C₂₋₃-alkylene;
R^{z} is hydrogen, alkyl, haloalkyl, haloalkenyl, hydroxyalkyl, alkylsulfonyl, hydroxy, alkoxy, alkoxycarbonyl, —C(O)R¹², —C(O)NR¹¹R^{11a}, optionally substituted cycloalkyl, optionally substituted cycloalkylalkyl, optionally substituted phenyl, optionally substituted phenylalkyl, optionally substituted heterocycloalkyl, optionally substituted heterocycloalkylalkyl, optionally substituted heteroaryl, or optionally substituted heteroarylalkyl;
R¹⁰, R^{10a}, R^{10b}, R^{10c}R^{10d}, R^{10e}, and R^{10f} are independently hydrogen; halo; alkyl; haloalkyl; haloalkenyl; hydroxyalkyl; alkylthio; alkylsulfonyl; hydroxy; alkoxy; haloalkoxy; cyano; alkoxycarbonyl; carboxy; amino; alkylamino; dialkylamino; —C(O)R¹²; —C(O)NR¹¹R^{11a}; optionally substituted cycloalkyl; optionally substituted cycloalkylalkyl; optionally substituted phenyl; optionally substituted phenylalkyl; optionally substituted phenyloxy; optionally substituted phenyloxyalkyl; optionally substituted heterocycloalkyl; optionally substituted heterocycloalkylalkyl; optionally substituted heteroaryl; or optionally substituted heteroarylalkyl; or R^{10a} and R^{10b} together form oxo; or R^{10e} and R^{10f} together form oxo;
R¹¹ hydrogen, alkyl, or alkenyl;
R^{11a} hydrogen, alkyl, or alkenyl;
R¹² is alkyl, or optionally substituted heteroaryl; and
each R¹⁴, when present, is halo, alkyl, or alkoxycarbonyl.

### Embodiments (H)

In another embodiment, the Compound is according to Formula I(a) where R² is —NR³R⁴ where R³ and R⁴ together with the nitrogen to which they are attached form HET according to formula (b):

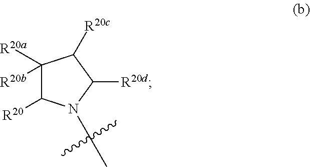

where
(a) R²⁰ and R^{20c} or R²⁰ and R^{20d} together with the carbons to which they are bonded form a cycloalkyl or hetercycloalkyl such that HET is a bridged bicyclic moiety; or
(b) R^{20a} and R^{20c} together with the carbons to which they are bonded form a cycloalkyl or hetercycloalkyl such that HET is a fused bicyclic moiety; or
(c) R^{20a} and R^{20b} together with the carbon to which they are attached form cycloalkyl or heterocycloalkyl such that HET is a spirocyclic bicyclic moiety;
   where the cycloalkyl and heterocycloalkyl are optionally substituted with R¹⁰ and R^{10a} and the R¹⁰ and R^{10a} are independently hydrogen, alkyl, halo, haloalkyl, haloalkenyl, hydroxyalkyl, alkylthio, alkylsulfonyl, hydroxy, alkoxy, haloalkoxy, cyano, alkoxycarbonyl, carboxy, amino, alkylamino, dialkylamino, —C(O)R¹², —C(O)NR¹¹R^{11a}, optionally substituted cycloalkyl, optionally substituted cycloalkylalkyl, optionally substituted phenyl, optionally substituted phenylalkyl, optionally substituted phenyloxy, optionally substituted phenyloxyalkyl, optionally substituted heterocycloalkyl, optionally substituted heterocycloalkylalkyl, optionally substituted heteroaryl, or optionally substituted heteroarylalkyl; and the remaining of R²⁰, R^{20a}, R^{20b}, R^{20c}, and R^{20d} are hydrogen; and all other groups are as defined in the Summary of the Invention for a Compound of Formula I or as defined in any one of embodiments B, B1, B1a, B2, B2a, B3, (C)-C(8), and (C8a).

### Embodiments (H1)

In another embodiment, the Compound is according to Formula I(a) where R² is —NR³R⁴ where R³ and R⁴ together with the nitrogen to which they are attached form HET according to formula (b) where R^{20a} and R^{20c} together with the carbons to which they are bonded form cycloalkyl or heterocycloalkyl such that HET is a fused bicyclic moiety and where the cycloalkyl and heterocycloalkyl are optionally substituted with R¹⁰ and R^{10a}; R²⁰, R^{20b}, and R^{20d} are hydrogen; R¹⁰ and R^{10a} are independently hydrogen, alkyl, halo, haloalkyl, haloalkenyl, hydroxyalkyl, alkylthio, alkylsulfonyl, hydroxy, alkoxy, haloalkoxy, cyano, alkoxycarbonyl, carboxy, amino, alkylamino, dialkylamino, —C(O)R¹², —C(O)NR¹¹R^{11a}, optionally substituted cycloalkyl, optionally substituted cycloalkylalkyl, optionally substituted phenyl, optionally substituted phenylalkyl, optionally substituted phenyloxy, optionally substituted phenyloxyalkyl, optionally substituted heterocycloalkyl, optionally substituted heterocycloalkylalkyl, optionally substituted heteroaryl, or optionally substituted heteroarylalkyl; and all other groups are as defined in the Summary of the Invention for a Compound of Formula I or as defined in any one of embodiments B, B1, B1a, B2, B2a, B3, (C)-C(8), and (C8a). In another embodiment, the Compound is according to Formula I(a) where R² is —NR³R⁴ where R³ and

R⁴ together with the nitrogen to which they are attached form HET according to formula (b) where R^{20a} and R^{20c} together with the carbons to which they are attached form cycloalkyl or heterocycloalkyl such that HET is a fused bicyclic moiety and where the cycloalkyl and heterocycloalkyl are optionally substituted with R¹⁰ and R^{10a}; R²⁰, R^{20b}, and R^{20d} are hydrogen; R¹⁰ is hydrogen, alkyl, or phenyl; and R^{10a} is hydrogen; and all other groups are as defined in the Summary of the Invention for a Compound of Formula I or as defined in any one of embodiments B, B1, B1a, B2, B2a, B3, (C)-C(8), and (C8a). In another embodiment, the Compound is according to Formula I(a) where R² is —NR³R⁴ where R³ and R⁴ together with the nitrogen to which they are attached form HET according to formula (b) and is octahydrocyclopenta[c]pyrrolyl, octahydropyrrolo[3,4-c]pyrrolyl, (3aR,6aS)-5-methyloctahydrocyclopenta[c]pyrrolyl, or (3aS,6aR)-5-methyl-1,2,3,3a,4,6a-hexahydrocyclopenta[c]pyrrolyl; and all other groups are as defined in the Summary of the Invention for a Compound of Formula I or as defined in any one of embodiments B, B1, B1a, B2, B2a, B3, (C)-C(8), and (C8a).

### Embodiments (H2)

In another embodiment, the Compound is according to Formula I(a) where R² is —NR³R⁴ where R³ and R⁴ together with the nitrogen to which they are attached form HET according to formula (b) where R²⁰ and R^{20d} together with the carbons to which they are bonded form a cycloalkyl or hetercycloalkyl such that HET is a bridged bicyclic moiety and where the cycloalkyl and heterocycloalkyl are optionally substituted with R¹⁰ and R^{10a}; R^{20a}, R^{20b}, and R^{20c} are hydrogen; R¹⁰ and R^{10a} are independently hydrogen, alkyl, halo, haloalkyl, haloalkenyl, hydroxyalkyl, alkylthio, alkylsulfonyl, hydroxy, alkoxy, haloalkoxy, cyano, alkoxycarbonyl, carboxy, amino, alkylamino, dialkylamino, —C(O)R¹², —C(O)NR¹¹R^{11a}, optionally substituted cycloalkyl, optionally substituted cycloalkylalkyl, optionally substituted phenyl, optionally substituted phenylalkyl, optionally substituted phenyloxy, optionally substituted phenyloxyalkyl, optionally substituted heterocycloalkyl, optionally substituted heterocycloalkylalkyl, optionally substituted heteroaryl, or optionally substituted heteroarylalkyl; and all other groups are as defined in the Summary of the Invention for a Compound of Formula I or as defined in any one of embodiments B, B1, B1a, B2, B2a, B3, (C)-C(8), and (C8a). In another embodiment, the Compound is according to Formula I(a) where R² is —NR³R⁴ where R³ and R⁴ together with the nitrogen to which they are attached form HET according to formula (b) where R²⁰ and R^{20d} together with the carbons to which they are bonded form a cycloalkyl or hetercycloalkyl such that HET is a bridged bicyclic moiety and where the cycloalkyl and heterocycloalkyl are optionally substituted with R¹⁰ and R^{10a}; and the R¹⁰, R^{10a}, R^{20a}, R^{20b}, and R^{20c} are hydrogen; and all other groups are as defined in the Summary of the Invention for a Compound of Formula I or as defined in any one of embodiments B, B1, B1a, B2, B2a, B3, (C)-C(8), and (C8a).

### Embodiments (H3)

In another embodiment, the Compound is according to Formula I(a) where R² is —NR³R⁴ where R³ and R⁴ together with the nitrogen to which they are attached form HET according to formula (b) where R^{20a} and R^{20b} together with the carbon to which they are bonded form cycloalkyl or heterocycloalkyl such that HET is a spirocyclic bicyclic moiety, where the cycloalkyl and heterocycloalkyl are optionally substituted with R¹⁰ and R^{10a}; and R²⁰, R^{20c}, and R^{20d} are hydrogen; R¹⁰ and R^{10a} are independently hydrogen, alkyl, halo, haloalkyl, haloalkenyl, hydroxyalkyl, alkylthio, alkylsulfonyl, hydroxy, alkoxy, haloalkoxy, cyano, alkoxycarbonyl, carboxy, amino, alkylamino, dialkylamino, —C(O)R¹², —C(O)NR¹¹R^{11a}, optionally substituted cycloalkyl, optionally substituted cycloalkylalkyl, optionally substituted phenyl, optionally substituted phenylalkyl, optionally substituted phenyloxy, optionally substituted phenyloxyalkyl, optionally substituted heterocycloalkyl, optionally substituted heterocycloalkylalkyl, optionally substituted heteroaryl, or optionally substituted heteroarylalkyl; and all other groups are as defined in the Summary of the Invention for a Compound of Formula I or as defined in any one of embodiments B, B1, B1a, B2, B2a, B3, (C)-C(8), and (C8a). In another embodiment, the Compound is according to Formula I(a) where R² is —NR³R⁴ where R³ and R⁴ together with the nitrogen to which they are attached form HET according to formula (b) where R^{20a} and R^{20b} together with the carbon to which they are attached form cycloalkyl or heterocycloalkyl such that HET is a spirocyclic bicyclic moiety, where the cycloalkyl and heterocycloalkyl are optionally substituted with R¹⁰ and R^{10a}; and R¹⁰, R^{10a}, R²⁰, R^{20c}, and R^{20d} are hydrogen; and all other groups are as defined in the Summary of the Invention for a Compound of Formula I or as defined in any one of embodiments B, B1, B1a, B2, B2a, B3, (C)-C(8), and (C8a).

### Embodiments (H4)

In another embodiment, the Compound is according to Formula I(a) where R² is —NR³R⁴ where R³ and R⁴ together with the nitrogen to which they are attached form HET according to formula (b) where R²⁰ and R^{20c} together with the carbons to which they are attached form cycloalkyl or heterocycloalkyl such that HET is a bridged bicyclic moiety, where the cycloalkyl and heterocycloalkyl are optionally substituted with R¹⁰ and R^{10a}; and R^{20a}, R^{20b}, and R^{20d} are hydrogen; R¹⁰ and R^{10a} are independently hydrogen, alkyl, halo, haloalkyl, haloalkenyl, hydroxyalkyl, alkylthio, alkylsulfonyl, hydroxy, alkoxy, haloalkoxy, cyano, alkoxycarbonyl, carboxy, amino, alkylamino, dialkylamino, —C(O)R¹², —C(O)NR¹¹R^{11a}, optionally substituted cycloalkyl, optionally substituted cycloalkylalkyl, optionally substituted phenyl, optionally substituted phenylalkyl, optionally substituted phenyloxy, optionally substituted phenyloxyalkyl, optionally substituted heterocycloalkyl, optionally substituted heterocycloalkylalkyl, optionally substituted heteroaryl, or optionally substituted heteroarylalkyl; and all other groups are as defined in the Summary of the Invention for a Compound of Formula I or as defined in any one of embodiments B, B1, B1a, B2, B2a, B3, (C)-C(8), and (C8a). In another embodiment, the Compound is according to Formula I(a) where R² is —NR³R⁴ where R³ and R⁴ together with the nitrogen to which they are attached form HET according to formula (b) where R²⁰ and R^{20c} together with the carbons to which they are attached form cycloalkyl or heterocycloalkyl such that HET is a bridged bicyclic moiety, where the cycloalkyl and heterocycloalkyl are optionally substituted with R¹⁰ and R^{10a}; and R¹⁰, R^{10a}, R^{20a}, R^{20b}, and R^{20d} are hydrogen; and all other groups are as defined in the Summary of the Invention for a Compound of Formula I or as defined in any one of embodiments B, B1, B1a, B2, B2a, B3, (C)-C(8), and (C8a).

### Embodiments (H5)

In another embodiment, the Compound is according to Formula I(a) where
R¹ is phenyl substituted with one or two R⁶ groups which are independently nitro, —NR⁸R^{8a}, —C(O)NR⁸R^{8a}, —NR⁸C(O)OR⁹, or heteroaryl optionally substituted with 1, 2, or 3 R¹⁴; or
R¹ is heteroaryl optionally substituted with one, two, or three R⁷;
R² is —NR³R⁴ where R³ and R⁴ together with the nitrogen to which they are attached form HET according to formula (b):

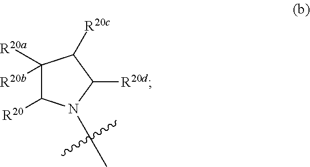

where
(a) R²⁰ and R^{20c} or R²⁰ and R^{20d} together with the carbons to which they are bonded form a cycloalkyl or hetercycloalkyl such that HET is a bridged bicyclic moiety; or
(b) R^{20a} and R^{20c} together with the carbons to which they are bonded form a cycloalkyl or hetercycloalkyl such that BET is a fused bicyclic moiety; or
(c) R^{20a} and R^{20b} together with the carbon to which they are attached form cycloalkyl or heterocycloalkyl such that BET is a spirocyclic bicyclic moiety;
where the cycloalkyl and heterocycloalkyl are optionally substituted with R¹⁰ and R^{10a} where R¹⁰ and R^{10a} are independently hydroxy, alkyl, haloalkyl, or optionally substituted phenyl; and the remaining of R²⁰, R^{20a}, R^{20b}, R^{20c}, and R^{20d} are hydrogen;
each R⁷, when present, is independently alkyl, —NR⁸R^{8a}, —C(O)NR⁸R^{8a}, —NR⁸C(O)OR⁹, or —NR⁸C(O)R⁹;
R⁸ is hydrogen, alkyl, or alkenyl;
R^{8a} is hydrogen, alkyl, haloalkyl, optionally substituted heterocycloalkyl, or optionally substituted phenylalkyl;
R⁹ is alkyl or haloalkyl; and
each R¹⁴, when present, is halo, alkyl, or alkoxycarbonyl.

### Embodiments (R)

In another embodiment, the Compound is according to Formula I(a) where R² is —NR³R⁴ where R³ and R⁴ together with the nitrogen to which they are attached form HET according to formula (b):

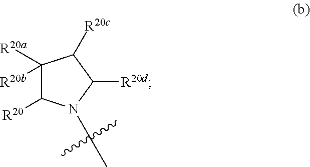

where R²⁰ and R^{20d} together with the carbons to which they are bonded form a cycloalkyl or hetercycloalkyl and R^{20a} and R^{20c} together with the carbons to which they are bonded form a cycloalkyl or hetercycloalkyl such that HET is a tricyclic moiety, where the cycloalkyl and heterocycloalkyl are optionally substituted with R¹⁰ and R^{10a}; and R^{20b} is hydrogen; and all other groups are independently as defined in the Summary of the Invention for a Compound of Formula I or as defined in any one of embodiments B, B1, B1a, B2, B2a, B3, (C)-C(8), and (C8a).

### Embodiments (J)

In another embodiment, the Compound is according to Formula I(a) where R² is —NR³R⁴ where R³ and R⁴ together with the nitrogen to which they are attached form HET according to formula (c):

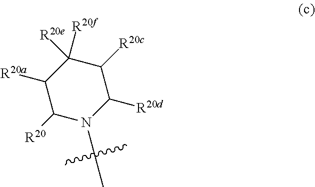

(a) R²⁰ and R^{20d} or R²⁰ and R^{20c} together with the carbons to which they are bonded form a cycloalkyl or hetercycloalkyl such that HET is a bridged bicyclic moiety
(b) R^{20e} and R^{20f} together with the carbons to which they are bonded form cycloalkyl or heterocycloalkyl such that HET is a spirocyclic bicyclic moiety,
(c) R²⁰ and R^{20a} or R^{20a} and R^{20e} together with the carbons to which they are bonded form a cycloalkyl or hetercycloalkyl such that HET is a fused bicyclic moiety;
   where the cycloalkyl and heterocycloalkyl are optionally substituted with R¹⁰ and R^{10a}; and the remaining of R²⁰, R^{20a}, R^{20c}, R^{20d}, R^{20e}, and R^{20f} are R¹⁰, R^{10a}, R^{10c}, R^{10d}, R^{10e}, and R^{10f}, respectively; each R¹⁰, each R^{10a}, R^{10c}, R^{10d}, R^{10e}, R^{10f}, and all other groups are independently as defined in the Summary of the Invention for a Compound of Formula I or as defined in any one of embodiments B, B1, B1a, B2, B2a, B3, (C)-C(8), and (C8a).

### Embodiments (J1)

In another embodiment, the Compound is according to Formula I(a) where R² is —NR³R⁴ where R³ and R⁴ together with the nitrogen to which they are attached form HET according to formula (c) where R²⁰ and R^{20d} together with the carbons to which they are attached form cycloalkyl or heterocycloalkyl such that HET forms a bridged bicyclic moiety and where the cycloalkyl and heterocycloalkyl are optionally substituted with R¹⁰ and R^{10a}; and R^{20a}, R^{20c}, R^{20e}, and R^{20f} are R^{10a}, R^{10c}, R^{10e}, and R^{10f}, respectively; R¹⁰, each R^{10a}, R^{10c}, R^{10e}, R^{10f}, and all other groups are independently as defined in the Summary of the Invention for a Compound of Formula I or as defined in any one of embodiments B, B1, B1a, B2, B2a, B3, (C)-C(8), and (C8a). In another embodiment, the Compound is according to Formula I(a) where R² is —NR³R⁴ where R³ and R⁴ together with the nitrogen to which they are attached form HET according to formula (c) where R²⁰ and R^{20d} together with the carbons to which they are attached form cycloalkyl or heterocycloalkyl such that HET forms a bridged bicyclic moiety; and R^{20a}, R^{20c}, R^{20e}, and R^{20f} are R^{10a}, R^{10c}, R^{10e}, and R^{10f}, respectively; R^{10a}, R^{10c}, R^{10e}, and R^{10f}, and all other groups are independently as defined in the Summary of the Invention for a Compound of Formula I or as defined in any one of embodiments B, B1, B1a, B2, B2a, B3, (C)-C(8), and (C8a).

Embodiments (Ea): In another embodiment, the Compound is according to Formula I(a) where R² is —NR³R⁴ where R³ and R⁴ together with the nitrogen to which they are attached form HET according to formula (c) where R²⁰ and R^{20d} together with the carbons to which they are attached form cycloalkyl or heterocycloalkyl such that HET forms a bridged bicyclic moiety; and R^{20a}, R^{20c}, R^{20e}, and R^{20f} are R^{10a}, R^{10c}, R^{10e}, and R^{10f}, respectively, where R^{10a} and R^{10c} are hydrogen, R^{10e} and R^{10f} together form oxo; and all other groups are as defined in the Summary of the Invention for a Compound of Formula I or as defined in any one of embodiments B, B1, B1a, B2, B2a, B3, (C)-C(8), and (C8a).

### Embodiments (J1b)

In another embodiment, the Compound is according to Formula I(a) where R² is —NR³R⁴ where R³ and R⁴ together with the nitrogen to which they are attached form HET according to formula (c) where R²⁰ and R^{20d} together with the carbons to which they are attached form cycloalkyl or heterocycloalkyl such that HET forms a bridged bicyclic moiety; and R^{20a}, R^{20c}, R^{20e}, and R^{20f} are R^{10a}, R^{10c}, R^{10e}, and R^{10f}, respectively, where R^{10a} and R^{10c} are hydrogen, R^{10e} is hydrogen, hydroxy, or alkyl, and R^{10f} is hydrogen, hydroxy, alkyl, haloalkyl, hydroxyalkyl, amino, halo, or optionally substituted phenyl; and all other groups are as defined in the Summary of the Invention for a Compound of Formula I or as defined in any one of embodiments B, B1, B1a, B2, B2a, B3, (C)-C(8), and (C8a).

### Embodiments (J1c)

In another embodiment, the Compound is according to Formula I(a) where R² is —NR³R⁴ where R³ and R⁴ together with the nitrogen to which they are attached form HET according to formula (c) where R²⁰ and R^{20d} together with the carbons to which they are attached form cycloalkyl or heterocycloalkyl such that HET forms a bridged bicyclic moiety; and R^{20a}, R^{20c}, R^{20e}, and R^{20f} are R^{10a}, R^{10c}, R^{10e}, and R^{10f}, respectively, where R^{10a} and R^{10c} are hydrogen, R^{10e} is hydrogen, and R^{10f} is hydroxy; and all other groups are as defined in the Summary of the Invention for a Compound of Formula I or as defined in any one of embodiments B, B1, B1a, B2, B2a, B3, (C)-C(8), and (C8a). In another embodiment, the Compound is according to Formula I(a) where R² is —NR³R⁴ where R³ and R⁴ together with the nitrogen to which they are attached form HET according to formula (c) where R²⁰ and R^{20d} together with the carbons to which they are attached form cycloalkyl or heterocycloalkyl such that HET forms a bridged bicyclic moiety; and R^{20a}, R^{20c}, R^{20e}, and R^{20f} are R^{10a}, R^{10c}, R^{10e}, and R^{10f}, respectively, where R^{10a} and R^{10c} are hydrogen, R^{10e} is hydrogen, and R^{10f} is alkyl; and all other groups are as defined in the Summary of the Invention for a Compound of Formula I or as defined in any one of embodiments B, B1, B1a, B2, B2a, B3, (C)-C(8), and (C8a). In another embodiment, the Compound is according to Formula I(a) where R² is —NR³R⁴ where R³ and R⁴ together with the nitrogen to which they are attached form HET according to formula (c) where R²⁰ and R^{20d} together with the carbons to which they are attached form cycloalkyl or heterocycloalkyl such that HET forms a bridged bicyclic moiety; and R^{20a}, R^{20c}, R^{20e}, and R^{20f} are R^{10a}, R^{10c}, R^{10e}, and R^{10f}, respectively, where R^{10a} and R^{10c} are hydrogen, R^{10e} is hydroxy, and R^{10f} is haloalkyl; and all other groups are as defined in the Summary of the Invention for a Compound of Formula I or as defined in any one of embodiments B, B1, B1a, B2, B2a, B3, (C)-C(8), and (C8a). In another embodiment, the Compound is according to Formula I(a) where R² is —NR³R⁴ where R³ and R⁴ together with the nitrogen to which they are attached form HET according to formula (c) where R²⁰ and R^{20d} together with the carbons to which they are attached form cycloalkyl or heterocycloalkyl such that HET forms a bridged bicyclic moiety; and R^{20a}, R^{20c}, R^{20e}, and R^{20f} are R^{10a}, R^{10c}, R^{10e}, and R^{10f}, respectively, where R^{10a} and R^{10c} are hydrogen, R^{10e} is hydroxy, and R^{10f} is alkyl; and all other groups are as defined in the Summary of the Invention for a Compound of Formula I or as defined in any one of embodiments B, B1, B1a, B2, B2a, B3, (C)-C(8), and (C8a). In another embodiment, the Compound is according to Formula I(a) where R² is —NR³R⁴ where R³ and R⁴ together with the nitrogen to which they are attached form HET according to formula (c) where R²⁰ and R^{20d} together with the carbons to which they are attached form cycloalkyl or heterocycloalkyl such that HET forms a bridged bicyclic moiety; and R^{20a}, R^{20c}, R^{20e}, and R^{20f} are R^{10a}, R^{10c}, R^{10e}, and R^{10f}, respectively, where R^{10a} and R^{10c} are hydrogen, R^{10e} is alkyl, and R^{10f} is halo; and all other groups are as defined in the Summary of the Invention for a Compound of Formula I or as defined in any one of embodiments B, B1, B1a, B2, B2a, B3, (C)-C(8), and (C8a). In another embodiment, the Compound is according to Formula I(a) where R² is —NR³R⁴ where R³ and R⁴ together with the nitrogen to which they are attached form HET according to formula (c) where R²⁰ and R^{20d} together with the carbons to which they are attached form cycloalkyl or heterocycloalkyl such that HET forms a bridged bicyclic moiety; and R^{20a}, R^{20c}, R^{20e}, and R^{20f} are R^{10a}, R^{10c}, R^{10e}, and R^{10f}, respectively, where R^{10a} and R^{10c} are hydrogen, R^{10e} is hydroxy, and R^{10f} phenyl optionally substituted with one or two halo or haloalkyl; and all other groups are as defined in the Summary of the Invention for a Compound of Formula I or as defined in any one of embodiments B, B1, B1a, B2, B2a, B3, (C)-C(8), and (C8a). In another embodiment, the Compound is according to Formula I(a) where R² is —NR³R⁴ where R³ and R⁴ together with the nitrogen to which they are attached form HET according to formula (c) where R²⁰ and R^{20d} together with the carbons to which they are attached form cycloalkyl or heterocycloalkyl such that HET forms a bridged bicyclic moiety; and R^{20a}, R^{20c}, R^{20e}, and R^{20f} are R^{10a}, R^{10c}, R^{10e}, and R^{10f}, respectively, where R^{10a} and R^{10c} are hydrogen, R^{10e} is hydrogen, and R^{10f} is haloalkyl; and all other groups are as defined in the Summary of the Invention for a Compound of Formula I or as defined in any one of embodiments B, B1, B1a, B2, B2a, B3, (C)-C(8), and (C8a). In another embodiment, the Compound is according to Formula I(a) where R² is —NR³R⁴ where R³ and R⁴ together with the nitrogen to which they are attached form HET according to formula (c) where R²⁰ and R^{20d} together with the carbons to which they are attached form cycloalkyl or heterocycloalkyl such that BET forms a bridged bicyclic moiety; and R^{20a}, R^{20c}, R^{20e}, and R^{20f} are R^{10a}, R^{10c}, R^{10e}, and R^{10f}, respectively, where R^{10a} and R^{10c} are hydrogen, R^{10e} is hydroxy, and R^{10f} is hydroxyalkyl; and all other groups are as defined in the Summary of the Invention for a Compound of Formula I or as defined in any one of embodiments B, B1, B1a, B2, B2a, B3, (C)-C(8), and (C8a). In another embodiment, the Compound is according to Formula I(a) where R² is —NR³R⁴ where R³ and R⁴ together with the nitrogen to which they are attached form HET according to formula (c) where R²⁰ and R^{20d} together with the carbons to which they are attached form cycloalkyl or heterocycloalkyl such that HET forms a bridged bicyclic moiety; and R^{20a}, R^{20c}, R^{20e}, and R^{20f} are R^{10a}, R^{10c}, R^{10e}, and R^{10f}, respectively, where R^{10a} and R^{10c} are hydrogen, R^{10e} is hydrogen, and R^{10f} is amino; and all other groups are as defined in the Summary of the Invention for a Compound of Formula I or as defined in any one of embodiments B, B1, B1a, B2, B2a, B3, (C)-C(8), and (C8a). In another embodiment, the Compound is according to Formula I(a) where R² is —NR³R⁴ where R³ and R⁴ together with the nitrogen to which they are attached form HET according to formula (c) where R²⁰ and R^{20d} together with the carbons to which they are attached form cycloalkyl or heterocycloalkyl such that HET forms a bridged bicyclic moiety; and R^{20a}, R^{20c}, R^{20e}, and R^{20f} are R^{10a}, R^{10c}, R^{10e}, and R^{10f}, respectively, where R^{10a}, R^{10c}, and R^{10e} are hydrogen, and R^{10f} is hydroxyalkyl; and all other groups are as defined in the Summary of the Invention for a Compound of Formula I or as defined in any one of embodiments B, B1, B1a, B2, B2a, B3, (C)-C(8), and (C8a).

### Embodiments (J2)

In another embodiment, the Compound is according to Formula I(a) where R² is —NR³R⁴ where R³ and R⁴ together with the nitrogen to which they are attached form HET according to formula (c) where R²⁰ and R^{20c} together with the carbons to which they are attached form cycloalkyl or heterocycloalkyl such that HET is a bridged bicyclic moiety, where the cycloalkyl and heterocycloalkyl are optionally substituted with R¹⁰ and R^{10a}; and R^{20a}, R^{20d}, R^{20e}, and R^{20f} are R^{10a}, R^{10d}, R^{10e}, and R^{10f}, respectively; R¹⁰, each R^{10a}, R^{10d}, R^{10e}, and R^{10f}, and all other groups are independently as defined in the Summary of the Invention for a Compound of Formula or as defined in any one of embodiments B, B1, B1a, B2, B2a, B3, (C)-C(8), and (C8a). In another embodiment, the Compound is according to Formula I(a) where R² is —NR³R⁴ where R³ and R⁴ together with the nitrogen to which they are attached form HET according to formula (c) where R²⁰ and R^{20c} together with the carbons to which they are attached form cycloalkyl or heterocycloalkyl such that HET is a bridged bicyclic moiety, where the cycloalkyl and heterocycloalkyl are optionally substituted with R¹⁰ and R^{10a}; and R^{20a}, R^{20d}, R^{20e}, and R^{20f} are R^{10a}, R^{10d}, R^{10e}, and R^{10f}, respectively where each R^{10a}, R^{10d}, R^{10e}, and R^{10f} are hydrogen; and all other groups are as defined in the Summary of the Invention for a Compound of Formula I or as defined in any one of embodiments B, B1, B1a, B2, B2a, B3, (C)-C(8), and (C8a).

### Embodiments (J3)

In another embodiment, the Compound is according to Formula I(a) where R² is —NR³R⁴ where R³ and R⁴ together with the nitrogen to which they are attached form HET according to formula (c) where R^{20e} and R^{20f} together with the carbons to which they are attached form cycloalkyl or heterocycloalkyl such that HET is a spirocyclic bicyclic moiety, where the cycloalkyl and heterocycloalkyl are optionally substituted with R¹⁰ and R^{10a}; and R²⁰, R^{20a}, R^{20c}, and R^{20d} are R¹⁰, R^{10a}, R^{10c}, and R^{10d}, respectively; each R¹⁰, each R^{10a}, R^{10c}, and R^{10d}, and all other groups are independently as defined in the Summary of the Invention for a Compound of Formula I or as defined in any one of embodiments B, B1, B1a, B2, B2a, B3, (C)-C(8), and (C8a).

### Embodiments (J4)

In another embodiment, the Compound is according to Formula I(a) where R² is —NR³R⁴ where R³ and R⁴ together with the nitrogen to which they are attached form HET according to formula (c) where R²⁰ and R^{20a} together with the carbons to which they are attached form cycloalkyl or heterocycloalkyl such that BET is a fused bicyclic moiety, where the cycloalkyl and heterocycloalkyl are optionally substituted with R¹⁰ and R^{10a}; and R^{20c}, R^{20d}, R^{20e}, and R^{20f} are R^{10c}, R^{10d}, R^{10e}, and R^{10f}, respectively; R¹⁰, R^{10a}, R^{10c}, R^{10d}, R^{10e}, R^{10f}, and all other groups are independently as defined in the Summary of the Invention for a Compound of Formula I or as defined in any one of embodiments B, B1, B1a, B2, B2a, B3, (C)-C(8), and (C8a). In another embodiment, the Compound is according to Formula I(a) where R² is —NR³R⁴ where R³ and R⁴ together with the nitrogen to which they are attached form HET according to formula (c) where R²⁰ and R^{20a} together with the carbons to which they are attached form cycloalkyl or heterocycloalkyl such that BET is a fused bicyclic moiety, where the cycloalkyl and heterocycloalkyl are optionally substituted with R¹⁰ and R^{10a}; R^{20c}, R^{20d}, R^{20e}, and R^{20f} are R^{10c}, R^{10d}, R^{10e}, and R^{10f}, respectively and R^{10c}, R^{10d}, R^{10e}, and R^{10f} are hydrogen; R¹⁰, R^{10a}, and all other groups are independently as defined in the Summary of the Invention for a Compound of Formula I or as defined in any one of embodiments B, B1, B1a, B2, B2a, B3, (C)-C(8), and (C8a).

### Embodiments (J5)

In another embodiment, the Compound is according to Formula I(a) where R² is —NR³R⁴ where R³ and R⁴ together with the nitrogen to which they are attached form HET according to formula (c) where R^{20a} and R^{20e} together with the carbons to which they are attached form cycloalkyl or heterocycloalkyl such that HET is a fused bicyclic moiety, where the cycloalkyl and heterocycloalkyl are optionally substituted with R¹⁰ and R^{10a}; and R²⁰, R^{20c}, R^{20d}, and R^{20f} are R¹⁰, R^{10c}, R^{10d}, and R^{10f}, respectively; each R¹⁰, R^{10a}, R^{10c}, R^{10d}, R^{10f}, and all other groups are independently as defined in the Summary of the Invention for a Compound of Formula I or as defined in any one of embodiments B, B1, B1a, B2, B2a, B3, (C)-C(8), and (C8a). In another embodiment, the Compound is according to Formula I(a) where R² is —NR³R⁴ where R³ and R⁴ together with the nitrogen to which they are attached form HET according to formula (c) where R^{20a} and R^{20e} together with the carbons to which they are attached form cycloalkyl or heterocycloalkyl such that HET is a fused bicyclic moiety; and R²⁰, R^{20c}, R^{20d}, and R^{20f} are R¹⁰, R^{10c}, R^{10d}, and R^{10f}, respectively and R¹⁰, R^{10c}, R^{10d}, and R^{10f} are hydrogen; and all other groups are as defined in the Summary of the Invention for a Compound of Formula I or as defined in any one of embodiments B, B1, B1a, B2, B2a, B3, (C)-C(8), and (C8a).

### Embodiment (J6)

In another embodiment, the Compound is according to Formula I(a) where R² is —NR³R⁴ where R³ and R⁴ together with the nitrogen to which they are attached form HET according to formula (c) which is according to formula (g)

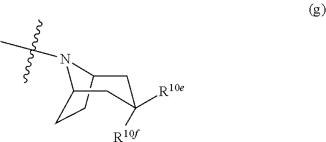

where R^{10e}, R^{10f}, and all other groups are independently as defined in the Summary of the Invention for a Compound of Formula I or as defined in any one of embodiments B, B1, B1a, B2, B2a, B3, (C)-C(8), and (C8a).

### Embodiment (J6a)

In another embodiment, the Compound is according to Formula I(a) where R² is —NR³R⁴ where R³ and R⁴ together with the nitrogen to which they are attached form HET according to formula (c) which is according to formula (g) where R^{10e} is hydrogen, alkyl, halo, haloalkyl, hydroxy, or optionally substituted phenyl; R^{10f} is hydrogen, hydroxy, amino, alkyl, hydroxyalkyl, or haloalkyl; and all other groups are as defined in the Summary of the Invention for a Compound of Formula I or as defined in any one of embodiments B, B1, B1a, B2, B2a, B3, (C)-C(8), and (C8a). In another embodiment, the Compound is according to Formula I(a) where R² is —NR³R⁴ where R³ and R⁴ together with the nitrogen to which they are attached form HET according to formula (c) which is according to formula (g) where R^{10e} is hydrogen, alkyl, halo, haloalkyl, hydroxy, or phenyl optionally substituted with one or two groups which are halo or haloalkyl; R^{10f} is hydrogen, hydroxy, amino, alkyl, hydroxyalkyl, or haloalkyl; and all other groups are as defined in the Summary of the Invention for a Compound of Formula I or as defined in any one of embodiments B, B1, B1a, B2, B2a, B3, (C)-C(8), and (C8a).

### Embodiment (J6b)

In another embodiment, the Compound is according to Formula I(a) where R² is —NR³R⁴ where R³ and R⁴ together with the nitrogen to which they are attached form HET according to formula (c) which is according to formula (g) where R^{10e} and R^{10f} together form oxo; and all other groups are as defined in the Summary of the Invention for a Compound of Formula I or as defined in any one of embodiments B, B1, Bla, B2, B2a, B3, (C)-C(8), and (C8a).

### Embodiment (J7)

In another embodiment, the Compound is according to Formula I(a) where R² is —NR³R⁴ where R³ and R⁴ together with the nitrogen to which they are attached form BET according to formula (c) which is further according to formula (h)

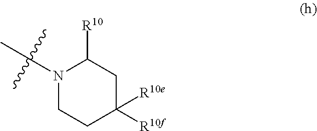

where R¹⁰, R^{10e}, R^{10f}, and all other groups are independently as defined in the Summary of the Invention for a Compound of Formula I or as defined in any one of embodiments B, B1, B1a, B2, B2a, B3, (C)-C(8), and (C8a). In another embodiment of embodiment (J7), the Compound is according to Formula I(a) where R² is —NR³R⁴ where R³ and R⁴ together with the nitrogen to which they are attached form BET according to formula (c) which is further according to formula (h) where one of R¹⁰, R^{10e}, and R^{10f} is not hydrogen and the others are as defined in embodiment (J7); and all other groups are as defined in the Summary of the Invention for a Compound of Formula I or as defined in any one of embodiments B, B1, B1a, B2, B2a, B3, (C)-C(8), and (C8a).

### Embodiment (J7a)

In another embodiment, the Compound is according to Formula I(a) where R² is —NR³R⁴ where R³ and R⁴ together with the nitrogen to which they are attached form HET according to formula (c) which is further according to formula (h) where R¹⁰ is hydrogen; R^{10e} is —C(O)NH₂, hydroxy, alkoxy, cyano, alkyl, haloalkyl, haloalkenyl, hydroxyalkyl, alkylthio, alkylsulfonyl, optionally substituted cycloalkyl, optionally substituted heterocycloalkyl, optionally substituted phenyl, optionally substituted phenylalkyl, optionally substituted phenyloxy, or optionally substituted heteroaryl; and R^{10f} is hydrogen; and all other groups are as defined in the Summary of the Invention for a Compound of Formula I or as defined in any one of embodiments B, B1, B1a, B2, B2a, B3, (C)-C(8), and (C8a). In another embodiment, the Compound is according to Formula I(a) where R² is —NR³R⁴ where R³ and R⁴ together with the nitrogen to which they are attached form HET according to formula (c) which is further according to formula (h) where R¹⁰ is hydrogen; R^{10e} is —C(O)NH₂, hydroxy, alkoxy, cyano, alkyl, haloalkyl, haloalkenyl, hydroxyalkyl, alkylthio, alkylsulfonyl, cycloalkyl, heterocycloalkyl, phenyl optionally substituted with one or two halo, phenylalkyl optionally substituted with one or two halo, phenyloxy optionally substituted with one or two halo, heteroaryl optionally substituted with one alkyl or cycloalkyl; and R^{10f} is hydrogen; and all other groups are as defined in the Summary of the Invention for a Compound of Formula I or as defined in any one of embodiments B, B1, B1a, B2, B2a, B3, (C)-C(8), and (C8a).

### Embodiment (J7b)

In another embodiment, the Compound is according to Formula I(a) where R² is —NR³R⁴ where R³ and R⁴ together with the nitrogen to which they are attached form HET according to formula (c) which is further according to formula (h) where R¹⁰ is alkyl, or optionally substituted phenyl; R^{10e} is hydroxy, alkyl, haloalkyl, or cyano; and R^{10f} is hydrogen; and all other groups are as defined in the Summary of the Invention for a Compound of Formula I or as defined in any one of embodiments B, B1, B1a, B2, B2a, B3, (C)-C(8), and (C8a). In another embodiment, the Compound is according to Formula I(a) where R² is —NR³R⁴ where R³ and R⁴ together with the nitrogen to which they are attached form HET according to formula (c) which is further according to formula (h) where R¹⁰ is alkyl, or phenyl optionally substituted with one or tow groups which are independently halo, or haloalkyl; R^{10e} is hydroxy, alkyl, haloalkyl, or cyano; and R^{10f} is hydrogen; and all other groups are as defined in the Summary of the Invention for a Compound of Formula I or as defined in any one of embodiments B, B1, B1a, B2, B2a, B3, (C)-C(8), and (C8a).

### Embodiment (J7c)

In another embodiment, the Compound is according to Formula I(a) where R² is —NR³R⁴ where R³ and R⁴ together with the nitrogen to which they are attached form HET according to formula (c) which is further according to formula (h) where R^{10e} and R^{10f} together form oxo; and R¹⁰ and all other groups are as defined in the Summary of the Invention for a Compound of Formula I or as defined in any one of embodiments B, B1, B1a, B2, B2a, B3, (C)-C(8), and (C8a). In another embodiment, the Compound is according to Formula I(a) where R² is —NR³R⁴ where R³ and R⁴ together with the nitrogen to which they are attached form HET according to formula (c) which is further according to formula (h) where R¹⁰ is hydrogen, or optionally substituted phenyl; R^{10e} and R^{10f} together form oxo; and all other groups are as defined in the Summary of the Invention for a Compound of Formula I or as defined in any one of embodiments B, B1, B1a, B2, B2a, B3, (C)-C(8), and (C8a). In another embodiment, the Compound is according to Formula I(a) where R² is —NR³R⁴ where R³ and R⁴ together with the nitrogen to which they are attached form HET according to formula (c) which is further according to formula (h) where R¹⁰ is hydrogen, or phenyl optionally substituted with one or two halo; R^{10e} and R^{10f} together form oxo; and all other groups are as defined in the Summary of the Invention for a Compound of Formula I or as defined in any one of embodiments B, B1, B1a, B2, B2a, B3, (C)-C(8), and (C8a).

### Embodiment (J7d)

In another embodiment, the Compound is according to Formula I(a) where R² is —NR³R⁴ where R³ and R⁴ together with the nitrogen to which they are attached form HET according to formula (c) which is further according to formula (h) where R¹⁰ is alkyl, haloalkyl, alkoxycarbonyl, or optionally substituted phenyl; R^{10e} and R^{10f} are hydrogen; and all other groups are as defined in the Summary of the Invention for a Compound of Formula I or as defined in any one of embodiments B, B1, B1a, B2, B2a, B3, (C)-C(8), and (C8a). In another embodiment, the Compound is according to Formula I(a) where R² is —NR³R⁴ where R³ and R⁴ together with the nitrogen to which they are attached form HET according to formula (c) which is further according to formula (h) where R¹⁰ is alkyl, haloalkyl, alkoxycarbonyl, or phenyl optionally substituted with one, two, or three groups which are independently dialkylamino, alkyl, halo, haloalkyl, or alkoxy; R^{10e} and R^{10f} are hydrogen; and all other groups are as defined in the Summary of the Invention for a Compound of Formula I or as defined in any one of embodiments B, B1, B1a, B2, B2a, B3, (C)-C(8), and (C8a).

### Embodiment (J7e)

In another embodiment, the Compound is according to Formula I(a) where R² is —NR³R⁴ where R³ and R⁴ together with the nitrogen to which they are attached form HET according to formula (c) which is further according to formula (h) where R¹⁰ is optionally substituted phenyl; R^{10e} is hydroxy, or halo; and R^{10f} is alkyl, halo, haloalkyl, or hydroxyalkyl; and all other groups are as defined in the Summary of the Invention for a Compound of Formula I or as defined in any one of embodiments B, B1, B1a, B2, B2a, B3, (C)-C(8), and (C8a). In another embodiment, the Compound is according to Formula I(a) where R² is —NR³R⁴ where R³ and R⁴ together with the nitrogen to which they are attached form HET according to formula (c) which is further according to formula (h) where R¹⁰ is phenyl optionally substituted with one or two halo; R^{10e} is hydroxy, or halo; and R^{10f} is alkyl, halo, haloalkyl, or hydroxyalkyl; and all other groups are as defined in the Summary of the Invention for a Compound of Formula I or as defined in any one of embodiments B, B1, B1a, B2, B2a, B3, (C)-C(8), and (C8a).

### Embodiment (J7f)

In another embodiment, the Compound is according to Formula I(a) where R² is —NR³R⁴ where R³ and R⁴ together with the nitrogen to which they are attached form HET according to formula (c) which is further according to formula (h) where R¹⁰ is hydrogen; R^{10e} is hydroxy, halo, alkyl, or cyano; and R^{10f} is alkyl, haloalkyl, halo, —C(O)NH₂, or optionally substituted phenyl; and all other groups are as defined in the Summary of the Invention for a Compound of Formula I or as defined in any one of embodiments B, B1, B1a, B2, B2a, B3, (C)-C(8), and (C8a). In another embodiment, the Compound is according to Formula I(a) where R² is —NR³R⁴ where R³ and R⁴ together with the nitrogen to which they are attached form HET according to formula (c) which is further according to formula (h) where R¹⁰ is hydrogen; R^{10e} is hydroxy, halo, alkyl, or cyano; and R^{10f} is alkyl, haloalkyl, halo, —C(O)NH₂, or phenyl optionally substituted with one or two groups which are independently halo, alkyl, haloalkyl, or alkoxy; and all other groups are as defined in the Summary of the Invention for a Compound of Formula I or as defined in any one of embodiments B, B1, B1a, B2, B2a, B3, (C)-C(8), and (C8a).

### Embodiments (J8)

In another embodiment, the Compound is according to Formula I(a) where R² is —NR³R⁴ where R³ and R⁴ together with the nitrogen to which they are attached form HET according to formula (c):

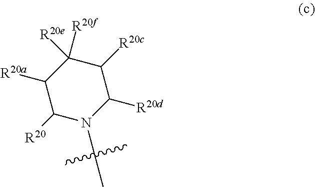

(a) R²⁰ and R^{20d} or R²⁰ and R^{20c} together with the carbons to which they are bonded form a cycloalkyl such that HET is a bridged moiety
(b) R^{20e} and R^{20f} together with the carbons to which they are bonded form cycloalkyl such that HET is a spirocyclic moiety,
(c) R²⁰ and R^{20a} or R^{20a} and R^{20e} together with the carbons to which they are bonded form a cycloalkyl such that HET is a fused bicyclic moiety;
where the cycloalkyl is optionally substituted with R¹⁰ and R^{10a} where R¹⁰ and R^{10a} are independently alkyl or together form oxo; and the remaining of R²⁰, R^{20a}, R^{20c}, R^{20d}, R^{20e}, and R^{20f} are R¹⁰, R^{10a}, R^{10c}, R^{10d}, R^{10e}, and R^{10f}, and R^{10f}, respectively, where R¹⁰, R^{10a}, R^{10c}, R^{10d}, R^{10e}, and R^{10f} are independently hydrogen, hydroxy, alkyl, halo, haloalkyl, hydroxyalkyl, optionally substituted phenyl, or amino, or R^{10e} and R^{10f} together form oxo;
each R⁷, when present, is independently alkyl, —NR⁸R^{8a}, —C(O)NR⁸R^{8a}, —NR⁸C(O)OR⁹, or —NR⁸C(O)R⁹;
R⁸ is hydrogen, alkyl, or alkenyl;
R^{8a} is hydrogen, alkyl, haloalkyl, optionally substituted heterocycloalkyl, or optionally substituted phenylalkyl;
R⁹ is alkyl or haloalkyl; and
each R¹⁴, when present, is halo, alkyl, or alkoxycarbonyl.

### Embodiments (K)

In another embodiment, the Compound of Formula is according to Formula I where R² is —NR³R⁴ where R³ and R⁴ together with the nitrogen to which they are attached form HET according to formula (d), (e), or (f):

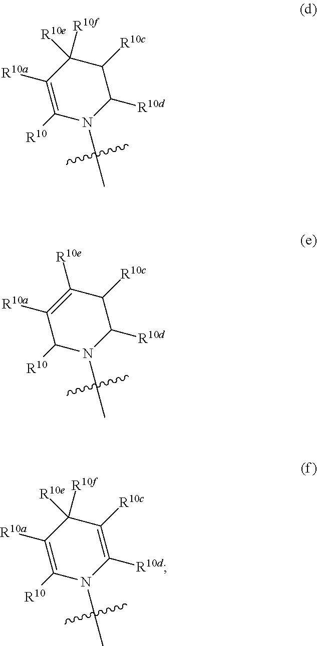

where all other groups are as defined in the Summary of the Invention for a Compound of Formula I or as defined in any one of embodiments B, B1, B1a, B2, B2a, B3, (C)-C(8), and (C8a). In another embodiment, the Compound of Formula is according to Formula I where R² is —NR³R⁴ where R³ and R⁴ together with the nitrogen to which they are attached form HET according to formula (d) or (f) where R¹⁰ is optionally substituted phenyl, R^{10e} and R^{10f} together form oxo, and R^{10a}, R^{10c}, and R^{10d} are hydrogen; and all other groups are as defined in the Summary of the Invention for a Compound of Formula I or as defined in any one of embodiments B, B1, B1a, B2, B2a, B3, (C)-C(8), and (C8a). In another embodiment, the Compound of Formula is according to Formula I where R² is —NR³R⁴ where R³ and R⁴ together with the nitrogen to which they are attached form HET according to formula (e) where R¹⁰ or R^{10e} is optionally substituted phenyl, and the remaining of R¹⁰, R^{10a}, R^{10c}, R^{10d}, R^{10e}, and R^{10f} are hydrogen; and all other groups are as defined in the Summary of the Invention for a Compound of Formula I or as defined in any one of embodiments B, B1, B1a, B2, B2a, B3, (C)-C(8), and (C8a).

Embodiments (K₁): In another embodiment, the Compound of Formula is according to Formula I where
R¹ is phenyl substituted with one or two R⁶ groups independently which are independently nitro, —NR⁸R^{8a}, —C(O)NR⁸R^{8a}, —NR⁸C(O)OR⁹, or heteroaryl optionally substituted with 1, 2, or 3 R¹⁴; or
R¹ is heteroaryl optionally substituted with one, two, or three R⁷;
R² is —NR³R⁴ where R³ and R⁴ together with the nitrogen to which they are attached form HET according to formula (d), (e), or (f):

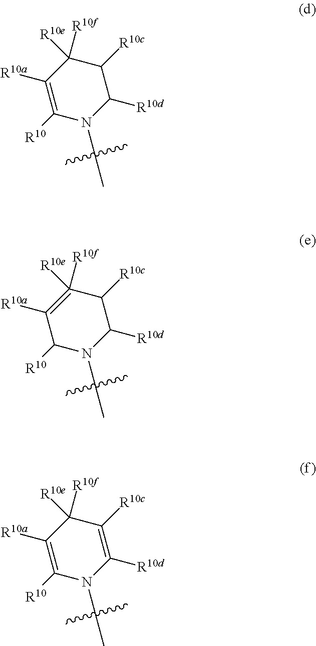

where R¹⁰, R^{10a}, R^{10c}, R^{10d}, R^{10e}, and R^{10f} are independently hydrogen, hydroxy, alkyl, haloalkyl, or optionally substituted phenyl; or, in formula (d) or (f), R^{10e} and R^{10f} together form oxo;
each R⁷, when present, is independently alkyl, —NR⁸R^{8a}, —C(O)NR⁸R^{8a}, —NR⁸C(O)OR⁹, or —NR⁸C(O)R⁹;
R⁸ is hydrogen, alkyl, or alkenyl;
R^{8e} is hydrogen, alkyl, haloalkyl, optionally substituted heterocycloalkyl, or optionally substituted phenylalkyl;
R⁹ is alkyl or haloalkyl; and
each R¹⁴, when present, is halo, alkyl, or alkoxycarbonyl.

In another embodiment (L), the Compound is according to Formula I(e)

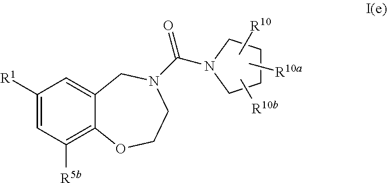

where R¹⁰, R^{10a}, R^{10b}, and all other groups are independently as defined in the Summary of the Invention for a Compound of Formula I or as defined in any one of embodiments B, B1, B1a, B2, B2a, B3, (C)-C(8), and (C8a).

In another embodiment (M), the Compound of Formula I is according to Formula I(f)

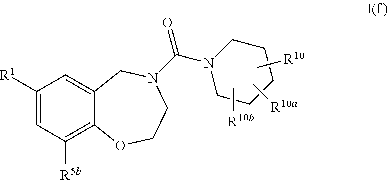

where R¹⁰, R^{10a}, R^{10b}, and all other groups are independently as defined in the Summary of the Invention for a Compound of Formula I or as defined in any one of embodiments B, B1, B1a, B2, B2a, B3, (C)-C(8), and (C8a).

In another embodiment (N), the Compound of Formula I is according to Formula I(g)

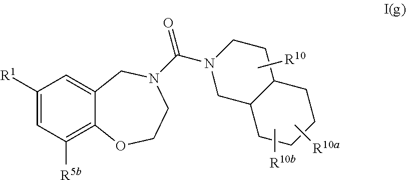

where R¹⁰, R^{10a}, R^{10b}, and all other groups are independently as defined in the Summary of the Invention for a Compound of Formula I or as defined in any one of embodiments B, B1, B1a, B2, B2a, B3, (C)-C(8), and (C8a).

In another embodiment (P), the Compound of Formula I is according to Formula I(h)

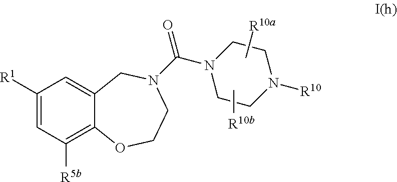

where R¹⁰, R^{10a}, R^{10b}, and all other groups are independently as defined in the Summary of the Invention for a Compound of Formula I or as defined in any one of embodiments B, B1, B1a, B2, B2a, B3, (C)-C(8), and (C8a).

In another embodiment (O), the Compound of Formula I is according to Formula I(p)

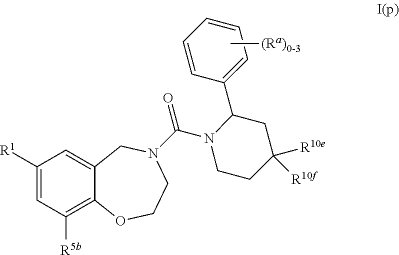

where each R^{a}, when R^{a} is present, is independently alkyl, alkoxy, or halo; and R^{10e}, R^{10f}, and all other groups are independently as defined in the Summary of the Invention for a Compound of Formula I or as defined in any one of embodiments B, B1, B1a, B2, B2a, B3, (C)-C(8), and (C8a).

In another embodiment (Q1), the Compound of Formula I is according to Formula I(n)

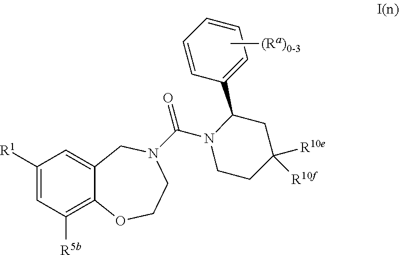

where each R^{a}, when R^{a} is present, is independently alkyl, alkoxy, or halo; and R^{10e}, R^{10f}, and all other groups are independently as defined in the Summary of the Invention for a Compound of Formula I or as defined in any one of embodiments B, B1, B1a, B2, B2a, B3, (C)-C(8), and (C8a).

In another embodiment, any one of the Compound of Formulae I, I(a), I(b), I(c), I(d), I(e), I(f), I(g), I(h), I(p), and I(n) is that where R¹ and/or R² are independently as defined in any one of the above embodiments.

### Embodiment (U)

Another embodiment provides a pharmaceutical composition which comprises 1) a compound, as a single stereoisomer or mixture of isomers thereof, according to any one of Formula I, (I(a), I(b), I(c), I(d), I(e), I(f), I(g), I(h), I(p), and I(n) or according to any one of the above embodiments or a compound in Table 1, optionally as a pharmaceutically acceptable salt thereof, and 2) a pharmaceutically acceptable carrier, excipient, and/or diluent thereof.

### Embodiment (V)

Another embodiment is a method of treating disease, disorder, or syndrome where the disease is associated with uncontrolled, abnormal, and/or unwanted cellular activities effected directly or indirectly by PI3K and/or mTOR which method comprises administering to a human in need thereof a therapeutically effective amount of a Compound of any of Formula I, (I(a), I(b), I(c), I(d), I(e), I(f), I(g), I(h), I(p), and I(n), a Compound of any one of the above embodiments, or a Compound from Table 1, optionally as a pharmaceutically acceptable salt or pharmaceutical composition thereof. In another embodiment of embodiment (V), the disease is cancer. In another embodiment of embodiment (V), the disease is cancer and the Compound is of Formula I(a) or a Compound from Table 1.

### Embodiment (W)

Another embodiment is directed to a method of treating a disease, disorder, or syndrome which method comprises administering to a patient a therapeutically effective amount of a Compound of any of Formula I, (I(a), I(b), I(c), I(d), I(e), I(f), I(g), I(h), I(p), and I(n), a Compound of any one of the above embodiments, or a Compound from Table 1, optionally as a pharmaceutically acceptable salt thereof, or a pharmaceutical composition comprising a therapeutically effective amount of a Compound of Formula I, (I(a), I(b), I(c), I(d), I(e), I(f), I(g), I(h), I(p), and I(n), a Compound of any one of the above embodiments, or a Compound from Table 1, and a pharmaceutically acceptable carrier, excipient, or diluent. In another embodiment of embodiment (W), the disease is cancer. In another embodiment of embodiment (W), the disease is cancer and the Compound is of Formula I(a) or a Compound from Table 1.

In another embodiment of any of the embodiments of Embodiment (W), the cancer is breast cancer, mantle cell lymphoma, renal cell carcinoma, acute myelogenous leukemia, chronic myelogenous leukemia, NPM/ALK-transformed anaplastic large cell lymphoma, diffuse large B cell lymphoma, rhabdomyosarcoma, ovarian cancer, endometrial cancer, cervical cancer, non small cell lung carcinoma, small cell lung carcinoma, adenocarcinoma, colon cancer, rectal cancer, gastric carcinoma, hepatocellular carcinoma, melanoma, pancreatic cancer, prostate carcinoma, thyroid carcinoma, anaplastic large cell lymphoma, hemangioma, glioblastoma, or head and neck cancer.

Another embodiment is directed to a method for identifying a selective inhibitor of a PI3K isozyme, the method comprising: (a) contacting a first cell bearing a first mutation in a PI3K-α with a candidate inhibitor; (b) contacting a second cell bearing a wild type PI3K-α, a PTEN null mutation, or a second mutation in said PI3K-α with the candidate inhibitor; and (c) measuring AKT phosphorylation in said first and said second cells, wherein decreased AKT phosphorylation in said first cell when compared to said second cell identifies said candidate inhibitor as a selective PI3K-α inhibitor.

As noted above, the newly discovered association between selective genetic mutations and increased sensitivities of some cancers to specific inhibitors renders a particular genetic background more susceptible to one or more types of inhibitors than others. This association between genetic backgrounds and susceptibilities of certain cancers provides an attractive and convenient cellular platform for identification of new selective inhibitors to PI3K kinases (e.g. via screening assays to detect compounds or entities that inhibit phosphorylation in a PI3K-αdependent manner). As will be appreciated by those of ordinary skill in the art, any kind of compounds or agents can be tested using the inventive screening methods. A candidate inhibitor compound may be a synthetic or natural compound; it may be a single molecule, a mixture of different molecules or a complex of at least two molecules. A candidate inhibitor can comprise functional groups necessary for structural interaction with proteins, particularly hydrogen bonding and lipophilic binding, and typically include at least an amine, carbonyl, hydroxyl, ether, or carboxyl group, for example at least two of the functional chemical groups. The candidate inhibitor often comprises cyclical carbon or heterocycloalkyl structures and/or aromatic or heteroaromatic structures substituted with one or more of the above functional groups. Candidate inhibitors are also found among biomolecules including peptides, saccharides, fatty acids, steroids, purines, pyrimidines, derivatives, structural analogs, or combinations thereof. In certain embodiments, the inventive methods are used for testing one or more candidate inhibitor compounds. In other embodiments, the inventive methods are used for screening collections or libraries of candidate inhibitor compounds. As used herein, the term “collection” refers to any set of compounds, molecules or agents, while the term “library” refers to any set of compounds, molecules or agents that are structural analogs.

Libraries of candidate inhibitor compounds that can be screened using the methods of the present invention may be either prepared or purchased from a number of companies. Synthetic compound libraries are commercially available from, for example, Comgenex (Princeton, N.J.), Brandon Associates (Merrimack, N.H.), Microsource (New Milford, Conn.), and Aldrich (Milwaukee, Wis.). Libraries of candidate inhibitor compounds have also been developed by and are commercially available from large chemical companies. Additionally, natural collections, synthetically produced libraries and compounds are readily modified through conventional chemical, physical, and biochemical means.

Cells to be used in the practice of the screening methods described herein may be primary cells, secondary cells, or immortalized cells (e.g., established cell lines). They may be prepared by techniques well known in the art (for example, cells may be obtained by fine needle biopsy from a patient or a healthy donor) or purchased from immunological and microbiological commercial resources (for example, from the American Type Culture Collection (ATCC), Manassas, Va.). Alternatively or additionally, cells may be genetically engineered to contain, for example, a gene of interest. In a first set of cells, the cells possess a genetic mutation in PI3K-α kinase domain, for example, H1047R. In a second set of cells to be used in the screening assays, the second set of cells possess a genetic mutation in a different kinase catalytic subunit, (for example, a mutation in a helical domain, for example, E545K, or in a different regulatory protein, for example Phosphatase and Tensin Homolog (PTEN). When a candidate inhibitor inhibits phosphorylation, (for example AKT phosphorylation) to a higher degree in the cell possessing the PI3K-α kinase domain genetic mutation when compared to a cell possessing a genetic mutation in a different kinase catalytic subunit, (for example a mutation in a helical domain, for example, E545K, or in a different regulatory protein), then the candidate inhibitor is a selective inhibitor for cancers or tumors that harbor activation mutations in PI3K-α. Conversely, PI3K-α-selective compounds inhibit AKT phosphorylation, PI3K pathway activation, and cell proliferation with greater potency in tumor cells harboring the PI3K-α-H1047R mutation compared to PTEN negative, PI3K-α wild-type, and PI3K-α-E545K backgrounds. Both PTEN inactivation and KRAS activation desensitize cells to the growth inhibitory effects of PI3K-α-selective compounds. A wild-type PI3K-α is illustratively provided in SEQ ID NO: 1 and is encoded by a mRNA of SEQ ID NO: 2.

In some embodiments, the first and second cells used in the screening assay have different genetic backgrounds. In one embodiment, the first cell group has a genetic mutation in a PI3K-α kinase domain. In an illustrative embodiment, the genetic mutation in the first cell group includes a mutation in a mRNA (GenBank Accession No. NM 006218, version NM 006218.2 GI: 54792081 herein disclosed as SEQ ID NO: 2 which encodes a full length PI3K-α having a mutation in the kinase domain. In one embodiment, an exemplary mutation is at a codon (3296, 3297 and 3298), in the kinase domain of SEQ ID NO: 2, wherein the codon is mutated to provide an amino acid other than a histidine at position 1047 of PI3K-α provided in SEQ ID NO: 1. In one exemplary mutation, the histidine at 1047 is mutated to arginine (H1047R). This mutation has been previously reported to be a particularly oncogenic mutation in the PI3K/AKT signaling pathway. The second cell group lacks the mutation of the first test cell group. In one embodiment, an exemplary mutation is at a codon (1790, 1791 and 1792), in the helical domain of SEQ ID NO: 2, wherein the codon is mutated to provide an amino acid other than a glutamic acid at position 542 or 545 of PI3K-α provided in SEQ ID NO: 1. In one exemplary mutation, the glutamic acid at 545 is mutated to lysine (for example, E542K or E545K). This mutation has also been previously reported to be a particularly oncogenic mutation in the PI3K/AKT signaling pathway.

In some embodiments, the second cell group can harbor a mutation in PTEN.

In some embodiments, the first cell group can include various cell lines, including cancer cell lines, for example breast cancer cell lines that may be commercially available from the American Type Culture Collection ((ATCC) American Type Culture Collection, Manassas, Va.) bearing the H1047R het genetic mutation of PI3K-α. In some embodiments, the first cell can include HCT-116, T-47D, MDA-MB-453, SIGOV-3, BT-20 or LS H74T cell lines. In some embodiments, the second cell can include MCF-7, PC3 MCI-H460, SK-BR-3, PC-3, MDA-MB-468, SK-BR-3, MDA-MB-231T, or A549. Each specific cell line can be maintained according to instructions provided upon purchase and are commonly available through the ATCC.

In some embodiments, the first cell group and second cell group can also include non-tumor cell lines that have been transformed with a mutant PI3K-α catalytic subunit, for example. H1047R het or E545K PI3K-α catalytic subunit. Methods of introducing nucleic acids and vectors into isolated cells and the culture and selection of transformed host cells in vitro are known in the art and include the use of calcium chloride-mediated transformation, transduction, conjugation, triparental mating, DEAE, dextran-mediated transfection, infection, membrane fusion with liposomes, high velocity bombardment with DNA-coated microprojectiles, direct microinjection into single cells, and electroporation (see, e.g., Sambrook et al., supra; Davis et al., Basic Methods in Molecular Biology, 2′ ed., McGraw-Hill Professional, 1995; and Neumann et al., EMBO J., 1: 841 (1982)). There are several methods for eukaryotic cell transformation, either transiently or stably using a variety of expression vectors. Methods for mutating a cell-line, for example NIH 3T3 cells by amplifying a sequence of DNA encoding the mutated PI3K-α catalytic subunit of interest. The amplified PCR mutant PI3K-α construct can be cloned into a viral expression vector, for example, pSX2neo, a Moloney murine leukemia virus (MLV) long terminal repeat-driven expression vector made by inserting a simian virus 40 early promoter-neomycin phosphotransferase gene into pSX2, designed to express high levels of 10A1 MLV Env. Transformation of NIH 3T3 cells can be performed by transfection with a different CaPO₄ coprecipitation technique. After reaching confluence the cells can be transferred into a medium containing 5% FBS without dexamethasone. Morphologically transformed cells can be separated and isolated from mixtures of transformed and nontransformed Env-plasmid-transfected cells by excising the transformed foci from the cell layer with a small-bore pipette (a Pasteur pipette drawn out over a flame to give a fine tip) and aspiration of the foci by the use of a rubber bulb attached to a pipette.

In some embodiments, the methods described herein require that the cells be tested in the presence of a candidate inhibitor, wherein the candidate inhibitor is added to separate exemplary assay wells, each well containing either the first or second cells. The amount of candidate inhibitor can vary, such that a range of inhibitory activities can be determined for the determination of an IC₅₀ for that candidate inhibitor. This can easily be achieved by serially diluting the compound in an appropriate solvent, for example, DMSO and then in the culture medium in which the first and second cells are being incubated in. In some embodiments, the concentration of the candidate inhibitor can range from about 1 pM to about 1 mM concentration. In some embodiments, the candidate inhibitors are added in amounts ranging from about 0.5 nM to about 10 μM. The incubation of candidate inhibitor with first and second cell groups can vary, typically ranging from about 30 minutes to about 60 hours.

In some embodiments, particularly with PI3K-α mediated activity, the cells are stimulated with a growth factor. The selection of growth factor is mediated by the requirements of the cell line, for example, illustrative growth factors can include VEGF, IGF, insulin and heregulin.

In some embodiments, the inhibitory activity of the candidate compounds can be measured using a variety of cellular activities. When cancer cell lines are being used, the inhibition of PI3K mediated activity, e.g. AKT phosphorylation (both at residues S473 and T308), AKT activation, cellular proliferation, and apoptosis resistance in the cells can all be measured. In some embodiments, the amount of AKT phosphorylation in the first and second cell groups can be measured using a phopho-specific antibody (for example AKT1 (phospho S473, Cat. No. ab8932, AKT1 (phospho T308) Cat. No. ab66134) which are commercially available from AbCam, Cambridge, Mass. Other methods for measuring the inhibition of PI3K-α activity in the first and second cell groups are described in Donahue, A. C. et al., Measuring phosphorylated Akt and other phosphoinositide 3-kinase-regulated phosphoproteins in primary lymphocytes. Methods Enzymol. 2007(434):131-154 which is incorporated herein by reference in its entirety.

In another embodiment, the invention provides a method for determining a treatment regimen for a cancer patient having a tumor comprising a PI3K-α, the method comprising:

determining the presence or absence of a mutation in amino acids 1047 and/or 545 of the PI3K-α;

wherein if the PI3K-α has a mutation at position 1047, the method comprises administering to the cancer patient a therapeutically effective amount of a PI3K-α selective inhibitor compound; or

wherein if the PI3K-α has a mutation at position 545, the method comprises administering to the cancer patient a therapeutically effective amount of a combination of a PI3K-α selective inhibitor and a PI3K-β selective inhibitor, a dual PI3K-α/mTOR selective inhibitor, or a combination of a PI3K-α selective inhibitor and a mTOR selective inhibitor.

In another embodiment, the invention provides a method for determining a treatment regimen for a cancer patient having a tumor comprising a PI3K-α, the method comprising:

determining the presence or absence of a mutation in amino acids 1047 and/or 545 of the PI3K-α;

wherein if the PI3K-α has a mutation at position 1047, the method comprises administering to the cancer patient a therapeutically effective amount of a PI3K-α selective inhibitor compound, a dual PI3K-α/mTOR selective inhibitor, a combination of a PI3K-α selective inhibitor and a mTOR selective inhibitor to the subject; or

wherein if the PI3K-α has a mutation at position 545, the method comprises administering to the cancer patient a therapeutically effective amount of a combination of a PI3K-α selective inhibitor and a PI3K-β selective inhibitor, a dual PI3K-α/mTOR selective inhibitor, or a combination of a PI3K-α selective inhibitor and a mTOR selective inhibitor.

The method of the invention can be used to identify cancer patient populations more likely to benefit from treatment with PI3Kα-selective inhibitors as well as patient populations less likely to benefit.

The invention can be used to further define genetic markers or gene expression signatures which identify PI3Kα inhibitor sensitive tumor subtypes by extended in vitro cell line profiling and in vivo pharmacodynamic and efficacy studies.

In some embodiments, a method for determining a treatment regimen for a cancer patient having the exemplified cancers herein can be readily performed on the basis of the differential activity of PI3K-α selective inhibitors in cancers having a PI3K-α mutated background described herein. In patients in which a tumor cell has been analyzed and assayed to determine whether the tumor harbors a PI3Kα mutation in the kinase domain, for example, a mutation resulting in H1047R, greater efficacy and treatment improvement can be achieved by tailoring a treatment comprising a PI3K-α selective inhibitor. For patients, who have a tumor which does not harbor a mutation in PI3Kα kinase domain, the treatment may require adopting a different treatment regimen, for example, by focusing on delivery of a combination of PI3K-α selective inhibitors and a PI3K-β selective inhibitor, a dual PI3K-α/mTOR selective inhibitor, or a combination of a PI3K-α selective inhibitor and a mTOR selective inhibitor. As indicated above, the PI3K-α selective inhibitors, mTOR selective inhibitors and dual PI3K-α/mTOR selective inhibitors are exemplified in Tables 1, or 2, or 3, and in the detailed description herein.

In some embodiments, methods for determining a treatment regimen comprises determining the presence of a mutation in amino acids 1047 and/or 545 of the PI3K-α in the subject's tumor. This step can be achieved in a variety of ways, using nucleic acid approaches, protein separation approaches or direct immunological approaches using mutation specific antibodies. In some embodiments, presence of a mutation in amino acids 1047 and/or 545 of the PI3K-α in the subject's tumor can be determined using any suitable method for the sequence analysis of amino acids. Examples of suitable techniques include, but are not limited to, western blot analysis, immunoprecipitation, radioimmunoassay (RIA) or enzyme-linked immunoabsorbent assay (ELISA).

In the present invention, reference to position within the amino acid sequence of PI3Kα is made referring to SEQ ID NO: 1. Reference to positions within the nucleotide sequence of the PI3Kα is made referring to SEQ ID NO:2. Specific amino acids in the wild type protein sequence are described using single letter amino acid designation followed by the position in the protein sequence, for example E545 indicates that position 545 is glutamic acid. To represent a substitution at a particular position, the substituted amino acid follows the position, for example E545K indicates that the glutamic acid at position 545 is replaced with a lysine.

Determining the presence or absence of mutations in the sequence of the PI3K-α peptide sequence is generally determined using in vitro methods wherein a tumor sample is used which has been removed from the body of a patient.

Determining the presence or absence of mutations in the amino acid sequence of PI3Kα or a portion thereof, can be done using any suitable method. For example the nucleotide sequence of PI3Kα or a portion thereof maybe determined and the amino acid sequence deduced from the nucleotide sequence or a PI3K-α protein can be interrogated directly.

The nucleotide sequence of the PI3K-α, or a portion thereof, may be determined using any method for the sequence analysis of nucleic acids. Methods for identification of sequence mutation in genes are well known in the art and the mutations in the PI3Kα can be identified by any suitable method. These methods include, but are not limited to, dynamic allele-specific hybridization; the use of molecular beacons; enzyme-based methods, using for example DNA ligase, DNA polymerase or nucleases; PCR based methods, whole genome sequencing; partial genome sequencing; exome sequencing; nucleic acid probe hybridization; and restriction enzyme digestion analysis.

Methods of Direct DNA sequencing are well known in the art, (see for example: Current Protocols in Molecular Biology, edited by Fred M. Ausubel, Roger Brent, Robert E. Kingston, David D. Moore, J. G. Seidman, John A. Smith, Kevin Struhl, and Molecular Cloning: A Laboratory Manual, Joe Sambrook, David W Russel, 3^{rd} edition, Cold Spring Harbor Laboratory Press).) These sequencing protocols include for example, the use of radioactively labeled nucleotides, and nucleotides labeled with a fluorescent dye.

For example, Barbi, S. et al., used the following protocol to sequence the helical domain (exon 9) and the kinase domain (exon 20) of PI3K-α. Normal and tumor DNA was extracted from paraffin-embedded tissue. and amplified using fluorescent dye-labeled primers. the following primer pairs. Primer sequences need to be chosen to uniquely select for a region of DNA, avoiding the possibility of mishybridization to a similar sequence nearby. A commonly used method is BLAST search whereby all the possible regions to which a primer may bind can be seen. Both the nucleotide sequence as well as the primer itself can be BLAST searched. The free NCBI tool Primer-BLAST integrates primer design tool and BLAST search into one application, so does commercial software product such as Beacon Designer, (Premier Biosoft International, Palo Alto Calif.). Mononucleotide repeats should be avoided, as loop formation can occur and contribute to mishybridization. In addition, computer programs are readily available to aid in design of suitable primers. In certain embodiments the nucleic acid probe is labeled for use in a Southern hybridization assay. The nucleic acid probe may be radioactively labeled, fluorescently labeled or is immunologically detectable, in particular is a digoxygenin-labeled (Roche Diagnostics GmbH, Mannheim).

In some embodiments, determining the presence of a helical domain mutation in exon 9 can include the use of forward primer and reverse primers: GGGAAAAATATGACAAAGAAAGC (SEQ ID NO: 3) and CTGAGATCAGCCAAATTCAGIT (SEQ ID NO: 4) respectively and a sequencing primer can include TAGCTAGAGACAATGAATTAAGGGAAA (SEQ ID NO: 5).

For determining a mutation in the kinase domain in exon 20, an exemplary set of primers can include: forward and reverse primers CTCAATGATGCTTGGCTCTG (SEQ ID NO: 6) and TGGAATCCAGAGTGAGCTTTC (SEQ ID NO: 7) respectively and the sequencing primer can include TTGATGACATTGCATACATTCG (SEQ ID NO: 8). The amplification products can then be sequenced. (Barbi, S. et al. J. Experimental and Clinical Cancer Research 2010, 29:32) The sequences are then compared and differences between the wild type PI3K-α sequence and the sequence of the tumor PI3K-α. are determined. The assay could also be performed by only amplifying the tumor DNA and comparing the PI3K-α sequence in the tumor with the sequence of SEQ ID NO:1.

In some embodiments, the present invention provides polynucleotide sequences comprising polynucleotide sequences in whole or in part from SEQ ID NO: 2 that are capable of hybridizing to the helical region, or the kinase domain of PI3K-α under conditions of high stringency. In some embodiments, the polynucleotides can include sequences complementary to nucleic acid sequences that encode in whole or in part PI3K-α or PI3K-α having specific mutations as described herein. The terms “complementary” and “complementarity” refer to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods which depend upon binding between nucleic acids.

In some embodiments, the present invention provides polynucleotide sequences comprising polynucleotide sequences in whole or in part from SEQ ID NO: 2 that are capable of hybridizing to the helical region, or the kinase domain oPI3K-α under conditions of high stringency. In some embodiments, the present method includes using isolated RNA from a subject's tumor in an assay to determine whether there is a mutation at amino acid at position 1047, 542, or 545 of SEQ ID NO:1, the assay further comprises: (a) reverse transcribing said RNA sample into an equivalent cDNA; (b) amplifying a predetermined region of the cDNA using a pair of nucleic acid probes directed to a predetermined region of the PI3K-α gene; (c) sequencing said amplified cDNA region to obtain a polynucleotide sequence of said amplified cDNA region; and (d) determining whether said amplified cDNA region contains a gene mutation in a codon encoding the amino acid at position 1047, 542, or 545 of SEQ ID NO:1.

In some embodiments, the present methods can employ amplifying a predetermined region of the cDNA by amplifying the cDNA using a pair of nucleic acid primers, a first primer capable of hybridizing stringently to the cDNA upstream of a DNA codon encoding the amino acid at either amino acid 1047 or 542 or 545 of SEQ ID NO:1, and second a nucleic acid primer operable to hybridize stringently to the cDNA downstream of a DNA codon encoding the amino acid at either amino acid 1047 or 542 or 545 of SEQ ID NO:1

In some embodiments, the polynucleotides can include sequences complementary to nucleic acid sequences that encode in whole or in part PI3K-α or PI3K-α having specific mutations as described herein. The terms “complementary” and “complementarity” refer to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods which depend upon binding between nucleic acids.

“High stringency conditions” when used in reference to nucleic acid hybridization comprise conditions equivalent to binding or hybridization at 42C.° in a solution consisting of 5×SSPE (43.8 g/l NaCl, 6.9 g/l NaH₂PO₄.H₂O and 1.85 g/l EDTA, pH adjusted to 7.4 with NaOH), 0.5% SDS, 5×Denhardt's reagent and 100 μg/mL denatured salmon sperm DNA followed by washing in a solution comprising 0.1×SSPE, 1.0% SDS at 42C.° when a probe of about 500 nucleotides in length is employed.

The term “homology” when used in relation to nucleic acids refers to a degree of complementarity. There may be partial homology or complete homology (i.e., identity). “Sequence identity” refers to a measure of relatedness between two or more nucleic acids or proteins, and is given as a percentage with reference to the total comparison length. The identity calculation takes into account those nucleotide or amino acid residues that are identical and in the same relative positions in their respective larger sequences. Calculations of identity may be performed by algorithms contained within computer programs such as “GAP” (Genetics Computer Group, Madison, Wis.) and “ALIGN” (DNAStar, Madison, Wis.). A partially complementary sequence is one that at least partially inhibits (or competes with) a completely complementary sequence from hybridizing to a target nucleic acid is referred to using the functional term “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a sequence which is completely homologous to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target which lacks even a partial degree of complementarity (e.g., less than about 30% identity); in the absence of non-specific binding the probe will not hybridize to the second non-complementary target.

In preferred embodiments, hybridization conditions are based on the melting temperature (Tm) of the nucleic acid binding complex and confer a defined “stringency” The term “hybridization” refers to the pairing of complementary nucleic acids. Hybridization and the strength of hybridization (i.e., the strength of the association between the nucleic acids) is impacted by such factors as the degree of complementary between the nucleic acids, stringency of the conditions involved, the Tm of the formed hybrid, and the G:C ratio within the nucleic acids. A single molecule that contains pairing of complementary nucleic acids within its structure is said to be “self-hybridized.”

The term “Tm” refers to the “melting temperature” of a nucleic acid. The melting temperature is the temperature at which a population of double-stranded nucleic acid molecules becomes half dissociated into single strands. The equation for calculating the Tm of nucleic acids is well known in the art. As indicated by standard references, a simple estimate of the Tm value may be calculated by the equation: Tm=81.5+0.41(% G+C), when a nucleic acid is in aqueous solution at 1 M NaCl. The term “stringency” refers to the conditions of temperature, ionic strength, and the presence of other compounds such as organic solvents, under which nucleic acid hybridizations are conducted. With “high stringency” conditions, nucleic acid base pairing will occur only between nucleic acid fragments that have a high frequency of complementary base sequences.

In addition, sequence mutations in the PI3Kα can be determined using any sequence-specific nucleic acid detection method allowing detection of single-nucleotide variation, in particular any such method involving complementary base pairing. For example, to determine if the PI3K-α comprises a E545 mutation, the sequence of PI3K-α peptide or a portion thereof comprising nucleotides 1790, 1791 and 1792 of SEQ ID NO:2 (codon corresponding with position 545 in the amino acid sequence), is used in a polymerase chain reaction (PCR) where the oligonucleotide primers allow the amplification of PI3Kα only if the nucleotide at position 1790 is G. If no reaction product is formed then the amino acid at position 545 is mutated. In another example the oligonucleotide primers are designed to allow the amplification of the to allow amplification if the nucleotide at position 3297 is A (codon comprising nucleotides 3296, 3297 and 3298 corresponds with position 1047 of the amino acid sequence). If no reaction product is formed using those primers then the amino acid at position 545 is mutated. Methods for performing PCR are known in the art (see Current Protocols in Molecular Biology, edited by Fred M. Ausubel, Roger Brent, Robert E. Kingston, David D. Moore, J. G. Seidman, John A. Smith, Kevin Struhl. and; Molecular Cloning: A Laboratory Manual, Joe Sambrook, David W Russel, 3^{rd} edition, Cold Spring Harbor Laboratory Press).

Dynamic allele-specific hybridization (DASH) genotyping takes advantage of the differences in the melting temperature in DNA that results from the instability of mismatched base pairs. This technique is well suited to automation. In the first step, a DNA segment is amplified and attached to a bead through a PCR reaction with a biotinylated primer. In the second step, the amplified product is attached to a streptavidin column and washed with NaOH to remove the un-biotinylated strand. An sequence-specific oligonucleotide is then added in the presence of a molecule that fluoresces when bound to double-stranded DNA. The intensity is then measured as temperature is increased until the Tm can be determined. A single nucleotide change will result in a lower than expected Tm (Howell W., Jobs M., Gyllensten U., Brookes A. (1999) Dynamic allele-specific hybridization. A new method for scoring single nucleotide polymorphisms. Nat. Biotechnol. 17(1):87-8). Because DASH genotyping is measuring a quantifiable change in Tm, it is capable of measuring all types of mutations, not just SNPs. Other benefits of DASH include its ability to work with label free probes and its simple design and performance conditions.

Molecular beacons can also be used to detect mutations in a DNA sequences Molecular beacons makes use of a specifically engineered single-stranded oligonucleotide probe. The oligonucleotide is designed such that there are complementary regions at each end and a probe sequence located in between. This design allows the probe to take on a hairpin, or stem-loop, structure in its natural, isolated state. Attached to one end of the probe is a fluorophore and to the other end a fluorescence quencher. Because of the stem-loop structure of the probe, the fluorophore is in close proximity to the quencher, thus preventing the molecule from emitting any fluorescence. The molecule is also engineered such that only the probe sequence is complementary to the to the genomic DNA that will be used in the assay (Abravaya K., Huff J., Marshall R., Merchant B., Mullen C., Schneider G., and Robinson J. (2003) Molecular beacons as diagnostic tools: technology and applications. Clin Chem Lab Med. 41:468-474). If the probe sequence of the molecular beacon encounters its target genomic DNA during the assay, it will anneal and hybridize. Because of the length of the probe sequence, the hairpin segment of the probe will denatured in favor of forming a longer, more stable probe-target hybrid. This conformational change permits the fluorophore and quencher to be free of their tight proximity due to the hairpin association, allowing the molecule to fluoresce. If on the other hand, the probe sequence encounters a target sequence with as little as one non-complementary nucleotide, the molecular beacon will preferentially stay in its natural hairpin state and no fluorescence will be observed, as the fluorophore remains quenched. The unique design of these molecular beacons allows for a simple diagnostic assay to identify SNPs at a given location. If a molecular beacon is designed to match a wild-type allele and another to match a mutant of the allele, the two can be used to identify the genotype of an individual. If only the first probe's fluorophore wavelength is detected during the assay then the individual is homozygous to the wild type. If only the second probe's wavelength is detected then the individual is homozygous to the mutant allele. Finally, if both wavelengths are detected, then both molecular beacons must be hybridizing to their complements and thus the individual must contain both alleles and be heterozygous.

Enzyme-based nucleic acid methods are also suitable and contemplated for determining mutations in the PI3K-α nucleotide sequence. For example, Restriction fragment length polymorphism (RFLP) (discussed in greater detail below) can be used to detect single nucleotide differences. SNP-RFLP makes use of the many different restriction endonucleases and their high affinity to unique and specific restriction sites. By performing a digestion on a genomic sample and determining fragment lengths through a gel assay it is possible to ascertain whether or not the enzymes cut the expected restriction sites. A failure to cut the genomic sample results in an identifiably larger than expected fragment implying that there is a mutation at the point of the restriction site which is rendering it protected from nuclease activity.

The term “functionally equivalent codon” is used herein to refer to codons that encode the same amino acid, such as the six codons for arginine.

In one embodiment of the invention the method comprises at least one nucleic acid probe or oligonucleotide for determining the sequence of the codon that encodes amino acid 1047. In another embodiment the method comprises at least one nucleic acid probe or oligonucleotide for determining the sequence of the codon that encodes amino acid 545. The oligonucleotide is a PCR primer, preferably a set of PCR primers which allows amplification of a PI3Kα nucleic acid sequence fragment only if the codon which encodes amino acid 1047 encodes a histidine. In another method, the PCR primer or set of PCR primers allows the amplification of nucleic acid sequence fragment only if the codon which encodes amino acid 545 encodes a glutamic acid. Determination of suitable PCR primers is routine in the art, (Current Protocols in Molecular Biology, edited by Fred M. Ausubel, Roger Brent, Robert E. Kingston, David D. Moore, J. G. Seidman, John A. Smith, Kevin Struhl; Looseleaf: 0-471-650338-X; CD-ROM: 0-471-30661-4). In addition, computer programs are readily available to aid in design of suitable primers. In certain embodiments the nucleic acid probe is labeled for use in a Southern hybridization assay. The nucleic acid probe may be radioactively labeled, fluorescently labeled or is immunologically detectable, in particular is a digoxygenin-labeled (Roche Diagnostics GmbH, Mannheim).

U.S. Patent Publication 20010016323 discloses methods for detecting point mutations using a fluorescently labeled oligonucleotidemeric probe and fluorescence resonance energy transfer. A point mutation leading to a base mismatch between the probe and the target DNA strand causes the melting temperature of the complex to be lower than the melting temperature for the probe and the target if the probe and target were perfectly matched.

Other suitable methods for detecting single point mutations include those disclosed in, for example, U.S. Patent Publication 2002010665, which involves the use of oligonucleotide probes in array format. Such arrays can include one or more of SEQ ID NOs:3-8. U.S. Patent Publication 20020177157 discloses additional methods for detecting point mutations.

A polynucleotide carrying a point mutation leading to a mutation of PI3K-α kinase domain, for example, H1047R that is the subject of this invention can be identified using one or more of a number of available techniques. However, detection is not limited to the techniques described herein and the methods and compositions of the invention are not limited to these methods, which are provided for exemplary purposes only. Polynucleotide and oligonucleotide probes are also disclosed herein and are within the scope of the invention, and these probes are suitable for one or more of the techniques described below. These include allele-specific oligonucleotide hybridization (ASO), which, in one embodiment, is a diagnostic mutation detection method wherein hybridization with a pair of oligonucleotides corresponding to alleles of a known mutation is used to detect the mutation. Another suitable method is denaturing high performance liquid chromatography (DHPLC), which is a liquid chromatography method designed to identify mutations and polymorphisms based on detection of heteroduplex formation between mismatched nucleotides. Under specified conditions, heteroduplexes elute from the column earlier than homoduplexes because of reduced melting temperature. Analysis can then be performed on individual samples.

An amplified region of the DNA containing the mutation or the wild-type sequence can be analyzed by DHPLC. Use of DHPLC is described in U.S. Pat. Nos. 5,795,976 and 6,453,244, both of which are incorporated herein by reference. A suitable method is that provided by Transgenomic, Inc. (Omaha, Nebr.) using the Transgenomic WAVE® System.

For ASO, a region of genomic DNA or cDNA containing the PI3K-α mutation (H1047R and/or E545K) is amplified by PCR and transferred onto duplicating membranes. This can be performed by dot/slot blotting, spotting by hand, or digestion and Southern blotting. The membranes are prehybridized, then hybridized with a radiolabeled or deoxygenin (DIG) labeled oligonucleotide to either the mutant or wild-type sequences. For the DIG label, detection is performed using chemiluminescent or colorimetric methods. The membranes are then washed with increasing stringency until the ASO is washed from the non-specific sequence. Following autoradiographic exposure, the products are scored for the level of hybridization to each oligonucleotide. Optimally, controls are included for the normal and mutant sequence on each filter to confirm correct stringency, and a negative PCR control is used to check for contamination in the PCR.

The size of the ASO probe is not limited except by technical parameters of the art. Generally, too short a probe will not be unique to the location, and too long a probe may cause loss of sensitivity. The oligonucleotides are preferably 15-21 nucleotides in length, with the mismatch towards the center of the oligonucleotide.

The region of sample DNA on which ASO hybridization is performed to detect the mutation of this invention is preferably amplified by PCR using a forward primer, For exon 9 the forward primer and reverse primers were GGGAAAAATATGACAAAGAAAGC (SEQ ID NO: 3) and CTGAGATCAGCCAAATTCAGTT (SEQ ID NO: 4) respectively and the sequencing primer was TAGCTAGAGACAATGAATTAAGGGAAA (SEQ ID NO: 5), for exon 20 the forward and reverse primers were CTCAATGATGCTTGGCTCTG (SEQ ID NO: 6) and TGGAATCCAGAGTGAGCTTTC (SEQ ID NO: 7) respectively. In this case, amplification by PCR or a comparable method is not necessary but can optionally be performed.

Optionally, one or more than one of the amplified regions described above, (including the 306 nucleotide region generated using primers of SEQ ID NO:3-8, or shorter portions of either of these regions, can be analyzed by sequencing in order to detect the mutation. Sequencing can be performed as is routine in the art. The only limitation on choice of the region to be sequenced, in order to identify the presence of the mutation, is that the region selected for sequencing must include the nucleotide that is the subject of the mutation. The size of the region selected for sequencing is not limited except by technical parameters as is known in the art, and longer regions comprising part or all of the DNA or RNA between selected amplified regions using the primers SEQ ID NOs: 3 & 4 and 6 & 7 disclosed herein can be sequenced.

Variations of the methods disclosed above are also suitable for detecting the mutation. For example, in a variation of ASO, the ASO's are given homopolymer tails with terminal deoxyribonucleotidyl transferase, spotted onto nylon membrane, and covalently bound by UV irradiation. The target DNA is amplified with biotinylated primers and hybridized to the membrane containing the immobilized oligonucleotides, followed by detection. An example of this reverse dot blot technique is the INNO-LIPA kit from Innogenetics (Belgium).

With the identification and sequencing of the mutated gene and the gene product, i.e. SEQ ID NO:1 having a mutation at E545K and H1047R, probes and antibodies raised to the gene product can be used in a variety of hybridization and immunological assays to screen for and detect the presence of either a normal or mutated gene or gene product.

Expression of the mutated gene in heterologous cell systems can be used to demonstrate structure function relationships. Ligating the DNA sequence into a plasmid expression vector to transfect cells is a useful method to test the influence of the mutation on various cellular biochemical parameters. Plasmid expression vectors containing either the entire normal or mutant human or mouse sequence or portions thereof, can be used in in vitro mutagenesis experiments which will identify portions of the protein crucial for regulatory function.

The DNA sequence can be manipulated in studies to understand the expression of the gene and its product, and to achieve production of large quantities of the protein for functional analysis, for antibody production, and for patient therapy. Changes in the sequence may or may not alter the expression pattern in terms of relative quantities, tissue-specificity and functional properties.

A number of methods are available for analysis of variant (e.g., mutant or polymorphic) nucleic acid sequences. Assays for detections polymorphisms or mutations fall into several categories, including, but not limited to direct sequencing assays, fragment polymorphism assays, hybridization assays, and computer based data analysis. Protocols and commercially available kits or services for performing multiple variations of these assays are commercially available and known to those of skill in the art. In some embodiments, assays are performed in combination or in combined parts (e.g., different reagents or technologies from several assays are combined to yield one assay). The following illustrative assays may be used to screen and identify nucleic acid molecules containing the mutations of PI3K-α mutation of interest.

### Fragment Length Polymorphism Assays

In some embodiments of the present invention, variant sequences are detected using a fragment length polymorphism assay. In a fragment length polymorphism assay, a unique DNA banding pattern based on cleaving the DNA at a series of positions is generated using an enzyme (e.g., a restriction enzyme or a CLEAVASE I [Third Wave Technologies, Madison, Wis.] enzyme). DNA fragments from a sample containing a SNP or a mutation will have a different banding pattern than wild type.

### PCR Assays

In some embodiments of the present invention, variant sequences are detected using a PCR-based assay. In some embodiments, the PCR assay comprises the use of oligonucleotide nucleic acid primers that hybridize only to the variant or wild type allele of PI3Kα (e.g., to the region of mutation or multiple mutations). Both sets of primers are used to amplify a sample of DNA. If only the mutant primers result in a PCR product, then the subject's tumor or cancer expresses a somatic mutation in an PI3K-α mutation allele. PCR amplification conditions are tailored to the specific oligonucleotide primers or oligonucleotide probes used, the quality and type of DNA or RNA being screened, and other well known variables that can be controlled using appropriate reagents and/or PCR cycling conditions known to those of ordinary skill in the art.

### RFLP Assays

In some embodiments of the present invention, variant sequences are detected using a restriction fragment length polymorphism assay (RFLP). The region of interest is first isolated using PCR. The PCR products are then cleaved with restriction enzymes known to give a unique length fragment for a given polymorphism. The restriction-enzyme digested PCR products are separated by agarose gel electrophoresis and visualized by ethidium bromide staining. The length of the fragments is compared to molecular weight markers and fragments generated from wild-type and mutant controls.

### Direct Sequencing Assays

In some embodiments of the present invention, variant sequences are detected using a direct sequencing technique. In these assays, DNA samples are first isolated from a subject using any suitable method. In some embodiments, the region of interest is cloned into a suitable vector and amplified by growth in a host cell (e.g., a bacteria). In other embodiments, DNA in the region of interest is amplified using PCR.

Following amplification, DNA in the region of interest (e.g., the region containing the SNP or mutation of interest) is sequenced using any suitable method, including but not limited to manual sequencing using radioactive marker nucleotides, or automated sequencing. The results of the sequencing are displayed using any suitable method. The sequence is examined and the presence or absence of a given SNP or mutation is determined.

### CFLP Assays

In other embodiments, variant sequences are detected using a CLEAVASE fragment length polymorphism assay (CFLP; Third Wave Technologies, Madison, Wis.; See e.g., U.S. Pat. Nos. 5,843,654; 5,843,669; 5,719,208; and 5,888,780; each of which is herein incorporated by reference). This assay is based on the observation that when single strands of DNA fold on themselves, they assume higher order structures that are highly individual to the precise sequence of the DNA molecule. These secondary structures involve partially duplexed regions of DNA such that single stranded regions are juxtaposed with double stranded DNA hairpins. The CLEAVASE I enzyme, is a structure-specific, thermostable nuclease that recognizes and cleaves the junctions between these single-stranded and double-stranded regions. The region of interest is first isolated, for example, using PCR. Then, DNA strands are separated by heating. Next, the reactions are cooled to allow intra-strand secondary structure to form. The PCR products are then treated with the CLEAVASE I enzyme to generate a series of fragments that are unique to a given SNP or mutation. The CLEAVASE enzyme treated PCR products are separated and detected (e.g., by agarose gel electrophoresis) and visualized (e.g., by ethidium bromide staining). The length of the fragments is compared to molecular weight markers and fragments generated from wild-type and mutant controls.

### Hybridization Assays

In some embodiments of the present invention, variant sequences are detected by hybridization analysis in a hybridization assay. In a hybridization assay, the presence or absence of a given mutation is determined based on the ability of the DNA from the sample to hybridize to a complementary DNA molecule (e.g., a oligonucleotide probe or probes as illustrated herein). A variety of hybridization assays using a variety of technologies for hybridization and detection are available. Relevant and useful hybridization assays for practicing the methods of the present invention are provided below.

### Direct Detection of Hybridization

In some embodiments, hybridization of a probe to the sequence of interest (e.g., a SNP or mutation) is detected directly by visualizing a bound probe (e.g., a Northern or Southern assay; See e.g., Ausabel et al. (eds.) (1991) Current Protocols in Molecular Biology, John Wiley & Sons, NY). In a these assays, genomic DNA (Southern) or RNA (Northern) is isolated from a subject. The DNA or RNA is then cleaved with a series of restriction enzymes that cleave infrequently in the genome and not near any of the markers being assayed. The DNA or RNA is then separated (e.g., on an agarose gel) and transferred to a membrane. A labeled (e.g., by incorporating a radionucleotide) probe or probes specific for the SNP or mutation being detected is allowed to contact the membrane under a condition or low, medium, or high stringency conditions. The unbound probe is removed and the presence of binding is detected by visualizing the labeled probe.

### Detection of Hybridization Using “DNA Chip” Assays

In some embodiments of the present invention, variant sequences are detected using a DNA chip hybridization assay. In this assay, a series of oligonucleotide probes are affixed to a solid support. The oligonucleotide probes are designed to be unique to a given SNP or mutation. The DNA sample of interest is contacted with the DNA “chip” and hybridization is detected.

In some embodiments, an illustrative and commercially available DNA chip assay can include a GENECHIP® (commercially available from Affymetrix, Santa Clara, Calif., USA); See e.g., U.S. Pat. Nos. 6,045,996; 5,925,525; and 5,858,659; each of which is herein incorporated by reference) assay. The GENECHIP® technology uses miniaturized, high-density arrays of oligonucleotide probes affixed to a “chip.” Probe arrays are manufactured by Affymetrix's light-directed chemical synthesis process, which combines solid-phase chemical synthesis with photolithographic fabrication techniques employed in the semiconductor industry. Using a series of photolithographic masks to define chip exposure sites, followed by specific chemical synthesis steps, the process constructs high-density arrays of oligonucleotides, with each probe in a predefined position in the array. Multiple probe arrays are synthesized simultaneously on a large glass wafer. The wafers are then diced, and individual probe arrays are packaged in injection-molded plastic cartridges, which protect them from the environment and serve as chambers for hybridization.

The nucleic acid to be analyzed is isolated, amplified by PCR, and labeled with a fluorescent reporter group. The labeled DNA is then incubated with the array using a fluidics station. The array is then inserted into the scanner, where patterns of hybridization are detected. The hybridization data are collected as light emitted from the fluorescent reporter groups already incorporated into the target, which is bound to the probe array. Probes that perfectly match the target generally produce stronger signals than those that have mismatches. Since the sequence and position of each probe on the array are known, by complementarity, the identity of the target nucleic acid applied to the probe array can be determined.

### Enzymatic Detection of Hybridization

In some embodiments of the present invention, hybridization can be detected by enzymatic cleavage of specific structures (INVADER assay, Third Wave Technologies; See e.g., U.S. Pat. Nos. 5,846,717, 6,090,543; 6,001,567; 5,985,557; and 5,994,069; each of which is herein incorporated by reference). The INVADER assay detects specific DNA and RNA sequences by using structure-specific enzymes to cleave a complex formed by the hybridization of overlapping oligonucleotide probes. Elevated temperature and an excess of one of the probes enable multiple probes to be cleaved for each target sequence present without temperature cycling. These cleaved probes then direct cleavage of a second labeled probe. The secondary probe oligonucleotide can be 5′-end labeled with fluorescein that is quenched by an internal dye. Upon cleavage, the de-quenched fluorescein labeled product may be detected using a standard fluorescence plate reader. The INVADER assay detects specific mutations in unamplified genomic DNA. The isolated DNA sample is contacted with the first probe specific either for a mutation of the present invention or wild type PI3K-α sequence and allowed to hybridize. Then a secondary probe, specific to the first probe, and containing the fluorescein label, is hybridized and the enzyme is added. Binding is detected by using a fluorescent plate reader and comparing the signal of the test sample to known positive and negative controls.

In some embodiments, hybridization of a bound probe is detected using a TaqMan assay (PE Biosystems, Foster City, Calif.; See e.g., U.S. Pat. Nos. 5,962,233 and 5,538,848, each of which is herein incorporated by reference). The assay is performed during a PCR reaction. The TaqMan assay exploits the 5′-3′ exonuclease activity of the AMPLITAQ GOLD DNA polymerase. A probe, specific for a given allele or mutation, is included in the PCR reaction. The probe consists of an oligonucleotide with a 5′-reporter dye (e.g., a fluorescent dye) and a 3′-quencher dye. During PCR, if the probe is bound to its target, the 5′-3′ nucleolytic activity of the AMPLITAQ GOLD polymerase cleaves the probe between the reporter and the quencher dye. The separation of the reporter dye from the quencher dye results in an increase of fluorescence. The signal accumulates with each cycle of PCR and can be monitored with a fluorometer.

In accordance with the present invention, diagnostic kits are also provided which will include the reagents necessary for the above-described diagnostic screens. For example, kits may be provided which include oligonucleotide probes or PCR primers are present for the detection and/or amplification of mutant PI3K-α, and comparable wild-type PI3K-α-related nucleotide sequences. Again, such probes may be labeled for easier detection of specific hybridization. As appropriate to the various diagnostic embodiments described above, the oligonucleotide probes in such kits may be immobilized to substrates and appropriate controls may be provided. Examples of such oligonucleotide probes include oligonucleotides comprising or consisting of at least one of SEQ ID NOs:3&4 and 6&7.

Determining the presence or absence of mutations in the amino acid sequence of PI3Kα can be determined using any method for the sequence analysis of amino acids. Non-limiting examples include: western blot analysis or ELISA assays, or direct protein sequencing of the PI3Kα in the subject's tumor. In some embodiments, particularly useful antibodies have selectivity for wild type PI3K-α versus the mutant PI3Kα, for example, an antibody useful in the assay would bind to wild type PI3K-α, or a portion wild type PI3Kα, but not to a PI3Kα having a mutation at the amino acid of interest. Particularly useful antibodies could include antibodies which bind the wild type PI3Kα which has histidine at position 1047 but does not bind a mutant PI3Kα which has an amino acid other than histidine, such as arginine, in other words the antibody specifically bind to an epitope comprising histidine at position 1047. Likewise, particularly useful are antibodies which bind the wild type PI3Kα which has glutamic acid at position 545 but does not bind a mutant PI3Kα which has an amino acid other than glutamic acid at position 545, such as lysine at that position.

Another embodiment of the invention provides a method comprising the use of at least one antibody which binds selectively to the wild type PI3Kα protein as compared with binding to a mutated form of PI3Kα. Alternately the antibody binds selectively to a mutated form of PI3Kα as compared with binding to the wild type PI3Kα protein and can differentiate between wild-type PI3Kα and PI3Kα-H1047R or between wild-type PI3Kα and PI3Kα-E545K. Methods for isolating suitable amounts of target protein from a complex mixture in relatively small amounts (less than 1 mg) are commonly known by those skilled in the art. In one illustrative embodiment, a tumor cell or plurality of tumor cells from a subject's tumor or cancer are lysed using commonly available lysing reagents in the presence of protease inhibitors. The lysate is cleared and the supernatant is either electrophoresed and subjected to a Western Blot using mutation specific antibodies, or alternatively, the mutated PI3Kα-H1047R or PI3Kα-E545K are selectively immunoprecipitated and further dissociated from the capture antibody and subjected to Western Blotting or protein sequenced directly.

“Antibody” includes, any immunoglobulin molecule that recognizes and specifically binds to a target, such as a protein, polypeptide, peptide, carbohydrate, polynucleotide, lipid, etc., through at least one antigen recognition site within the variable region of the immunoglobulin molecule. As used herein, the term is used in the broadest sense and encompasses intact polyclonal antibodies, intact monoclonal antibodies, antibody fragments (such as Fab, Fab′, F(ab′)₂, and Fv fragments), single chain Fv (scFv) mutants, multispecific antibodies such as bispecific antibodies generated from at least two intact antibodies, fusion proteins comprising an antibody portion, and any other modified immunoglobulin molecule comprising an antigen recognition site so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes and the like.

“Antibody fragment” can refer to a portion of an intact antibody. Examples of antibody fragments include, but are not limited to, linear antibodies; single-chain antibody molecules; Fc or Fc′ peptides, Fab and Fab fragments, and multispecific antibodies formed from antibody fragments.

“Chimeric antibodies” refers to antibodies wherein the amino acid sequence of the immunoglobulin molecule is derived from two or more species. Typically, the variable region of both light and heavy chains corresponds to the variable region of antibodies derived from one species of mammals (e.g. mouse, rat, rabbit, etc) with the desired specificity, affinity, and capability while the constant regions are homologous to the sequences in antibodies derived from another (usually human) to avoid eliciting an immune response in that species.

“Humanized” forms of non-human (e.g., rabbit) antibodies include chimeric antibodies that contain minimal sequence, or no sequence, derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies can comprise residues that are not found in the recipient antibody or in the donor antibody. Most often, the humanized antibody can comprise substantially all of at least one, and typically two, variable domains, in which all or substantially all of the hypervariable loops correspond to those of a nonhuman immunoglobulin and all or substantially all of the FR residues are those of a human immunoglobulin sequence. The humanized antibody can also comprise at least a portion of an immunoglobulin constant region (Fc), typically that of a human immunoglobulin. Methods used to generate humanized antibodies are well known in the field of immunology and molecular biology.

“Hybrid antibodies” can include immunoglobulin molecules in which pairs of heavy and light chains from antibodies with different antigenic determinant regions are assembled together so that two different epitopes or two different antigens can be recognized and bound by the resulting tetramer.

The term “epitope” or “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids are typically retained upon protein denaturing, whereas epitopes formed by tertiary folding are typically lost upon protein denaturing. An epitope typically includes at least 3-5, and more usually, at least 5 or 8-10 amino acids in a unique spatial conformation.

“Specifically binds” to or shows “specific binding” towards an epitope means that the antibody reacts or associates more frequently, and/or more rapidly, and/or greater duration, and/or with greater affinity with the epitope than with alternative substances.

### Preparation of Antibodies

### Polyclonal Antibodies

Polyclonal antibodies are preferably raised in animals by multiple subcutaneous (sc) or intraperitoneal (ip) injections of the relevant antigen and an adjuvant. Alternatively, antigen may be injected directly into the animal's lymph node (see Kilpatrick et al., Hybridoma, 16:381-389, 1997). An improved antibody response may be obtained by conjugating the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride or other agents known in the art.

Animals are immunized against the antigen, immunogenic conjugates or derivatives by combining, e.g., 100 μg of the protein or conjugate (for mice) with 3 volumes of Freund's complete adjuvant and injecting the solution intradermally at multiple sites. One month later, the animals are boosted with ⅕ to 1/10 the original amount of peptide or conjugate in Freund's complete adjuvant by subcutaneous injection at multiple sites. At 7-14 days post-booster injection, the animals are bled and the serum is assayed for antibody titer. Animals are boosted until the titer plateaus. Preferably, the animal is boosted with the conjugate of the same antigen, but conjugated through a different cross-linking reagent. Conjugates also can be made in recombinant cell culture as protein fusions. Also, aggregating agents such as alum are suitably used to enhance the immune response.

### Monoclonal Antibodies

Monoclonal antibodies can be made using the hybridoma method first described by Kohler et al., Nature, 256:495 (1975), or by recombinant DNA methods. In the hybridoma method, a mouse or other appropriate host animal, such as rats, hamster or macaque monkey, is immunized to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)). The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells and are sensitive to a medium. Human myeloma and mouse-human heteromyeloma cell lines also have been described for the production of human monoclonal antibodies (Kozbor, J. Immunol., 133: 3001 (1984); Brodeur et al., Monoclonal Antibody Production Techniques and Applications, pp. 51-63 (Marcel Dekker, Inc., New York, 1987)). Exemplary murine myeloma lines include those derived from MOP-21 and M. C.-11 mouse tumors available from the Salk Institute Cell Distribution Center, San Diego, Calif. USA, and SP-2 or X63-Ag8-653 cells available from the American Type Culture Collection, Rockville, Md. USA. Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA). The binding affinity of the monoclonal antibody can be determined, for example, by BIAcore or Scatchard analysis (Munson et al., Anal. Biochem., 107:220 (1980)).

After hybridoma cells are identified that produce antibodies of the desired specificity, affinity, and/or activity, the clones can be subcloned by limiting dilution procedures and grown by standard methods (Goding, Monoclonal Antibodies: Principles and Practice, pp. 59-103 (Academic Press, 1986)). Suitable culture media for this purpose include, for example, D-MEMO or RPMI 1640 medium. In addition, the hybridoma cells can be grown in vivo as ascites tumors in an animal. The monoclonal antibodies secreted by the subclones are suitably separated from the culture medium, ascites fluid, or serum by conventional immunoglobulin purification procedures such as protein A-Sepharose, hydroxylapatite chromatography, gel electrophoresis, dialysis, or affinity chromatography.

### Recombinant Production of Antibodies

The amino acid sequence of an immunoglobulin of interest can be determined by direct protein sequencing, and suitable encoding nucleotide sequences can be designed according to a universal codon table.

Alternatively, DNA encoding the monoclonal antibodies can be isolated and sequenced from the hybridoma cells using conventional procedures (e.g., by using oligonucleotide probes that are capable of binding specifically to genes encoding the heavy and light chains of the monoclonal antibodies). Sequence determination will generally require isolation of at least a portion of the gene or cDNA of interest. Usually this requires cloning the DNA or mRNA encoding the monoclonal antibodies. Cloning is carried out using standard techniques (see, e.g., Sambrook et al. (1989) Molecular Cloning: A Laboratory Guide, Vols 1-3, Cold Spring Harbor Press, which is incorporated herein by reference). For example, a cDNA library can be constructed by reverse transcription of polyA+ mRNA, preferably membrane-associated mRNA, and the library screened using probes specific for human immunoglobulin polypeptide gene sequences. In a preferred embodiment, the polymerase chain reaction (PCR) is used to amplify cDNAs (or portions of full-length cDNAs) encoding an immunoglobulin gene segment of interest (e.g., a light chain variable segment). The amplified sequences can be cloned readily into any suitable vector, e.g., expression vectors, minigene vectors, or phage display vectors. It will be appreciated that the particular method of cloning used is not critical, so long as it is possible to determine the sequence of some portion of the immunoglobulin polypeptide of interest.

One source for RNA used for cloning and sequencing is a hybridoma produced by obtaining a B cell from the transgenic mouse and fusing the B cell to an immortal cell. An advantage of using hybridomas is that they can be easily screened, and a hybridoma that produces a human monoclonal antibody of interest selected. Alternatively, RNA can be isolated from B cells (or whole spleen) of the immunized animal. When sources other than hybridomas are used, it may be desirable to screen for sequences encoding immunoglobulins or immunoglobulin polypeptides with specific binding characteristics. One method for such screening is the use of phage display technology. Phage display is described in e.g., Dower et al., WO 91/17271, McCafferty et al., WO 92/01047, and Caton and Koprowski, Proc. Natl. Acad. Sci. USA, 87:6450-6454 (1990), each of which is incorporated herein by reference. In one embodiment using phage display technology, cDNA from an immunized transgenic mouse (e.g., total spleen cDNA) is isolated, PCR is used to amplify cDNA sequences that encode a portion of an immunoglobulin polypeptide, e.g., CDR regions, and the amplified sequences are inserted into a phage vector. cDNAs encoding peptides of interest, e.g., variable region peptides with desired binding characteristics, are identified by standard techniques such as panning. The sequence of the amplified or cloned nucleic acid is then determined. Typically the sequence encoding an entire variable region of the immunoglobulin polypeptide is determined, however, sometimes only a portion of a variable region need be sequenced, for example, the CDR-encoding portion. Typically the sequenced portion will be at least 30 bases in length, and more often bases coding for at least about one-third or at least about one-half of the length of the variable region will be sequenced. Sequencing can be carried out on clones isolated from a cDNA library or, when PCR is used, after subcloning the amplified sequence or by direct PCR sequencing of the amplified segment. Sequencing is carried out using standard techniques (see, e.g., Sambrook et al. (1989) Molecular Cloning: A Laboratory Guide, Vols 1-3, Cold Spring Harbor Press, and Sanger, F. et al. (1977) Proc. Natl. Acad. Sci. USA 74: 5463-5467, which is incorporated herein by reference). By comparing the sequence of the cloned nucleic acid with published sequences of human immunoglobulin genes and cDNAs, an artisan can determine readily, depending on the region sequenced, (i) the germline segment usage of the hybridoma immunoglobulin polypeptide (including the isotype of the heavy chain) and (ii) the sequence of the heavy and light chain variable regions, including sequences resulting from N-region addition and the process of somatic mutation. One source of immunoglobulin gene sequence information is the National Center for Biotechnology Information, National Library of Medicine, National Institutes of Health, Bethesda, Md.

Once isolated, the DNA may be operably linked to expression control sequences or placed into expression vectors, which are then transfected into host cells such as E. coli cells, simian COS cells, Chinese hamster ovary (CHO) cells, or myeloma cells that do not otherwise produce immunoglobulin protein, to direct the synthesis of monoclonal antibodies in the recombinant host cells.

Expression control sequences denote DNA sequences necessary for the expression of an operably linked coding sequence in a particular host organism. The control sequences that are suitable for prokaryotes, for example, include a promoter, optionally an operator sequence, and a ribosome-binding site. Eukaryotic cells are known to utilize promoters, polyadenylation signals, and enhancers.

Nucleic acid is operably linked when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome-binding site is operably linked to a coding sequence if it is positioned so as to facilitate translation. Generally, operably linked means that the DNA sequences being linked are contiguous, and, in the case of a secretory leader, contiguous and in reading phase. However, enhancers do not have to be contiguous. Linking can be accomplished by ligation at convenient restriction sites. If such sites do not exist, synthetic oligonucleotide adaptors or linkers can be used in accordance with conventional practice.

Cell, cell line, and cell culture are often used interchangeably and all such designations include progeny. Transformants and transformed cells include the primary subject cell and cultures derived therefrom without regard for the number of transfers. It also is understood that all progeny may not be precisely identical in DNA content, due to deliberate or inadvertent mutations. Mutant progeny that have the same function or biological activity as screened for in the originally transformed cell are included.

Isolated nucleic acids also are provided that encode specific antibodies, optionally operably linked to control sequences recognized by a host cell, vectors and host cells comprising the nucleic acids, and recombinant techniques for the production of the antibodies, which may comprise culturing the host cell so that the nucleic acid is expressed and, optionally, recovering the antibody from the host cell culture or culture medium.

A variety of vectors are known in the art. Vector components can include one or more of the following: a signal sequence (that, for example, can direct secretion of the antibody), an origin of replication, one or more selective marker genes (that, for example, can confer antibiotic or other drug resistance, complement auxotrophic deficiencies, or supply critical nutrients not available in the media), an enhancer element, a promoter, and a transcription termination sequence, all of which are well known in the art.

Suitable host cells include prokaryote, yeast, or higher eukaryote cells. Suitable prokaryotes include eubacteria, such as Gram-negative or Gram-positive organisms, for example, Enterohacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas, and Streptomyces. In addition to prokaryotes, eukaryotic microbes such as filamentous fungi or yeast are suitable cloning or expression hosts for antibody-encoding vectors. Saccharomyces cerevisiae, or common baker's yeast, is the most commonly used among lower eukaryotic host microorganisms. However, a number of other genera, species, and strains are commonly available, such as Pichia, e.g. P. pastoris, Schizosaccharomyces pombe; Kluyveromyces, Yarrowia; Candida; Trichoderma reesia; Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated antibodies are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection of such cells are publicly available, e.g., the L-I variant of Autographa californica NPV and the Bm-5 strain of Bombyx mori NPV.

However, interest has been greatest in vertebrate cells, and propagation of vertebrate cells in culture (tissue culture) has become routine. Examples of useful mammalian host cell-lines are Chinese hamster ovary cells, including CHOKI cells (ATCC CCL61) and Chinese hamster ovary cells/−DHFR (DXB-11, DG-44; Urlaub et al, Proc. Natl. Acad. Sci. USA 77: 4216 (1980)); monkey kidney CV1 line transformed by SV40 (COS-7, ATCC CRL 1651); human embryonic kidney line (293 or 293 cells subcloned for growth in suspension culture, [Graham et al., J. Gen Virol. 36: 59 (1977)]; baby hamster kidney cells (BHK, ATCC CCL 10); mouse Sertoli cells (TM4, Mather, Biol. Reprod. 23: 243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (WI38, ATCC CCL 75); human hepatoma cells (Hep G2, HB 8065); mouse mammary tumor (MMT 060562, ATCC CCL51); TRI cells (Mather et al., Annals N.Y. Acad. Sci. 383: 44-68 (1982)); MRC 5 cells and FS4 cells.

The host cells can be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58: 44 (1979), Barnes et al., Anal. Biochem. 102: 255 (1980), U.S. Pat. Nos. 4,767,704; 4,657,866; 4,927,762; 4,560,655; or 5,122,469; WO90103430; WO 87/00195; or U.S. Pat. R^{e}. No. 30,985 can be used as culture media for the host cells. Any of these media can be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as Gentamycin™ drug), trace elements (defined as inorganic compounds usually present at final concentrations in the micromolar range), and glucose or an equivalent energy source. Any other necessary supplements also can be included at appropriate concentrations that would be known to those skilled in the art. The culture conditions, such as temperature, pH, and the like, are those previously used with the host cell selected for expression, and will be apparent to the artisan.

The antibody composition can be purified using, for example, hydroxylapatite chromatography, cation or anion exchange chromatography, or preferably affinity chromatography, using the antigen of interest or protein A or protein G as an affinity ligand. Protein A can be used to purify antibodies that are based on human .gamma.1, .gamma.2, or .gamma.4 heavy chains (Lindmark et al., J. Immunol. Meth. 62: 1-13 (1983)). Protein G is recommended for all mouse isotypes and for human .gamma.3 (Guss et al., 20 EMBO J. 5: 15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™ resin (J. T. Baker, Phillipsburg, 25 NJ.) is useful for purification. Other techniques for protein purification such as ethanol precipitation, Reverse Phase HPLC, chromatofocusing, SDS-PAGE, and ammonium sulfate precipitation are also possible depending on the specific binding agent or antibody to be recovered.

The term “epitope” or “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids are typically retained upon protein denaturing, whereas epitopes formed by tertiary folding are typically lost upon protein denaturing. An epitope typically includes at least 3-5, and more usually, at least 5 or 8-10 amino acids in a unique spatial conformation.

“Specifically binds” to or shows “specific binding” towards an epitope means that the antibody reacts or associates more frequently, and/or more rapidly, and/or greater duration, and/or with greater affinity with the epitope than with alternative substances.

In some embodiments, once the subject's tumor has been analyzed to determine whether the tumor harbors a wild type PI3K-α versus a mutant PI3K-α, for example, PI3K-α E545K or PI3K-α H1047R, using any one or more of the assays and methods described above, a treatment regimen can be prepared for the subject. If the subject's tumor harbors a PI3K-α having a mutation at position 1047, (for example, H1047R), the treatment regimen comprises administering to the subject a therapeutically effective amount of a PI3K-α selective inhibitor compound, or a dual PI3K-α/mTOR selective inhibitor, or a combination of a PI3K-α selective inhibitor or a mTOR selective inhibitor. If the subject's tumor harbors a PI3K-α having a mutation at position 545, (for example, E545K), the treatment regimen comprises administering to the subject a therapeutically effective amount of a combination of a PI3K-α selective inhibitor and a PI3K-β selective inhibitor, a dual PI3K-α/mTOR selective inhibitor, or a combination of a PI3K-α selective inhibitor and a mTOR selective inhibitor.

In another embodiment, the present invention provides kits comprising materials useful for carrying out the methods of the invention. The diagnostic/screening procedures described herein may be performed by diagnostic laboratories, experimental laboratories, or practitioners. The invention provides kits which can be used in these different settings.

Bagic materials and reagents required for identifying a PI3K-α mutation in a subject's tumor or cancer according to methods of the present invention may be assembled together in a kit. In certain embodiments, the kit comprises at least one PI3K-α amino acid sequence determining reagent that specifically detects a mutation in a nucleic acid or protein obtained from a subject's tumor disclosed herein, and instructions for using the kit according to one or more methods of the invention. Each kit necessarily comprises reagents which render the procedure specific. Thus, for detecting mRNA harboring the PI3K-α H1047R or E545K mutation, the reagent will comprise a nucleic acid probe complementary to mRNA, such as, for example, a cDNA or an oligonucleotide. The nucleic acid probe may or may not be inunobilized on a substrate surface (e.g., a microarray). For detecting a polypeptide product encoded by at least one PI3K-α mutation gene, the reagent will comprise an antibody that specifically binds to the mutated PI3K-α or a wild-type PI3K-α.

Depending on the procedure, the kit may further comprise one or more of: extraction buffer and/or reagents, amplification buffer and/or reagents, hybridization buffer and/or reagents, immunodetection buffer and/or reagents, labeling buffer and/or reagents, and detection means. Protocols for using these buffers and reagents for performing different steps of the procedure may also be included in the kit.

Reagents may be supplied in a solid (e.g., lyophilized) or liquid form. Kits of the present invention may optionally comprise one or more receptacles for mixing samples and/or reagents (e.g., vial, ampoule, test tube, ELISA plate, culture plate, flask or bottle) for each individual buffer and/or reagent. Each component will generally be suitable as aliquoted in its respective container or provided in a concentrated form. Other containers suitable for conducting certain steps for the disclosed methods may also be provided. The individual containers of the kit are preferably maintained in close confinement for commercial sale.

In certain embodiments, the kits of the present invention further comprise control samples. For example, a kit may include samples of total mRNA derived from tissue of various physiological states, such as, for example, wild-type PI3K-α, PI3K-α H1047R mRNA or PI3K-α E545K mRNA to be used as controls. In other embodiments, the inventive kits comprise at least one prostate disease expression profile map as described herein for use as comparison template. Preferably, the expression profile map is digital information stored in a computer-readable medium.

Instructions for using the kit according to one or more methods of the invention may comprise instructions for processing the prostate tissue sample and/or performing the test, instructions for interpreting the results as well as a notice in the form prescribed by a governmental agency (e.g., FDA) regulating the manufacture, use or sale of pharmaceuticals or biological products.

### Representative Compounds

Representative compounds of Formula I are depicted below. The examples are merely illustrative and do not limit the scope of the invention in any way. Compounds of the invention are named according to systematic application of the nomenclature rules agreed upon by the International Union of Pure and Applied Chemistry (IUPAC), International Union of Biochemistry and Molecular Biology (IUBMB), and the Chemical Abstracts Service (CAS). Specifically, names in Table 1 were generated using ACD/Labs naming software 8.00 release, product version 8.08 or higher.

TABLE 1

| Entry | |
|---|---|
| No. | Structure |
| 1 | 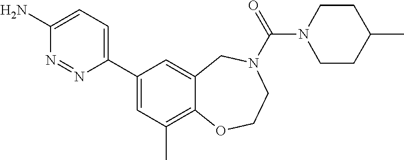 [SMILES: Cc1cc(-c2ccc(N)nn2)cc2c1OCCN(C(=O)N1CCC(C)CC1)C2] |
| 2 | 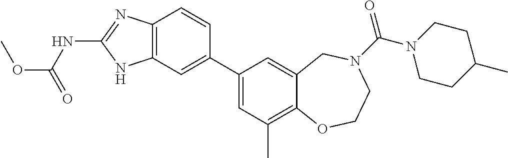 |
| 3 | 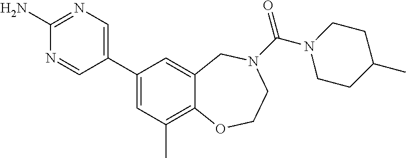 [SMILES: Cc1cc(-c2cnc(N)nc2)cc2c1OCCN(C(=O)N1CCC(C)CC1)C2] |
| 4 | 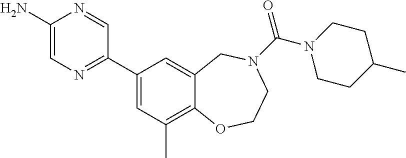 [SMILES: Cc1cc(-c2cnc(N)cn2)cc2c1OCCN(C(=O)N1CCC(C)CC1)C2] |
| 5 | 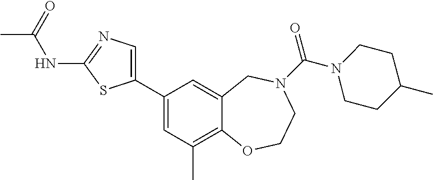 [SMILES: CC(=O)Nc1ncc(-c2cc(C)c3c(c2)CN(C(=O)N2CCC(C)CC2)CCO3)s1] |
| 6 | 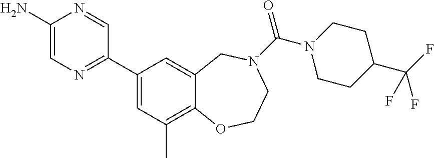 [SMILES: Cc1cc(-c2cnc(N)cn2)cc2c1OCCN(C(=O)N1CCC(C(F)(F)F)CC1)C2] |
| 7 | 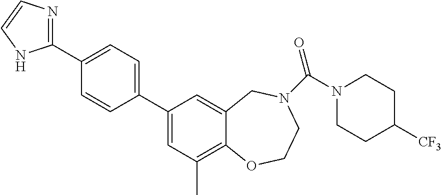 [SMILES: Cc1cc(-c2ccc(-c3ncc[nH]3)cc2)cc2c1OCCN(C(=O)N1CCC(C(F)(F)F)CC1)C2] |
| 8 | 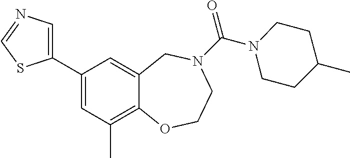 [SMILES: Cc1cc(-c2cncs2)cc2c1OCCN(C(=O)N1CCC(C)CC1)C2] |
| 9 | 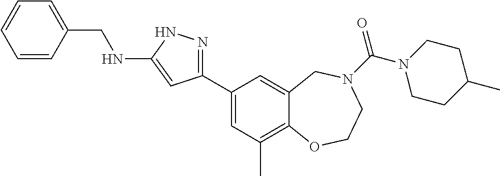 [SMILES: Cc1cc(-c2cc(NCc3ccccc3)[nH]n2)cc2c1OCCN(C(=O)N1CCC(C)CC1)C2] |
| 10 | 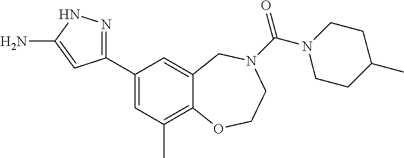 [SMILES: Cc1cc(-c2cc(N)[nH]n2)cc2c1OCCN(C(=O)N1CCC(C)CC1)C2] |
| 11 | 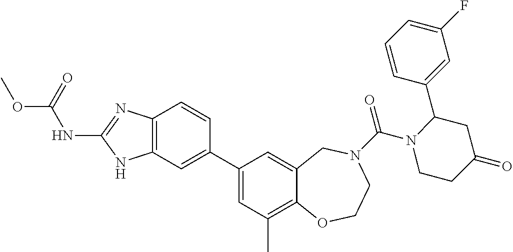 [SMILES: COC(=O)Nc1nc2ccc(-c3cc(C)c4c(c3)CN(C(=O)N3CCC(=O)CC3c3cccc(F)c3)CCO4)cc2[nH]1] |
| 12 | 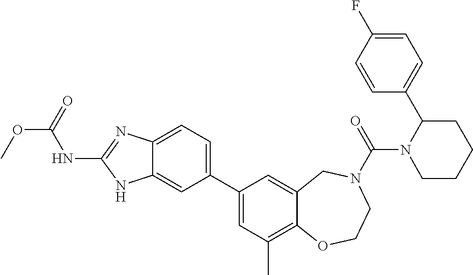 [SMILES: COC(=O)Nc1nc2ccc(-c3cc(C)c4c(c3)CN(C(=O)N3CCCCC3c3ccc(F)cc3)CCO4)cc2[nH]1] |
| 13 | 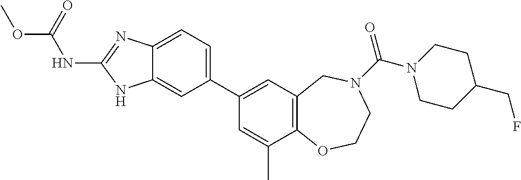 [SMILES: COC(=O)Nc1nc2ccc(-c3cc(C)c4c(c3)CN(C(=O)N3CCC(CF)CC3)CCO4)cc2[nH]1] |
| 14 | 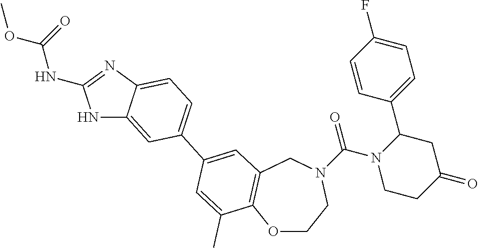 [SMILES: COC(=O)Nc1nc2ccc(-c3cc(C)c4c(c3)CN(C(=O)N3CCC(=O)CC3c3ccc(F)cc3)CCO4)cc2[nH]1] |
| 15 | 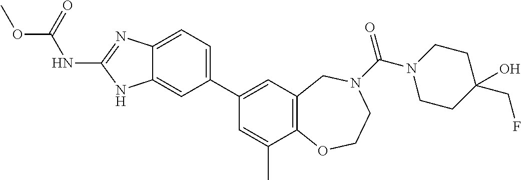 [SMILES: COC(=O)Nc1nc2ccc(-c3cc(C)c4c(c3)CN(C(=O)N3CCC(O)(CF)CC3)CCO4)cc2[nH]1] |
| 16 | 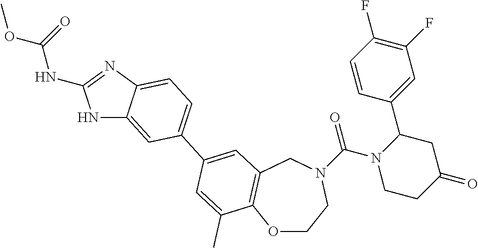 [SMILES: COC(=O)Nc1nc2ccc(-c3cc(C)c4c(c3)CN(C(=O)N3CCC(=O)CC3c3ccc(F)c(F)c3)CCO4)cc2[nH]1] |
| 17 | 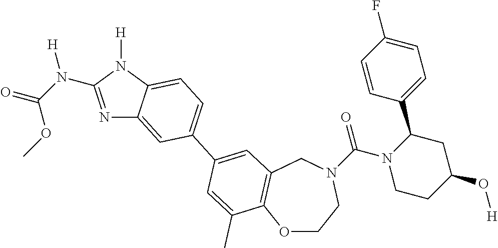 [SMILES: COC(=O)Nc1nc2cc(-c3cc(C)c4c(c3)CN(C(=O)N3CC[C@H](O)C[C@@H]3c3ccc(F)cc3)CCO4)ccc2[nH]1] |
| 18 | 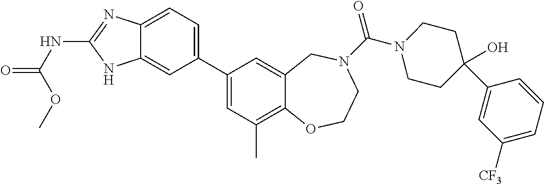 [SMILES: COC(=O)Nc1nc2ccc(-c3cc(C)c4c(c3)CN(C(=O)N3CCC(O)(c5cccc(C(F)(F)F)c5)CC3)CCO4)cc2[nH]1] |
| 19 | 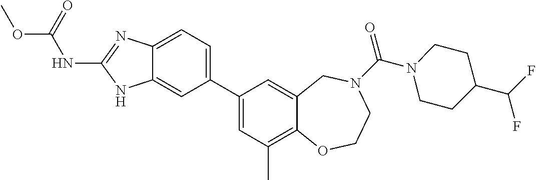 [SMILES: COC(=O)Nc1nc2ccc(-c3cc(C)c4c(c3)CN(C(=O)N3CCC(C(F)F)CC3)CCO4)cc2[nH]1] |
| 20 | 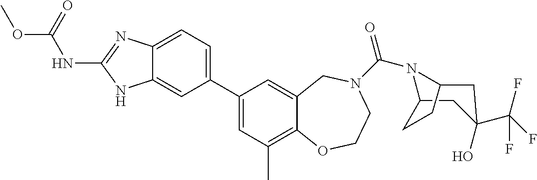 [SMILES: COC(=O)Nc1nc2ccc(-c3cc(C)c4c(c3)CN(C(=O)N3C5CCC3CC(O)(C(F)(F)F)C5)CCO4)cc2[nH]1] |
| 21 | 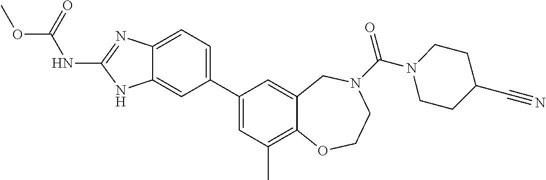 [SMILES: COC(=O)Nc1nc2ccc(-c3cc(C)c4c(c3)CN(C(=O)N3CCC(C#N)CC3)CCO4)cc2[nH]1] |
| 22 | 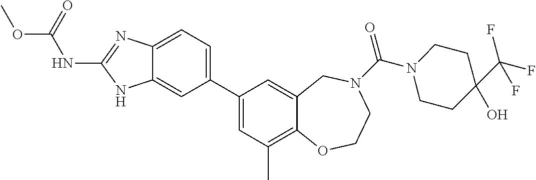 [SMILES: COC(=O)Nc1nc2ccc(-c3cc(C)c4c(c3)CN(C(=O)N3CCC(O)(C(F)(F)F)CC3)CCO4)cc2[nH]1] |
| 23 | 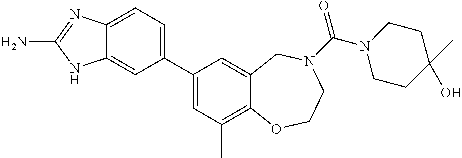 [SMILES: Cc1cc(-c2ccc3nc(N)[nH]c3c2)cc2c1OCCN(C(=O)N1CCC(C)(O)CC1)C2] |
| 24 | 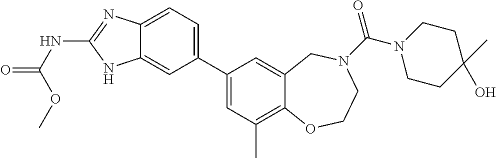 [SMILES: COC(=O)Nc1nc2ccc(-c3cc(C)c4c(c3)CN(C(=O)N3CCC(C)(O)CC3)CCO4)cc2[nH]1] |
| 25 | 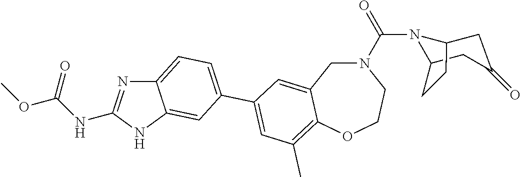 [SMILES: COC(=O)Nc1nc2ccc(-c3cc(C)c4c(c3)CN(C(=O)N3C5CCC3CC(=O)C5)CCO4)cc2[nH]1] |
| 26 | 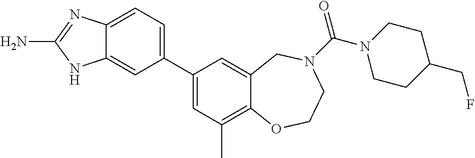 [SMILES: Cc1cc(-c2ccc3nc(N)[nH]c3c2)cc2c1OCCN(C(=O)N1CCC(CF)CC1)C2] |
| 27 | 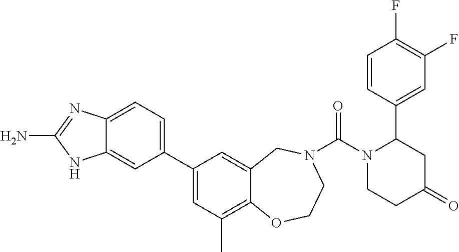 [SMILES: Cc1cc(-c2ccc3nc(N)[nH]c3c2)cc2c1OCCN(C(=O)N1CCC(=O)CC1c1ccc(F)c(F)c1)C2] |
| 28 | 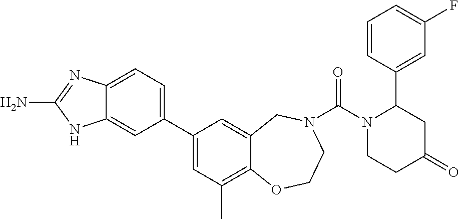 [SMILES: Cc1cc(-c2ccc3nc(N)[nH]c3c2)cc2c1OCCN(C(=O)N1CCC(=O)CC1c1cccc(F)c1)C2] |
| 29 | 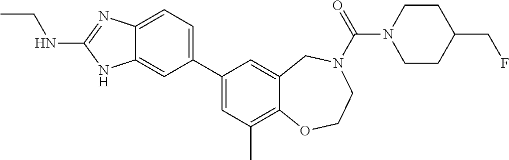 [SMILES: CCNc1nc2ccc(-c3cc(C)c4c(c3)CN(C(=O)N3CCC(CF)CC3)CCO4)cc2[nH]1] |
| 30 | 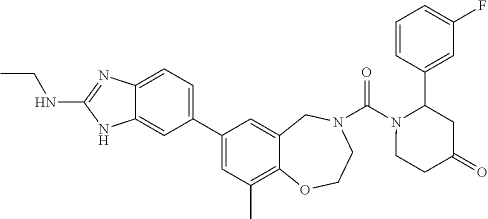 [SMILES: CCNc1nc2ccc(-c3cc(C)c4c(c3)CN(C(=O)N3CCC(=O)CC3c3cccc(F)c3)CCO4)cc2[nH]1] |
| 31 | 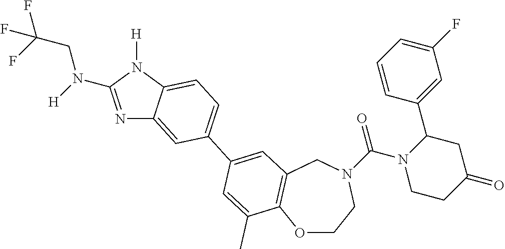 [SMILES: Cc1cc(-c2ccc3[nH]c(NCC(F)(F)F)nc3c2)cc2c1OCCN(C(=O)N1CCC(=O)CC1c1cccc(F)c1)C2] |
| 32 | 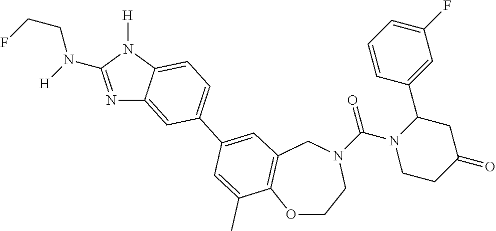 [SMILES: Cc1cc(-c2ccc3[nH]c(NCCF)nc3c2)cc2c1OCCN(C(=O)N1CCC(=O)CC1c1cccc(F)c1)C2] |
| 33 | 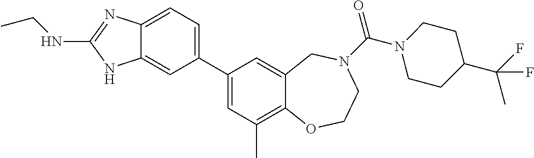 [SMILES: CCNc1nc2ccc(-c3cc(C)c4c(c3)CN(C(=O)N3CCC(C(C)(F)F)CC3)CCO4)cc2[nH]1] |
| 34 | 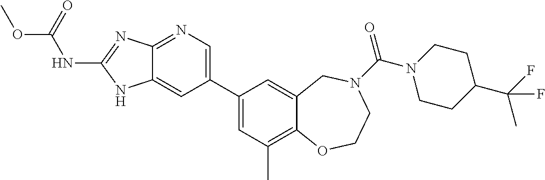 [SMILES: COC(=O)Nc1nc2ncc(-c3cc(C)c4c(c3)CN(C(=O)N3CCC(C(C)(F)F)CC3)CCO4)cc2[nH]1] |
| 35 | 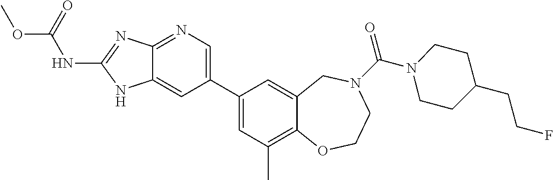 [SMILES: COC(=O)Nc1nc2ncc(-c3cc(C)c4c(c3)CN(C(=O)N3CCC(CCF)CC3)CCO4)cc2[nH]1] |
| 36 | 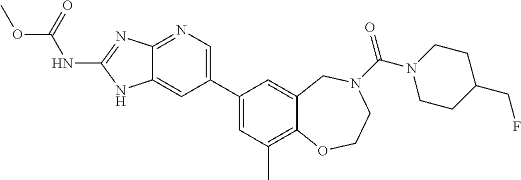 [SMILES: COC(=O)Nc1nc2ncc(-c3cc(C)c4c(c3)CN(C(=O)N3CCC(CF)CC3)CCO4)cc2[nH]1] |
| 37 | 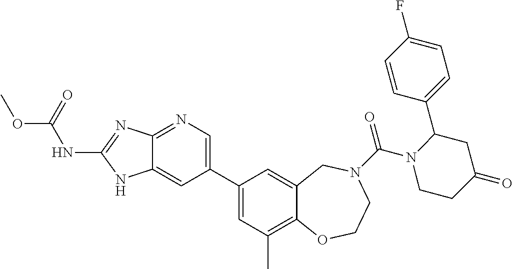 [SMILES: COC(=O)Nc1nc2ncc(-c3cc(C)c4c(c3)CN(C(=O)N3CCC(=O)CC3c3ccc(F)cc3)CCO4)cc2[nH]1] |
| 38 | 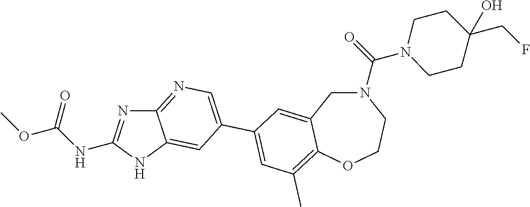 [SMILES: COC(=O)Nc1nc2ncc(-c3cc(C)c4c(c3)CN(C(=O)N3CCC(O)(CF)CC3)CCO4)cc2[nH]1] |
| 39 | 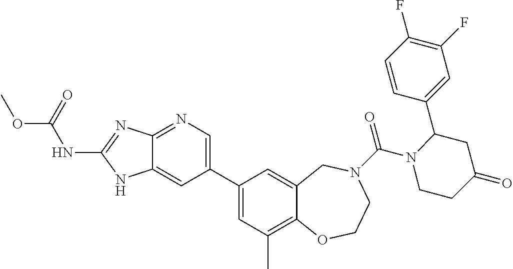 [SMILES: COC(=O)Nc1nc2ncc(-c3cc(C)c4c(c3)CN(C(=O)N3CCC(=O)CC3c3ccc(F)c(F)c3)CCO4)cc2[nH]1] |
| 40 | 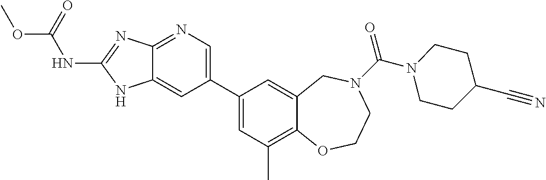 [SMILES: COC(=O)Nc1nc2ncc(-c3cc(C)c4c(c3)CN(C(=O)N3CCC(C#N)CC3)CCO4)cc2[nH]1] |
| 41 | 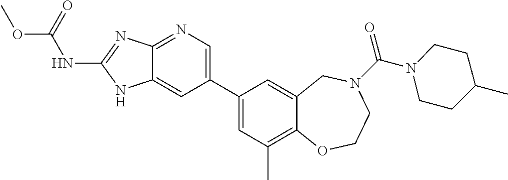 |
| 42 | 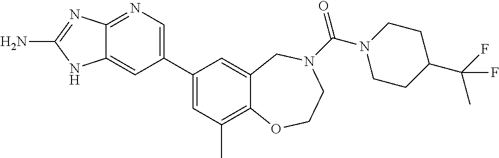 [SMILES: Cc1cc(-c2cnc3nc(N)[nH]c3c2)cc2c1OCCN(C(=O)N1CCC(C(C)(F)F)CC1)C2] |
| 43 | 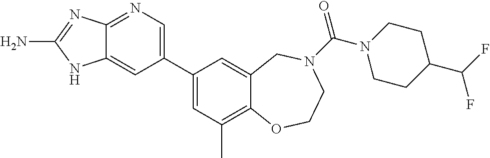 [SMILES: Cc1cc(-c2cnc3nc(N)[nH]c3c2)cc2c1OCCN(C(=O)N1CCC(C(F)F)CC1)C2] |
| 44 | 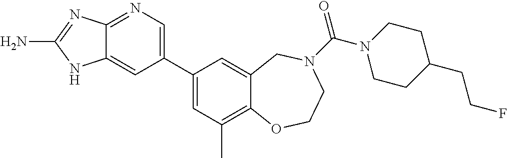 [SMILES: Cc1cc(-c2cnc3nc(N)[nH]c3c2)cc2c1OCCN(C(=O)N1CCC(CCF)CC1)C2] |
| 45 | 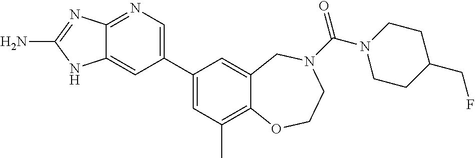 [SMILES: Cc1cc(-c2cnc3nc(N)[nH]c3c2)cc2c1OCCN(C(=O)N1CCC(CF)CC1)C2] |
| 46 | 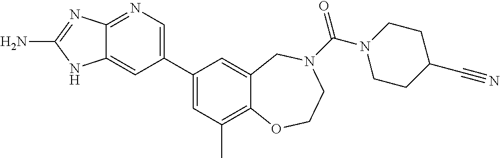 [SMILES: Cc1cc(-c2cnc3nc(N)[nH]c3c2)cc2c1OCCN(C(=O)N1CCC(C#N)CC1)C2] |
| 47 | 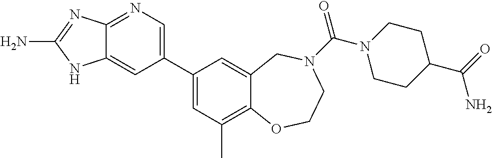 [SMILES: Cc1cc(-c2cnc3nc(N)[nH]c3c2)cc2c1OCCN(C(=O)N1CCC(C(N)=O)CC1)C2] |
| 48 | 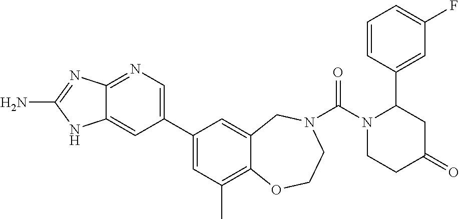 [SMILES: Cc1cc(-c2cnc3nc(N)[nH]c3c2)cc2c1OCCN(C(=O)N1CCC(=O)CC1c1cccc(F)c1)C2] |
| 49 | 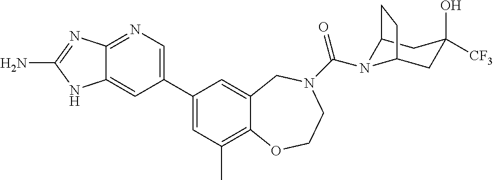 [SMILES: Cc1cc(-c2cnc3nc(N)[nH]c3c2)cc2c1OCCN(C(=O)N1C3CCC1CC(O)(C(F)(F)F)C3)C2] |
| 50 | 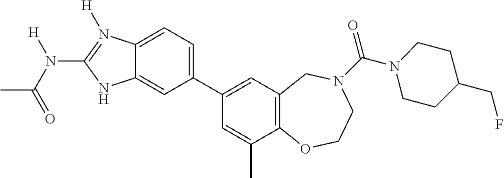 |
| 51 | 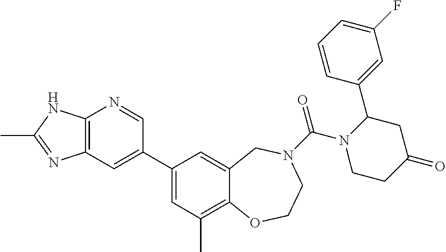 [SMILES: Cc1nc2cc(-c3cc(C)c4c(c3)CN(C(=O)N3CCC(=O)CC3c3cccc(F)c3)CCO4)cnc2[nH]1] |
| 52 | 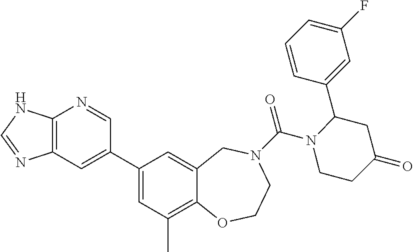 [SMILES: Cc1cc(-c2cnc3[nH]cnc3c2)cc2c1OCCN(C(=O)N1CCC(=O)CC1c1cccc(F)c1)C2] |
| 53 | 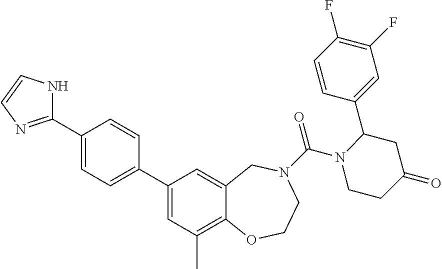 [SMILES: Cc1cc(-c2ccc(-c3ncc[nH]3)cc2)cc2c1OCCN(C(=O)N1CCC(=O)CC1c1ccc(F)c(F)c1)C2] |
| 54 | 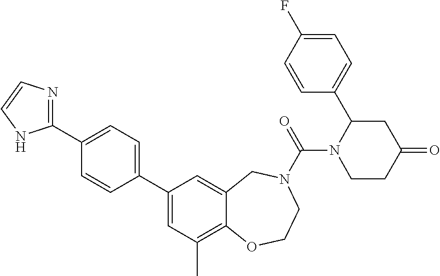 [SMILES: Cc1cc(-c2ccc(-c3ncc[nH]3)cc2)cc2c1OCCN(C(=O)N1CCC(=O)CC1c1ccc(F)cc1)C2] |
| 55 | 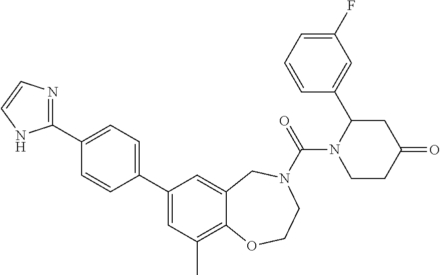 [SMILES: Cc1cc(-c2ccc(-c3ncc[nH]3)cc2)cc2c1OCCN(C(=O)N1CCC(=O)CC1c1cccc(F)c1)C2] |
| 56 | 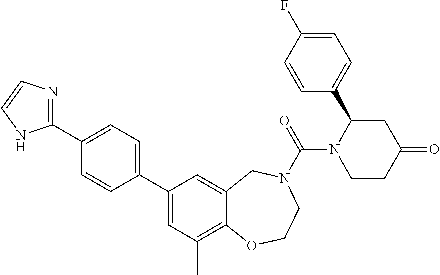 [SMILES: Cc1cc(-c2ccc(-c3ncc[nH]3)cc2)cc2c1OCCN(C(=O)N1CCC(=O)C[C@@H]1c1ccc(F)cc1)C2] |
| 57 | 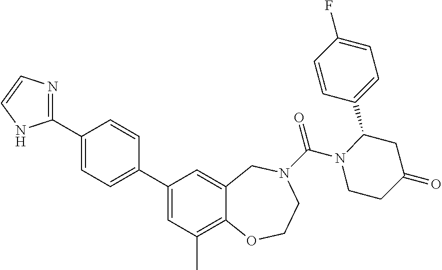 [SMILES: Cc1cc(-c2ccc(-c3ncc[nH]3)cc2)cc2c1OCCN(C(=O)N1CCC(=O)C[C@H]1c1ccc(F)cc1)C2] |
| 58 | 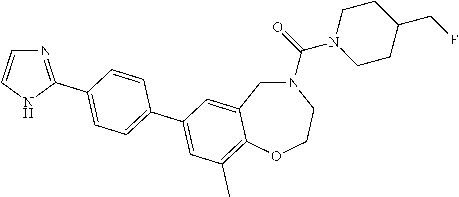 [SMILES: Cc1cc(-c2ccc(-c3ncc[nH]3)cc2)cc2c1OCCN(C(=O)N1CCC(CF)CC1)C2] |
| 59 | 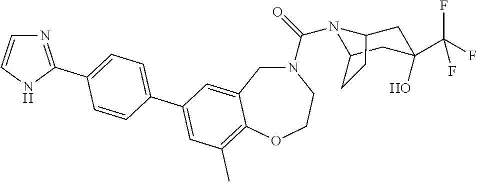 [SMILES: Cc1cc(-c2ccc(-c3ncc[nH]3)cc2)cc2c1OCCN(C(=O)N1C3CCC1CC(O)(C(F)(F)F)C3)C2] |
| 60 | 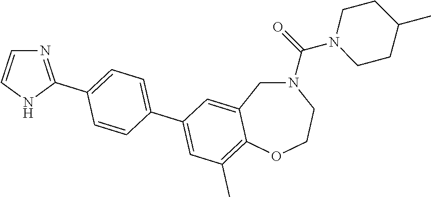 [SMILES: Cc1cc(-c2ccc(-c3ncc[nH]3)cc2)cc2c1OCCN(C(=O)N1CCC(C)CC1)C2] |
| 61 | 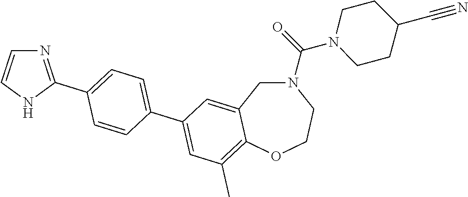 [SMILES: Cc1cc(-c2ccc(-c3ncc[nH]3)cc2)cc2c1OCCN(C(=O)N1CCC(C#N)CC1)C2] |
| 62 | 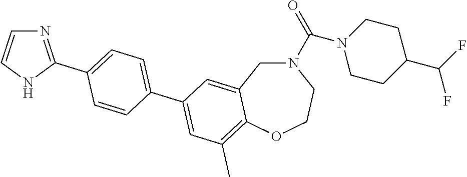 [SMILES: Cc1cc(-c2ccc(-c3ncc[nH]3)cc2)cc2c1OCCN(C(=O)N1CCC(C(F)F)CC1)C2] |
| 63 | 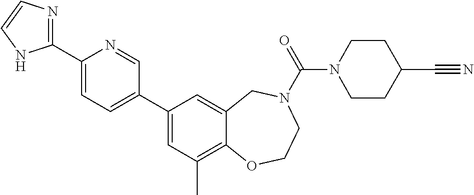 [SMILES: Cc1cc(-c2ccc(-c3ncc[nH]3)nc2)cc2c1OCCN(C(=O)N1CCC(C#N)CC1)C2] |
| 64 | 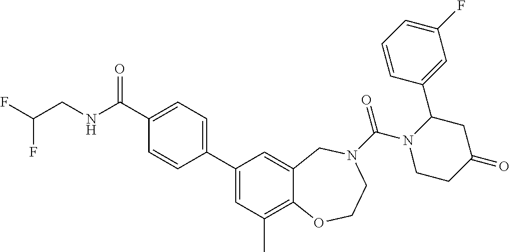 [SMILES: Cc1cc(-c2ccc(C(=O)NCC(F)F)cc2)cc2c1OCCN(C(=O)N1CCC(=O)CC1c1cccc(F)c1)C2] |
| 65 | 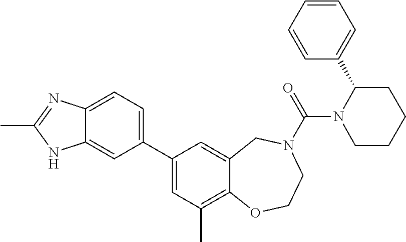 [SMILES: Cc1nc2ccc(-c3cc(C)c4c(c3)CN(C(=O)N3CCCC[C@H]3c3ccccc3)CCO4)cc2[nH]1] |
| 66 | 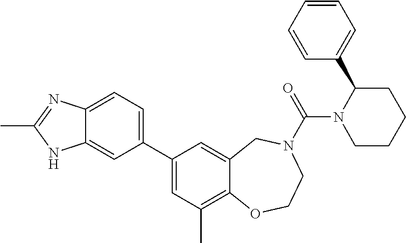 [SMILES: Cc1nc2ccc(-c3cc(C)c4c(c3)CN(C(=O)N3CCCC[C@@H]3c3ccccc3)CCO4)cc2[nH]1] |
| 67 | 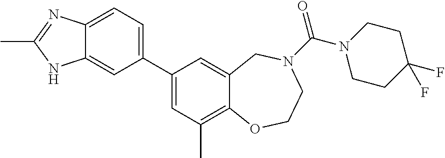 [SMILES: Cc1nc2ccc(-c3cc(C)c4c(c3)CN(C(=O)N3CCC(F)(F)CC3)CCO4)cc2[nH]1] |
| 68 | 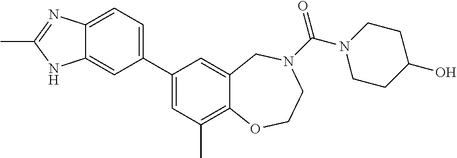 [SMILES: Cc1nc2ccc(-c3cc(C)c4c(c3)CN(C(=O)N3CCC(O)CC3)CCO4)cc2[nH]1] |
| 69 | 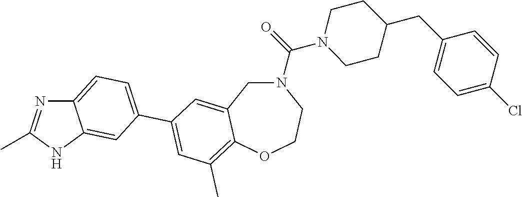 [SMILES: Cc1nc2ccc(-c3cc(C)c4c(c3)CN(C(=O)N3CCC(Cc5ccc(Cl)cc5)CC3)CCO4)cc2[nH]1] |
| 70 | 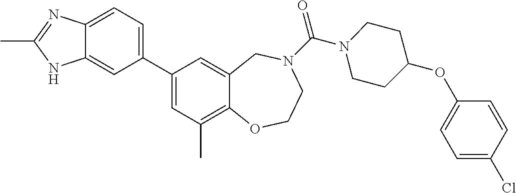 [SMILES: Cc1nc2ccc(-c3cc(C)c4c(c3)CN(C(=O)N3CCC(Oc5ccc(Cl)cc5)CC3)CCO4)cc2[nH]1] |
| 71 | 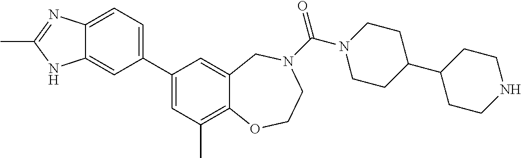 [SMILES: Cc1nc2ccc(-c3cc(C)c4c(c3)CN(C(=O)N3CCC(C5CCNCC5)CC3)CCO4)cc2[nH]1] |
| 72 | 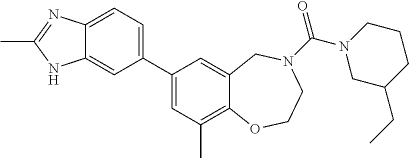 [SMILES: CCC1CCCN(C(=O)N2CCOc3c(C)cc(-c4ccc5nc(C)[nH]c5c4)cc3C2)C1] |
| 73 | 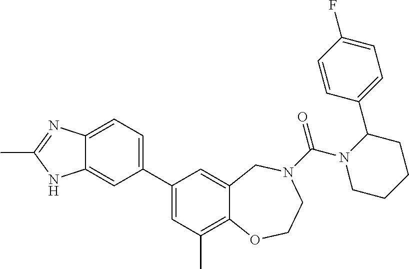 [SMILES: Cc1nc2ccc(-c3cc(C)c4c(c3)CN(C(=O)N3CCCCC3c3ccc(F)cc3)CCO4)cc2[nH]1] |
| 74 | 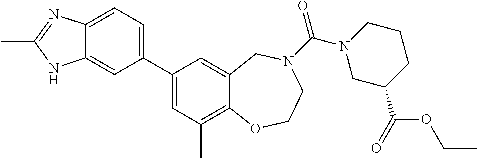 [SMILES: CCOC(=O)[C@H]1CCCN(C(=O)N2CCOc3c(C)cc(-c4ccc5nc(C)[nH]c5c4)cc3C2)C1] |
| 75 | 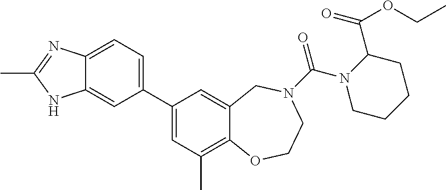 [SMILES: CCOC(=O)C1CCCCN1C(=O)N1CCOc2c(C)cc(-c3ccc4nc(C)[nH]c4c3)cc2C1] |
| 76 | 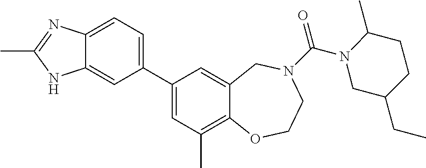 [SMILES: CCC1CCC(C)N(C(=O)N2CCOc3c(C)cc(-c4ccc5nc(C)[nH]c5c4)cc3C2)C1] |
| 77 | 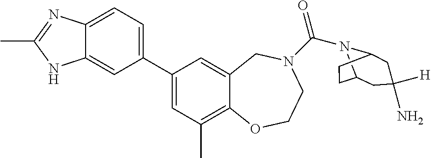 [SMILES: Cc1nc2ccc(-c3cc(C)c4c(c3)CN(C(=O)N3C5CCC3CC(N)C5)CCO4)cc2[nH]1] |
| 78 | 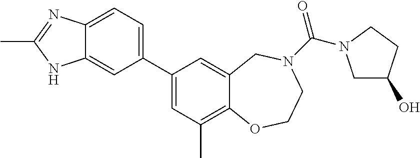 [SMILES: Cc1nc2ccc(-c3cc(C)c4c(c3)CN(C(=O)N3CC[C@@H](O)C3)CCO4)cc2[nH]1] |
| 79 | 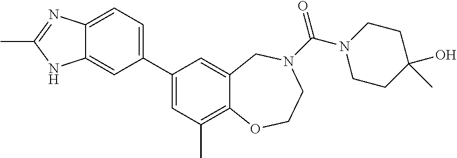 [SMILES: Cc1nc2ccc(-c3cc(C)c4c(c3)CN(C(=O)N3CCC(C)(O)CC3)CCO4)cc2[nH]1] |
| 80 | 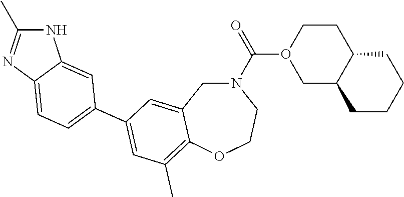 |
| 81 | 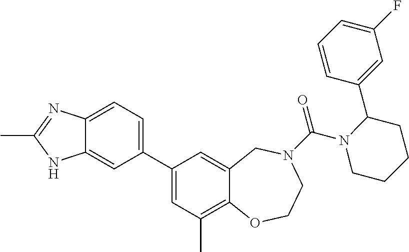 [SMILES: Cc1nc2ccc(-c3cc(C)c4c(c3)CN(C(=O)N3CCCCC3c3cccc(F)c3)CCO4)cc2[nH]1] |
| 82 | 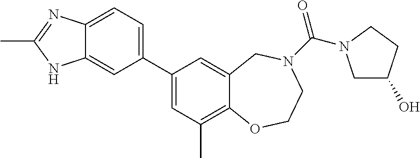 [SMILES: Cc1nc2ccc(-c3cc(C)c4c(c3)CN(C(=O)N3CC[C@H](O)C3)CCO4)cc2[nH]1] |
| 83 | 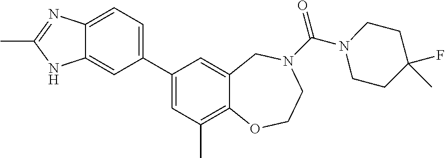 [SMILES: Cc1nc2ccc(-c3cc(C)c4c(c3)CN(C(=O)N3CCC(C)(F)CC3)CCO4)cc2[nH]1] |
| 84 | 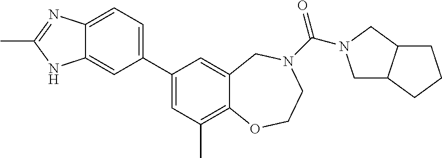 [SMILES: Cc1nc2ccc(-c3cc(C)c4c(c3)CN(C(=O)N3CC5CCCC5C3)CCO4)cc2[nH]1] |
| 85 | 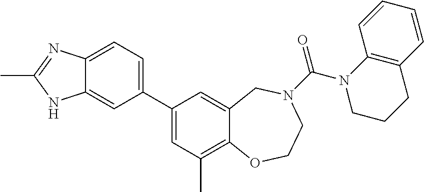 [SMILES: Cc1nc2ccc(-c3cc(C)c4c(c3)CN(C(=O)N3CCCc5ccccc53)CCO4)cc2[nH]1] |
| 86 | 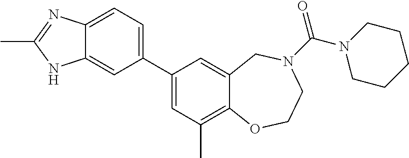 [SMILES: Cc1nc2ccc(-c3cc(C)c4c(c3)CN(C(=O)N3CCCCC3)CCO4)cc2[nH]1] |
| 87 | 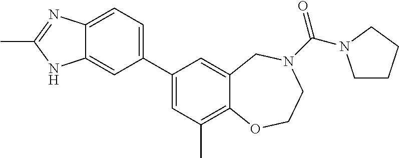 [SMILES: Cc1nc2ccc(-c3cc(C)c4c(c3)CN(C(=O)N3CCCC3)CCO4)cc2[nH]1] |
| 88 | 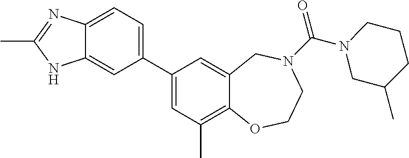 [SMILES: Cc1nc2ccc(-c3cc(C)c4c(c3)CN(C(=O)N3CCCC(C)C3)CCO4)cc2[nH]1] |
| 89 | 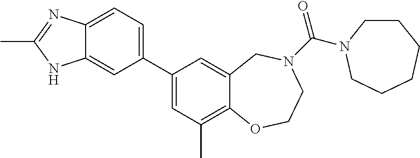 [SMILES: Cc1nc2ccc(-c3cc(C)c4c(c3)CN(C(=O)N3CCCCCC3)CCO4)cc2[nH]1] |
| 90 | 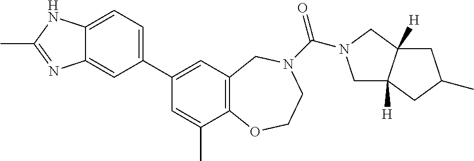 |
| 91 | 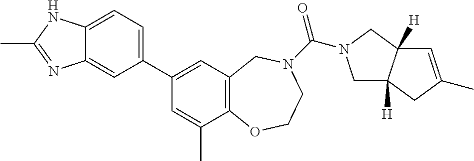 |
| 92 | 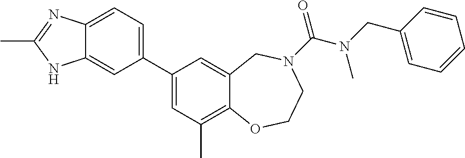 [SMILES: Cc1nc2ccc(-c3cc(C)c4c(c3)CN(C(=O)N(C)Cc3ccccc3)CCO4)cc2[nH]1] |
| 93 | 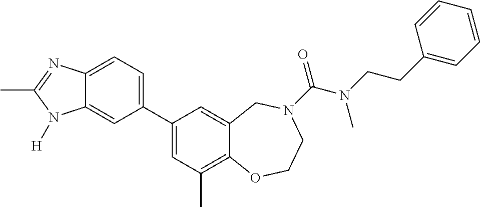 [SMILES: Cc1nc2ccc(-c3cc(C)c4c(c3)CN(C(=O)N(C)CCc3ccccc3)CCO4)cc2[nH]1] |
| 94 | 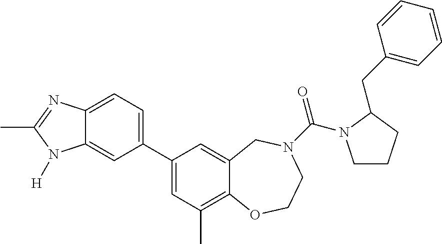 [SMILES: Cc1nc2ccc(-c3cc(C)c4c(c3)CN(C(=O)N3CCCC3Cc3ccccc3)CCO4)cc2[nH]1] |
| 95 | 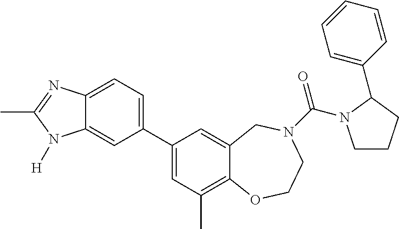 [SMILES: Cc1nc2ccc(-c3cc(C)c4c(c3)CN(C(=O)N3CCCC3c3ccccc3)CCO4)cc2[nH]1] |
| 96 | 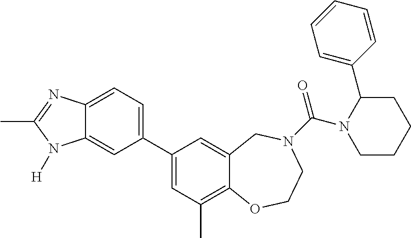 [SMILES: Cc1nc2ccc(-c3cc(C)c4c(c3)CN(C(=O)N3CCCCC3c3ccccc3)CCO4)cc2[nH]1] |
| 97 | 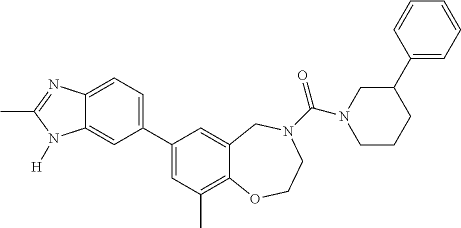 [SMILES: Cc1nc2ccc(-c3cc(C)c4c(c3)CN(C(=O)N3CCCC(c5ccccc5)C3)CCO4)cc2[nH]1] |
| 98 | 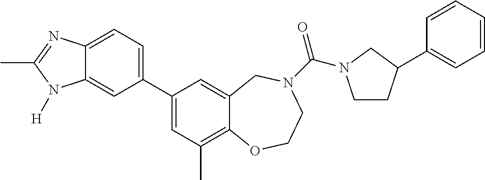 [SMILES: Cc1nc2ccc(-c3cc(C)c4c(c3)CN(C(=O)N3CCC(c5ccccc5)C3)CCO4)cc2[nH]1] |
| 99 | 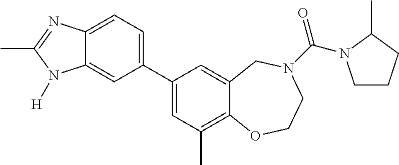 [SMILES: Cc1nc2ccc(-c3cc(C)c4c(c3)CN(C(=O)N3CCCC3C)CCO4)cc2[nH]1] |
| 100 | 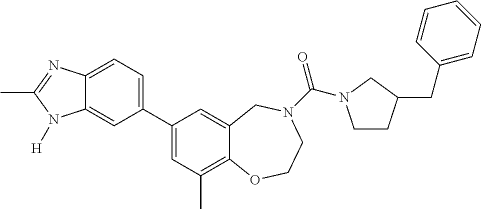 [SMILES: Cc1nc2ccc(-c3cc(C)c4c(c3)CN(C(=O)N3CCC(Cc5ccccc5)C3)CCO4)cc2[nH]1] |
| 101 | 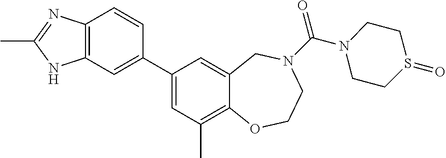 [SMILES: Cc1nc2ccc(-c3cc(C)c4c(c3)CN(C(=O)N3CCS(=O)CC3)CCO4)cc2[nH]1] |
| 102 | 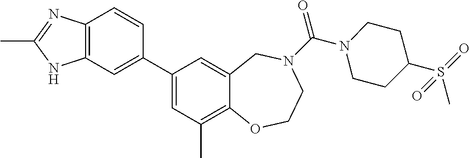 [SMILES: Cc1nc2ccc(-c3cc(C)c4c(c3)CN(C(=O)N3CCC(S(C)(=O)=O)CC3)CCO4)cc2[nH]1] |
| 103 | 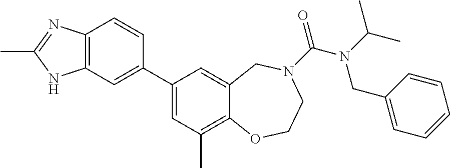 [SMILES: Cc1nc2ccc(-c3cc(C)c4c(c3)CN(C(=O)N(Cc3ccccc3)C(C)C)CCO4)cc2[nH]1] |
| 104 | 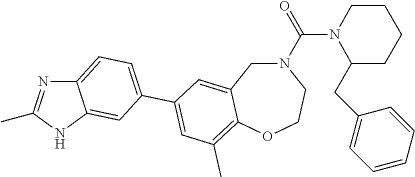 [SMILES: Cc1nc2ccc(-c3cc(C)c4c(c3)CN(C(=O)N3CCCCC3Cc3ccccc3)CCO4)cc2[nH]1] |
| 105 | 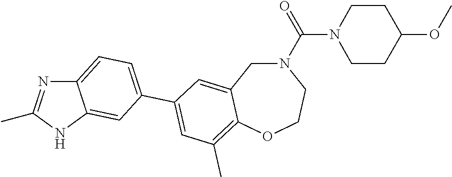 |
| 106 | 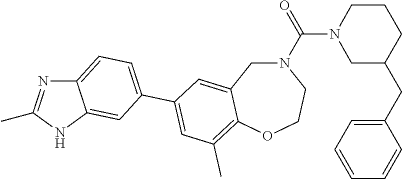 [SMILES: Cc1nc2ccc(-c3cc(C)c4c(c3)CN(C(=O)N3CCCC(Cc5ccccc5)C3)CCO4)cc2[nH]1] |
| 107 | 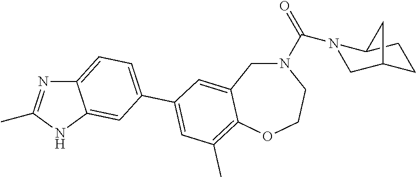 [SMILES: Cc1nc2ccc(-c3cc(C)c4c(c3)CN(C(=O)N3CC5CCC3C5)CCO4)cc2[nH]1] |
| 108 | 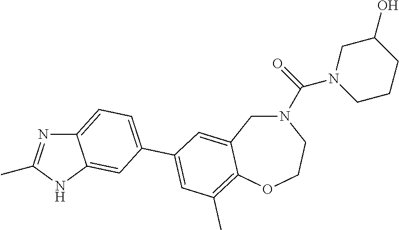 [SMILES: Cc1nc2ccc(-c3cc(C)c4c(c3)CN(C(=O)N3CCCC(O)C3)CCO4)cc2[nH]1] |
| 109 | 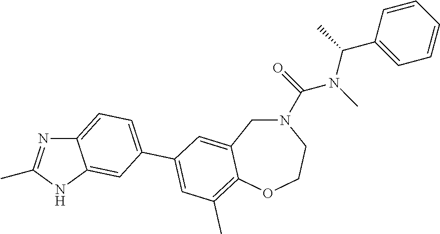 [SMILES: Cc1nc2ccc(-c3cc(C)c4c(c3)CN(C(=O)N(C)[C@H](C)c3ccccc3)CCO4)cc2[nH]1] |
| 110 | 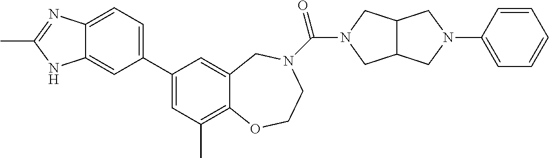 [SMILES: Cc1nc2ccc(-c3cc(C)c4c(c3)CN(C(=O)N3CC5CN(c6ccccc6)CC5C3)CCO4)cc2[nH]1] |
| 111 | 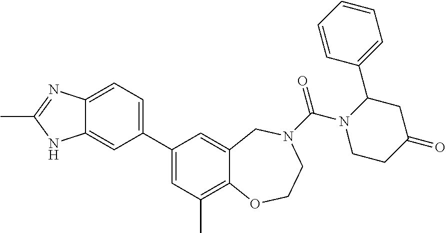 [SMILES: Cc1nc2ccc(-c3cc(C)c4c(c3)CN(C(=O)N3CCC(=O)CC3c3ccccc3)CCO4)cc2[nH]1] |
| 112 | 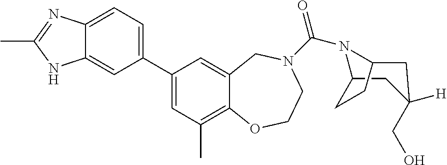 [SMILES: Cc1nc2ccc(-c3cc(C)c4c(c3)CN(C(=O)N3C5CCC3CC(CO)C5)CCO4)cc2[nH]1] |
| 113 | 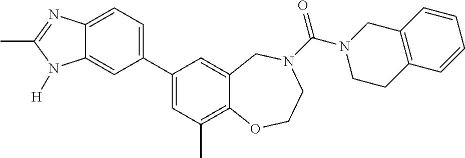 [SMILES: Cc1nc2ccc(-c3cc(C)c4c(c3)CN(C(=O)N3CCc5ccccc5C3)CCO4)cc2[nH]1] |
| 114 | 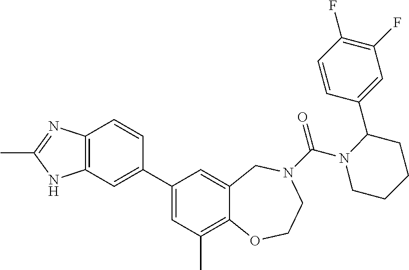 [SMILES: Cc1nc2ccc(-c3cc(C)c4c(c3)CN(C(=O)N3CCCCC3c3ccc(F)c(F)c3)CCO4)cc2[nH]1] |
| 115 | 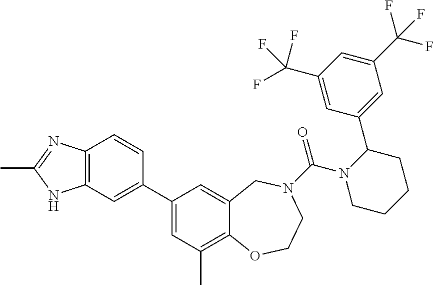 [SMILES: Cc1nc2ccc(-c3cc(C)c4c(c3)CN(C(=O)N3CCCCC3c3cc(C(F)(F)F)cc(C(F)(F)F)c3)CCO4)cc2[nH]1] |
| 116 | 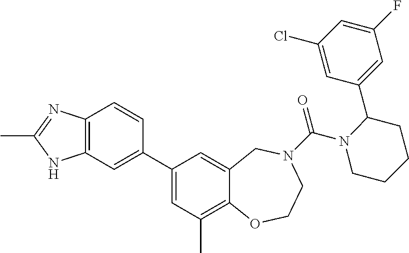 [SMILES: Cc1nc2ccc(-c3cc(C)c4c(c3)CN(C(=O)N3CCCCC3c3cc(F)cc(Cl)c3)CCO4)cc2[nH]1] |
| 117 | 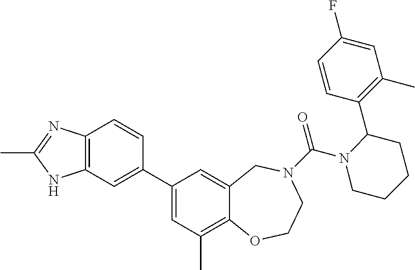 |
| 118 | 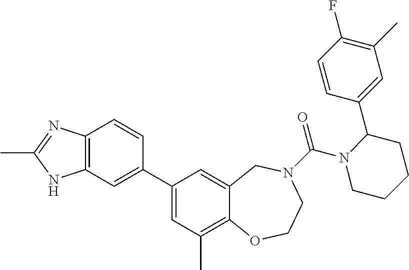 [SMILES: Cc1nc2ccc(-c3cc(C)c4c(c3)CN(C(=O)N3CCCCC3c3ccc(F)c(C)c3)CCO4)cc2[nH]1] |
| 119 | 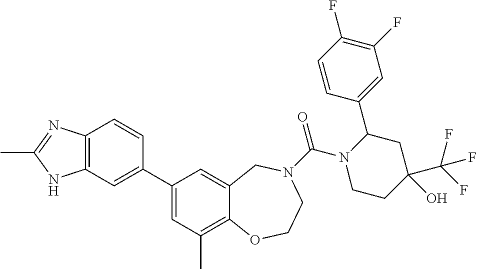 [SMILES: Cc1nc2ccc(-c3cc(C)c4c(c3)CN(C(=O)N3CCC(O)(C(F)(F)F)CC3c3ccc(F)c(F)c3)CCO4)cc2[nH]1] |
| 120 | 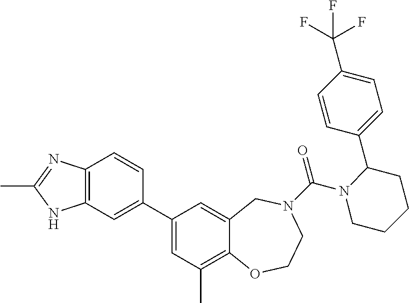 [SMILES: Cc1nc2ccc(-c3cc(C)c4c(c3)CN(C(=O)N3CCCCC3c3ccc(C(F)(F)F)cc3)CCO4)cc2[nH]1] |
| 121 | 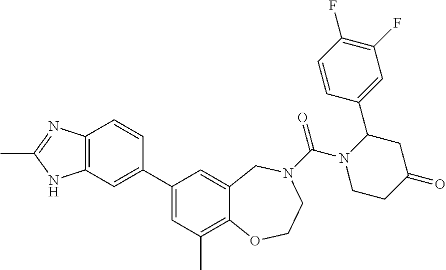 [SMILES: Cc1nc2ccc(-c3cc(C)c4c(c3)CN(C(=O)N3CCC(=O)CC3c3ccc(F)c(F)c3)CCO4)cc2[nH]1] |
| 122 | 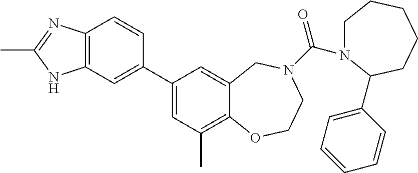 [SMILES: Cc1nc2ccc(-c3cc(C)c4c(c3)CN(C(=O)N3CCCCCC3c3ccccc3)CCO4)cc2[nH]1] |
| 123 | 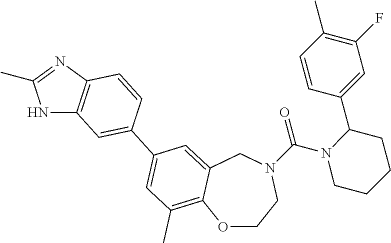 [SMILES: Cc1nc2ccc(-c3cc(C)c4c(c3)CN(C(=O)N3CCCCC3c3ccc(C)c(F)c3)CCO4)cc2[nH]1] |
| 124 | 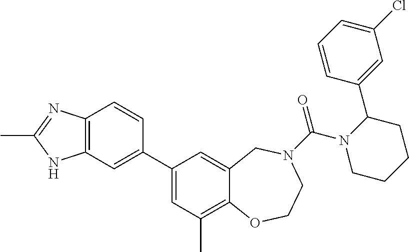 [SMILES: Cc1nc2ccc(-c3cc(C)c4c(c3)CN(C(=O)N3CCCCC3c3cccc(Cl)c3)CCO4)cc2[nH]1] |
| 125 | 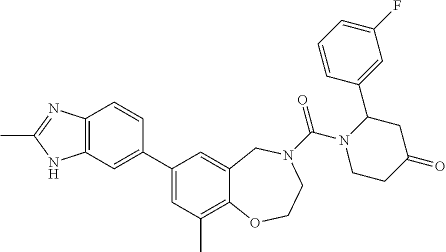 [SMILES: Cc1nc2ccc(-c3cc(C)c4c(c3)CN(C(=O)N3CCC(=O)CC3c3cccc(F)c3)CCO4)cc2[nH]1] |
| 126 | 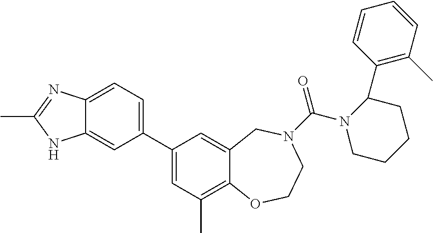 |
| 127 | 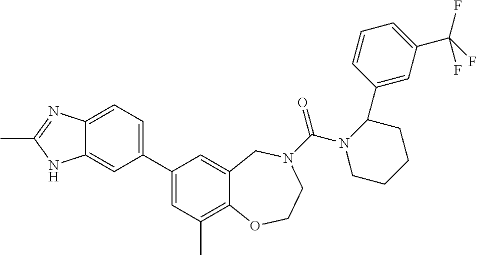 [SMILES: Cc1nc2ccc(-c3cc(C)c4c(c3)CN(C(=O)N3CCCCC3c3cccc(C(F)(F)F)c3)CCO4)cc2[nH]1] |
| 128 | 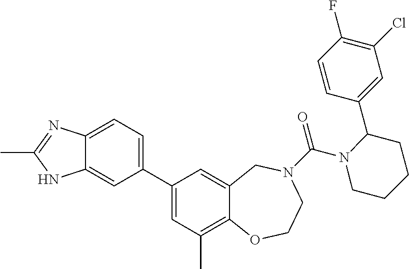 [SMILES: Cc1nc2ccc(-c3cc(C)c4c(c3)CN(C(=O)N3CCCCC3c3ccc(F)c(Cl)c3)CCO4)cc2[nH]1] |
| 129 | 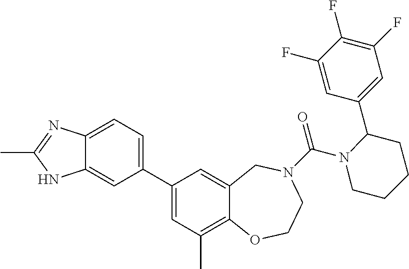 [SMILES: Cc1nc2ccc(-c3cc(C)c4c(c3)CN(C(=O)N3CCCCC3c3cc(F)c(F)c(F)c3)CCO4)cc2[nH]1] |
| 130 | 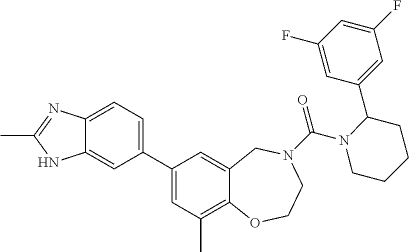 [SMILES: Cc1nc2ccc(-c3cc(C)c4c(c3)CN(C(=O)N3CCCCC3c3cc(F)cc(F)c3)CCO4)cc2[nH]1] |
| 131 | 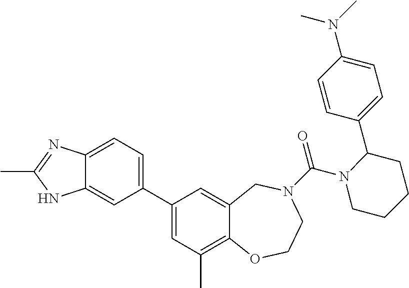 [SMILES: Cc1nc2ccc(-c3cc(C)c4c(c3)CN(C(=O)N3CCCCC3c3ccc(N(C)C)cc3)CCO4)cc2[nH]1] |
| 132 | 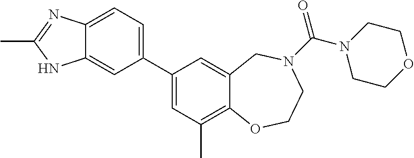 [SMILES: Cc1nc2ccc(-c3cc(C)c4c(c3)CN(C(=O)N3CCOCC3)CCO4)cc2[nH]1] |
| 133 | 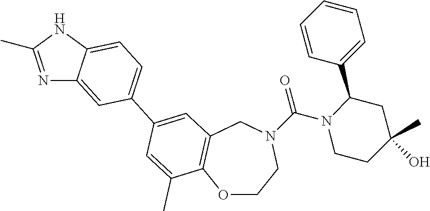 |
| 134 | 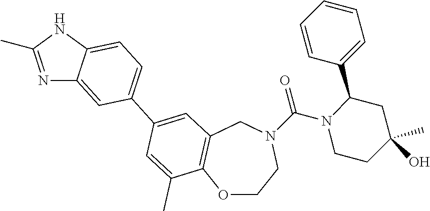 |
| 135 | 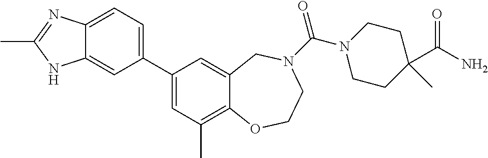 [SMILES: Cc1nc2ccc(-c3cc(C)c4c(c3)CN(C(=O)N3CCC(C)(C(N)=O)CC3)CCO4)cc2[nH]1] |
| 136 | 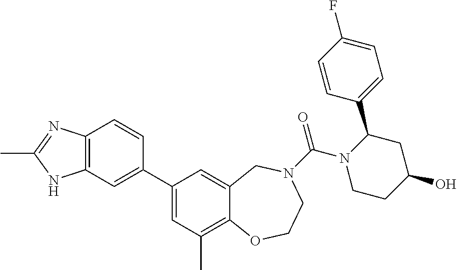 [SMILES: Cc1nc2ccc(-c3cc(C)c4c(c3)CN(C(=O)N3CC[C@H](O)C[C@@H]3c3ccc(F)cc3)CCO4)cc2[nH]1] |
| 137 | 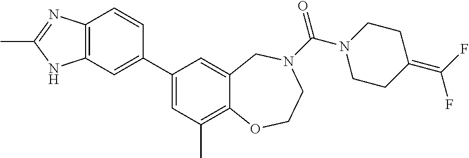 [SMILES: Cc1nc2ccc(-c3cc(C)c4c(c3)CN(C(=O)N3CCC(=C(F)F)CC3)CCO4)cc2[nH]1] |
| 138 | 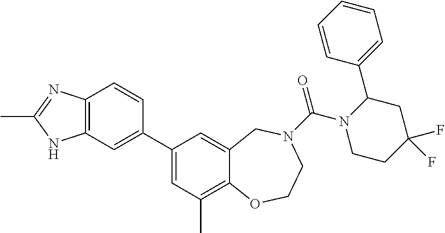 [SMILES: Cc1nc2ccc(-c3cc(C)c4c(c3)CN(C(=O)N3CCC(F)(F)CC3c3ccccc3)CCO4)cc2[nH]1] |
| 139 | 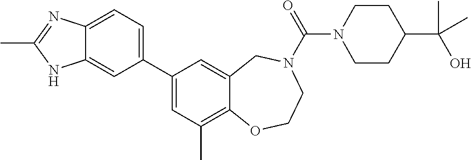 [SMILES: Cc1nc2ccc(-c3cc(C)c4c(c3)CN(C(=O)N3CCC(C(C)(C)O)CC3)CCO4)cc2[nH]1] |
| 140 | 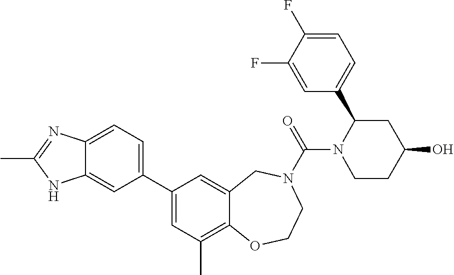 [SMILES: Cc1nc2ccc(-c3cc(C)c4c(c3)CN(C(=O)N3CC[C@H](O)C[C@@H]3c3ccc(F)c(F)c3)CCO4)cc2[nH]1] |
| 141 | 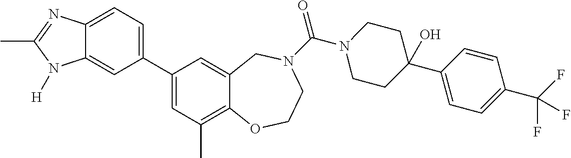 [SMILES: Cc1nc2ccc(-c3cc(C)c4c(c3)CN(C(=O)N3CCC(O)(c5ccc(C(F)(F)F)cc5)CC3)CCO4)cc2[nH]1] |
| 142 | 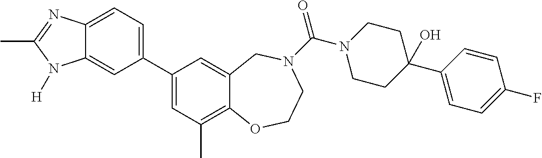 [SMILES: Cc1nc2ccc(-c3cc(C)c4c(c3)CN(C(=O)N3CCC(O)(c5ccc(F)cc5)CC3)CCO4)cc2[nH]1] |
| 143 | 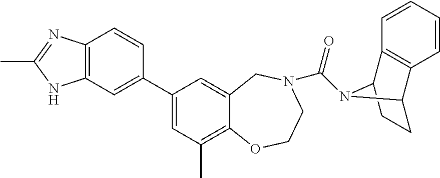 [SMILES: Cc1nc2ccc(-c3cc(C)c4c(c3)CN(C(=O)N3C5CCC3c3ccccc35)CCO4)cc2[nH]1] |
| 144 | 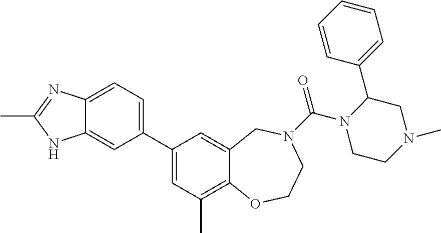 |
| 145 | 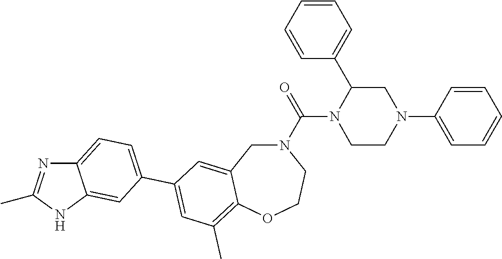 [SMILES: Cc1nc2ccc(-c3cc(C)c4c(c3)CN(C(=O)N3CCN(c5ccccc5)CC3c3ccccc3)CCO4)cc2[nH]1] |
| 146 | 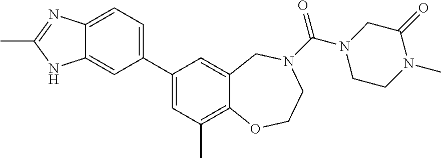 |
| 147 | 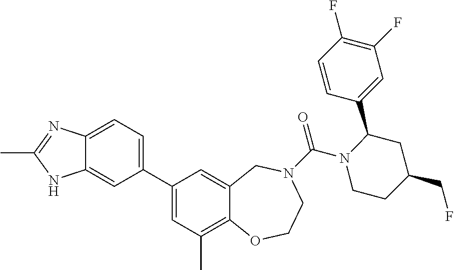 [SMILES: Cc1nc2ccc(-c3cc(C)c4c(c3)CN(C(=O)N3CC[C@H](CF)C[C@@H]3c3ccc(F)c(F)c3)CCO4)cc2[nH]1] |
| 148 | 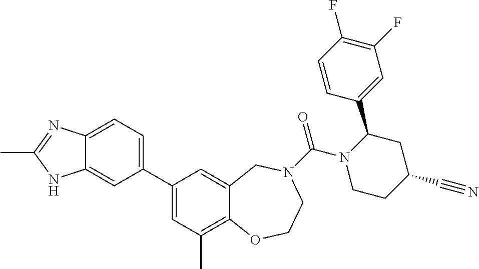 [SMILES: Cc1nc2ccc(-c3cc(C)c4c(c3)CN(C(=O)N3CC[C@@H](C#N)C[C@@H]3c3ccc(F)c(F)c3)CCO4)cc2[nH]1] |
| 149 | 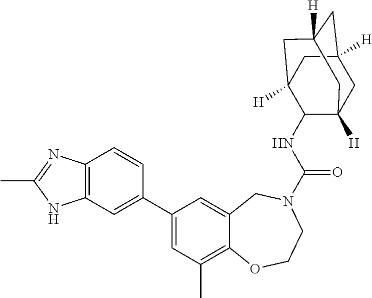 [SMILES: Cc1nc2ccc(-c3cc(C)c4c(c3)CN(C(=O)NC3[C@H]5C[C@@H]6C[C@@H](C[C@H]3C6)C5)CCO4)cc2[nH]1] |
| 150 | 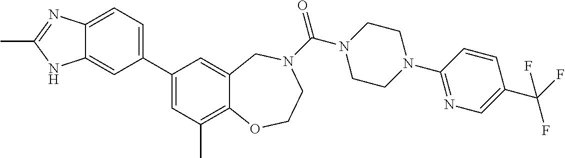 [SMILES: Cc1nc2ccc(-c3cc(C)c4c(c3)CN(C(=O)N3CCN(c5ccc(C(F)(F)F)cn5)CC3)CCO4)cc2[nH]1] |
| 151 | 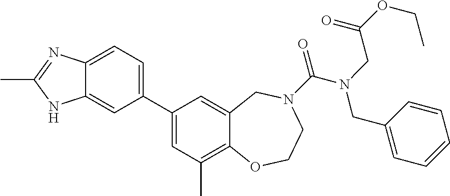 [SMILES: CCOC(=O)CN(Cc1ccccc1)C(=O)N1CCOc2c(C)cc(-c3ccc4nc(C)[nH]c4c3)cc2C1] |
| 152 | 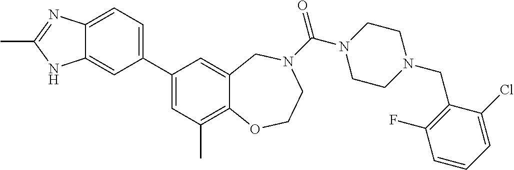 [SMILES: Cc1nc2ccc(-c3cc(C)c4c(c3)CN(C(=O)N3CCN(Cc5c(F)cccc5Cl)CC3)CCO4)cc2[nH]1] |
| 153 | 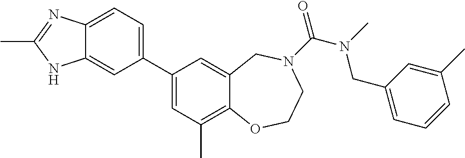 |
| 154 | 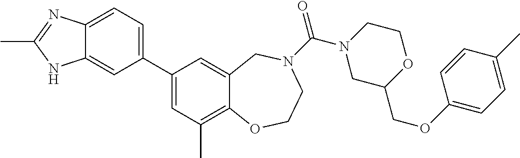 |
| 155 | 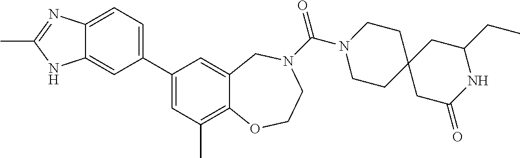 [SMILES: CCC1CC2(CCN(C(=O)N3CCOc4c(C)cc(-c5ccc6nc(C)[nH]c6c5)cc4C3)CC2)CC(=O)N1] |
| 156 | 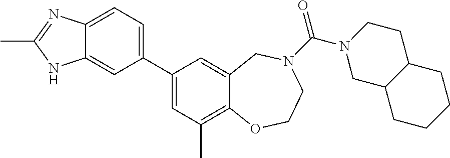 [SMILES: Cc1nc2ccc(-c3cc(C)c4c(c3)CN(C(=O)N3CCC5CCCCC5C3)CCO4)cc2[nH]1] |
| 157 | 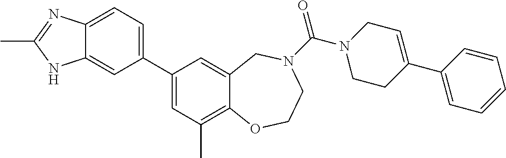 [SMILES: Cc1nc2ccc(-c3cc(C)c4c(c3)CN(C(=O)N3CC=C(c5ccccc5)CC3)CCO4)cc2[nH]1] |
| 158 | 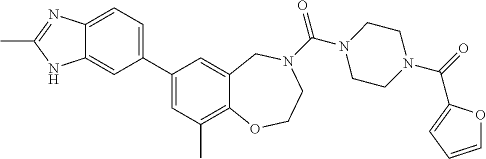 [SMILES: Cc1nc2ccc(-c3cc(C)c4c(c3)CN(C(=O)N3CCN(C(=O)c5ccco5)CC3)CCO4)cc2[nH]1] |
| 159 | 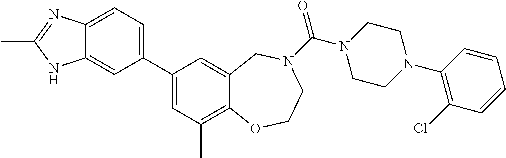 [SMILES: Cc1nc2ccc(-c3cc(C)c4c(c3)CN(C(=O)N3CCN(c5ccccc5Cl)CC3)CCO4)cc2[nH]1] |
| 160 | 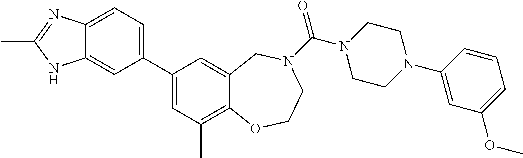 |
| 161 | 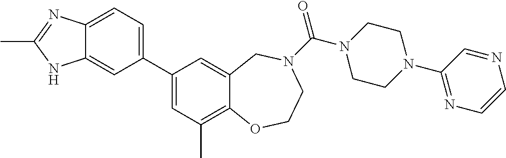 [SMILES: Cc1nc2ccc(-c3cc(C)c4c(c3)CN(C(=O)N3CCN(c5cnccn5)CC3)CCO4)cc2[nH]1] |
| 162 | 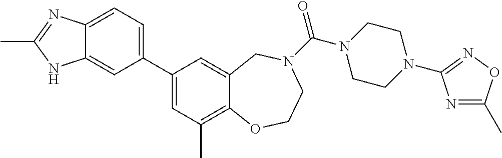 |
| 163 | 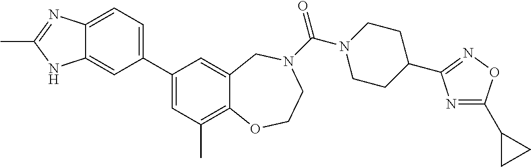 [SMILES: Cc1nc2ccc(-c3cc(C)c4c(c3)CN(C(=O)N3CCC(c5noc(C6CC6)n5)CC3)CCO4)cc2[nH]1] |
| 164 | 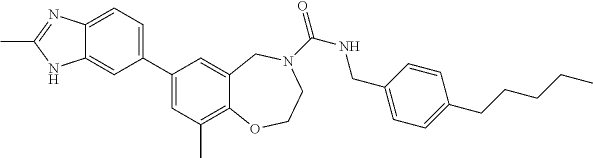 [SMILES: CCCCCc1ccc(CNC(=O)N2CCOc3c(C)cc(-c4ccc5nc(C)[nH]c5c4)cc3C2)cc1] |
| 165 | 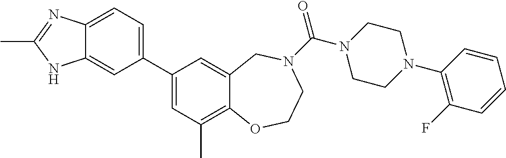 [SMILES: Cc1nc2ccc(-c3cc(C)c4c(c3)CN(C(=O)N3CCN(c5ccccc5F)CC3)CCO4)cc2[nH]1] |
| 166 | 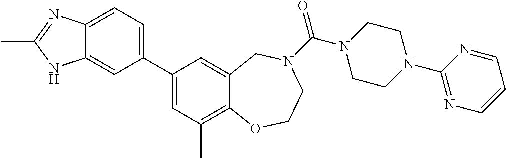 [SMILES: Cc1nc2ccc(-c3cc(C)c4c(c3)CN(C(=O)N3CCN(c5ncccn5)CC3)CCO4)cc2[nH]1] |
| 167 | 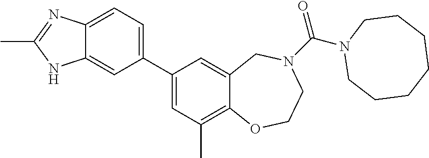 [SMILES: Cc1nc2ccc(-c3cc(C)c4c(c3)CN(C(=O)N3CCCCCCC3)CCO4)cc2[nH]1] |
| 168 | 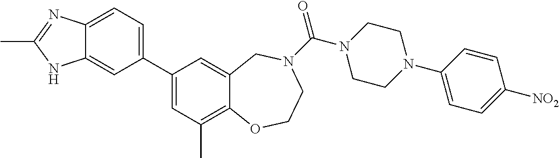 [SMILES: Cc1nc2ccc(-c3cc(C)c4c(c3)CN(C(=O)N3CCN(c5ccc([N+](=O)[O-])cc5)CC3)CCO4)cc2[nH]1] |
| 169 | 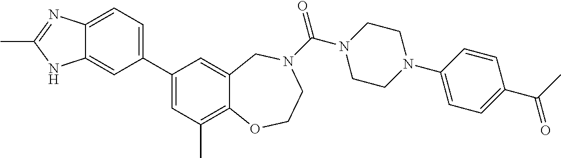 [SMILES: CC(=O)c1ccc(N2CCN(C(=O)N3CCOc4c(C)cc(-c5ccc6nc(C)[nH]c6c5)cc4C3)CC2)cc1] |
| 170 | 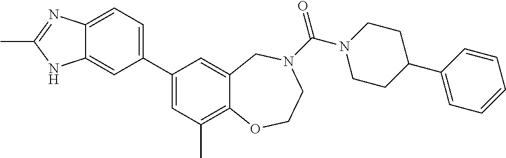 [SMILES: Cc1nc2ccc(-c3cc(C)c4c(c3)CN(C(=O)N3CCC(c5ccccc5)CC3)CCO4)cc2[nH]1] |
| 171 | 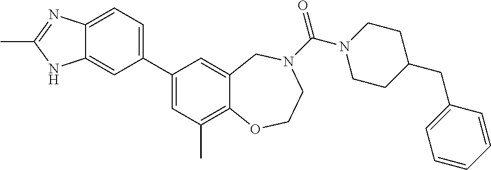 [SMILES: Cc1nc2ccc(-c3cc(C)c4c(c3)CN(C(=O)N3CCC(Cc5ccccc5)CC3)CCO4)cc2[nH]1] |
| 172 | 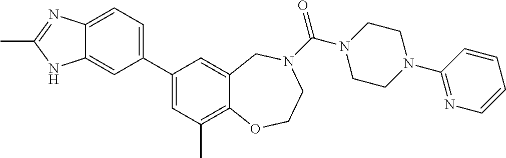 [SMILES: Cc1nc2ccc(-c3cc(C)c4c(c3)CN(C(=O)N3CCN(c5ccccn5)CC3)CCO4)cc2[nH]1] |
| 173 | 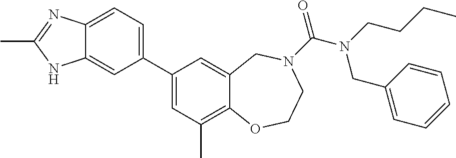 [SMILES: CCCCN(Cc1ccccc1)C(=O)N1CCOc2c(C)cc(-c3ccc4nc(C)[nH]c4c3)cc2C1] |
| 174 | 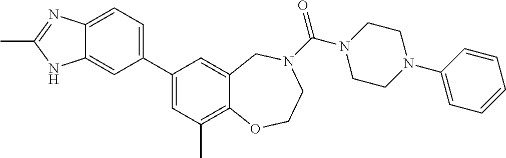 [SMILES: Cc1nc2ccc(-c3cc(C)c4c(c3)CN(C(=O)N3CCN(c5ccccc5)CC3)CCO4)cc2[nH]1] |
| 175 | 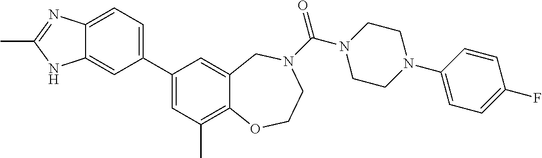 [SMILES: Cc1nc2ccc(-c3cc(C)c4c(c3)CN(C(=O)N3CCN(c5ccc(F)cc5)CC3)CCO4)cc2[nH]1] |
| 176 | 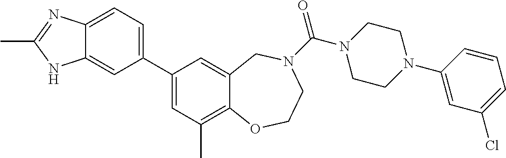 [SMILES: Cc1nc2ccc(-c3cc(C)c4c(c3)CN(C(=O)N3CCN(c5cccc(Cl)c5)CC3)CCO4)cc2[nH]1] |
| 177 | 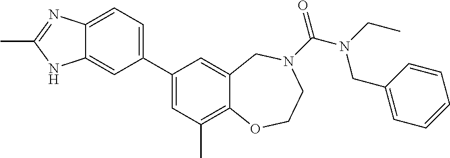 [SMILES: CCN(Cc1ccccc1)C(=O)N1CCOc2c(C)cc(-c3ccc4nc(C)[nH]c4c3)cc2C1] |
| 178 | 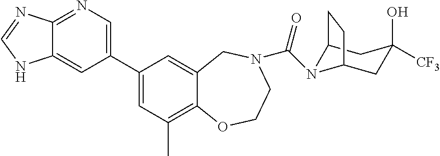 [SMILES: Cc1cc(-c2cnc3nc[nH]c3c2)cc2c1OCCN(C(=O)N1C3CCC1CC(O)(C(F)(F)F)C3)C2] |
| 179 | 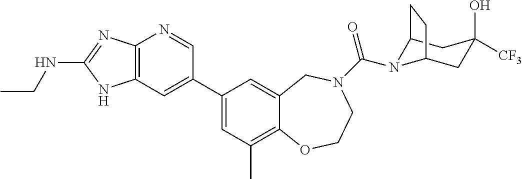 [SMILES: CCNc1nc2ncc(-c3cc(C)c4c(c3)CN(C(=O)N3C5CCC3CC(O)(C(F)(F)F)C5)CCO4)cc2[nH]1] |
| 180 | 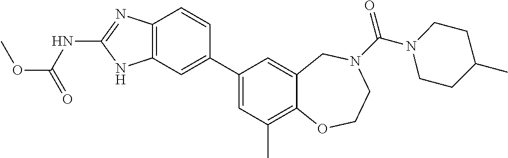 |
| 181 | 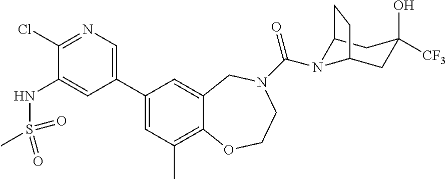 [SMILES: Cc1cc(-c2cnc(Cl)c(NS(C)(=O)=O)c2)cc2c1OCCN(C(=O)N1C3CCC1CC(O)(C(F)(F)F)C3)C2] |

### General Administration

In one aspect, the invention provides pharmaceutical compositions comprising an inhibitor of PI3K and/or mTOR according to the invention and a pharmaceutically acceptable carrier, excipient, or diluent. In certain other specific embodiments, administration is by the oral route. Administration of the compounds of the invention, or their pharmaceutically acceptable salts, in pure form or in an appropriate pharmaceutical composition, can be carried out via any of the accepted modes of administration or agents for serving similar utilities. Thus, administration can be, for example, orally, nasally, parenterally (intravenous, intramuscular, or subcutaneous), topically, transdermally, intravaginally, intravesically, intracistemally, or rectally, in the form of solid, semi-solid, lyophilized powder, or liquid dosage forms, such as for example, tablets, suppositories, pills, soft elastic and hard gelatin capsules, powders, solutions, suspensions, or aerosols, or the like, specifically in unit dosage forms suitable for simple administration of precise dosages.

The compositions will include a conventional pharmaceutical carrier or excipient and a compound of the invention as the/an active agent, and, in addition, may include carriers and adjuvants, etc.

Adjuvants include preserving, wetting, suspending, sweetening, flavoring, perfuming, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate and gelatin.

If desired, a pharmaceutical composition of the invention may also contain minor amounts of auxiliary substances such as wetting or emulsifying agents, pH buffering agents, antioxidants, and the like, such as, for example, citric acid, sorbitan monolaurate, triethanolamine oleate, butylalted hydroxytoluene, etc.

The choice of formulation depends on various factors such as the mode of drug administration (e.g., for oral administration, formulations in the form of tablets, pills or capsules) and the bioavailability of the drug substance. Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

One specific route of administration is oral, using a convenient daily dosage regimen that can be adjusted according to the degree of severity of the disease-state to be treated.

Solid dosage forms for oral administration include capsules, tablets, pills, powders, and granules. In such solid dosage forms, the active compound is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, cellulose derivatives, starch, alignates, gelatin, polyvinylpyrrolidone, sucrose, and gum acacia, (c) humectants, as for example, glycerol, (d) disintegrating agents, as for example, agar-agar, calcium carbonate, potato or tapioca starch, alginic acid, croscarmellose sodium, complex silicates, and sodium carbonate, (e) solution retarders, as for example paraffin, (f) absorption accelerators, as for example, quaternary ammonium compounds, (g) wetting agents, as for example, cetyl alcohol, and glycerol monostearate, magnesium stearate and the like (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid dosage forms as described above can be prepared with coatings and shells, such as enteric coatings and others well known in the art. They may contain pacifying agents, and can also be of such composition that they release the active compound or compounds in a certain part of the intestinal tract in a delayed manner. Examples of embedded compositions that can be used are polymeric substances and waxes. The active compounds can also be in microencapsulated form, if appropriate, with one or more of the above-mentioned excipients.

Liquid dosage forms for oral administration include pharmaceutically acceptable emulsions, solutions, suspensions, syrups, and elixirs. Such dosage forms are prepared, for example, by dissolving, dispersing, etc., a compound(s) of the invention, or a pharmaceutically acceptable salt thereof, and optional pharmaceutical adjuvants in a carrier, such as, for example, water, saline, aqueous dextrose, glycerol, ethanol and the like; solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide; oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan; or mixtures of these substances, and the like, to thereby form a solution or suspension.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions for rectal administrations are, for example, suppositories that can be prepared by mixing the compounds of the present invention with for example suitable non-irritating excipients or carriers such as cocoa butter, polyethyleneglycol or a suppository wax, which are solid at ordinary temperatures but liquid at body temperature and therefore, melt while in a suitable body cavity and release the active component therein.

Dosage forms for topical administration of a compound of this invention include ointments, powders, sprays, and inhalants. The active component is admixed under sterile conditions with a physiologically acceptable carrier and any preservatives, buffers, or propellants as may be required. Ophthalmic formulations, eye ointments, powders, and solutions are also contemplated as being within the scope of this invention.

Compressed gases may be used to disperse a compound of this invention in aerosol form. Inert gases suitable for this purpose are nitrogen, carbon dioxide, etc.

Generally, depending on the intended mode of administration, the pharmaceutically acceptable compositions will contain about 1% to about 99% by weight of a compound(s) of the invention, or a pharmaceutically acceptable salt thereof, and 99% to 1% by weight of a suitable pharmaceutical excipient. In one example, the composition will be between about 5% and about 75% by weight of a compound(s) of the invention, or a pharmaceutically acceptable salt thereof, with the rest being suitable pharmaceutical excipients.

Actual methods of preparing such dosage forms are known, or will be apparent, to those skilled in this art; for example, see Remington's Pharmaceutical Sciences, 18th Ed., (Mack Publishing Company, Easton, Pa., 1990). The composition to be administered will, in any event, contain a therapeutically effective amount of a compound of the invention, or a pharmaceutically acceptable salt thereof, for treatment of a disease-state in accordance with the teachings of this invention.

The compounds of the invention, or their pharmaceutically acceptable salts or solvates, are administered in a therapeutically effective amount which will vary depending upon a variety of factors including the activity of the specific compound employed, the metabolic stability and length of action of the compound, the age, body weight, general health, sex, diet, mode and time of administration, rate of excretion, drug combination, the severity of the particular disease-states, and the host undergoing therapy. The compounds of the present invention can be administered to a patient at dosage levels in the range of about 0.1 to about 1,000 mg per day. For a normal human adult having a body weight of about 70 kilograms, a dosage in the range of about 0.01 to about 100 mg per kilogram of body weight per day is an example. The specific dosage used, however, can vary. For example, the dosage can depend on a number of factors including the requirements of the patient, the severity of the condition being treated, and the pharmacological activity of the compound being used. The determination of optimum dosages for a particular patient is well known to one of ordinary skill in the art.

If formulated as a fixed dose, such combination products employ the compounds of this invention within the dosage range described above and the other pharmaceutically active agent(s) within its approved dosage range. Compounds of the instant invention may alternatively be used sequentially with known pharmaceutically acceptable agent(s) when a combination formulation is inappropriate.

### Utility

Compounds of the Invention have activity for PI3K-alpha, mTOR, or for both. Compounds of this invention have been tested using the assays described in Biological Examples 1 and 3 and have been determined to be inhibitors of PI3K-alpha, mTOR, or for both. Suitable in vitro assays for measuring PI3K, mTORc1, and mTORc2 activity and the inhibition thereof by compounds are known in the art. For further details of an in vitro assay for measuring PI3K and mTOR activity see Biological Examples, Example 1, 2, and 3 infra. Cell-based assays for measurement of in vitro efficacy in treatment of cancer are known in the art. In addition, assays are described in Biological Examples, Example 5 and 6, infra. Suitable in vivo models for cancer are known to those of ordinary skill in the art. For further details of in vivo models for prostate adenocarcinoma, glioblastoma, lung carcinoma, and melanoma, see Biological Examples 7, 8, 9, 10, 11, 12, and 13, infra. Following the examples disclosed herein, as well as that disclosed in the art, a person of ordinary skill in the art can determine the activity of a compound of this invention.

Compounds of Formula I are useful for treating diseases, particularly cancer in which activity against PI3K-alpha, mTOR, or both contributes to the pathology and/or symptomatology of the disease. For example, cancer in which activity against PI3K-alpha, mTOR, or both contributes to its pathology and/or symptomatology include breast cancer, mantle cell lymphoma, renal cell carcinoma, acute myelogenous leukemia, chronic myelogenous leukemia, NPM/ALK-transformed anaplastic large cell lymphoma, diffuse large B cell lymphoma, rhabdomyosarcoma, ovarian cancer, endometrial cancer, cervical cancer, non small cell lung carcinoma, small cell lung carcinoma, adenocarcinoma, colon cancer, rectal cancer, gastric carcinoma, hepatocellular carcinoma, melanoma, pancreatic cancer, prostate carcinoma, thyroid carcinoma, anaplastic large cell lymphoma, hemangioma, glioblastoma, or head and neck cancer.

Compounds of the invention are also useful as inhibitors of PI3Kα and/or mTOR in vivo for studying the in vivo role of PI3Kα and/or mTOR in biological processes, including the diseases described herein. Accordingly, the invention also comprises a method of inhibiting PI3Kα and/or mTOR in vivo comprising administering a compound or composition of the invention to a mammal.

### General Synthesis

Compounds of this invention can be made by the synthetic procedures described below. The starting materials and reagents used in preparing these compounds are either available from commercial suppliers such as Aldrich Chemical Co. (Milwaukee, Wis.), or Bachem (Torrance, Calif.), or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4^{th} Edition) and Larock's Comprehensive Organic Transformations (VCH Publishers Inc., 1989). These schemes are merely illustrative of some methods by which the compounds of this invention can be synthesized, and various modifications to these schemes can be made and will be suggested to one skilled in the art having referred to this disclosure. The starting materials and the intermediates of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure and over a temperature range from about −78° C. to about 150° C., more specifically from about 0° C. to about 125° C. and more specifically at about room (or ambient) temperature, e.g., about 20° C. Unless otherwise stated (as in the case of hydrogenation), all reactions are performed under an atmosphere of nitrogen.

Prodrugs can be prepared by techniques known to one skilled in the art. These techniques generally modify appropriate functional groups in a given compound. These modified functional groups regenerate original functional groups by routine manipulation or in vivo. Amides and esters of the compounds of the present invention may be prepared according to conventional methods. A thorough discussion of prodrugs is provided in T. Higuchi and V. Stella, “Pro-drugs as Novel Delivery Systems,” Vol 14 of the A.C.S. Symposium Series, and in Bioreversible Carriers in Drug Design, ed. Edward B. Roche, American Pharmaceutical Association and Pergamon Press, 1987, both of which are incorporated herein by reference for all purposes.

The compounds of the invention, or their pharmaceutically acceptable salts, may have asymmetric carbon atoms or quaternized nitrogen atoms in their structure. Compounds of the Invention that may be prepared through the syntheses described herein may exist as single stereoisomers, racemates, and as mixtures of enantiomers and diastereomers. The compounds may also exist as geometric isomers. All such single stereoisomers, racemates and mixtures thereof, and geometric isomers are intended to be within the scope of this invention.

Some of the compounds of the invention contain an active ketone —C(O)CF₃ and may exist in part or in whole as the —C(OH₂)CF₃ form. Regardless of whether the compound is drawn as the —C(O)CF₃ or —C(OH₂)CF₃ form, both are included within the scope of the Invention. Although an individual compound may be drawn as the —C(O)CF₃ form, one of ordinary skill in the art would understand that the compound may exist in part or in whole as the —C(OH₂)CF₃ form and that the ratio of the two forms may vary depending on the compound and the conditions in which it exists.

Some of the compounds of the invention may exist as tautomers. For example, where a ketone or aldehyde is present, the molecule may exist in the enol form; where an amide is present, the molecule may exist as the imidic acid; and where an enamine is present, the molecule may exist as an imine. All such tautomers are within the scope of the invention. Further, for example, in this application R¹ can be 5-oxo-1H-1,2,4-triazol-3-yl, depicted structurally below:

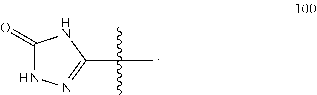 [SMILES: *c1n[nH]c(=O)[nH]1]

Both 5-oxo-1H-1,2,4-triazol-3-yl and the above structure 1 include, and are equivalent to, 3-hydroxy-4H-1,2,4-triazol-5-yl and its structure 2:

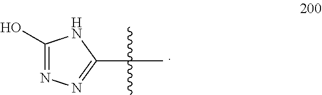 [SMILES: *c1nnc(O)[nH]1]

Regardless of which structure or which terminology is used, each tautomer is included within the scope of the Invention.

The present invention also includes N-oxide derivatives and protected derivatives of compounds of the Invention. For example, when compounds of the Invention contain an oxidizable nitrogen atom, the nitrogen atom can be converted to an N-oxide by methods well known in the art. When compounds of the Invention contain groups such as hydroxy, carboxy, thiol or any group containing a nitrogen atom(s), these groups can be protected with a suitable “protecting group” or “protective group”. A comprehensive list of suitable protective groups can be found in T. W. Greene, Protective Groups in Organic Synthesis, John Wiley & Sons, Inc. 1991, the disclosure of which is incorporated herein by reference in its entirety. The protected derivatives of compounds of the Invention can be prepared by methods well known in the art.

Methods for the preparation and/or separation and isolation of single stereoisomers from racemic mixtures or non-racemic mixtures of stereoisomers are well known in the art. For example, optically active (R)- and (S)-isomers may be prepared using chiral synthons or chiral reagents, or resolved using conventional techniques. Enantiomers (R- and S-isomers) may be resolved by methods known to one of ordinary skill in the art, for example by: formation of diastereoisomeric salts or complexes which may be separated, for example, by crystallization; via formation of diastereoisomeric derivatives which may be separated, for example, by crystallization, selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. It will be appreciated that where a desired enantiomer is converted into another chemical entity by one of the separation procedures described above, a further step may be required to liberate the desired enantiomeric form. Alternatively, specific enantiomer may be synthesized by asymmetric synthesis using optically active reagents, substrates, catalysts or solvents or by converting on enantiomer to the other by asymmetric transformation. For a mixture of enantiomers, enriched in a particular enantiomer, the major component enantiomer may be further enriched (with concomitant loss in yield) by recrystallization.

In addition, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

The chemistry for the preparation of the compounds of this invention is known to those skilled in the art. In fact, there may be more than one process to prepare the compounds of the invention. The following examples illustrate but do not limit the invention. All references cited herein are incorporated by reference in their entirety.

An intermediate of formula 4 where PG is a nitrogen-protecting group, R^{5a} and R^{5c} are independently hydrogen or alkyl, R^{5h} is hydrogen or halo, R^{5b} is (C₁₋₃)alkyl, and R^{5d}, R^{5e}, R^{5f}, and R^{5g} are hydrogen can be prepared according to Scheme 1.

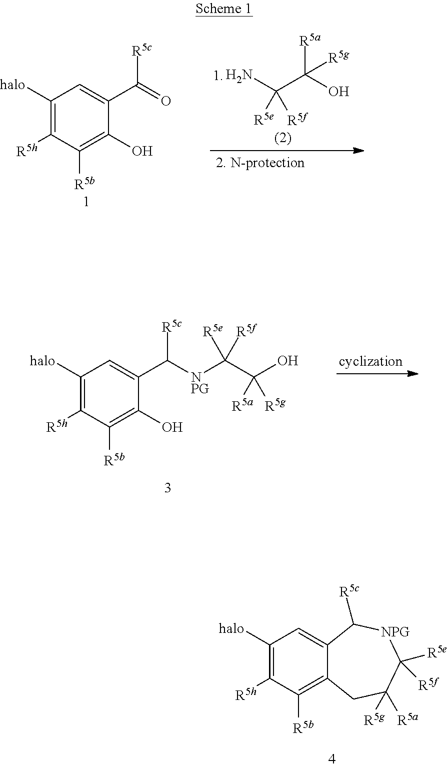

In particular, an intermediate of formula 4a can be prepared according to Scheme 1a

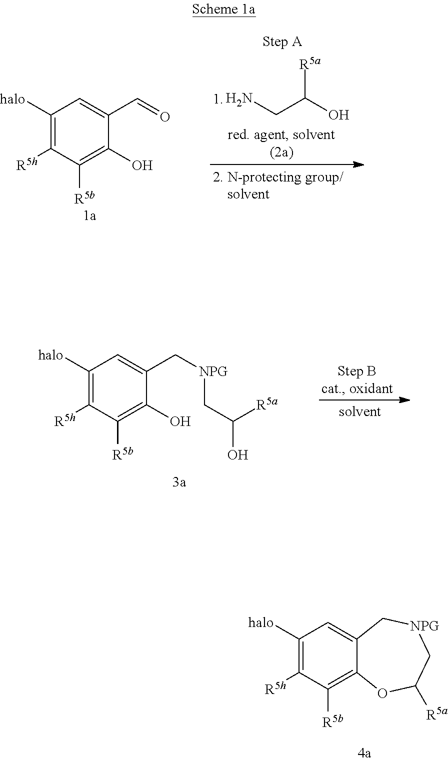

An intermediate of formula Ia is commercially available or can be prepared using methods known to one of ordinary skill in the art.

An intermediate of formula 2a where R^{5a} is hydrogen or methyl is commercially available. The intermediate of formula Ia is treated with an intermediate of formula 2a in the presence of a reducing agent such as sodium borohydride, in a solvent(s) such as tetrahydrofuran and/or methanol and allowed to react at a temperature of about 40° C. for approximately 4 hours. The solvent is then removed and the reaction is taken up in a solvent(s) such as ethyl acetate and/or saturated sodium bicarbonate. To this suspension a nitrogen-protecting group precursor, such as di-tert-butyl dicarbonate, is added and the mixture is allowed to stir at room temperature overnight to yield an intermediate of formula 3a where PG is a nitrogen-protecting group.

Intermediate 3a is then treated with a catalyst, such as triphenylphosphine, in the presence of a dehydrating agent such as diisopropyl azodicarboxylate, in a solvent such as DCM. The reaction is allowed to proceed at room temperature for approximately 12 hours and the resulting product is optionally purified by column chromatography to yield an intermediate of formula 4a. Alternatively, the intermediate of formula 4a can be prepared by treating the intermediate of formula 3a with Burgess' reagent.

An intermediate of formula 5 where PG is a nitrogen-protecting group, R^{5a} and R^{5c} are independently hydrogen or alkyl, R^{5h} is hydrogen or halo, R^{5b} is (C₁₋₃)alkyl, R^{5e}, R^{5f}, and R^{5g} are hydrogen, and R¹ is as defined in the Summary of the Invention for a Compound of Formula I can be prepared according to Scheme 2.

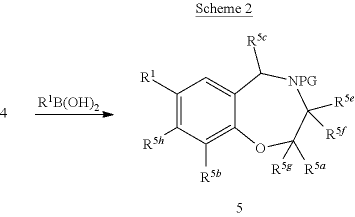

where the intermediate of formula 4 is prepared as described in Scheme 1.

In particular, an intermediate of formula 5a where R^{5a} is hydrogen or alkyl, R^{5b} is hydrogen or halo, R^{5b} is (C₁₋₃)alkyl, and R¹ is as defined in the Summary of the Invention for a Compound of Formula I, can be prepared according to Scheme 2a.

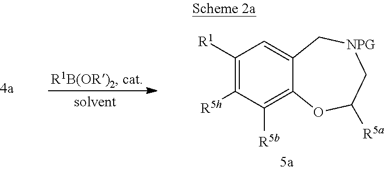

The intermediate of formula 4a, prepared as described in Scheme 1a, is treated with a boronic acid of formula —B(OR′)₂ (where both R′ are hydrogen or the two R′ together form a boronic ester), which is commercially available or can be prepared using procedures known to one of ordinary skill in the art. The reaction is carried out in the presence of a catalyst such as Pd(dppf)₂Cl₂, a base such as potassium carbonate, and in a solvent such as DME at about 80° C. for about 2 hours. The product can then be purified by chromatography to yield an intermediate of formula 5a.

Alternatively, an intermediate of formula 5, as defined above, can be prepared as described in Scheme 4.

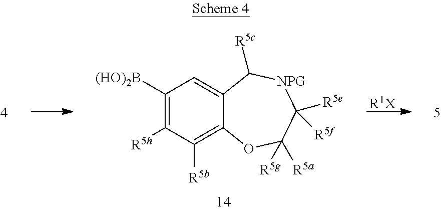

In particular, an intermediate of formula 5b where PG is a nitrogen-protecting group and R¹ is as defined in the Summary of the Invention for a Compound of Formula I can be prepared according to Scheme 4a.

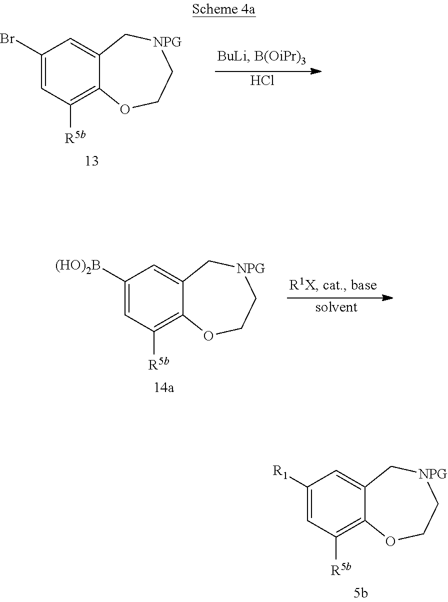

An intermediate of formula 13, where PG is a nitrogen-protecting group, is prepared as described in Scheme 1a. 13 is treated with triisopropylborate in a solvent such as THF at a temperature of about −60° C., followed by dropwise addition of a base such as n-butyllithium in tetrahydrofuran. The reaction was allowed to proceed for about 30 minutes, was treated with an acid such as hydrochloric acid, and allowed to warm to room temperature to yield an intermediate of formula 14a. Intermediate 14a is then treated with an intermediate of formula R¹X (where X is a halide, and which is commercially available or can be prepared using procedures known to one of ordinary skill in the art), in the presence of a base such as potassium carbonate, in the presence of a catalyst such as tetrakis(triphenylphosphine) palladium(0), and in a solvent(s) such as 1,2-dimethoxyethane and/or water. The reaction is allowed to proceed under nitrogen and stirred at reflux for about 3 hours to yield an intermediate of formula 5b.

A Compound of the Invention of Formula I where R^{5a} and R^{5c} are independently hydrogen or alkyl, R^{5h} is hydrogen or halo, R^{5b} is (C₁₋₃)alkyl, R^{5e}, R^{5f}, and R^{5g} are hydrogen, and R¹ and R² are as defined in the Summary of the Invention for a Compound of Formula I can be prepared as described in Scheme 5,

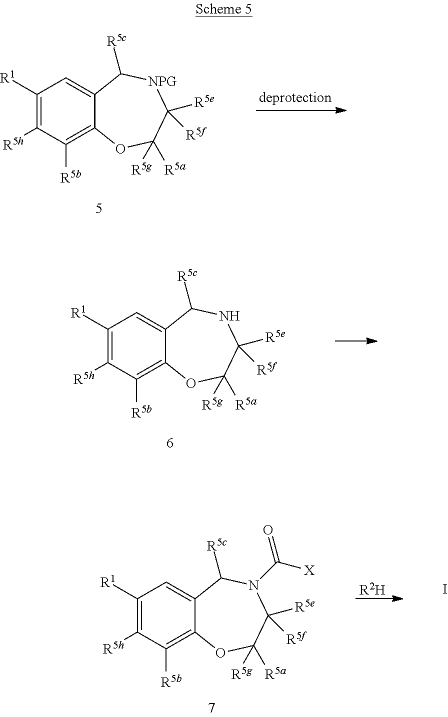

In particular, a Compound of Formula I(j) where R^{5a} is hydrogen or alkyl, R^{5h} is hydrogen or halo, and R¹, R^{5b}, and R² are as defined in the Summary of the Invention for a Compound of Formula I can be prepared as described in Scheme 5a.

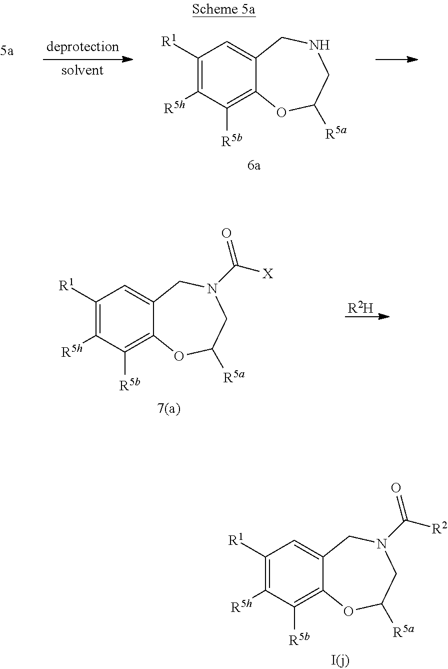

The protecting group on the intermediate of formula 5a is removed. When the protecting group is Boc, it can be removed with HCl to yield an intermediate of formula 6a. The intermediate of formula 7(a) where X is halo is prepared using procedures known to one of ordinary skill in the art. The intermediate of formula R²H is commercially available or can be prepared using procedures described herein or procedures known to one of ordinary skill in the art. The intermediate of formula 6a is then treated with R²H, in the presence of a base such as Hiinig's base, in a solvent such as DMF, at a temperature of about 50° C. The product can be purified by column chromatography to yield an intermediate of Formula I(j).

In particular, a Compound of Formula I(k) where R¹ and R² are as defined in the Summary of the Invention for a Compound of Formula I can be prepared according to Scheme 5b.

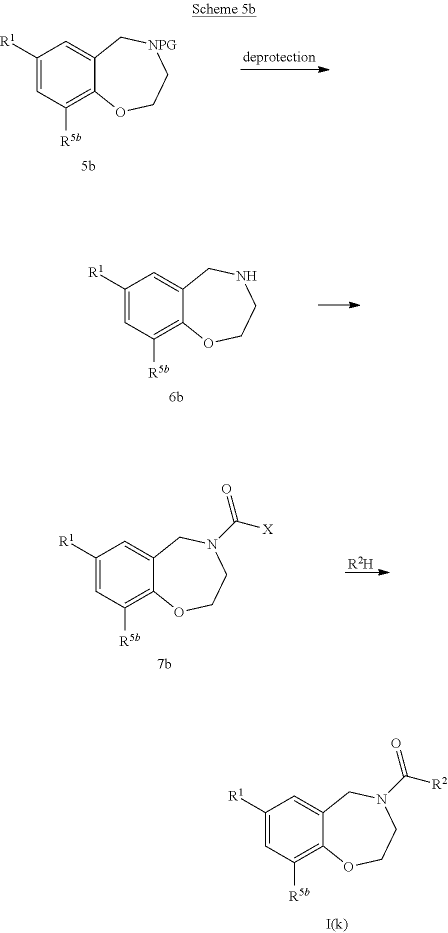

The protecting group on intermediate of formula 5b, prepared as described in Scheme 4a, is removed. When the protecting group is Boc, it can be removed with HCl to yield an intermediate of formula 6b. Intermediate 7b, where X is a leaving group, is then prepared using procedures known to one of ordinary skill in the art. Intermediate 7b is then treated with an intermediate of R²H using conditions known to one or ordinary skill in the art to yield a Compound of Formula I(k).

A compound of the invention where R^{5a}, R^{5c}, R^{5d}, R^{5e}, R^{5f}, R^{5g}, and R^{5h} are hydrogen; R¹ is benzimidazol-6-yl substituted at the 2-position with one R⁷; R⁷ is alkyl; and R^{5b}, and R² are as defined in the Summary of the Invention for a Compound of Formula I can be prepared according to Scheme 6.

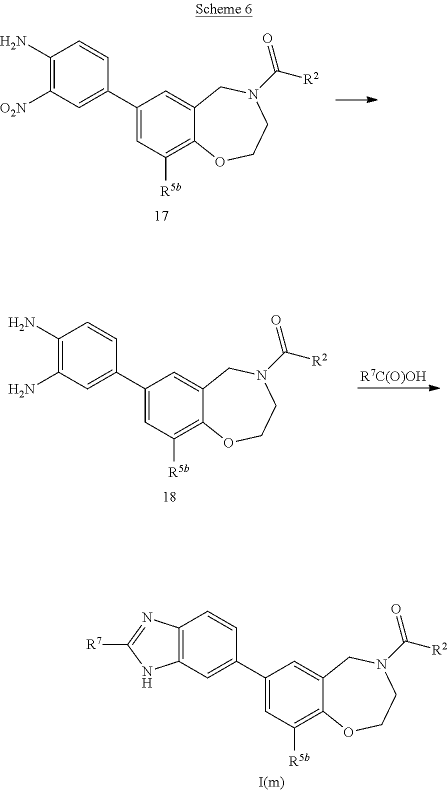

A Compound of Formula I(y) where R^{5b} and R² are as defined in the Summary of the Invention for a Compound of Formula I can be prepared according to Scheme 7a.

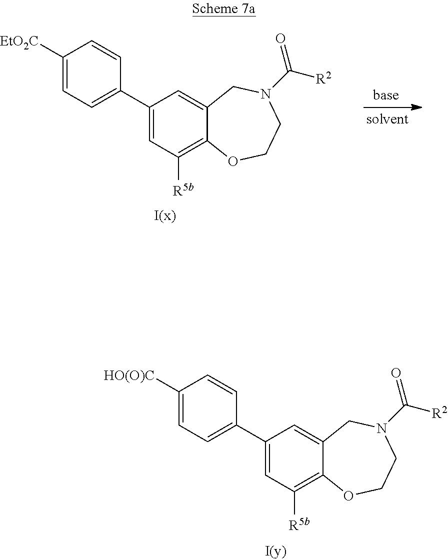

The Compound of Formula I(x), prepared using procedures according to Scheme 5b, is treated with a base such as LiOH, in a solvent(s) such as THF and/or water to yield the hydrolyzed Compound of Formula I(y).

A Compound of Formula I where R¹, R², R^{5b}, R^{5a}, R^{5c}, R^{5d}, R^{5e}, R^{5f}, R^{5g}, and R^{5h} are as defined in the Summary of the Invention for a Compound of Formula I can be prepared according to the following scheme (where X is halo) using procedures known to one of ordinary skill in the art.

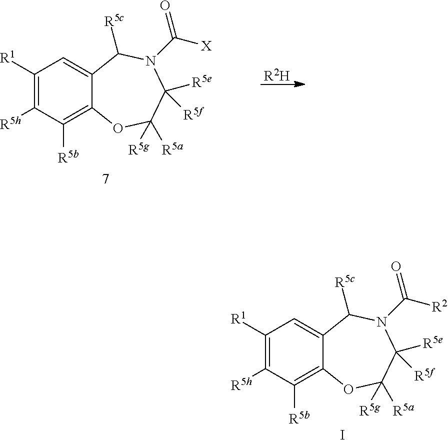

A Compound of Formula I where R¹, R², R^{5a}, R^{5b}, R^{5c}, R^{5d}, R^{5e}, R^{5f}, R^{5g}, and R^{5h} are as defined in the Summary of the Ivention for a Compound of Formula I can be prepared according to the following scheme where R is —B(OR′)₂ (where both R¹ are hydrogen or the two R¹ together form a boronic ester) and Y is halo, or R is halo and Y is —B(OR′)₂ (where both R¹ are hydrogen or the two R¹ together form a boronic ester) using Suzuki coupling procedures known to one of ordinary skill in the art.

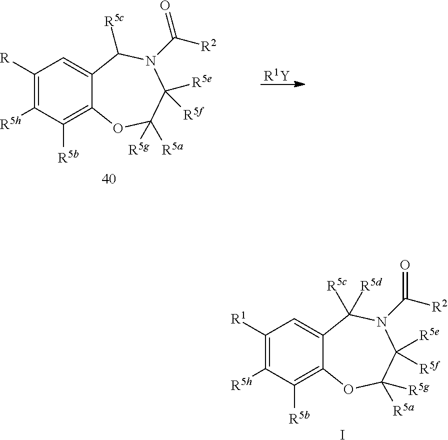

### Synthetic Examples

### Reagent Preparation 1

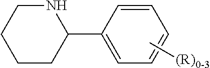

STEP 1: To a solution of tert-butyl 2-oxopiperidine-1-carboxylate (0.30 g, 1.51 mmol) in tetrahydrofuran (8 mL) cooled to −78° C. was added slowly over 15 minutes 0.3 M 3,4,5-trifluorophenylmagnesium bromide in tetrahydrofuran (3.30 mL, 1.66 mmol) and the mixture was then allowed to warm to 25° C. over 30 minutes. The reaction mixture was poured slowly into an ice cold solution of 0.5 N hydrochloric acid (100 mL), and extracted twice with ethyl acetate (2×50 mL). The combined organic extracts were dried over anhydrous sodium sulfate then filtered and concentrated. The residue was purified by silica gel column chromatography (diethyl ether/hexanes 1:4) to give tert-butyl 5-oxo-5-(3,4,5-trifluorophenyl)pentylcarbamate (0.18 g, 36% yield). MS (EI) for C₁₆H₂₀F₃NO₃: 332 (M11⁴).

STEP 2: Tert-butyl 5-oxo-5-(3,4,5-trifluorophenyl)pentylcarbamate (0.18 g, 0.54 mmol) was stirred in trifluoroacetic acid/dichloromethane 1:1 (8 mL) for 1 hour then concentrated. The residue was dissolved in ethyl acetate (40 mL) and washed with saturated sodium chloride/2M aqueous sodium hydroxide 10:1 (11 mL), then dried over anhydrous sodium sulfate, filtered and concentrated to provide 5-amino-1-(3,4,5-trifluorophenyl)pentan-1-one (0.11 g, 88% yield) as an oil. MS (EI) for C₁₁H₁₂F₃NO: 232 (MH⁺).

STEP 3: To 5-amino-1-(3,4,5-trifluorophenyl)pentan-1-one (0.11 g, 0.48 mmol) in tetrahydrofuran/methanol 4:1 (10 mL) was added in portions over 20 minutes solid sodium borohydride (0.20 g, 5.0 mmol) and stirring was continued 18 hours at 25° C. The reaction mixture was concentrated then taken into ethyl acetate (40 mL), washed with saturated sodium chloride/2 N aqueous sodium hydroxide 10:1 (11 mL) then dried over anhydrous sodium sulfate, filtered and concentrated. The residue was purified by silica gel column chromatography (ethyl acetate/hexanes, 1:1) to give 2-(3,4,5-trifluorophenyl)piperidine (0.70 g, 68% yield) as an oil. ¹H NMR (400 MHz, CDCl₃): 7.01 (m, 2H), 3.52 (m, 1H), 3.17 (m, 1H) 2.77 (m, 1H), 2.07 (br s, 1H), 1.88 (m, 1H), 1.74 (m, 1H), 1.64 (m, 1H), 1.55-1.35 (m, 3H).

Using analogous synthetic techniques and substituting with alternative starting materials in step 1 the following reagents were prepared. Alternative starting materials were purchased from commercial sources unless otherwise indicated.

2-(3-chloro-4-fluorophenyl)piperidine. Prepared according to the method of reagent preparation 1 using 3-chloro-4-fluorphenylmagnesium bromide in step 1. MS (EI) for C₁₁H₁₃ClFN: 214 (MH⁺).

2-(3,5-difluorophenyl)piperidine. Prepared according to the method of reagent preparation 1 using 3,4-difluorphenylmagnesium bromide in step 1. MS (EI) for C₁₋₁₉₈ (MH⁺).

2-(4-fluoro-3-methylphenyl)piperidine. Prepared according to the method of reagent preparation 1 using 4-fluoro-3-methylphenylmagnesium bromide in step 1. ¹H NMR (400 MHz, CDCl₃): 7.19 (dd, 1H), 7.11 (m, 1H), 6.92 (t, 1H), 3.54 (m, 1H), 3.17 (m, 1H), 2.76 (m, 1H), 2.25 (d, 3H), 1.89 (m, 2H), 1.75 (m, 1H), 1.66 (m, 1H), 1.48 (m, 2H).

2-(4-chlorophenyl)piperidine. Synthesized according to the method of reagent preparation 1 using 4-chlorophenylmagnesium bromide in step 1. MS (EI) for C₁₁H₁₃F₂N: 196 (MH⁺).

2-(3,4-difluorophenyl)piperidine. Synthesized according to the method of reagent preparation 1 using 3,4-difluorophenylmagnesium bromide in step 1. ¹H NMR (400 MHz, CDCl₃): 7.64 (m, 1H), 7.49 (m, 1H), 7.15 (m, 1H), 3.83 (m, 2H), 2.57 (m, 2H), 1.84 (m, 2H), 1.67 (m, 2H).

2-(4-chloro-3-fluorophenyl)piperidine. Synthesized according to the method of reagent preparation 1 using 4-chloro-3-fluorophenylmagnesium bromide in step 1. ¹H NMR (400 MHz, CDCl₃): 7.59 (dd, 1H), 7.49 (dd, 1H), 7.38 (tr, 1H), 3.84 (m, 2H), 2.56 (m, 2H), 1.84 (m, 2H), 1.67 (m, 2H).

2-(3,5-bis(trifluoromethyl)phenyl)piperidine. Synthesized according to the method of reagent preparation 1 using 3,5-bis(trifluoromethyl)phenylmagnesium bromide in step 1. MS (EI) for C₁₁H₁₃F₆N: 298 (MH⁺).

2-(3-chloro-5-fluorophenyl)piperidine. Synthesized according to the method of reagent preparation 1 using 3-chloro-5-fluorophenylmagnesium bromide in step 1. MS (EI) for C₁₁H₁₃ClFN: 214 (MH⁺).

2-(4-(trifluoromethoxy)phenyl)piperidine. Synthesized according to the method of reagent preparation 1 using 4-trifluoromethoxyphenylmagnesium bromide in step 1. MS (EI) for C₁₂H₁₄F₃NO: 246 (MH⁺).

2-(3-fluoro-4-methoxyphenyl)piperidine. Synthesized according to the method of reagent preparation 1 using 3-fluoro-4-methoxyphenylmagnesium bromide in step 1. MS (EI) for C₁₂H₁₆FNO: 210 (MH⁺).

2-(2-fluorophenyl)piperidine. Synthesized according to the method of reagent preparation 1 using 2-fluorophenylmagnesium bromide in step 1. MS (EI) for C₁₁H₁₄FN: 180 (MH⁺).

2-(4-(trifluoromethyl)phenyl)piperidine. Synthesized according to the method of reagent preparation 1 using 4-trifluorophenylmagnesium chloride in step 1. MS (EI) for C₁₂H₁₄F₃N: 230 (MH⁺).

2-(3-fluoro-4-methylphenyl)piperidine. Synthesized according to the method of reagent preparation 1 using 3-fluoro-4-methylphenylmagnesium bromide in step 1. MS (EI) for C₁₂H₁₆FN: 194 (MH⁺).

2-(3,4-dichlorophenyl)piperidine. Synthesized according to the method of reagent preparation 1 using 3,4-dichlorophenylmagnesium bromide in step 1. MS (EI) for C₁₁H₁₃Cl₂N: 230 (MH⁺).

2-(4-fluoro-2-methylphenyl)piperidine. Synthesized according to the method of reagent preparation 1 using 4-fluoro-2-methylphenylmagnesium bromide in step 1. MS (EI) for C₁₂H₁₆FN: 194 (MW).

### Reagent Preparation 2

### (±)-(2R,4S)-2-phenylpiperidin-4-ylmethanol

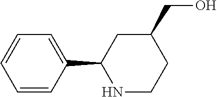 [SMILES: OC[C@H]1CCN[C@@H](c2ccccc2)C1]

STEP 1: A suspension of potassium tert-butoxide (1.25 g, 11.1 mmol) and methyltriphenylphosphonium bromide (3.86 g, 1.1 mmol) in tetrahydrofuran (100 mL) was stirred at 40° C. for 30 minutes. The mixture was then cooled to room temperature and a solution of tert-butyl 4-oxo-2-phenylpiperidine-1-carboxylate (2.35 g, 8.5 mmol) in tetrahydrofuran (30 mL) was added slowly. The reaction mixture was stirred at 40° C. for 24 hours. The mixture was cooled to room temperature and quenched by the addition of water and diluted with ethyl acetate (250 mL). The organic layer was separated then washed with water, 10% aqueous citric acid and brine, dried over anhydrous sodium sulfate, filtered and concentrated. Column chromatography on silica gel (hexane:ethyl acetate 95:5 to 9:1) provided tert-butyl 4-methylene-2-phenylpiperidine-1-carboxylate (2.24 g, 96%). ¹H NMR (400 MHz, CDCl₃): 7.31 (m, 4H), 7.21 (m, 1H), 5.48 (br d, 1H), 4.84 (dd, 2H), 4.07 (br dd, 1H), 2.85 (br, t, 1H), 2.78 (dtr, 1H), 2.64 (dd, 1H), 2.28 (dtr, 1H), 2.20 (br d, 1H), 1.42 (s, 9H). GC/MS (EI) for C₁₇H₂₃NO₂: 273 (M⁺).

STEP 2: To solution of tert-butyl 4-methylene-2-phenylpiperidine-1-carboxylate (2.20 g, 8.04 mmol) in tetrahydrofuran (50 mL) at 0° C. was added borane-tetrahydrofuran complex (1M solution in in tetrahydrofuran) (12.1 mL, 12.1 mmol) and the reaction mixture was stirred at 0° C. for 1 hour. The reaction mixture was allowed to warm to room temperature then stirred for an additional 2 hours. It was cooled to 0° C. and 2M aqueous sodium hydroxide (8.0 mL, 16.0 mmol) was added slowly followed by the slow addition of 30% aqueous hydrogen peroxide (5.5 mL, 48.4 mmol). The mixture was stirred for another hour then diluted with water (100 mL) and partitioned with ethyl acetate (250 mL). The organic layer was separated and washed with 2M aqueous sodium thiosulfate (100 mL), brine, dried over anhydrous sodium sulfate, filtered and concentrated. Column chromatography in silica gel (chloroform:methanol 9:1 to 4:1) provided tert-butyl 4-(hydroxymethyl)-2-phenylpiperidine-1-carboxylate (1.86 g, 79%). ¹H NMR (400 MHz, CDCl₃): 7.30 (m, 2H), 7.20 (m, 3H), 4.86 (dd, 1H), 4.04 (m, 1H), 3.62 (m, 0.5H), 3.44 (m, 3H), 3.24 (m, 1H), 2.12 (m, 0.5H), 1.93 (m, 1H), 1.64 (m, 2H), 1.42 (m, 1H), 1.26 (s, 9H). GC/MS (EI) for C₁₇H₂₅NO₃: 235 (M-tBu⁺).

STEP 3: To a solution of tert-butyl 4-(hydroxymethyl)-2-phenylpiperidine-1-carboxylate (0.29 g, 1.00 mmol) in dichloromethane (50 mL) was added trifluoroacetic acid (10 mL) and the reaction mixture was heated to reflux. After cooling to room temperature the solvent was evaporated. The residue was twice taken into 50% ethyl acetate in toluene then concentrated (2×100 mL) and the resulting solid then dried to give (±)-(2R,4S)-2-phenylpiperidin-4-ylmethanol as the trifluoroacetic acid salt (0.26 g, quantitative). MS (EI) for C₁₂H₁₇NO: 192 (MW).

### Reagent Preparation 3

### 2-(trifluoromethyl)piperidine

A mixture of 2-(trifluoromethyl)pyridine (0.38 g, 2.60 mmol) and platinum oxide (0.04 g, 0.18 mmol) in acetic acid (15 mL) and concentrated hydrochloric acid (2 mL) was hydrogenated in a Parr apparatus at 40 psi for 3 d. Filtration through celite and concentration of the filtrate provided 2-(trifluoromethyl)piperidine as hydrochloride salt which was used without further purification. ¹H NMR (400 MHz, methanol-d₄): 4.18 (m, 1H), 3.50 (m, 1H), 3.15 (m, 1H), 2.16 (m, 1H), 1.99 (m, 2H), 1.71 (m, 3H).

Using analogous synthetic techniques and substituting with alternative starting reagents the following reagents were prepared. Alternative starting materials were obtained commercially unless otherwise indicated.

4-cyclopropylpiperidine. Prepared as hydrochloride salt according to reagent preparation 3 by using 4-cyclopropylpyridine. MS (EI) for C₈H₁₅N: 125 (M⁺).

### Reagent Preparation 4

### tert-butyl 8-azabicyclo[3.2.1]octan-3-(endo)-ylcarbamate

STEP 1: To a 5 L round-bottom flask was added 8-methyl-8-azabicyclo[3.2.1]octan-3-endo-amine (432 g, 3.1 mol), 2 L of dry 1,4-dioxane, 675 mL of deionized water and 468 g of dry triethylamine. Di-tert-butyl dicarbonate (solution in 1.2 L of dioxane) was added dropwise to the stirring solution at room temperature over 16 h. The reaction mixture was concentrated and the resulting residue suspended in 2.5 L of methylene chloride. then washed twice with 1 L of water, dried with anhydrous magnesium sulfate, filtered, and volatile organics removed by rotary evaporation to yield 617 g (83%) of tert-butyl 8-methyl-8-azabicyclo[3.2.1]octan-3-ylcarbamate (mp 79-81° C.).

STEP 2: To a 5 L round-bottom flask was added 480 g (2.0 mol) of tert-butyl 8-methyl-8-azabicyclo[3.2.1]octan-3-endo-ylcarbamate, 2 L of toluene, and 69 g (0.5 mol) of potassium carbonate. 2,2,2-Trichloroethyl chloroformate (347 mL, 2.4 mol) was added dropwise at room temperature over 6 h and the reaction heated at reflux temperature for 8 h. After the solution was cooled to room temperature, 1.2 L of water was added to the reaction solution and stirred 0.5 h. The organic layer was separated and washed with 1 L of brine, dried with anhydrous magnesium sulfate, filtered, and concentrated to yield a cloudy oil. The oil was titruated with 700 mL of a 3:2 ethyl ether/hexanes solution to yield 280 g (mp 131-135° C.) of 2,2,2-trichloroethyl 3-endo-(tert-butoxycarbonylamino)-8-azabicyclo[3.2.1]octane-8-carboxylate as a solid that was collected by filtration. The mother liquour was concentrated and titruated further to yield a less pure sample of the Troc protected diamine (129 g, mp 116-118° C.).

STEP 3: To a 5 L round-bottom flask was added 360 g (0.9 mol) of 2,2,2-trichloroethyl 3-endo-(tert-butoxycarbonylamino)-8-azabicyclo[3.2.1]octane-8-carboxylate, 2.8 L of methanol and 675 g (12.6 mol) of ammonium chloride. The solution was heated to reflux and 387 g (7.5 mol) of zinc dust was carefully added in small portions over 0.5 h. Upon complete addition of the zinc dust, the reaction was heated at reflux temperature for 2 h then cooled to room temperature. The reaction filtered through a thin pad a Celite 545, and the methanol removed by rotary evaporation. The resulting solid was dissolved in 800 mL of methylene chloride and stirred with 600 mL of concentrated ammonium hydroxide for 0.5 h. The organic layer was separated, washed with 600 mL of water, dried with anhydrous magnesium sulfate, filtered, and concentrated to yield an oil. The residue was dissolved in 200 mL of methylene chloride and 1 L of ethyl ether then filtered. The resulting solution was chilled to 0° C. and 215 mL of 4 N hydrogen chloride in dioxane was added slowly, dropwise over 0.5 h, being sure to maintain the reaction solution temperature close to 0° C. After the addition was complete, 200 mL of methylene chloride and 1.4 L of ethyl ether were added to the cooled solution and a pale white precipitate formed. The resulting solid was collected by filtration to yield 173 g (85%) of tert-butyl 8-azabicyclo[3.2.1]octan-3-endo-ylcarbamate hydrochloride salt.

### Reagent Preparation 5

### 4-methylpiperidin-4-ol

STEP 1: To a solution of methyl magnesium bromide (6.00 mmol) in ethyl ether (27 mL) was added 1-benzyl-piperidin-4-one (0.53 g, 0.28 mmol) at 0° C. followed by tetrahydrofuran (10 mL). The reaction mixture was warmed to room temperature and stirred for 18 h. Saturated ammonium chloride was added and the aqueous layer was extracted with ethyl acetate (3×). The combined organic extracts were dried over sodium sulfate, filtered and concentrated. Column chromatography on silica (2-10% methanol in dichloromethane) afforded 1-benzyl-4-methylpiperidin-4-ol (0.42 g, 72% yield).

STEP 2: A mixture of 1-benzyl-4-methylpiperidin-4-ol (0.20 g, 0.97 mmol) and 10% palladium on carbon in methanol was hydrogenated in a Parr apparatus at 35 psi for 18 h. Then a solution of 4M hydrochloric acid in dioxane (0.1 mL) was added and the mixture was filtered through celite. The filtrate was concentrated and dried to give 4-methylpiperidin-4-ol as hydrochloride salt (0.10 g, 89% yield). ¹H NMR (400 MHz, methanol-d₄): 3.23 (m, 4H), 1.77 (m, 4H), 1.29 (s, 3H).

### Reagent Preparation 6

### 4-(difluoromethyl)piperidine

STEP 1: To a solution of tert-butyl (4-hydroxymethyl)piperidine-1-carboxylate (0.52 g, 2.40 mmol, (J. Labelled Compounds and Radiopharmaceuticals 1999, 42, 1289-1300) in dichloromethane (20 mL) was added Dess-Martin-periodinane (1.13 g, 2.66 mmol), and the mixture was stirred at room temperature for 2 h. A 10% aqueous solution of sodium thiosulfate (20 mL) was added followed by saturated sodium bicarbonate (20 mL), and the biphasic mixture was stirred at room temperature for 45 min. The layers were separated and the aqueous layer was extracted with dichloromethane (2×). The combined organic layers were washed with saturated sodium bicarbonate, brine, dried over sodium sulfate then filtered and concentrated to afford tert-butyl 4-formylpiperidine-1-carboxylate. ¹H NMR (400 MHz, CDCl₃): 9.67 (s, 1H), 3.99 (m, 2H), 2.93 (m, 2H), 2.42 (m, 1H), 1.89 (m, 2H), 1.55 (m, 2H), 1.46 (s, 9H).

STEP 2: To a solution of DAST (1.16 g, 7.20 mmol) in dichloromethane (30 mL) was added a solution of tert-butyl 4-formylpiperidine-1-carboxylate (0.51 g, 2.40 mmol) in dichloromethane (5 mL) at 0° C. The reaction mixture was warmed to room temperature and stirred for 18 h. A 5% aqueous solution of sodium bicarbonate was added, the layers were separated, the organic layer was washed with saturated sodium bicarbonate, and brine, dried over sodium sulfate, filtered and concentrated to provide tert-butyl 4-(difluoromethyl)piperidine-1-carboxylate. NMR (400 MHz, CDCl₃): 5.59 (m, 1H), 4.20 (m, 2H), 2.69 (m, 2H), 1.91 (m, 1H), 1.74 (m, 2H), 1.46 (s, 9H), 1.34 (m, 2H).

STEP 3: A solution of tert-butyl 4-(difluoromethyl)piperidine-1-carboxylate in trifluoroacetic acid was stirred at room temperature for 1 h then concentrated and dried to give 4-(difluoromethyl)piperidine as the trifluoroacetate salt. ¹H NMR (400 MHz, CDCl₃): 5.67 (m, 1H), 3.55 (m, 2H), 2.96 (m, 2H), 2.04 (m, 3H), 1.80 (m, 2H).

### Reagent Preparation 7

### 4-(fluoromethyl)piperidine

A solution of tert-butyl 4-(fluoromethyl)piperidine-1-carboxylate (J. Labelled Compounds and Radiopharmaceuticals 1999, 42, 1289-1300) in trifluoroacetic acid was stirred at room temperature for 1 h and then concentrated and dried to give 4-(fluoromethyl)-piperidine as the trifluoroacetate salt. NMR (400 MHz, CDCl₃): 4.33 (dd, 2H), 3.49 (m, 2H), 2.92 (m, 2H), 2.07 (m, 1H), 1.97 (m, 2H), 1.64 (m, 2H).

### Reagent Preparation 8

### 4-fluoro-4-methylpiperidine

STEP 1: To a solution of 1-benzyl-4-methylpiperidine-4-ol (0.16 g, 0.76 mmol) (reagent preparation 5, step 1) in dichloromethane (10 mL)was added DAST (0.37 g, 2.30 mmol) at 0° C. The reaction mixture was warmed to room temperature and stirred for 16 h. A 5% aqueous solution of sodium bicarbonate was added, the layers were separated, the organic layer was washed with saturated sodium bicarbonate, and brine, dried over sodium sulfate, filtered and concentrated to provide a mixture of 1-benzyl-4-fluoro-4-methylpiperidine and 1-benzyl-4-methyl-1,2,3,6-tetrahydropyridine. The mixture was dissolved in acetone (15 mL) and water (3 mL) then osmium tetroxide (0.25 mL of a 4% aqueous solution, 0.04 mmol) and N-methylmorpholine N-oxide (0.11 g, 0.91 mmol) were added at 0° C. The solution was kept in a freezer at −20° C. for 3 d then warmed to room temperature and 10% aqueous sodium thiosulfate was added. The biphasic mixture was stirred for 90 min at room temperature. Dichloromethane was added, the mixture was filtered through celite and the organic layer was washed with 1M hydrochloric acid, dried over sodium sulfate, filtered and concentrated to give a 1-benzyl-4-fluoro-4-methylpiperidine.

STEP 2: A suspension of 1-benzyl-4-fluoro-4-methylpiperidine as obtained in step 1 and 10% palladium on carbon in methanol was hydrogenated in a Parr apparatus at 40 psi for 18 h. The mixture was filtered through celite and the filtrate concentrated to give 4-fluoro-4-methylpiperidine which was used without further purification. MS (EI) for C₆H₁₂FN: 118 (MH⁺).

### Reagent Preparation 9

### 4-(1,1-difluoroethyl)piperidine

STEP 1: To a solution of DAST (1.83 g, 11.35 mmol) in dichloromethane (30 mL) was added 4-acetylpyridine (1.00 g, 8.25 mmol) at 0° C. The reaction mixture was warmed to room temperature and stirred for 2 d. More DAST (0.61 g, 3.78 mmol) was added and stirring was continued for 1d. A 5% aqueous solution of sodium bicarbonate was added, the layers were separated and the organic layer was washed with saturated sodium bicarbonate, and brine then dried over sodium sulfate, filtered and concentrated to provide a 5:1 mixture of 4-(1,1-difluoroethyl)pyridine and 4-acetylpyridine.

STEP 2: The mixture was dissolved in methanol (10 mL) and 1 M hydrochloric acid (10 mL) then catalytic platinum oxide was added and the resulting suspension was hydrogenated in a Parr apparatus at 40 psi for 3 d. Filtration through celite and concentration of the filtrate gave a complex mixture containing 20% of the desired 4-(1,1-difluoroethyppiperidine as the hydrochloride salt which was used without further purification.

### Reagent Preparation 10

### (3aR,6aS)-5-methyloctahydrocydopenta[c]pyrrole

STEP 1: (3aR,6aS)-tert-Butyl 5-methylenehexahydrocyclopenta[c]pyrrole-2(1H)-carboxylate (Tetrahedron 1993, 49(23), 5047-54) (107 mg, 0.48 mmol) was taken into methanol (1 mL) followed by addition of platinum oxide (10 mg) and the mixture was sparged with hydrogen gas at 1 atm for 10 minutes then allowed to stir under an atmosphere of hydrogen for 12 h. The mixture was filtered through a celite pad and the filtrate concentrated. The residue was taken into a minimum of ethyl acetate then filtered through a silica gel pad using 100% ethyl acetate. The filtrate was concentrated and dried to give (3aR,6aS)-tert-butyl 5-methyl hexahydrocyclopenta[c]pyrrole-2(1H)-carboxylate as a colorless oil, 5-methyl endo/exo isomer mixture (98.6 mg, 92% yield). GC-MS (EI) for C₁₃H₂₃NO₂: 225 (M⁺)

STEP 2: (3aR,6aS)-tert-butyl 5-methyl hexahydrocyclopenta[c]pyrrole-2(111)-carboxylate (98.6 mg, 0.44 mmol) was taken into a minimum of neat TFA and the solution was allowed to stand for 30 minutes at room temperature. The mixture was then concentrated and the residue taken into methanol and concentrated again then dried. The residue thus obtained was taken taken into methanol (5 mL) and basified using Bio-Rad AG-1× hydroxide form resin. The mixture was then filtered and concentrated and dried to give (3aR,6aS)-5-methyloctahydrocyclopenta[c]pyrrole (27.9 mg, 55%) as an amorphous residue.

### Reagent Preparation 11

### (±)-(3aR,6aS)-5-methyl-1,2,3,3a,4,6a-hexahydrocyclopenta[c]pyrrole

STEP 1: (3aR,6aS)-tert-Butyl 5-methylenehexahydrocyclopenta[c]pyrrole-2(1H)-carboxylate (Tetrahedron 1993, 49(23), 5047-54) (114 mg, 0.51 mmol) was taken into a minimum of neat TFA and the solution was allowed to stand for 30 minutes at room temperature. The mixture was then concentrated and the residue taken into methanol and concentrated again then dried. The residue thus obtained was taken taken into methanol (5 mL) and basified using Bio-Rad AG-1× hydroxide form resin. The mixture was then filtered and concentrated and dried to give impure (±)-(3aR,6aS)-5-methyl-1,2,3,3a,4,6a-hexahydrocyclopenta[c]pyrrole (93 mg) as an amorphous residue that was used without further purification.

### Reagent Preparation 12

### 4-(methylthio)piperidine

STEP 1: To a solution of tert-butyl 4-hydroxypiperidine-1-carboxylate (4.0 g, 20.0 mmol) and triethylamine (4.0 g, 40 mmol) in dichloromethane (50 mL) was added methanesulfonyl chloride (2.8 g, 24.4 mmol) at 0° C. The solution was stirred at 0° C. for 10 min, then at room temperature for 2 h. The reaction mixture was partitioned between 10% citric acid and ethyl acetate. The organic layer was washed with sodium bicarbonate, and brine, dried over sodium sulfate, filtered and concentrated to give tert-butyl 4-(methylsulfonyloxy)piperidine-1-carboxylate (6.4 g, quantitative yield). MS (EI) for C₁₁H₂₁NO₅S: 279 (M⁺).

STEP 2: A solution of tert-butyl 4-(methylsulfonyloxy)piperidine-1-carboxylate (2.0 g, 7.2 mmol) and sodium thiomethoxide (1.0 g, 14.4 mmol) in methanol (30 mL) was refluxed for 15 h and then concentrated. The residue was partitioned between water and ethyl acetate. The aqueous layer was extracted twice with ethyl acetate and the combined organic extracts washed with brine, dried over sodium sulfate, filtered and concentrated. Column chromatography on silica (3% ethyl acetate in hexanes) afforded tert-butyl 4-(methylthio)piperidine-1-carboxylate (0.98 g, 58% yield) as a colorless oil. MS (EI) for C₁₁H₂₁NO₂S: 231 (M⁺).

STEP 3: A solution of tert-butyl 4-(methylthio)piperidine-1-carboxylate (63 mg, 0.27 mmol) in methanol (1 mL) and 4 N hydrogen chloride in dioxane (4 mL) was refluxed for 2 min and then concentrated and dried to provide 4-(methylthio)piperidine hydrochloride as a colorless oil.

### Reagent Preparation 13

### thiomorpholine-1-oxide

Thiomorpholine-1-oxide was prepared according to the literature procedure given in J. Med. Chem. (1983), 26, 916-922. MS (EI) for C₄H₉NOS: 119 (M⁺).

### Reagent Preparation 14

### 4-(methylsulfonyl)piperidine

STEP 1: To a solution of tert-butyl 4-(methylthio)piperidine-1-carboxylate (280 mg, 1.2 mmol) (reagent preparation 12, step 2) in dichloromethane (8 mL) was added m-chloroperbenzoic acid (835 mg, 4.8 mmol) at 0° C. The solution was warmed to room temperature and stirred for 15 h. The reaction mixture was partitioned between 1N sodium hydroxide and ethyl acetate. The organic layer was washed with brine, dried over sodium sulfate, filtered and concentrated to give tert-butyl 4-(methylsulfonyl)piperidine-1-carboxylate (290 mg, 92% yield). MS (EI) for C₁₁H₂₁NO₄S: 206 (M-tBu⁺).

STEP 2: A solution tert-butyl 4-(methylsulfonyl)piperidine-1-carboxylate (100 mg, 0.38 mmol) in methanol (1 mL) and 4 N hydrogen chloride in dioxane (4 mL) was refluxed for 2 min and then concentrated to provide 4-(methylthio)piperidine hydrochloride salt as a colorless solid. MS (EI) for C₆H₁₃NO₂S: 163 (M+).

### Reagent Preparation 15

### 3-(trifluoromethyl)-8-azabicyclo[3.2.1]octan-3-(endo)-ol

Step 1: Trimethyl(trifluoromethyl)silane (0.32 g, 2.25 mmol) was added to a mixture of tert-butyl 3-oxo-8-azabicyclo[3.2.1]octane-8-carboxylate (0.50 g, 2.2 mmol), cesium carbonate (1.1 g, 3.4 mmol) in N,N-dimethylformamide (5 mL) at 0° C. The resulting mixture was warmed to room temperature and stirred for two hours. The mixture was diluted with ethyl acetate (80 mL), washed with water (3×50 mL) then brine (50 mL), dried over sodium sulfate, filtered, and concentrated. The residue was taken into methanol (20 mL) and potassium carbonate (0.62 g, 4.5 mmol) was added then stirred at room temperature for 18 hours. The mixture was diluted with ethyl acetate (150 mL) then filtered and concentrated. The residue was purified by silica gel chromatography (10% to 25% ethyl acetate in hexanes gradient) to give tert-butyl 3-(endo)-hydroxy-3-(trifluoromethyl)-8-azabicyclo[3.2.1]octane-8-carboxylate (0.36 g, 55% yield), GC-MS (EI) for C₁₃H₂₀F₃NO₃: 295 (MH⁺).

Step 2: tert-Butyl 3-(endo)-hydroxy-3-(trifluoromethyl)-8-azabicyclo[3.2.1]octane-8-carboxylate 1 (0.36 g, 1.2 mmol) was taken into acetonitrile (2 mL) and 4 M hydrogen chloride in 1,4-dioxane (2 mL) then stirred at 70° C. for 15 minutes. The reaction mixture was concentrated and dried to give 3-(trifluoromethyl)-8-azabicyclo[3.2.1]octan-3-(endo)-ol hydrochloride (0.28 g, 100% yield). MS (EI) for C₈H₁₂F₃NO: 196 (MH⁺).

### Reagent Preparation 16

### 3-methyl-8-azabicyclo[3.2.1]octan-3-(endo)-ol

Step 1: Methylmagnesium bromide (3 M solution in ether, 2.7 mmol) was added to a solution of tert-butyl 3-oxo-8-azabicyclo[3.2.1]octane-8-carboxylate (0.50 g, 2.2 mmol), in tetrahydrofuran (20 mL) at 0° C. and the resulting mixture was stirred one hour. The reaction mixture was quenched with saturated aqueous ammonium chloride solution (20 mL) then partitioned with ethyl acetate (80 mL). The organic portion was separated, washed with water, then brine, dried over sodium sulfate, filtered and concentrated. The residue was purified by silica gel chromatography (5% to 35% ethyl acetate in hexanes gradient) to give tert-butyl 3-(endo)-hydroxy-3-methyl-8-azabicyclo[3.2.1]octane-8-carboxylate (0.22 g, 41% yield), GC-MS (EI) for C₁₃H₂₃NO₃: 241 (M⁺).

Step 2: tert-Butyl 3-(endo)-hydroxy-3-methyl-8-azabicyclo[3.2.1]octane-8-carboxylate (0.22 g, 1.2 mmol) was taken into acetonitrile (1 mL), and 4 M hydrogen chloride in 1,4-dioxane (1 mL) then stirred at 70° C. for 15 minutes. The reaction mixture was concentrated and dried to give 3-methyl-8-azabicyclo[3.2.1]octan-3-(endo)-ol hydrochloride salt (0.16 g, 100% yield). MS (EI) for C₈K₂F₃NO: 142 (MI-r).

### Reagent Preparation 17

### 3-fluoro-3-(endo)-methyl-8-azabicyclo[3.2.1]octane

Step 1: Dimethylaminosulfur trifluoride (81 mg, 0.61 mmol) was added to a solution of tert-butyl 3-(endo)-(hydroxymethyl)-8-azabicyclo[3.2.1]octane-8-carboxylate (50 mg, 0.21 mmol) (reagent preparation 18, step 2) in dichloromethane (2 mL) at 0° C., and the resulting mixture was stirred one hour. The reaction mixture was quenched with saturated aqueous sodium bicarbonate solution (10 mL) then partitioned with dichloromethane (20 mL). The organic portion was separated, washed with water, then brine, dried over sodium sulfate, filtered and concentrated. The residue was purified by silica gel chromatography (5% to 35% ethyl acetate in hexanes gradient) to give tert-butyl 3-fluoro-3-(endo)-methyl-8-azabicyclo[3.2.1]octane-8-carboxylate (28 mg, 56% yield), GC-MS (EI) for C₁₃H₂₂FNO₂: 243 (M⁺).

Step 2: A mixture of tert-butyl 3-fluoro-3-(endo)-methyl-8-azabicyclo[3.2.1]octane-8-carboxylate (0.22 g, 1.2 mmol), acetonitrile (1 mL) and 4 M hydrogen chloride in 1,4-dioxane (1 mL) was stirred at 70° C. for 15 minutes. The reaction mixture was concentrated and dried to give 3-fluoro-3-(endo)-methyl-8-azabicyclo[3.2.1]octane hydrochloride salt (20 mg, 100% yield). MS (EI) for C₈H₁₄FN: 144 (MH⁺).

### Reagent Preparation 18

### 8-azabicyclo[3.2.1]octan-3-(endo)-ylmethanol

Step 1: Potassium tert-butoxide (0.62 g, 5.5 mmol) was added to a suspension of methyltriphenylphosphonium bromide (1.98 g, 5.5 mmol) in tetrahydrofuran (20 mL) and the resulting mixture was stirred at room temperature for one hour. A solution of tert-butyl 3-oxo-8-azabicyclo[3.2.1]octane-8-carboxylate (0.50 g, 2.2 mmol) in tetrahydrofuran (5 mL) was then added and the resulting mixture was stirred at 35° C. for two hours. The mixture was cooled, diluted with hexane (100 mL), filtered, and the filtrate was washed with water then brine, dried over sodium sulfate, filtered and concentrated. The residue was purified by silica gel chromatography (20% ethyl acetate in hexanes) to give tert-butyl 3-methylene-8-azabicyclo[3.2.1]octane-8-carboxylate (0.45 g, 91% yield). GC-MS (EI) for C₁₃H₂₁NO₂: 223 (M⁺).

Step 2: Borane (1 M solution in tetrahydrofuran, 1.79 mL) was added to a solution of tert-butyl 3-methylene-8-azabicyclo[3.2.1]octane-8-carboxylate (0.20 g, 0.87 mmol) in tetrahydrofuran (20 mL) at 0° C. The reaction mixture was slowly warmed to room temperature and stirred for 18 hours. It was then cooled to 0° C., followed by sequential addition of 2 M sodium hydroxide solution (1 mL) and hydrogen peroxide solution (30% in water, 0.46 mL). The mixture was warmed to room temperature and stirred for 1.5 hours. The reaction mixture was quenched with saturated sodium bicarbonate solution (10 mL), diluted with water (20 mL) and partitioned with ethyl acetate (20 mL). The organic portion was separated and washed twice with saturated sodium bisulfite solution (20 mL), water then brine, dried over sodium sulfate, filtered and concentrated. The residue was purified by silica gel chromatography (20% to 90% ethyl acetate hexanes gradient) to give tert-butyl 3-(endo)-(hydroxymethyl)-8-azabicyclo[3.2.1]octane-8-carboxylate (0.19 g, 88% yield), GC-MS (EI) for C₁₃H₂₃NO₃: 241 (M⁺).

Step 3: A mixture of tert-butyl 3-(endo)-(hydroxymethyl)-8-azabicyclo[3.2.1]octane-8-carboxylate (50 mg, 0.21 mmol), acetonitrile (1 mL), and 4 M hydrogen chloride in 1,4-dioxane (1 mL) was stirred at 70° C. for 15 minutes. The reaction mixture was concentrated and dried to give 8-azabicyclo[3.2.1]octan-3-(endo)-ylmethanol hydrochloride salt (36 mg, 100% yield). MS (EI) for C₈H₁₅NO: 142 (MH⁺).

### Reagent Preparation 19

### 3-(endo)-(fluoromethyl)-8-azabicyclo[3.2.1]octane

Step 1: Methanesulfonyl chloride (154 mg, 1.35 mmol) was added to a mixture of tert-butyl 3-(endo)-(hydroxymethyl)-8-azabicyclo[3.2.1]octane-8-carboxylate (325 mg, 1.4 mmol) (reagent preparation 18, step 2), triethylamine (136 mg, 1.4 mmol), and 1,4-diazabicyclo[2.2.2]octane (31 mg, 0.28 mmol) in toluene (10 mL) at 0° C. The resulting mixture was stirred at 0° C. for 15 minutes, and at room temperature for another 15 minutes. The reaction mixture was quenched with a cold mixture of water and ethyl acetate. The organic portion was separated, washed with water, then brine, dried over sodium sulfate, filtered and concentrated. The residue was purified by silica gel chromatography (5% to 25% ethyl acetate in hexanes gradient) to give tert-butyl 3-((endo-methylsulfonyloxy)methyl)-8-azabicyclo[3.2.1]octane-8-carboxylate (330 mg, 77% yield), GC-MS (EI) for C₁₄H₂₅NO₅S: 319 (M⁺).

Step 2: A mixture of tert-butyl 3-((endo)-methylsulfonyloxy)methyl)-8-azabicyclo[3.2.1]octane-8-carboxylate (330 mg, 1.0 mmol), triethylamine (136 mg, 1.4 mmol), and tetrabutylammonium fluoride hexahydrate (489 mg, 1.3 mmol) in tetrahydrofuran (10 mL) was stirred at 60° C. for 18 hours. The reaction mixture was cooled, concentrated and the residue purified by silica gel chromatography (5% to 15% ethyl acetate in hexanes gradient) to give tert-butyl 3-(endo)-(fluoromethyl)-8-azabicyclo[3.2.1]octane-8-carboxylate (120 mg, 36% yield), GC-MS (EI) for C₁₃H₂₂FNO₂: 243 (M⁺).

Step 3: A mixture of tert-butyl 3-(endo)-(fluoromethyl)-8-azabicyclo[3.2.1]octane-8-carboxylate (50 mg, 0.21 mmol), acetonitrile (1 mL), and 4 M hydrogen chloride in 1,4-dioxane (1 mL) was stirred at 70° C. for 15 minutes. The reaction mixture was concentrated and dried to give 3-(endo)-(fluoromethyl)-8-azabicyclo[3.2.1]octane hydrochloride salt (37 mg, 100% yield). MS (EI) for C₈H₁₅FN: 144 (MH⁺).

### Reagent Preparation 20

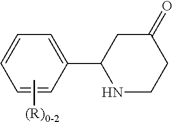

STEP 1: Benzyl 2-(4-fluorophenyl)-4-oxo-3,4-dihydropyridine-1(2H)-carboxylate was prepared according to the method in (Tetrahedron Lett., 1986, 27, 4549-4552) using 4-methoxypyridine (29.8 mL, 290 mmol), benzyl chloroformate (50.0 mL, 350 mmol) and 4-fluorophenyl magnesium bromide (0.8 M solution in THF), (450 mL, 0.36 mmol), to yield (81 g, 86% yield) of the title compound. MS (EI) for C₁₉H₁₆FNO₃: 326 (MH⁺).

STEP 2: Benzyl 2-(4-fluorophenyl)-4-oxopiperidine-1-carboxylate was prepared according to the method described in (J. Am. Chem. Soc., 2001, 66, 2181-2182) using benzyl 2-(4-fluorophenyl)-4-oxo-3,4-dihydropyridine-1(2H)-carboxylate (16.5 g, 50.7 mmol) and zinc dust (9.8 g, 150 mmol) to afford (16.0 g, 96% yield) of the title compound. ¹H NMR (400 MHz, CDCl₃): 7.39-7.32 (m, 5H), 7.21 (m, 2H), 7.00 (t, 2H), 5.82 (br s, 1H), 5.21 (dd, 2H), 4.28 (br s, 1H), 3.15 (m, 1H), 2.92 (m, 1H), 2.88 (dd, 1H), 2.54 (m, 1H), 2.37 (m, 1H). MS (EI) for C₁₉H₁₈FNO₃: 328 (MH⁺).

STEP 3: A solution of benzyl 2-(4-fluorophenyl)-4-oxopiperidine-1-carboxylate (4.75 g, 14.50 mmol) in a mixture of ethyl acetate and tetrahydrofuran (1:1, 100 mL) was hydrogenated in the presence of 10% Pd/C at atmospheric pressure over 12 h. The catalyst was filtered off and the filtrate was concentrated. The residue was dissolved in ethyl acetate (250 mL) and washed twice with saturated aqueous bicarbonate (100 mL), brine, then dried over anhydrous sodium sulfate, filtered and concentrated. The residue was dried to give 2-(4-fluorophenyl)piperidin-4-one (2.8 g, quantitative). MS (E) for C₁₁H₁₂FNO: 194 (MH⁺).

Using analogous synthetic techniques and substituting with alternative starting reagents in step 1 the following reagents were prepared. Alternative starting materials were obtained commercially unless otherwise indicated.

2-(3,4-difluorophenyl)piperidin-4-one. Synthesized according to the method of reagent preparation 20 using 3,4-difluorophenylmagnesium bromide in step 1. MS (EI) for C₁₁H₁₂F₂NO: 212 (MH⁺).

2-(3-fluorophenyl)piperidin-4-one. Synthesized according to the method of reagent preparation 20 using 3-fluorophenylmagnesium bromide in step 1. MS (EI) for C₁₁H₁₂FNO: 194 (MH⁺).

### Reagent Preparation 21

### 2-(3,4-difluorophenyl)-4-(trifluoromethyl)piperidin-4-ol

STEP 1: To a solution of benzyl 2-(3,4-difluorophenyl)-4-oxopiperidine-1-carboxylate (0.21 g, 0.60 mmol) (reagent preparation 20, step 2) in dimethylformamide (4.0 mL) at 0° C. was added cesium carbonate (0.30 g, 0.90 mmol), followed by the addition of trimethyl(trifluoromethyl)silane (0.35 mL, 2.40 mmol). The reaction mixture was stirred at room temperature for 12 hours then partitioned between ethyl acetate and water. The organic layer was separated, washed with brine, dried over anhydrous magnesium sulfate then filtered and concentrated. To a solution of the residue in methanol was added potassium carbonate (0.16 g, 1.19 mmol) and the reaction mixture was stirred at room temperature for 24 hours. The mixture was diluted with ethyl acetate and washed with 1M aqueous hydrochloric acid, brine, dried over anhydrous magnesium sulfate then filtered and concentrated to give benzyl 2-(3,4-difluorophenyl)-4-hydroxy-4-(trifluoromethyl)piperidine-1-carboxylate (0.24 g, quantitative).

STEP 2: A solution of benzyl 2-(3,4-difluorophenyl)-4-hydroxy-4-(trifluoromethyl)piperidine-1-carboxylate (0.24 g, 0.60 mmol) in methanol (100 mL) was hydrogenated in the presence of catalytic 10% palladium on carbon at atmospheric pressure for 12 h. The catalyst was filtered off and the filtrate was concentrated and dried to give 2-(3,4-difluorophenyl)-4-(trifluoromethyl)piperidin-4-ol (0.13 g, 78%). MS (EI) for C₁₂H₁₂F₅NO: 282 (MH⁺).

### Reagent Preparation 22

### 4-(2,2-difluoroethyl)piperidine

STEP 1: To a solution of tert-butyl 4-(2-hydroxyethyl)piperidine-1-carboxylate (0.6 g, 2.6 mmol) in dichloromethane (30 mL) was added Dess-Martin-periodinane (1.2 g, 2.8 mmol), and the mixture was stirred at room temperature for 90 min. A 10% aqueous solution of sodium thiosulfate (15 mL) was added followed by saturated sodium bicarbonate (15 mL), and the biphasic mixture was stirred at room temperature for 1 h. The layers were separated, the aqueous layer was extracted twice with dichloromethane. The combined organic layers were washed with saturated sodium bicarbonate, and brine, dried over sodium sulfate, filtered and concentrated to afford tert-butyl 4-(oxoethyl)piperidine-1-carboxylate that was used directly without further purification.

STEP 2: To a solution of ten-butyl 4-(oxoethyl)piperidine-1-carboxylate as obtained in step 1 in dichloromethane (50 mL) was added DAST (1.2 g, 7.8 mmol) at 0° C. The reaction mixture was warmed to room temperature and stirred for 17 h. A 5% aqueous solution of sodium bicarbonate was added and the layers were separated. The organic layer was washed with saturated sodium bicarbonate, and brine, dried over sodium sulfate, filtered and concentrated to provide ten-butyl 4-(2,2-difluoroethyl)piperidine-1-carboxylate that was used directly without further purification.

STEP 3: tert-Butyl 4-(2,2-difluoroethyl)piperidine-1-carboxylate as obtained in step 2 was dissolved in a minimum of trifluoroacetic acid and the resulting solution was stirred at room temperature for 2 h. The solution was then concentrated to give 4-(2,2-difluoroethyl)piperidine as the trifluoroacetate salt. MS (EI) for C₇H₁₃F₂N: 150 (MH⁺).

### Reagent Preparation 23

### (±)-(2R,4R)-4-methyl-2-phenylpiperidin-4-ol

### (±)-(2R,4S)-4-methyl-2-phenylpiperidin-4-ol

STEP 1: Methylmagrtesium bromide (3 M solution in ether, 1.2 mL, 3.6 mmol) was added to a solution of tert-butyl 4-oxo-2-phenylpiperidine-1-carboxylate (328 mg, 1.2 mmol), in tetrahydrofuran (20 mL) at 0° C. and the resulting mixture was stirred at this temperature one hour. The reaction mixture was then quenched with saturated aqueous ammonium chloride solution (20 mL) and diluted with ethyl acetate (80 mL). The organic portion was separated, washed with water, then brine solution, dried over sodium sulfate, filtered and concentrated. The residue purified by silica gel chromatography (25% to 70% ethyl acetate in hexane gradient) to give the first elueting isomer assigned as (±)-tert-butyl (2R,4S)-4-methyl-2-phenylpiperidin-4-ol-1-carboxylate (100 mg, 29% yield), LC-MS for C₁₇H₂₅NO₃: 292 (MH⁺); and the second elueting isomer assigned as (±)-tert-butyl (2R,4R)-4-methyl-2-phenylpiperidin-4-ol-1-carboxylate (120 mg, 35% yield), MS (EI) for C₁₇H₂₅NO₃: 292 (MH⁺).

STEP 2: (±)-tert-butyl (2R,4S)-4-methyl-2-phenylpiperidin-4-ol-1-carboxylate (37 mg, 0.13 mmol) was taken into a minimum of neat TFA and allowed to stand at room temperature for 15 minutes. The solution was concentrated and taken into ethanol (5 mL) then concentrated and the residue dried to give (2R,4S)-4-methyl-2-phenylpiperidin-4-ol trifluoroacetate salt as an amorphous residue. MS (EI) for C₁₂H₁₇NO: 192 (MH⁺).

In the same manner (±)-(2R,4R)-4-methyl-2-phenylpiperidin-4-ol trifluoroacetate salt was prepared. MS (EI) for C₁₂H₁₇NO: 192 (MH⁺).

### Reagent Preparation 24

### 4-(trifluoromethyl)piperidin-4-ol

STEP 1: To a solution of tert-butyl 4-oxopiperidine-1-carboxylate (0.6 g, 3.0 mmol) and cesium carbonate (1.1 g, 3.3 mmol) in dimethylformamide (10 mL) was added dropwise trimethyl(trifluoromethyl)silane (2 mL, 13.5 mmol) at 0° C. The resulting mixture was stirred at room temperature for 2 hours. The reaction mixture was diluted with diethyl ether (100 ml) washed with water (50 mL) and brine (50 mL). The organic layer was dried over anhydrous sodium sulfate, filtered and concentrated to afford tert-butyl 4-(trifluoromethyl)-4-(trimethylsilyloxy)piperidine-1-carboxylate (0.512 g, 50% yield) as an orange residue that was used without further purification. MS (EI) for C₁₄H₂₆F₃NO₃Si: 341

STEP 2: To a solution of tert-butyl 4-(trifluoromethyl)-4-(trimethylsilyloxy)piperidine-1-carboxylate (0.512 g, 1.50 mmol), in methanol (10 mL) was potassium carbonate (0.25 g, 1.81 mmol). The resulting mixture was stirred at room temperature for 12 hours. Filtration and concentration provided an orange residue that was purified by silica gel chromatography (97:3 dichloromethane:methanol) to give tert-butyl 4-hydroxy-4-(trifluoromethyl)piperidine-1-carboxylate (0.07 g, 14% yield). MS (EI) for C₁₁H₁₈F₃NO₃: 269 (MH⁺).

STEP 3: To a solution of tert-butyl 4-hydroxy-4-(trifluoromethyl)piperidine-1-carboxylate (0.07 g, 0.26 mmol) in dichloromethane (1 mL) was added trifluoroacetic acid (1 mL). The resulting mixture was stirred at room temperature for 30 minutes. Concentration and drying afforded 4-(trifluoromethyl)piperidin-4-ol (0.044 g, 100%). MS (EI) for C₆H₁₀F₃NO: 269 (MH⁺).

### Reagent Preparation 25

### 4-methylpiperidine-4-carbonitrile

STEP 1: Trifluoroacetic acid anhydride (75 uL, 0.82 mmol) was added to a mixture of tert-butyl 4-carbamoyl-4-methylpiperidine-1-carboxylate (100 mg, 0.41 mmol) and pyridine (118 uL, 1.6 mmol) in tetrahydrofuran (2 mL), and the resulting mixture was stirred at room temperature for one hour. The mixture was concentrated then taken into ethyl acetate (20 mL) and partitioned with 0.5 M hydrochloric acid. The organic layer was washed with water then brine, dried over sodium sulfate, filtered, and concentrated to provide a 1:1 mixture of tert-butyl 4-cyano-4-methylpiperidine-1-carboxylate and tert-butyl 4-carbamoyl-4-methylpiperidine-1-carboxylate (100 mg) that was carried forward without further purification. GC-MS (EI) for C₁₂H₂₀N₂O₂ (tert-butyl 4-cyano-4-methylpiperidine-1-carboxylate): 224 (M⁺).

STEP 2: tert-Butyl 4-cyano-4-methylpiperidine-1-carboxylate as obtained in step 1 (100 mg, 0.21 mmol), acetonitrile (1 mL), and 4 M hydrogen chloride in 1,4-dioxane (1 mL) were combined and stirred at 70° C. for 15 minutes. The reaction mixture was concentrated and dried to give 4-methylpiperidine-4-carbonitrile hydrochloride salt (56 mg) contaminated with 4-methylpiperidine-4-carboxamide hydrochloride salt. MS (EI) for C₇H₁₂N₂ (4-methylpiperidine-4-carbonitrile): 125 (MH⁺).

### Reagent Preparation 26

### 8-azabicyclo[3.2.1]octan-3-ol

STEP 1: Sodium borohydride (178 mg, 4.7 mmol) was added to a solution of tert-butyl 3-oxo-8-azabicyclo[3.2.1]octane-8-carboxylate (0.50 g, 2.2 mmol) in ethanol (10 mL), and the resulting mixture was stirred at room temperature for one hour. The mixture was quenched with saturated ammonium chloride solution (30 mL), and extracted with ethyl acetate (3×20 mL). The combined extract was washed with water then brine, dried over sodium sulfate, filtered and concentrated to give tert-butyl 3-hydroxy-8-azabicyclo[3.2.1]octane-8-carboxylate (463 mg, 92% yield) as a mixture of endo and exo stereoisomers. GC-MS (EI) for C₁₂H₂₁NO₃: 227 (M⁺).

STEP 2: tent-Butyl 3-hydroxy-8-azabicyclo[3.2.1]octane-8-carboxylate as obtained in step 1 (100 mg, 2.0 mmol), acetonitrile (2 mL) and 4 M hydrogen chloride in 1,4-dioxane (2 mL) were combined and stirred at 70° C. for 15 minutes. The reaction mixture was concentrated and dried to give 8-azabicyclo[3.2.1]octan-3-ol hydrochloride salt (71 mg, 100% yield). MS (EI) for C₇H₁₃NO: 128 (MH⁺).

### Reagent Preparation 27

### 3-(endo)-methyl-8-azabicyclo[3.2.1]octane

STEP 1: A mixture of tert-butyl 3-methylene-8-azabicyclo[3.2.1]octane-8-carboxylate (0.10 g, 0.44 mmol) (reagent preparation 18), 10% palladium on charcoal (10 mg) and ethanol (15 mL) was hydrogenated in a Parr apparatus at 40 psi for 18 hours. The mixture was filtered and concentrated then dried to give tert-butyl 3-(endo)-methyl-8-azabicyclo[3.2.1]octane-8-carboxylate (96 mg, 95% yield); GC-MS (EI) for C₁₃H₂₃NO₂: 225 (M⁺).

STEP 2: A mixture of tert-butyl 3-(endo)-methyl-8-azabicyclo[3.2.1]octane-8-carboxylate (96 mg, 0.43 mmol), acetonitrile (1 mL), and 4 M hydrogen chloride in 1,4-dioxane (1 mL) was stirred at 70° C. for 15 minutes. The reaction mixture was concentrated and dried to give 3-(endo)-methyl-8-azabicyclo[3.2.1]octane hydrochloride salt (68 mg, 100% yield). MS (EI) for C₈H₁₅N: 126 (MH⁺).

### Reagent Preparation 28

### (±)-(2R,4S)-2-(3,4-difluorophenyl)piperidin-4-ol

STEP 1: A solution of benzyl 2-(3,4-difluorophenyl)-4-oxo-3,4-dihydropyridine-1(2H)-carboxylate (6.70 g, 19.50 mmol) (reagent preparation 20) in methanol (100 mL) was hydrogenated with catalytic 10% palladium on carbon in a Parr shaker at 35 psi. The catalyst was filtered off and the filtrate was concentrated then dried to give (±)-(2R,4S)-2-(3,4-difluorophenyl)piperidin-4-ol (4.2 g, quantitative). ¹H NMR (400 MHz, d₆-DMSO): 7.33 (m, 1H), 7.28 (m, 1H), 7.02 (m, 1H), 5.00 (t, 1H), 4.49 (d, 1H), 3.91 (m, 1H), 3.77 (m, 1H), 3.21 (m, 1H), 2.11 (2t, 1H), 1.95 (2q, 1H), 1.70 (m, 1H), 1.50 (m, 1H). MS (EI) for C₁₁H₁₃F₂NO: 214 (MH⁺).

Using analogous synthetic techniques and substituting with alternative starting reagents in step 1 the following reagents were prepared. Alternative starting materials were obtained commercially unless otherwise indicated.

(±)-(2R,4S)-2-(4-fluorophenyl)piperidin-4-ol. Synthesized according to the method of reagent preparation 28 starting with benzyl 6-(4-fluorophenyl)-4-oxo-3,4-dihydropyridine-1(2H)-carboxylate (reagent preparation 20). MS (EI) for C₁₁H₁₄FNO: 194 (M⁻).

### Reagent Preparation 29

### 4,4-difluoro-2-phenylpiperidine

STEP 1: To a solution of tert-butyl 4-oxo-2-phenylpiperidine-1-carboxylate (0.20 g, 0.73 mmol), in dichloromethane (50 mL) at 0° C. was slowly added bis(2-methoxyethyl) aminosulfur trifluoride (0.16 mL, 0.87 mmol) and the reaction mixture was allowed to warm to room temperature. The mixture was stirred for 12 hours, then quenched by the addition of saturated aqueous ammonium chloride and partitioned with ethyl acetate. The organic layer was separated, washed with brine, dried over anhydrous magnesium sulfate, filtered and concentrated. Silica gel chromatography of the residue (hexanes:ethyl acetate 4:1) provided tert-butyl 4,4-difluoro-2-phenylpiperidine-1-carboxylate (0.17 g, 81%). GC-MS (EI) for C₁₆H₂₁F₂NO₂: 241 (M-tBe).

STEP 2: To a solution of tert-butyl 4,4-difluoro-2-phenylpiperidine-1-carboxylate (0.17 g, 0.57 mmol) in methanol (5 mL) was added 4M hydrogen chloride in dioxane (5 mL). The reaction mixture was stirred at room temperature for 4 hours then concentrated and the residue was triturated with diethyl ether. The white solid was collected by filtration and dried to give 4,4-difluoro-2-phenylpiperidine as the hydrochloride salt salt (93 mg, 70%). GC-MS (EI) for C₁₁H₁₃F₂N: 197 (MH⁺).

### Reagent Preparation 30

### 1,3-diphenylpiperizine

STEP 1: A solution of tert-butyl 3-phenylpiperazine-1-carboxylate (0.95 g, 3.6 mmol), benzyl chloroformate (0.85 g, 5.0 mmol) and diisopropylethylamine (1.0 g, 7.7 mmol) in dioxane (20 mL) was heated to 95° C. for 3 hours. After cooling, the reaction mixture was diluted with ethyl acetate (100 mL), and washed with saturated aqueous sodium bicarbonate (50 mL) and brine (25 mL). After drying over anhydrous sodium sulfate, filtration and concentration, the residue was purified by silica gel column chromatography (ethyl acetate/hexanes, 1:8) to give 1-benzyl 4-tert-butyl 2-phenylpiperazine-1,4-dicarboxylate (0.84 g, 59% yield).

STEP 2: To 1-benzyl 4-tert-butyl 2-phenylpiperazine-1,4-dicarboxylate (0.84 g, 2.12 mmol) in dichloromethane (5.0 mL) added drop wise trifluoroacetic acid (5.0 mL) and maintained at 25° C. for 90 minutes. The reaction mixture was concentrated, and the residue dissolved in ethyl acetate (60 mL). The solution was washed with saturated aqueous sodium carbonate (30 mL) and brine (20 mL), and then dried over anhydrous sodium sulfate, filtered and concentrated to yield benzyl 2-phenylpiperazine-1-carboxylate (0.59 g, 94% yield). MS (EI) for C₁₈H₂₀N₂O₂: 297 (MH⁺).

STEP 3: A solution of benzyl 2-phenylpiperazine-1-carboxylate (0.17 g, 0.58 mmol), bromobenzene (0.37 g, 2.37 mmol), tris(dibenzylideneacetone)dipalladium(0) (0.06 g, 0.06 mmol), sodium tert-butoxide (0.20 g, 2.0 mmol) and 2-dicyclohexylphosphino-7-(NN-dimethylamino)biphenyl (0.25 g, 0.64 mmol) in benzene (20 mL) was heated to 80° C. for 4.5 hours. After cooling, the reaction was diluted with ethyl acetate (60 mL), and washed with water (2×30 mL), then dried over anhydrous sodium sulfate, filtered and concentrated. The residue was purified by silica gel column chromatography (ethyl acetate/hexanes, 1:4) to give benzyl 2,4-diphenylpiperazine-1-carboxylate (0.17 g, 79% yield) as an oil. MS (EI) for C₂₄H₂₄N₂O₂: 373 (MH⁺).

STEP 4: A solution of benzyl 2,4-diphenylpiperazine-1-carboxylate (0.17 g, 0.45 mmol) and 5% Pd on carbon (0.1 g) in tetrahydrofuran/methanol 5:1 (10 mL) was stirred under hydrogen (1 atm) for 4.5 hours. The reaction was filtered through celite and concentrated to give the title compound 1,3-diphenylpiperizine (0.10 g, 93% yield) as an oil. MS (EI) for C₁₆H₁₈N₂: 239 (MH⁺).

### Reagent Preparation 31

### (±)-(2R,4R)-2-(4-fluorophenyl)piperidin-4-ol

STEP 1: A mixture of benzyl 2-(4-fluorophenyl)-4-oxo-3,4-dihydropyridine-1(2H)-carboxylate (1.00 g, 3.07 mmol) (reagent preparation 20) and 5% Pd on carbon (0.1 g) in acetic acid:methanol 1:10 (20 mL) was hydrogenated at 45 psi using a Parr apparatus for 16 hours. The catalyst was removed by filtering through Celite, and the filtrate concentrated to give (±)-(2S,4R)-2-(4-fluorophenyl)piperidin-4-ol as an oil. The material was taken into chloroform (100 mL) and di-tert-butyl dicarbonate (0.74 g, 3.4 mmol) was added, followed by the dropwise addition of diisopropylethylamine (1.5 g, 12 mmol). The reaction was warmed to reflux for 10 minutes, then allowed to cool to 25° C. over 30 minutes. The organic solution was washed with 0.1 M aqueous hydrochloric acid (45 mL), water (50 mL) and saturated sodium bicarbonate (50 mL), then dried over anhydrous sodium sulfate, filtered and concentrated. The residue was purified by silica gel column chromatography (ethyl acetate:hexanes, 1:1) to give (±)-(2S,4R)-tert-butyl 2-(4-fluorophenyl)-4-hydroxypiperidine-1-carboxylate (0.59 g, 65% yield). ¹H NMR (400 MHz, d₆-DMSO): 7.25 (m, 2H), 7.10 (m, 2H), 4.96 (t, 1H), 4.46 (d, 1H), 3.90 (m, 1H), 3.77 (m, 1H), 3.23 (dt, 1H), 2.06 (m, 1H), 1.95 (m, 1H) 1.73 (m, 1H), 1.45 (m, 1H), 1.29 (s, 9H).

STEP 2: To (±)-(2S,4R)-tert-butyl 2-(4-fluorophenyl)-4-hydroxypiperidine-1-carboxylate (0.55 g, 1.90 mmol) in tetrahydrofuran (20 mL) was added methanesulfonyl chloride (0.158 mL, 2.05 mmol), followed by dropwise addition of diisopropylethylamine (0.50 g, 3.9 mmol) and N,N-dimethylpyridin-4-amine (10 mg). After 30 minutes the reaction was diluted with ethyl acetate (50 mL) and washed with 0.1 M hydrochloric acid (25 mL) then saturated sodium bicarbonate (50 mL), dried over anhydrous sodium sulfate, filtered and concentrated. The residue was purified by silica gel chromatography (ethyl acetate:hexanes, 1:4) to give (±)-(2S,4R)-tert-butyl 2-(4-fluorophenyl)-4-(methylsulfonyloxy)piperidine-1-carboxylate (0.62 g, 88% yield). ¹H NMR (400 MHz, CDCl₃): 7.19 (dd, 1H), 7.05 (t, 2H), 5.38 (d, 1H), 5.14 (m, 1H), 4.14 (m, 1H), 3.25 (m, 1H), 2.68 (m, 1H), 2.59 (s, 3H), 2.219M, 1H), 1.93 (m, 2H), 1.42 (s, 9H).

STEP 3: A solution of (±)-(2S,4R)-tert-butyl 2-(4-fluorophenyl)-4-(methylsulfonyloxy)piperidine-1-carboxylate (0.30 g, 0.80 mmol) and sodium acetate (0.33 g, 4.0 mmol) in dimethylsulfoxide (15 mL) was heated to 90° C. for 2.5 hours. After cooling, the reaction mixture was diluted with ethyl acetate (40 mL), and washed with water (25 mL) and brine (25 mL). The organic layer was dried over anhydrous sodium sulfate then filtered and concetrated. The residue was purified by silica gel column chromatography (ethyl acetate:hexanes 1:10) to give (±)-(2R,4R)-tert-butyl 4-acetoxy-2-(4-fluorophenyl)piperidine-1-carboxylate (150 mg, 49% yield). ¹H NMR (400 MHz, d₆-DMSO): 7.24 (m, 4H), 5.14 (br s, 1H), 4.63 (m, 1H), 4.00 (br d, 1H), 2.72 (m, 1H), 2.56 (br d, 1H), 1.88 (s, 3H), 1.84 (br d 1H), 1.78 (m, 1H), 1.44 (m, 1H), 1.39 (s, 9H).

STEP 4: A suspention of (±)-(2R,4R)-tert-butyl 4-acetoxy-2-(4-fluorophenyl)piperidine-1-carboxylate (150 mg, 0.40 mmol) and potassium carbonate (1.0 g) in methanol:water 10:1 (11 mL) was stirred for 1 hour then diluted with ethyl acetate (40 mL) and washed with water (25 mL) and brine (25 mL). The organic layer was dried over anhydrous sodium sulfate, filtered and concetrated to give (±)-(2R,4R)-tert-butyl 2-(4-fluorophenyl)-4-hydroxypiperidine-1-carboxylate (117 mg, 99% yield). NMR (400 MHz, d₆-DMSO): 7.17 (m, 4H), 5.34 (br d, 1H), 4.73 (d, 1H), 4.34 (br d, 1H), 3.41 (m, 1H), 2.67 (m, 1H), 2.42 (br d, 1H), 1.57 (m, 1H), 1.38 (s, 9H).

STEP 5: To (±)-(2R,4R)-tert-butyl 2-(4-fluorophenyl)-4-hydroxypiperidine-1-carboxylate (0.10 g, 0.34 mmol) in dichloromethane (10 mL) added trifluoroacetic acid: dichloromethane 1:4 (5 mL) and the mixture was stirred at 25° C. for 30 minutes. The solution was concentrated and dried to give title compound (±)-(2R,4R)-2-(4-fluorophenyl)piperidin-4-01 (105 mg, 99% yield) as the trifluoracetic acid salt. ¹HNMR (400 MHz, d₆-DMSO): 7.56 (m, 2H), 7.31 (m, 2H), 4.53 (t, 1H), 4.12 (br s, 1H), 3.32 (q, 1H), 3.20 (d, 1H), 2.10 (t, 1H), 1.85 (br d, 2H), 1.79 (dd, 1H). MS (EI) for C₁₁H₁₄FNO: 196 (MH⁺).

### Reagent Preparation 32

### 3-(endo)-(hydroxymethyl)-8-azabicyclo[3.2.1]octan-3-ol

STEP 1: To a solution of tert-butyl 3-methylene-8-azabicyclo[3.2.1]octane-carboxylate (0.9 g, 4.0 mmol) (reagent preparation 18, step 1) in acetone (16 mL) and water (4 mL) was added osmium tetroxide (0.25 mL of a 4% aqueous solution, 0.04 mmol) and N-methylmorpholine N-oxide (1.4 g, 12.0 mmol). The reaction mixture was stirred at room temperature for 15 h, concentrated, and the residue was partitioned between 20% citric acid and ethyl acetate. The organic layer was washed twice with brine, dried over sodium sulfate, filtered and concentrated to give tert-butyl 3-(hydroxy)-3-(endo)-(hydroxymethyl)-8-azabicyclo[3.2.1]octane-carboxylate (1.0 g, quantitative yield). MS (EI) for C₁₃H₂₃NO₄: 257 (M⁺).

STEP 2: A solution of tert-butyl 3-(hydroxy)-3-(endo)-(hydroxymethyl)-8-azabicyclo[3.2.1]octane-carboxylate (50 mg, 0.20 mmol) in dichloromethane (1 mL) and trifluoroacetic acid (1 mL) was stirred at room temperature for 1 h and then concentrated and dried to give 3-(endo)-(hydroxymethyl)-8-azabicyclo[3.2.1]octan-3-ol as the trifluoroacetate salt, which was used without further purification.

### Reagent Preparation 33

### (±)-(2R,4S)-2-(3,4-difluorophenyl)-4-(fluoromethyl)piperidine

STEP 1: Potassium tert-butoxide (0.81 g, 7.2 mmol) was added to a suspension of methyltriphenylphosphonium bromide (2.58 g, 7.2 mmol) in tetrahydrofuran (20 mL) and the resulting mixture was stirred at room temperature for one hour. A solution of phenylmethyl 2-(3,4-difluorophenyl)-4-oxopiperidine-1-carboxylate (1.00 g, 2.9 mmol) (reagent preparation 20) in tetrahydrofuran (5 mL) was added and the resulting mixture was stirred at 35° C. for two hours. The mixture was cooled, diluted with hexane (100 mL), filtered, and the filtrate washed with water then brine, dried over sodium sulfate, filtered and concentrated. The residue was purified by silica gel chromatography (20% ethyl acetate in hexanes) to give phenylmethyl 2-(3,4-difluorophenyl)-4-methylidenepiperidine-1-carboxylate (0.79 g, 79% yield), MS (EI) for C₂₀H₁₉F₂NO₂: 344 (MH⁺).

STEP 2: A solution of borane (1 M in tetrahydrofuran, 4.58 mL) was added to a solution of phenylmethyl 2-(3,4-difluorophenyl)-4-methylidenepiperidine-1-carboxylate (0.79 g, 2.3 mmol) in tetrahydrofuran (20 mL) at 0° C. The reaction mixture was slowly warmed to room temperature and stirred for 18 hours. The mixture was then cooled to 0° C., and 2M sodium hydroxide solution (2.6 mL, 5.2 mmol) then hydrogen peroxide solution (30% in water, 1.2 mL) were added sequentially. The mixture was warmed to room temperature and stirred for 1.5 hours. The reaction mixture was quenched with saturated sodium bicarbonate solution (10 mL), diluted with water (20 mL), and partitioned with ethyl acetate (20 mL). The organic portion was separated and washed twice with saturated sodium bisulfite solution (20 mL), water, then brine, dried over sodium sulfate, filtered and concentrated. The residue was purified by silica gel chromatography (20% to 90% ethyl acetate in hexanes gradient) to give (±)-phenylmethyl (2R,4S)-2-(3,4-difluorophenyl)-4-(hydroxymethyl)piperidine-1-carboxylate (0.57 g, 69% yield), MS (EI) for C₂₀H₂₁F₂NO₃: 362 (MH⁺).

STEP 3: Methanesulfonyl chloride (74 mg, 0.65 mmol) was added to a mixture of (±)-phenylmethyl (2R,4S)-2-(3,4-difluorophenyl)-4-(hydroxymethyl)piperidine-1-carboxylate (233 mg, 0.64 mmol), triethylamine (233 mg, 1.7 mmol), and 1,4-diazabicyclo[2.2.2]octane (15 mg, 0.13 mmol) in toluene (10 mL) at 0° C., and the resulting mixture was stirred at 0° C. for 15 minutes, and at room temperature for another 15 minutes. The reaction mixture was then quenched with a cold mixture of water and ethyl acetate. The organic portion was separated, washed with water, then brine, dried over sodium sulfate, filtered and concentrated. The residue was purified by silica gel chromatography (5% to 25% ethyl acetate in hexanes gradient) to give (±)-phenylmethyl (2R,4S)-2-(3,4-difluorophenyl)-4-{[(methylsulfonyl)oxy]methyl}piperidine-1-carboxylate (271 mg, 96% yield). MS (EI) for C₂₁H₂₃F₂NO₅S: 440 (MH⁺).

STEP 4: A mixture of (±)-phenylmethyl (2R,4S)-2-(3,4-difluorophenyl)-4-{[(methylsulfonyl)oxy]methyl}piperidine-1-carboxylate (200 mg, 0.46 mmol), and cesium fluoride (190 mg, 1.3 mmol) in dimethyl sulfoxide (2 mL) was stirred at 100° C. for 18 hours. The reaction mixture was cooled and purified directly by silica gel chromatography (5% to 25% ethyl acetate in hexanes gradient) to give (±)-phenylmethyl (2R,4S)-2-(3,4-difluorophenyl)-4-(fluoromethyl)piperidine-1-carboxylate (85 mg, 51% yield). MS (EI) for C₂₀H₂₀F₃NO₂: 364 (MH⁺).

STEP 5: A mixture of (t)-phenylmethyl (2R,4S)-2-(3,4-difluorophenyl)-4-(fluoromethyl)piperidine-1-carboxylate (85 mg, 0.23 mmol), 10% palladium on carbon (85 mg) and ethyl acetate (5 mL) in a 100 mL flask was stirred under 1 atmosphere of hydrogen at room temperature for three days. The mixture was filtered and the filtrate concentrated and dried to give (±)-(2R,4S)-2-(3,4-difluorophenyl)-4-(fluoromethyl)piperidine (39 mg, 73% yield), MS (EI) for C₁₂H₁₄F₃N: 230 (M1-1⁺).

### Reagent Preparation 34

### (±)-(2R,4R)-2-(3,4-difluorophenyl)piperidine-4-carbonitrile

Step 1: Methanesulfonyl chloride (1.0 g, 3.2 mmol) was added to a mixture of (±)-1,1-dimethylethyl (2R,4S)-2-(3,4-difluorophenyl)-4-hydroxypiperidine-1-carboxylate (1.00 g, 3.0 mmol) (obtained by conducting reagent preparation 28 in the presence of di-tert-butyl dicarbonate) and triethylamine (0.70 g, 7.0 mmol), in tetrahydrofuran (25 mL) at 0° C., and the resulting mixture was at room temperature for two hours. The reaction mixture was then quenched with a cold mixture of water and ethyl acetate. The organic portion was separated, washed with 5% sodium hydroxide, 0.5M hydrochloric acid, water then brine, dried over sodium sulfate, filtered and concentrated. The residue was purified by silica gel chromatography (5% to 75% ethyl acetate in hexanes gradient) to give (±)-1,1-dimethylethyl (2R,4S)-2-(3,4-difluorophenyl)-4-[(methylsulfonyl)oxy]piperidine-1-carboxylate (1.2 g, 88% yield). MS (EI) for C₁₇H₂₃F₂NO₅S: 392 (MH⁺).

STEP 2: A mixture of (±)-1,1-dimethylethyl (2R,4S)-2-(3,4-difluorophenyl)-4-[(methylsulfonyl)oxy]piperidine-1-carboxylate (0.72 g, 1.8 mmol), and potassium cyanide (0.33 g, 3.7 mmol) in N,N-dimethylformamide (3.3 mL) was stirred at 90° C. for 18 hours. The reaction mixture was cooled, diluted with ethyl acetate (50 mL), washed twice with 5% lithium chloride solution (30 mL), then brine, dried over sodium sulfate, filtered and concentrated. The residue was purified by silica gel chromatography (5% to 25% ethyl acetate in hexanes gradient) to give (±)-1,1-dimethylethyl (2R,4R)-4-cyano-2-(3,4-difluorophenyl)piperidine-1-carboxylate (165 mg, 30% yield). MS (EI) for C₁₇H₂₃F₂N₂O₂: 323 (MH⁺).

STEP 3: A mixture of (±)-1,1-dimethylethyl (2R,4R)-4-cyano-2-(3,4-difluorophenyl)piperidine-1-carboxylate (65 mg, 0.20 mmol), acetonitrile (2 mL), and 4 M hydrogen chloride in 1,4-dioxane (2 mL) was stirred at 70° C. for 15 minutes. The reaction mixture was concentrated and dried to give (±)-(2R,4R)-2-(3,4-difluorophenyl)piperidine-4-carbonitrile hydrochloride salt (50 mg, 96% yield); MS (EI) for C₁₂H₁₂F₂N₂: 223 (MH⁺).

### Reagent Preparation 35

### tert-butyl 6-bromo-2-(tert-butoxycarbonyl(methoxycarbonypamino)-1H-benzo[d]imidazole-1-carboxylate

STEP 1: To a slurry of 4-bromobenzene-1,2-diamine (2.1 g, 11 mmol), 1,2-dimethoxyethane (20 mL) and methanol (5 mL) was added 1,3-bis(methoxycarbonyl)-2-methyl-2-thiopseudourea (4.0 g, 19 mmol). The reaction mixture was heated (105° C.) for 12 h and then diluted with ethyl ether (100 mL). The resulting precipitate was collected by filtration and rinsed with ethyl ether (2×25 mL) to provide methyl 6-bromo-1H-benzo[d]imidazol-2-ylcarbamate (2.3 g, 77% yield). MS (EI) for C₉H₈BrN₃O₂: 271 (MH⁺).

STEP 2: To a cooled (0° C.) slurry of 6-bromo-1H-benzo[d]imidazol-2-ylcarbamate (2.3 g, 8.5 mmol), di-tert-butyl dicarbonate (4.5 g, 20 mmol), DIPEA (5.9 mL, 34 mmol) and chloroform (30 mL) was added DMAP (0.36 g, 2.9 mmol). The reaction mixture was stirred for 2 h at ambient temperature and then partitioned between dichloromethane (50 mL) and saturated aqueous ammonium chloride (50 mL). The organic layer was then washed with brine (25 mL), dried over anhydrous magnesium sulfate, filtered and concentrated. Column chromatography on silica (10-25% ethyl acetate in hexanes) provided tert-butyl 6-bromo-2-(tert-butoxycarbonyl(methoxycarbonyl)amino)-1H-benzo[d]imidazole-1-carboxylate (2.3 g, 58% yield) as a red-brown solid. MS (EI) for C₁₉H₂₄BrN₃O₆: 471 (MIT^{I}).

### Reagent Preparation 36

### 3-(4-bromophenyl)-1-(tetrahydro-2H-pyran-2-yl)-1H-pyrazole

STEP 1: To a heated (80° C.) solution of 3-(4-bromophenyl)-1H-pyrazole (1.0 g, 4.5 mmol) and trifluoroacetic acid (0.02 mL, 0.23 mmol) in toluene (5 mL) was added 3,4-dihydro-2H-pyran (0.43 mL, 4.7 mol) over 1 hour. The reaction mixture was stirred for an additional hour and was then concentrated and dried to provide 3-(4-bromophenyl)-1-(tetrahydro-2H-pyran-2-yl)-1H-pyrazole (1.3 g, 94% yield). MS (EI) for C₁₄H₁₅BrN₂O: 308 (MH⁺).

### Reagent Preparation 37

### 4-(fluoromethyl)-4-hydroxypiperidine-1-carbonyl chloride

STEP 1: To a solution of tert-butyl 4-hydroxy-4-(hydroxymethyl)piperidine-1-carboxylate (Bioorganic & Medicinal Chemistry Letters 2008, 18(21), 5804-5808) (400 mg, 1.73 mmol) and DIPEA (1.2 mL, 7.0 mmol) in THF (10 mL) cooled to 0° C. was added thionyl chloride (0.65 mL, 8.6 mmol) in a dropwise manner and the mixture was stirred at this temperature for 1 h. The mixture was then parationed with saturated aqueous sodium bicarbonate and ethyl acetate. The organic phase was extracted with ethyl acetate (3×) and the combined organic layers were washed with brine then dried over anhydrous sodium sulfate, filtered and concentrated to afford 1,1-dimethylethyl 1,3-dioxa-2-thia-8-azaspiro[4.5]decane-8-carboxylate 2-oxide (562 mg) as an amber oil that was used without further purification. GC-MS (EI) for C₁₁H₁₉NO₅S: 277 (M⁺).

STEP 2: 1,1-dimethylethyl 1,3-dioxa-2-thia-8-azaspiro[4.5]decane-8-carboxylate 2-oxide as obtained in step 1 (555 mg) was taken into acetonitrile (20 mL) followed by addition of sodium periodate (642 mg, 3.0 mmol), water (5 mL), and ruthenium (III) chloride hydrate (5 mg) and the mixture was stirred for 3 h at room temperature. The mixture was then concentrated and the residue partitioned with ethyl acetate and water. The organic phase was washed with water (2×) and brine followed by drying over anhydrous sodium sulfate, filtration and concentration. The residue was purified by silica gel chromatography (30% ethyl acetate in hexanes) to give 1,1-dimethylethyl 1,3-dioxa-2-thia-8-azaspiro[4.5]decane-8-carboxylate 2,2-dioxide (500 mg, 98% yield) as a yellow crystalline solid. ¹H NMR (400 mHz, CDCl₃): 4.44 (s, 2H), 4.03 (br, 2H), 3.16 (br tr, 2H), 2.21 (d, 2H), 1.76 (m, 2H), 1.46 (s, 9H).

STEP 3: 1,1-dimethylethyl 1,3-dioxa-2-thia-8-azaspiro[4.5]decane-8-carboxylate 2,2-dioxide (500 mg, 1.7 mmol) was taken into THF (5 mL) followed by addition of TBAF (1M in THF, 1.8 mL) and the resulting solution was stirred for 3 h at 40° C. The mixture was then cooled and partitioned with ethyl acetate and 20% aqueous citric acid. The organic solution was washed with brine then dried over anhydrous sodium sulfate, filtered and concentrated to afford tert-butyl 4-(fluoromethyl)-4-hydroxypiperidine-1-carboxylate (350 mg, 88% yield). GC-MS (EI) for C₁₁H₂₀FNO₃: 233 (M⁺). BOC-group deprotection was carried out in a manner well established in the literature (see, Greene and Wuts, Protective Groups in Organic Synthesis, Wiley-Interscience) to give 4-(fluoromethyl)piperidin-4-ol hydrochloride salt as a colorless solid.

STEP 4: 4-(Fluoromethyl)piperidin-4-ol hydrochloride (233 mg, 1.37 mmol) was suspended in dichloromethane (3 mL) followed by addition of DIPEA (0.6 mL, 3.4 mmol) and the slurry obtained added in portions over several minutes to a solution of phosgene (20W % in toluene, 0.75 mL) diluted into dichloromethane (5 mL) and the mixture was allowed to stir at this temperature for 15 minutes. The mixture was then concentrated and the residue partitioned with ethyl acetate and water. The organic solution was washed 0.5M hydrochloric acid, brine then dried over anhydrous sodium sulfate, filtered and concentrated. The residue was purified by silica gel chromatography (3:1 ethyl ether:hexanes) to give 4-(fluoromethyl)-4-hydroxypiperidine-1-carbonyl chloride (100 mg, 37% yield) as a colorless amorphous residue. GC-MS (EI) for C₇H₁₁FNO₂Cl: 196 (M⁺).

Using analogous synthetic techniques and substituting with alternative starting materials in step 4 the following reagents were prepared. Alternative starting materials were purchased from commercial sources unless otherwise indicated.

4-methylpiperidine-1-carbonyl chloride. Synthesized according to the method of reagent preparation 37 by using 4-methylpiperidine in step 4. ¹H NMR (400 MHz, CDCl₃): 4.28, (d, 1H), 2.95 (dt, 2H), 1.75 to 1.56 (m, 3H), 1.27 to 1.10 (m, 2H), 0.97 (d, 3H), GC-MS for C₇H₁₂ClNO: 161 (M⁺).

4-cyanopiperidine-1-carbonyl chloride. Synthesized according to the method of reagent preparation 37 by using piperidine-4-carbonitrile in step 4. GC-MS for C₇H₉ClN₂O: 172 (M⁺).

4-(trifluoromethyl)piperidine-1-carbonyl chloride. Synthesized according to reagent preparation 37 by using 4-(trifluoromethyl)piperidine in step 4. GC-MS (EI) for C₇H₉ClF₃NO: 215 (M⁺).

4-(1,1-difluoroethyl)piperidine-1-carbonyl chloride. Synthesized according to reagent preparation 37 by using 4-(1,1-difluoroethyl)piperidine (reagent preparation 9) in step 4. GC-MS (EI) for C₈H₁₂ClF₂NO: 211 (M⁺).

4-(2-fluoroethyl)piperidine-1-carbonyl chloride. Synthesized according to reagent preparation 37 by using 4-(2-fluoroethyl)piperidine (WO 9746553) in step 4. GC-MS (EI) for C₈H₁₃ClFNO: 193 (M⁺).

3-(endo)-hydroxy-3-(trifluoromethyl)-8-azabicyclo[3.2.1]octane-8-carbonyl chloride. Synthesized according to the method of reagent preparation 37 using 3-(trifluoromethyl)-8-azabicyclo[3.2.1]octan-3-ol hydrochloride salt (reagent preparation 15) in step 4. GC-MS (EI) for C₉H₁₁ClF₃NO₂: 257 (M⁺)

2-(4-fluorophenyl)piperidine-1-carbonyl chloride. Synthesized according to the method of reagent preparation 37 using 2-(4-fluorophenyl)piperidine in step 4. GC-MS (EI) for C₁₂H₁₃ClFNO: 241 (M⁺).

2-(3-fluorophenyl)-4-oxopiperidine-1-carbonyl chloride. Synthesized according to the method of reagent preparation 37 using 2-(3-fluorophenyl)piperidin-4-one (reagent preparation 20) in step 4. ¹H NMR (400 MHz, CDCl₃): 7.37 (dd, 1H), 7.07 (d, 1H), 7.02 (t, 1H), 5.98 (br s, 1H), 4.40 (m, 1H), 3.36 (br d, 1H), 3.04 (t, 1H), 2.98 (dd, 1H), 2.64 (m, 1H), 2.46 (br d, 1H). GC-MS (EI) for C₁₂H₁₁ClFNO₂: 255 (M⁺).

2-(4-fluorophenyl)-4-oxopiperidine-1-carbonyl chloride. Synthesized according to the method of reagent preparation 37 using 2-(4-fluorophenyl)piperidin-4-one (reagent preparation 20) in step 4. GC-MS (EI) for C₁₂H₁₁ClFNO₂: 255 (M⁺).

2-(3,4-difluorophenyl)-4-oxopiperidine-1-carbonyl chloride. Synthesized according to the method of reagent preparation 37 using 2-(3,4-fluorophenyl)piperidin-4-one (reagent preparation 20) in step 4. ¹H NMR (400 MHz, CDCl₃): 7.18 (dd, 1H), 7.13 (m, 1H), 7.02 (m, 1H), 5.94 (br s, 1H), 4.42 (m, 1H), 3.33 (br d, 1H), 2.98 (m, 2H), 2.65 (m, 1H), 2.46 (br d, 1H). GC/MS (EI) for C₁₂H₁₁ClFNO₂: 255 (M⁺). GC-MS (EI) for C₁₂H₁₀ClP₂NO₂: 273 (M⁴).

4-(fluoromethyl)piperidine-1-carbonyl chloride. Synthesized according to the method of reagent preparation 37 using 4-(fluoromethyl)piperidine (reagent preparation 7) in step 4. GC-MS (EI) for C₇H₁₁ClFNO: 180 (M⁺).

### Reagent Preparation 38

### 6-bromo-N-ethyl-3-(methoxymethyl)-3H-imidazo[4,5-b]pyridin-2-amine

### 6-bromo-N-ethyl-N,3-bis(methoxymethyl)-3H-imidazo[4,5-b]pyridin-2-amine

Step 1: To a cooled (0° C.) solution of 5-bromopyridine-2,3-diamine (5.0 g, 27 mmol) in NMP (20 mL) was added isothiocyanatoethane (2.3 mL, 26 mmol). The resulting solution was heated (65° C.) for four hours and then cooled to ambient temperature before 1,3-diisopropylcarbodiimide (4.2 mL, 27 mmol) was added. The reaction mixture was stirred for 18 hours, diluted with water and the resulting suspension was collected by filtration. Trituration with ethyl acetate provided 6-bromo-N-ethyl-3H-imidazo[4,5-b]pyridin-2-amine (4.8 g, 75% yield) as a brown solid. NMR (400 MHz, d₆-DMSO) δ 11.41 (bs, 1H), 7.91 (s, 1H), 7.53 (s, 1H), 7.17 (s, 1H), 3.33 (q, 2H), 1.17 (t, 3H); MS (ES) for C₈H₉BrN₄: 241 (MH⁺).

Step 2: To a cooled (0° C.) solution of 6-bromo-N-ethyl-3H-imidazo[4,5-b]pyridin-2-amine (0.36 g, 1.5 mmol) in DMF was added NaH (60% dispersion in mineral oil, 0.060 g, 1.5 mmol) portionwise over 15 minutes. The reaction mixture was stirred for 15 minutes and then chloro(methoxy)methane (0.12 mL, 1.5 mmol) was added dropwise over 15 minutes. The resulting slurry was allowed to warm to ambient temperature and was stirred for two hours and was partitioned between ethyl acetate and saturated aqueous sodium bicarbonate. The organic layer was washed with brine, dried over magnesium sulfate, filtered and concentrated in vacuo. Purification by silica gel chromatography provided both 6-bromo-N-ethyl-N,3-bis(methoxymethyl)-3H-imidazo[4,5-b]pyridin-2-amine (0.091 g, 18%) and 6-bromo-N-ethyl-3-(methoxymethyl)-3H-imidazo[4,5-b]pyridin-2-amine (0.15 g, 35% yield). Bisprotected product: MS (ES) for C₁₂H₁₇BrN₄O₂: 329 (MH⁺). Monoprotected product: ¹H NMR (400 MHz, CDCl₃) δ 8.03 (d, 1H), 7.73 (d, 1H), 5.42 (s, 2H), 4.98 (s, 1H), 3.59 (q, 2H), 3.36 (s, 3H), 1.34 (t, 3H); MS (ES) for C₁₀H₁₃BrN₄O: 285 (MH⁺).

### Reagent Preparation 39

### N-(5-bromo-2-chloropyridin-3-yl)methanesulfonamide

STEP 1: A solution of 5-bromo-2-chloropyridin-3-amine (1.0 g, 4.8 mmol) and diisopropylethylamine (1.85 mL, 10.6 mmol) in dichloromethane (25 mL) was cooled to 0° C., and then methanesulfonyl chloride (750 uL, 9.6 mmol) was added slowly. The reaction mixture was stirred at 0° C. for 15 min and was then warmed to rt. After stirring for 2 h, water was added, and then the biphasic mixture was partitioned. The organic phase was dried over magnesium sulfate, filtered, and concentrated in vacuo. The residue was then dissolved in dioxane (10 mL) and water (10 mL). Potassium carbonate (2.76 g, 20 mmol) was added, and the reaction mixture was stirred for 15 h at rt. Water was then added to the mixture which was subsequently acidified with aqueous citric acid (10%). The aqueous mixture was extracted twice with ethyl acetate. The combined organic extracts were dried over magnesium sulfate, filtered, and concentrated in vacuo. The residue was purified by flash chromatography (gradient, 100% hexanes to 50% hexanes: 50% ethyl acetate) to provide N-(5-bromo-2-chloropyridin-3-yl)methanesulfonamide (520 mg, 1.82 mmol, 38% yield) as a light pink solid. ¹H NMR (400 MHz, CDCl₃) δ 8.27 (d, 1H), 8.14 (d, 1H), 6.83 (br s, 1H), 3.11 (s, 3H); MS (EI) for C₆H₆BrClN₂O₂S: 285, 287, 289 (Br+Cl isotopes, MH⁺).

### Reagent Preparation: 40

### tert-butyl 1-(2-amino-5-bromopyridin-3-ylsulfonypazetidin-3-ylcarbamate

To a solution of tert-butyl azetidin-3-ylcarbamate (64 mg, 0.37 mmol) and potassium carbonate (102 mg, 0.74 mmol) in dioxane (2 mL) and water (400 uL) was added 2-amino-5-bromopyridine-3-sulfonyl chloride (100 mg, 0.37 mmol, prepared according to the methods in WO2008144463). The reaction mixture was stirred for 1 h at room temperature then quenched by addition of saturated aqueous sodium bicarbonate, and the aqueous solution was extracted twice with ethyl acetate. The combined organic extracts were dried over magnesium sulfate, filtered and concentrated to provide tert-butyl 1-(2-amino-5-bromopyridin-3-ylsulfonyl)azetidin-3-ylcarbamate (120 mg, 0.30 mmol, 80% yield) as a white solid. ¹H NMR (400 MHz, CDCl₃) δ 8.31 (d, 1H), 8.00 (d, 1H), 5.76 (br s, 2H), 4.80 (br s, 1H), 4.50-4.36 (m, 1H), 4.11 (t, 2H), 3.75 (t, 2H), 1.42 (s, 9H).; MS (EI) for C₁₃H₁₉BrN₄O₄S: 407, 409 (Br isotopes, MW).

### Biological Examples

### Biological Example 1

### mTOR/GbL/Raptor (mTORC1) ELISA Assay

The measurement of mTORC1 enzyme activity is performed in an ELISA assay format following the phosphorylation of 4E-BP1 protein. All experiments are performed in the 384-well format. Generally, 0.5 μL DMSO containing varying concentrations of the test compound was mixed with 15 μL enzyme solution. Kinase reactions are initiated with the addition of 15 μL of substrates-containing solution. The assay conditions are as follows; 0.2 nM mTORCl, 10 μM ATP and 50 nM NHis-tagged 4E-BP1 in 20 mM Hepes, pH 7.2, 1 mM DTT, 50 mM NaCl, 10 mM MnCl₂, 0.02 mg/mL BSA, 0.01% CHAPS, 50 mM 1-glycerophosphate. Following an incubation of 120 minutes at ambient temperature, 20 μL of the reaction volume is transferred to a Ni-Chelate-coated 384-well plate. The binding step of the 4E-BP1 protein is proceeded for 60 minutes, followed by washing 4 times each with 50 pL of Tris-buffered saline solution (TBS). Anti-phospho-4E-BP1 rabbit-IgG (20 μL, 1:5000) in 5% BSA-TBST (0.2% Tween-20 in TBS) is added and further incubated for 60 minutes. Incubation with a secondary HRP-tagged anti-IgG is similarly performed after washing off the primary antibody (4 washes of 50 μL). Following the final wash step with TBST, 20 μL of SuperSignal ELISA Femto (Pierce. Biotechnology) is added and the luminescence measured using an EnVision plate reader.

### Biological Example 2

### Immune-Complex mTORC2 Kinase (mTORC2 IP-Kinase) Assay

HeLa (ATCC) cells are grown in suspension culture and lysed in ice-cold lysis buffer containing 40 mM HEPES pH 7.5, 120 mM NaCl, 1 mM EDTA, 10 mM sodium pyrophosphate, 10 mM β-glycerophosphate, 10 mM NaF, 10 mM NaN₃, one tablet of protease inhibitors (Complete-Mini, EDTA-free, Roche), 0.3% cholamidopropyldimethylammoniopropanesulfonate (CHAPS), 1 mM AEBSF, 0.5 mM benzamidine HCl, 20 μg/mL heparin, and 1.5 mM Na₃VO₄. The mTORC2 complex is immunoprecipitated with anti-RICTOR antibody for 2 h. The immune complexes are immobilized on Protein A sepharose (GE Healthcare, 17-5280-01), washed sequentially 3 times with wash buffer (40 mM HEPES pH 7.5, 120 mM NaCl, 10 mM (3-glycerophosphate, 0.3% CHAPS, 1 mM AEBSF, 20 μg/mL heparin, 1.5 mM Na₃VO₄, and Complete-Mini, EDTA-free) and resuspended in kinase buffer (40 mM HEPES, pH 7.5, 120 mM NaCl, 0.3% CHAPS, 20 μg/mL heparin, 4 mM MgCl₂, 4 mM MnCl₂, 10% Glycerol, and 10 mM DTT). The immune complexes (equivalent to 1×10⁷ cells) are pre-incubated at 37° C. with a test compound or 0.6% DMSO for 5 min, and then subjected to a kinase reaction for 8 min in a final volume of 33 μL (including 5 μL bed volume) containing kinase buffer, 50 μM ATP, and 0.75 μg full length dephosphorylated AKT1. Kinase reactions are terminated by addition of 11 μL 4×SDS sample buffer containing 20% β-mercaptoethanol and resolved in a 10% Tris Glycine gels. The gels are transferred onto PVDF membrane at 50 V for 20 h at 4° C. The membranes are blocked in 5% non-fat milk in TBST for 1 h and incubated overnight at 4° C. with 1/1000 dilution of rabbit anti-pAKT (S473) (Cell Signaling Technology, 4060) in 3% BSA/TBST. The membranes are washed 3 times in TBST and incubated for 1 h with a 1/10000 dilution of secondary goat anti-rabbit HRP antibody (Cell Signaling Technology, 2125) in 5% non-fat milk/TBST. The signal is detected using Amersham ECL-plus. The scanned data are analyzed using ImageQuant software. IC₅₀ for the test compound is determined relative to DMSO treated sample using XLfit4 software.

### Biological Example 3

### PI3K Biochemical Assays

PI3Kα activity was measured as the percent of ATP consumed following the kinase reaction using luciferase-luciferin-coupled chemiluminescence. Reactions were conducted in 384-well white, medium binding microtiter plates (Greiner). Kinase reactions were initiated by combining test compounds, ATP, substrate (PIP2), and kinase in a 20 μL volume in a buffer solution. The standard PI3Kalpha assay buffer was composed 50 mM Tris, pH 7.5, 1 mM EGTA, 10 mM MgCl₂, 1 mM DTT and 0.03% CHAPS. The standard assay concentrations for enzyme, ATP, and substrate were 3 nM, 1 μM, and 10 μM, respectively. The reaction mixture was incubated at ambient temperature for approximately 2 h. Following the kinase reaction, a 10 μL aliquot of luciferase-luciferin mix (Promega Kinase-Glo) was added and the chemiluminescence signal measured using a Victor2 or EnVision (Perkin Elmer). Total ATP consumption was limited to 40-60% and IC50 values of control compounds correlate well with literature references. Substituting PI3Kα with PI3Kβ, PI3Kγ, or PI3Kδ, the inhibitory activity of the compounds for the other isoforms of PI3K were measured. For the PI3Kβ and PI3Kδ assays, enzyme concentrations were 10 nM and 4 nM, respectively. For the PI3Kγ assay, enzyme concentration was 40 nM, the incubation time was 1 h, and the concentration of MgCl₂ in the assay buffer was 5 mM.

### Embodiments 1

In one embodiment the invention comprises a compound of the invention having a PI3K-alpha-inhibitory activity of about 0.5 μM or less and is inactive for mTOR (when tested at a concentration of 2.0 μM or greater) or is selective for PI3K-alpha over mTOR by about 5-fold or greater, about 7-fold or greater, or about 10-fold or greater. In another embodiment, the invention comprises a compound of the invention having a PI3K-alpha-inhibitory activity of about 0.35 μM or less and is inactive for mTOR (when tested at a concentration of 2.0 μM or greater) or is selective for PI3K-alpha over mTOR by about 5-fold or greater, about 7-fold or greater, or about 10-fold or greater. In another embodiment, the invention comprises a compound of the invention having a PI3K-alpha-inhibitory activity of about 0.25 μM or less and is inactive for mTOR (when tested at a concentration of 2.0 μM or greater) or is selective for PI3K-alpha over mTOR by about 5-fold or greater, about 7-fold or greater, or about 10-fold or greater. In another embodiment the compounds of the invention have an PI3K-alpha-inhibitory activity of about 0.1 μM or less and is inactive for mTOR (when tested at a concentration of 2.0 μM or greater) or is selective for PI3K-alpha over mTOR by about 5-fold or greater, about 7-fold or greater, or about 10-fold or greater. In another embodiment the invention comprises a compound of the invention having an PI3K-alpha-inhibitory activity of about 0.05 μM or less and is selective for PI3K-alpha over mTOR by about 5-fold or greater, about 7-fold or greater, or about 10-fold or greater.

### Embodiments 2

In one embodiment the invention comprises a compound of the invention having a PI3K-alpha-inhibitory activity of about 2.0 μM or less and an mTOR-inhibitory activity of about 2.0 μM or less and the selectivity for one of the targets over the other does not exceed 3-fold. In another embodiment the invention comprises a compound of the invention having a PI3K-alpha-inhibitory activity of about 1.0 μM or less and an mTOR-inhibitory activity of about 1.0 μM or less and the selectivity for one of the targets over the other does not exceed 3-fold. In another embodiment the invention comprises a compound of the invention having a PI3K-alpha-inhibitory activity of about 0.5 μM or less and an mTOR-inhibitory activity of about 0.5 μM or less and the selectivity for one of the targets over the other does not exceed 3-fold. In another embodiment the invention comprises a compound of the invention having a PI3K-alpha-inhibitory activity of about 0.3 μM or less and an mTOR-inhibitory activity of about 0.3 μM or less and the selectivity for one of the targets over the other does not exceed 3-fold. In another embodiment the invention comprises a compound of the invention having a PI3K-alpha-inhibitory activity of about 0.2 μM or less and an mTOR-inhibitory activity of about 0.2 μM or less and the selectivity for one of the targets over the other does not exceed 2-fold. In another embodiment the invention comprises a compound of the invention having a PI3K-alpha-inhibitory activity of about 0.15 μM or less and an mTOR-inhibitory activity of about 0.15 μM or less and the selectivity for one of the targets over the other does not exceed 2-fold. In another embodiment the invention comprises a compound of the invention having a PI3K-alpha-inhibitory activity of about 0.1 μM or less and an mTOR-inhibitory activity of about 0.1 μM or less. In another embodiment the invention comprises a compound of the invention having a PI3K-alpha-inhibitory activity of about 0.05 μM or less and an mTOR-inhibitory activity of about 0.05 μM or less. In another embodiment the invention comprises a compound of the invention have a PI3K-alpha-inhibitory activity of about 0.02 μM or less and an mTOR-inhibitory activity of about 0.02 μM or less. In another embodiment the invention comprises a compound of the invention have a PI3K-alpha-inhibitory activity of about 0.01 μM or less and an mTOR-inhibitory activity of about 0.01 μM or less.

### Biological Example 5

### pS6 (S240/244) ELISA Assay

MCF-7 cells (ATCC) cells are seeded at 24000 cells per well in 96-well plates (Corning, 3904) in DMEM (Cellgro) containing 10% FBS (Cellgro), 1% NEAA (Cellgro) and 1% penicillin-streptomycin (Cellgro). Cells are incubated at 37° C., 5% CO2 for 48 h, and the growth medium is replaced with serum-free DMEM or in medium containing 0.4% BSA. Serial dilutions of the test compound in 0.3% DMSO (vehicle) are added to the cells and incubated for 3 h. To fix the cells, medium is removed and 100gUwell of 4% formaldehyde (Sigma Aldrich, F8775) in TBS (20 mM Tris, 500 mM NaCl) is added to each well at RT for 30 min. Cells are washed 4 times with 200 μL TBS containing 0.1% Triton X-100 (Sigma, catalog #T9284). Plates are blocked with 100 μL Odyssey blocking buffer (Li-Cor Biosciences, 927-40000) for 1 h at RT. Anti-pS6 (S240/244) antibody (Cell Signaling Technology, 2215) and anti-total-S6 antibody (R&D systems, MAB5436) are diluted 1:400 in Odyssey blocking buffer, and 50 μL of the antibody solution containing both antibodies is added to one plate to detect pS6 and total S6. After incubation overnight at 4° C., plates are washed 4 times with 200 μL TBS containing 0.1% Tween20 (Bio-Rad, catalog #170-6351) (TBST). Goat anti-rabbit and Goat anti-mouse secondary antibody (Li-Cor Biosciences, catalog #926-32221 and 926-32210) conjugated to IRDye are diluted 1:400 in Odyssey blocking buffer containing 0.1% Tween20. 50 μL of antibody solution containing both antibodies is added to each well and incubated for 1 h at RT. Plates were washed 3 times with 2004, TBST and 2 times with 200 μL TBS. Fluorescence is read on an Odyssey plate reader. IC50 values are determined based on the ratio of pS6 to total S6 signal for compound treated wells, normalized to the DMSO-treated control wells.

In one embodiment, the Compounds of the Invention tested in this assay in MCF-7 cells had an inhibitory activity of about 1.0 μM or less. In another embodiment, the Compounds of the Invention tested in this assay in MCF-7 cells had an inhibitory activity of about 0.5 μM or less. In another embodiment, the Compounds of the Invention tested in this assay in MCF-7 cells had an inhibitory activity of about 0.25 μM or less. In another embodiment, the Compounds of the Invention tested in this assay in MCF-7 cells had an inhibitory activity of about 0.2 μM or less. In another embodiment, the Compounds of the Invention tested in this assay in MCF-7 cells had an inhibitory activity of about 0.15 μM or less.

### Biological Example 6

### pAKT (T308) ELISA Assay

MCF-7 cells (ATCC) cells are seeded at 24000 cells per well in 96-well plates (Corning, 3904) in DMEM (Cellgro) containing 10% FBS (Cellgro), 1% NEAA (Cellgro) and 1% penicillin-streptomycin (Cellgro). Cells are incubated at 37° C., 5% CO2 for 48 h, and the growth medium is replaced with serum-free DMEM or in medium containing 0.4% BSA. Serial dilutions of the test compound in 0.3% DMSO (vehicle) are added to the cells and incubated for 3 h. At the end of the incubation period, cells are stimulated for 10 minutes by the addition of L-IGF (Sigma, 1-1271) at a final concentration of 100 ng/ml. Afterwards, media is discarded from cell plates and 110p. 1/well of cold lysis buffer (see table below) are added. Cell plates are incubated on ice and then put on shaker in 4° C. cold room for 1 h. Two capture plates (Thermo Scientific, Reacti-bind plate, 15042) are prepared for each cell plate by pre-coating with capture Akt antibody from the two sandwich ELISA antibody pairs used (Cell Signaling Technology 7142 and 7144). The Akt capture antibodies are diluted 1:100 in PBS and 100t1 of diluted capture antibody is added per well. Capture plates are incubated at 4C overnight. Prior to use, capture plates are washed 3 times in TBS containing 0.1% Tween20 (Bio-Rad, 170-6351) (TBST) and blocked in blocking buffer (Thermo Scientific, Starting Block T20, 37543) for 1-2 h at room temperature. After 1 h of cell lysis, 85 μl of cell lysate/well is transferred to the capture plate for detection of pAkt(T308). 15 μl of cell lysate is transferred from same well to the second capture plate for detection of total Aktl. After incubation overnight at 4° C., plates are washed 3 times with 2004 TBST. Primary antibodies, diluted 1:100 in blocking buffer, are added to the corresponding capture plates for pAkt(T308) (Cell Signaling Technology, 7144) and total Aktl (Cell Signaling Technology, 7142) detection and incubated at room temperature for 1 h. Plates are washed 3 times with 200 μL of TBST. Goat anti-mouse secondary antibody (Cell Signaling Technology, 7076) conjugated to HRP is diluted 1:1000 in blocking buffer and 1000 are added to each well and incubated for 30 minutes at room temperature. Plates are then washed 3 times with 200 μL of TBST. 100 μL of SuperSignal ELISA Femto stable peroxidase solution (Thermo Scientific, 37075) is added to each well. After 1 minute incubation, chemiluminescence is read on a Wallac Victor2 1420 multilabel counter. IC50 values are determined based on the ratio of pAkt(T308) to total Aktl signal for compound treated wells, normalized to the DMSO-treated control wells.

| | Stock | Final | /10 mL | |
|---|---|---|---|---|
| Water | | | 6 | mL |
| Complete Protease Inhibitors | | | 1 mini- | |
| (Roche 1 836 170) | | | tablet | |
| 5x RIPA | 5x | 1x | 2 | mL |
| NaF | 200 mM | 1 mM | 50 | μL |
| B-glycerophosphate | 100 mM | 20 mM | 1.8 | mL |
| Phosphatase Inhibitor I | 100x | 1x | 100 | μL |
| (Sigma P2850) | | | | |
| Na orthovanadate | 200 mM | 1 mM | 50 | μL |
| EDTA, pH 8 | 500 mM | 1 mM | 20 | μL |

In one embodiment, the Compounds of the Invention tested in this assay in MCF-7 cells had an inhibitory activity of about 1.5 μM or less. In another embodiment, the Compounds of the Invention tested in this assay in MCF-7 cells had an inhibitory activity of about 1.0 μM or less. In another embodiment, the Compounds of the Invention tested in this assay in MCF-7 cells had an inhibitory activity of about 0.75 μM or less. In another embodiment, the Compounds of the Invention tested in this assay in MCF-7 cells had an inhibitory activity of about 0.5 μM or less. In another embodiment, the Compounds of the Invention tested in this assay in MCF-7 cells had an inhibitory activity of about 0.25 μM or less. In another embodiment, the Compounds of the Invention tested in this assay in MCF-7 cells had an inhibitory activity of about 0.2 μM or less. In another embodiment, the Compounds of the Invention tested in this assay in MCF-7 cells had an inhibitory activity of about 0.15 μM or less.

### Biological Example 7-13

### Pharmacodynamic Xenograft Tumor Models

Female and male athymic nude mice (NCr) 5-8 weeks of age and weighing approximately 20-25 g are used in the following models. Prior to initiation of a study, the animals are allowed to acclimate for a minimum of 48 h. During these studies, animals are provided food and water ad libitum and housed in a room conditioned at 70-75° F. and 60% relative humidity. A 12 h light and 12 h dark cycle is maintained with automatic timers. All animals are examined daily for compound-induced or tumor-related deaths.

### MCF-7 Breast Adenocarcinoma Model

MCF7 human mammary adenocarcinoma cells are cultured in vitro in DMEM (Cellgro) supplemented with 10% Fetal Bovine Serum (Cellgro), Penicillin-Streptomycin and non-essential amino acids at 37° C. in a humidified 5% CO₂ atmosphere. On day 0, cells are harvested by trypsinization, and 5×10⁶ cells in 100 μL of a solution made of 50% cold Hanks balanced salt solution with 50% growth factor reduced matrigel (Becton Dickinson) implanted subcutaneously into the hindflank of female nude mice. A transponder is implanted into each mouse for identification and data tracking, and animals are monitored daily for clinical symptoms and survival.

Tumors are established in female athymic nude mice and staged when the average tumor weight reached 100-200 mg. A Compound of the Invention is orally administered as a solution/fine suspension in water (with 1:1 molar ratio of 1 NHCL) once-daily (qd) or twice-daily (bid) at 10, 25, 50 and 100 mg/kg for 14 days. During the dosing period of 14-19 days, tumor weights are determined twice-weekly and body weights are recorded daily.

### Colo-205 Colon Model

Colo-205 human colorectal carcinoma cells are cultured in vitro in DMEM (Mediatech) supplemented with 10% Fetal Bovine Serum (Hyclone), Penicillin-Streptomycin and non-essential amino acids at 37° C. in a humidified, 5% CO₂ atmosphere. On day 0, cells are harvested by trypsinization, and 3×10⁶ cells (passage 10-15, >95% viability) in 0.1 mL ice-cold Hank's balanced salt solution are implanted intradermally in the hind-flank of 5-8 week old female athymic nude mice. A transponder is implanted in each mouse for identification, and animals are monitored daily for clinical symptoms and survival.

Tumors are established in female athymic nude mice and staged when the average tumor weight reached 100-200 mg. A Compound of the Invention is orally administered as a solution/fine suspension in water (with 1:1 molar ratio of 1 NHCL) once-daily (qd) or twice-daily (bid) at 10, 25, 50 and 100 mg/kg for 14 days. During the dosing period of 14 days, tumor weights are determined twice-weekly and body weights are recorded daily.

### PC-3 Prostate Adenocarcinoma Model

PC-3 human prostate adenocarcinoma cells are cultured in vitro in DMEM (Mediatech) supplemented with 20% Fetal Bovine Serum (Hyclone), Penicillin-Streptomycin and non-essential amino acids at 37° C. in a humidified 5% CO₂ atmosphere. On day 0, cells are harvested by trypsinization and 3×10⁶ cells (passage 10-14, >95% viability) in 0.1 mL of ice-cold Hank's balanced salt solution are implanted subcutaneously into the hindflank of 5-8 week old male nude mice. A transponder is implanted in each mouse for identification, and animals are monitored daily for clinical symptoms and survival.

Tumors are established in male athymic nude mice and staged when the average tumor weight reached 100-200 mg. A Compound of the Invention is orally administered as a solution/fine suspension in water (with 1:1 molar ratio of 1 N HCl) once-daily (qd) or twice-daily (bid) at 10, 25, 50, or 100-mg/kg for 19 days. During the dosing period of 14-19 days, tumor weights are determined twice-weekly and body weights are recorded daily.

### U-87 MG Human Glioblastoma Model

U-87 MG human glioblastoma cells are cultured in vitro in DMEM (Mediatech) supplemented with 10% Fetal Bovine Serum (Hyclone), Penicillin-Streptomycin and non-essential amino acids at 37° C. in a humidified 5% CO₂ atmosphere. On day 0, cells are harvested by trypsinization and 2×10⁶ cells (passage 5, 96% viability) in 0.1 mL of ice-cold Hank's balanced salt solution are implanted intradermally into the hindflank of 5-8 week old female nude mice. A transponder is implanted in each mouse for identification, and animals are monitored daily for clinical symptoms and survival. Body weights are recorded daily.

### A549 Human Lung Carcinoma Model

A549 human lung carcinoma cells are cultured in vitro in DMEM (Mediatech) supplemented with 10% Fetal Bovine Serum (Hyclone), Penicillin-Streptomycin and non-essential amino acids at 37° C. in a humidified 5% CO₂ atmosphere. On day 0, cells are harvested by trypsinization and 10×10⁶ cells (passage 12, 99% viability) in 0.1 mL of ice-cold Hank's balanced salt solution are implanted intradermally into the hindflank of 5-8 week old female nude mice. A transponder is implanted in each mouse for identification, and animals are monitored daily for clinical symptoms and survival. Body weights are recorded daily.

### A2058 Human Melanoma Model

A2058 human melanoma cells are cultured in vitro in DMEM (Mediatech) supplemented with 10% Fetal Bovine Serum (Hyclone), Penicillin-Streptomycin and non-essential amino acids at 37° C. in a humidified, 5% CO₂ atmosphere. On day 0, cells are harvested by trypsinization and 3×10⁶ cells (passage 3, 95% viability) in 0.1 mL ice-cold Hank's balanced salt solution are implanted intradermally in the hind-flank of 5-8 week old female athymic nude mice. A transponder is implanted in each mouse for identification, and animals are monitored daily for clinical symptoms and survival. Body weights are recorded daily.

### WM-266-4 Human Melanoma Model

WM-266-4 human melanoma cells are cultured in vitro in DMEM (Mediatech) supplemented with 10% Fetal Bovine Serum (Hyclone), Penicillin-Streptomycin and non-essential amino acids at 37° C. in a humidified, 5% CO₂ atmosphere. On day 0, cells are harvested by trypsinization and 3×10⁶ cells (passage 5, 99% viability) in 0.1 mL ice-cold Hank's balanced salt solution are implanted intradermally in the hind-flank of 5-8 week old female athymic nude mice. A transponder is implanted in each mouse for identification, and animals are monitored daily for clinical symptoms and survival. Body weights are recorded daily.

Tumor weight (TW) in the above models is determined by measuring perpendicular diameters with a caliper, using the following formula:

tumor weight (mg)=[tumor volume=length (mm)×width² (mm²)]/2

These data are recorded and plotted on a tumor weight vs. days post-implantation line graph and presented graphically as an indication of tumor growth rates. Percent inhibition of tumor growth (TGI) is determined with the following formula:

$$[ 1 - ( \frac{( {X}_{f} - {X}_{0} )}{( {Y}_{f} - {X}_{0} )} ) ] * 100$$

where X_{f}=average TW of all tumors on group day

X_{f}=TW of treated group on Day f

Y₁=TW of vehicle control group on Day f

If tumors regress below their starting sizes, then the percent tumor regression is determined with the following formula:

$$( \frac{{X}_{0} - {X}_{f}}{{X}_{0}} ) * 100$$

Tumor size is calculated individually for each tumor to obtain a mean±SEM value for each experimental group. Statistical significance is determined using the 2-tailed Student's t-test (significance defined as P<0.05).

The foregoing invention has been described in some detail by way of illustration and example, for purposes of clarity and understanding. The invention has been described with reference to various specific embodiments and techniques. However, it should be understood that many variations and modifications may be made while remaining within the spirit and scope of the invention. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled. All patents, patent applications and publications cited in this application are hereby incorporated by reference in their entirety for all purposes to the same extent as if each individual patent, patent application or publication were so individually denoted.

1. A compound of Formula I(a): 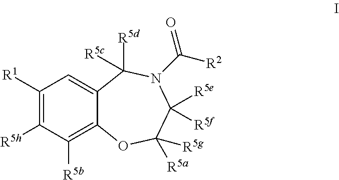
or a single stereoisomer or mixture of isomers thereof and additionally optionally as a pharmaceutically acceptable salt thereof, where
R¹ is phenyl optionally substituted with one, two, or three R⁶ groups; or
R¹ is heteroaryl optionally substituted with one, two, or three R⁷;
R² is —NR³R⁴;
R³ is hydrogen, alkyl, or alkoxycarbonylalkyl; and R⁴ is optionally substituted cycloalkyl, optionally substituted phenyl, optionally substituted phenylalkyl, optionally substituted heteroaryl, or optionally substituted heteroarylalkyl; or
R³ and R⁴ together with the nitrogen to which they are attached form HET optionally substituted on any substitutable atom of the ring with R¹⁰, R^{10a}, R^{10b}, R^{10c}, R^{10d}, R^{10e}, and R^{10f};
HET is
1) a saturated or partially unsaturated, but non-aromatic, monocyclic 5- to 8-membered ring optionally containing an additional one or two ring heteroatoms which are independently oxygen, sulfur, or nitrogen where the remaining ring atoms are carbon; or
2) a partially unsaturated, but not aromatic, monocyclic 5- to 8-membered ring optionally containing an additional one or two ring heteroatoms which are independently oxygen, sulfur, or nitrogen and the remaining ring atoms are carbon and which ring is fused to a benzo ring; or
3) a fused, bridged, or spirocyclic, bicyclic 7- to 11-membered ring optionally containing an additional one or two heteroatoms which are independently oxygen, sulfur, or nitrogen and the remaining ring atoms are carbon and where each ring of the 7- to 11-membered ring is saturated or partially unsaturated but not fully aromatic; or
4) a fused, bridged, or spirocyclic, bicyclic 7- to 11-membered ring optionally containing an additional one or two ring heteroatoms which are independently oxygen, sulfur, or nitrogen and the remaining ring atoms are carbon where each ring of the bicyclic 7- to 11-membered ring is saturated or partially unsaturated but not fully aromatic, and where the bicyclic 7- to 11-membered ring is fused to a benzo ring;
R^{5a} and R^{5c} are independently hydrogen or alkyl;
R^{5h} is hydrogen or halo;
R^{5b} is (C₁₋₃)alkyl, (C₁₋₃)alkoxy, halo(C₁₋₃)alkyl, (C₁₋₃)haloalkoxy;
R^{5d}, R^{5e}, R^{5f}, and R^{59g} are hydrogen;
each R⁶, when R⁶ is present, is independently nitro; cyano; halo; alkyl; alkenyl; alkynyl; halo; haloalkyl; —OR^{8a}; —NR⁸R^{8a}; —C(O)NR⁸C(O)OR⁹; —NR⁸C(O)R⁹; —NR⁸S(O)₂R^{8a}; —NR⁸C(O)NR^{8a}R⁹; carboxy, —C(O)OR⁹; alkylcarbonyl; alkyl substituted with one or two —C(O)NR⁸R^{8a}; heteroaryl optionally substituted with 1, 2, or 3 R¹⁴; or optionally substituted heterocycloalkyl;
each R⁷, when R⁷ is present, is independently oxo; nitro; cyano; alkyl; alkenyl; alkynyl; halo; haloalkyl; hydroxyalkyl; alkoxyalkyl; —OR^{8a}; —SR¹³; —S(O)R¹³; —S(O)₂R¹³; —NR⁸R^{8a}; —C(O)NR⁸R^{8a}; —NR⁸C(O)OR⁹; —NR⁸C(O)R⁹; —NR⁸S(O)₂R^{8a}; —NR⁸C(O)NR^{8a}R⁹; carboxy; —C(O)OR⁹; alkylcarbonyl; —S(O)₂NR⁸R⁹; alkyl substituted with one or two —NR⁸R^{8a}; alkyl substituted with one or two —NR⁸C(O)R^{8a}; optionally substituted cycloalkyl; optionally substituted cycloalkylalkyl; optionally substituted heterocycloalkyl; optionally substituted heterocycloalkylalkyl; optionally substituted heteroaryl; or optionally substituted heteroarylalkyl;
R⁸ is hydrogen, alkyl, alkenyl, alkynyl, hydroxyalkyl, or haloalkyl;
R^{8a} is hydrogen, alkyl, alkenyl, alkynyl, haloalkyl, hydroxyalkyl, cyanoalkyl, aminoalkyl, alkylaminoalkyl, dialkylaminoalkyl, alkoxyalkyl, optionally substituted cycloalkyl, optionally substituted cycloalkylalkyl, optionally substituted heterocycloalkyl, optionally substituted heterocycloalkylalkyl, optionally substituted phenyl, optionally substituted phenylalkyl, optionally substituted heteroaryl, or optionally substituted heteroarylalkyl;
R⁹ is alkyl, alkenyl, alkynyl, hydroxyalkyl, alkoxyalkyl, haloalkyl, or optionally substituted heterocycloalkylalkyl;
R¹⁰, R^{10a}, R^{10b}, R^{10c}, R^{10d}, R^{10e}, and R^{10f} are independently hydrogen; halo; alkyl; haloalkyl; haloalkenyl; hydroxyalkyl; alkylthio; alkylsulfonyl; hydroxy; alkoxy; haloalkoxy; cyano; alkoxycarbonyl; carboxy; amino; alkylamino; dialkylamino; —C(O)R¹²; —C(O)NR¹¹R^{11a}; optionally substituted cycloalkyl; optionally substituted cycloalkylalkyl; optionally substituted phenyl; optionally substituted phenylalkyl; optionally substituted phenyloxy; optionally substituted phenyloxyalkyl; optionally substituted heterocycloalkyl; optionally substituted heterocycloalkylalkyl; optionally substituted heteroaryl; or optionally substituted heteroarylalkyl; or two of R¹⁰, R^{10a}, R^{10b}, R^{10c}, R^{10d}, R^{10e}, and R^{10f} when attached to the same carbon form oxo, imino, or thiono;
R¹¹ hydrogen, alkyl, or alkenyl;
R^{11a} hydrogen, alkyl, or alkenyl;
R¹² is alkyl, or optionally substituted heteroaryl;
R¹³ is alkyl or haloalkyl; and
each R¹⁴, when R¹⁴ is present, is independently amino, alkylamino, dialkylamino, acylamino, halo, hydroxy, alkyl, haloalkyl, hydroxyalkyl, aminoalkyl, alkylaminoalkyl, dialkylaminoalkyl, alkoxycarbonyl, aminocarbonyl, alkylaminocarbonyl, dialkylaminocarbonyl, or optionally substituted phenyl.

2. The compound of claim 1, or a single stereoisomer or mixture of isomers thereof and additionally optionally as a pharmaceutically acceptable salt thereof, where
R¹ is phenyl substituted with one or two R⁶ groups; or
R¹ is heteroaryl optionally substituted with one, two, or three R⁷;
R² is —NR³R⁴;
R³ is hydrogen, alkyl, or alkoxycarbonylalkyl; and R⁴ is optionally substituted cycloalkyl, optionally substituted phenyl, optionally substituted phenylalkyl, or optionally substituted heteroarylalkyl; or
R³ and R⁴ together with the nitrogen to which they are attached form HET optionally substituted on any substitutable atom of the ring with R¹⁰, R^{10a}, and R^{10b};
HET is
(a) a saturated or partially unsaturated, but non-aromatic, monocyclic 5- to 8-membered ring optionally containing an additional one or two ring heteroatoms which are independently oxygen, sulfur, or nitrogen where the remaining ring atoms are carbon; or
(b) a partially unsaturated, but not aromatic, monocyclic 5- to 8-membered ring optionally containing an additional one or two ring heteroatoms which are independently oxygen, sulfur, or nitrogen and the remaining ring atoms are carbon and which ring is fused to a benzo ring; or
(c) a fused, bridged, or spirocyclic, bicyclic 7- to 11-membered ring optionally containing an additional one or two heteroatoms which are independently oxygen, sulfur, or nitrogen and the remaining ring atoms are carbon and where each ring of the 7- to 11-membered ring is saturated or partially unsaturated but not fully aromatic; or
(d) a fused, bridged, or spirocyclic, bicyclic 7- to 11-membered ring optionally containing an additional one or two ring heteroatoms which are independently oxygen, sulfur, or nitrogen and the remaining ring atoms are carbon where each ring of the bicyclic 7- to 11-membered ring is saturated or partially unsaturated but not fully aromatic, and where the bicyclic 7- to 11-membered ring is fused to a benzo ring;
R^{5a}, R^{5c}, R^{5h}, R^{5d}, R^{5e}, R^{5f}, and R^{5g} are hydrogen;
R^{5b} is (C₁₋₃)alkyl;
each R⁶, when R⁶ is present, is independently nitro, —C(O)NR⁸R^{8a}, —NR⁸C(O)OR⁹, or heteroaryl optionally substituted with 1, 2, or 3 R¹⁴;
each R⁷, when present, is independently alkyl, cycloalkyl, —NR⁸R^{8a}, —C(O)NR⁸R^{8a}, —NR⁸C(O)OR⁹, or —NR⁸C(O)R⁹;
R⁸ is hydrogen, alkyl, or alkenyl;
R^{8a} is hydrogen, alkyl, haloalkyl, optionally substituted heterocycloalkyl, or optionally substituted phenylalkyl;
R⁹ is alkyl or haloalkyl; and
R¹⁰, R^{10a}, R^{10b}, R^{10c}, R^{10d}, R^{10e}, and R^{10f} are independently hydrogen, alkyl, haloalkyl, haloalkenyl, hydroxyalkyl, alkylthio, alkylsulfonyl, hydroxy, alkoxy, haloalkoxy, cyano, alkoxycarbonyl, carboxy, amino, alkylamino, dialkylamino, —C(O)R¹², —C(O)NR¹¹R^{11a}, optionally substituted cycloalkyl, optionally substituted cycloalkylalkyl, optionally substituted phenyl, optionally substituted phenylalkyl, optionally substituted phenyloxy, optionally substituted phenyloxyalkyl, optionally substituted heterocycloalkyl, optionally substituted heterocycloalkylalkyl, optionally substituted heteroaryl, or optionally substituted heteroarylalkyl; or two of R¹⁰, R^{10a}, R^{10b}, R^{10c}, R^{10d}, R^{10e}, and R^{10f} when attached to the same carbon form oxo;
R¹¹ hydrogen, alkyl, or alkenyl;
R^{11a} hydrogen, alkyl, or alkenyl;
R¹² is alkyl, or optionally substituted heteroaryl; and
each R¹⁴, when present, is halo, alkyl, or alkoxycarbonyl.

3. The compound of claim 1, or a single stereoisomer or mixture of isomers thereof and additionally optionally as a pharmaceutically acceptable salt thereof, where
R¹ is phenyl substituted with one or two R⁶ groups; or
R¹ is heteroaryl optionally substituted with one, two, or three R⁷;
R² is —NR³R⁴ where R³ is hydrogen, alkyl, or alkoxycarbonylalkyl; and R⁴ is optionally substituted cycloalkyl, optionally substituted phenyl, optionally substituted phenylalkyl, or optionally substituted heteroarylalkyl; or
R² is —NR³R⁴ where R³ and R⁴ together with the nitrogen to which they are attached form indolin-1-yl, isoindolin-2-yl, 1,2,3,4-tetrahydroquinolin-1-yl, 1,2,3,4-tetrahydroisoquinolin-2-yl, or 1,2,3,4-tetrahydro-1,4-epiminonaphth-9-yl, where any substitutable atom on the ring is optionally substituted with R¹⁰, R^{10a}, and R^{10b}; or
R² is —NR³R⁴ where R³ and R⁴ together with the nitrogen to which they are attached form HET according to formula (a): 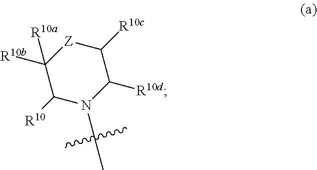
where Z is a bond, —C(O)—, —O—, —S—, —S(O)—, —S(O)₂—, —N(R^{z})—, —C(R^{10e})(R^{10f})—, or C₂₋₃-alkylene; or
R² is —NR³R⁴ where R³ and R⁴ together with the nitrogen to which they are attached form HET according to formula (b): 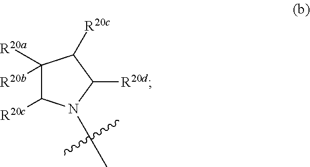
where
(a) R²⁰ and R^{20c} or R²⁰ and R^{20d} together with the carbons to which they are bonded form a cycloalkyl or hetercycloalkyl such that HET is a bridged moiety; or
(b) R^{20a} and R^{20c} together with the carbons to which they are bonded form a cycloalkyl or hetercycloalkyl such that HET is a fused bicyclic moiety; or
(c) R^{20a} and R^{20b} together with the carbon to which they are attached form cycloalkyl or heterocycloalkyl such that HET is a spirocyclic moiety;
and the remaining of R²⁰, R^{20a}, R^{20b}, R^{20c}, and R^{20d} are hydrogen; and where the cycloalkyl and heterocycloalkyl are optionally substituted with R¹⁰ and R^{10a}; or
R² is —NR³R⁴ where R³ and R⁴ together with the nitrogen to which they are attached form HET according to formula (c): 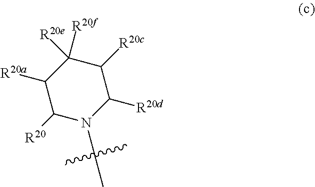
where
(a) R²⁰ and R^{20a} or R²⁰ and R^{20c} together with the carbons to which they are bonded form a cycloalkyl or hetercycloalkyl such that HET is a bridged moiety
(b) R^{20e} and R^{20f} together with the carbons to which they are bonded form cycloalkyl or heterocycloalkyl such that HET is a spirocyclic moiety,
(c) R²⁰ and R^{20a} or R^{20a} and R^{20e} together with the carbons to which they are bonded form a cycloalkyl or hetercycloalkyl such that HET is a fused bicyclic moiety;
and the remaining of R²⁰, R^{20a}, R^{20c}, R^{20d}, R^{20e}, and R^{20f} are R¹⁰, R^{10a}, R^{10c}, R^{10d}, R^{10e}, and R^{10f}, respectively; and where the cycloalkyl and heterocycloalkyl are optionally substituted with R¹⁰ and R^{10a};
each R⁶, when R⁶ is present, is independently nitro, —NR⁸R^{8a}, —C(O)NR⁸R^{8a}, —NR⁸C(O)OR⁹, or heteroaryl optionally substituted with 1, 2, or 3 R¹⁴;
each R⁷, when present, is independently alkyl, cycloalkyl, —NR⁸R^{8a}, —C(O)NR⁸R^{8a}, —NR⁸C(O)OR⁹, or —NR⁸C(O)R⁹;
R⁸ is hydrogen, alkyl, or alkenyl;
R^{8a} is hydrogen, alkyl, haloalkyl, optionally substituted heterocycloalkyl, or optionally substituted phenylalkyl;
R⁹ is alkyl or haloalkyl; and
R^{z} is hydrogen, alkyl, haloalkyl, haloalkenyl, hydroxyalkyl, alkylsulfonyl, hydroxy, alkoxy, alkoxycarbonyl, —C(O)R¹², —C(O)NR¹¹R^{11a}, optionally substituted cycloalkyl, optionally substituted cycloalkylalkyl, optionally substituted phenyl, optionally substituted phenylalkyl, optionally substituted heterocycloalkyl, optionally substituted heterocycloalkylalkyl, optionally substituted heteroaryl, or optionally substituted heteroarylalkyl;
each R¹⁰, each R^{10a}, R^{10b}, R^{10c}, R^{10d}, R^{10e}, and R^{10f} are independently hydrogen, alkyl, halo, haloalkyl, haloalkenyl, hydroxyalkyl, alkylthio, alkylsulfonyl, hydroxy, alkoxy, haloalkoxy, cyano, alkoxycarbonyl, carboxy, amino, alkylamino, dialkylamino, —C(O)R¹², —C(O)NR¹¹R^{11a}, optionally substituted cycloalkyl, optionally substituted cycloalkylalkyl, optionally substituted phenyl, optionally substituted phenylalkyl, optionally substituted phenyloxy, optionally substituted phenyloxyalkyl, optionally substituted heterocycloalkyl, optionally substituted heterocycloalkylalkyl, optionally substituted heteroaryl, or optionally substituted heteroarylalkyl; or two of R¹⁰, R^{10a}, R^{10b}, R^{10c}, R^{10d}, R^{10e}, and R^{10f} when attached to the same carbon form oxo;
R¹¹ is hydrogen, alkyl, alkenyl, or alkynyl;
R^{11a} is hydrogen, alkyl, alkenyl, or alkynyl;
R¹² is alkyl, or optionally substituted heteroaryl; and
each R¹⁴, when present, is halo, alkyl, or alkoxycarbonyl.

4. The compound of claim 1, or a single stereoisomer or mixture of isomers thereof and additionally optionally as a pharmaceutically acceptable salt thereof, where the Compound is according to Formula I(b)

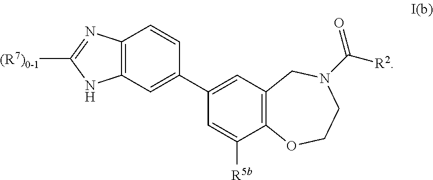

5. The compound of claim 1, or a single stereoisomer or mixture of isomers thereof and additionally optionally as a pharmaceutically acceptable salt thereof, where the Compound is according to Formula I(c1) or I(c2)

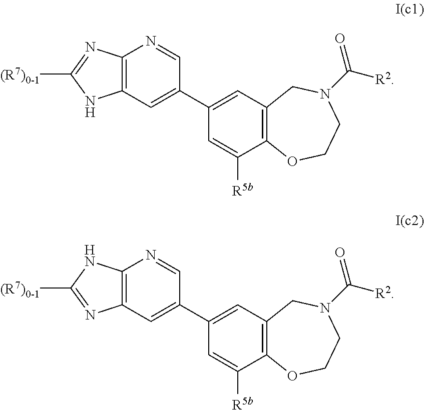

6. The compound of claim 1, or a single stereoisomer or mixture of isomers thereof and additionally optionally as a pharmaceutically acceptable salt thereof, where the Compound is according to Formula I(d1) or I(d2)

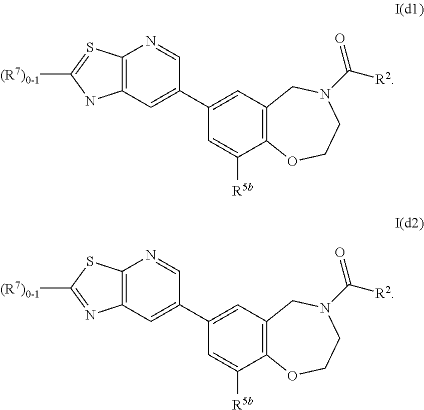

7. The compound of claim 1, or a single stereoisomer or mixture of isomers thereof and additionally optionally as a pharmaceutically acceptable salt thereof, where R¹ is a 6-membered heteroaryl optionally substituted with one or two R⁷.

8. The compound of claim 1, or a single stereoisomer or mixture of isomers thereof and additionally optionally as a pharmaceutically acceptable salt thereof, where R¹ is pyridin-3-yl optionally substituted with one or two R⁷.

9. The compound of claim 1, or a single stereoisomer or mixture of isomers thereof and additionally optionally as a pharmaceutically acceptable salt thereof, where R¹ is a 5-membered heteroaryl optionally substituted with one or two R⁷.

10. The compound of claim 4, or a single stereoisomer or mixture of isomers thereof and additionally optionally as a pharmaceutically acceptable salt thereof, where R⁷, when present, is alkyl, haloalkyl, cycloalkyl, —NR⁸R^{8a}, or —NR⁸C(O)OR⁹.

11. The compound of claim 1, or a single stereoisomer or mixture of isomers thereof and additionally optionally as a pharmaceutically acceptable salt thereof, where R¹ is phenyl substituted with one or two R⁶ groups.

12. The compound of claim 11, or a single stereoisomer or mixture of isomers thereof and additionally optionally as a pharmaceutically acceptable salt thereof, where R¹ is phenyl substituted with one R⁶ group which is —OR^{8a}; —NR⁸R^{8a}, —C(O)NR⁸R^{8a}; or heteroaryl optionally substituted with 1, 2, or 3 R¹⁴.

13. The compound of claim 1, or a single stereoisomer or mixture of isomers thereof and additionally optionally as a pharmaceutically acceptable salt thereof, where R² is —NR³R⁴ and R³ is hydrogen, alkyl, or alkoxycarbonylalkyl; and R⁴ is optionally substituted cycloalkyl, optionally substituted phenyl, optionally substituted phenylalkyl, or optionally substituted heteroarylalkyl.

14. The compound of claim 1, or a single stereoisomer or mixture of isomers thereof and additionally optionally as a pharmaceutically acceptable salt thereof, where R² is indolin-1-yl, isoindolin-2-yl, 1,2,3,4-tetrahydroquinolin-1-yl, 1,2,3,4-tetrahydroisoquinolin-2-yl, or 1,2,3,4-tetrahydro-1,4-epiminonaphth-9-yl, where any substitutable atom on HET is optionally substituted with R¹⁰, R^{10a}, and R^{10b}.

15. The compound of claim 1, or a single stereoisomer or mixture of isomers thereof and additionally optionally as a pharmaceutically acceptable salt thereof, where R² is —NR³R⁴ and R³ and R⁴ together with the nitrogen to which they are attached form HET according to formula (a): 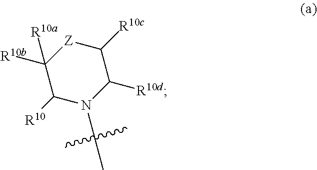
where Z is a bond, —C(O)—, —O—, —S—, —S(O)—, —S(O)₂, —N(R^{z})—, —C(R^{10e})(R^{10f}), or C₂₋₃-alkylene; R^{Z} is hydrogen, alkyl, haloalkyl, haloalkenyl, hydroxyalkyl, alkylsulfonyl, hydroxy, alkoxy, alkoxycarbonyl, —C(O)R¹², —C(O)NR¹¹R^{11a}, optionally substituted cycloalkyl, optionally substituted cycloalkylalkyl, optionally substituted phenyl, optionally substituted phenylalkyl, optionally substituted heterocycloalkyl, optionally substituted heterocycloalkylalkyl, optionally substituted heteroaryl, or optionally substituted heteroarylalkyl.

16. The compound of claim 1, or a single stereoisomer or mixture of isomers thereof and additionally optionally as a pharmaceutically acceptable salt thereof, where R² is —NR³R⁴ where R³ and R⁴ together with the nitrogen to which they are attached form HET according to formula (b): 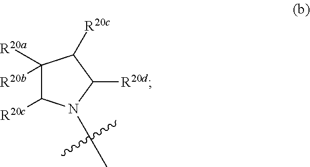
where
(a) R²⁰ and R^{20c} or R²⁰ and R^{20d} together with the carbons to which they are bonded form a cycloalkyl or hetercycloalkyl such that HET is a bridged moiety; or
(b) R^{20a} and R^{20c} together with the carbons to which they are bonded form a cycloalkyl or hetercycloalkyl such that HET is a fused bicyclic moiety; or
(c) R^{20a} and R^{20c} together with the carbon to which they are attached form cycloalkyl or heterocycloalkyl such that HET is a spirocyclic moiety;
where the cycloalkyl and heterocycloalkyl are optionally substituted with R¹⁰ and R^{10a}; and the remaining of R²⁰, R^{20a}, R^{20b}, R^{20c}, and R^{20d} are hydrogen.

17. The compound of claim 1, or a single stereoisomer or mixture of isomers thereof and additionally optionally as a pharmaceutically acceptable salt thereof, where R² is —NR³R⁴ where R³ and R⁴ together with the nitrogen to which they are attached form HET according to formula (c): 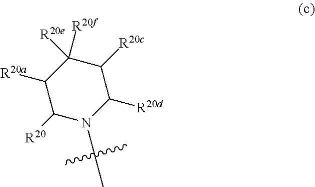
where
(a) R²⁰ and R^{20d} or R²⁰ and R^{20c} together with the carbons to which they are bonded form a cycloalkyl or hetercycloalkyl such that HET is a bridged moiety
(b) R^{20e} an dR^{20f} together with the carbons to which they are bonded form cycloalkyl or heterocycloalkyl such that HET is a spirocyclic moiety,
(c) R²⁰ and R^{20a} or R^{20a} and R^{20e} together with the carbons to which they are bonded form a cycloalkyl or hetercycloalkyl such that HET is a fused bicyclic moiety;
where the cycloalkyl and heterocycloalkyl are optionally substituted with R¹⁰ and R^{10a}; and the remaining of R²⁰, R^{20a}, R^{20c}, R^{10d}, R^{20e}, and R^{20f} are R¹⁰, R^{10a}, R^{10c}, R^{10d}, R^{10e}, and R^{10f}, respectively.

18. The compound of claim 1, or a single stereoisomer or mixture of isomers thereof and additionally optionally as a pharmaceutically acceptable salt thereof, where R² is —NR³R⁴ where R³ and R⁴ together with the nitrogen to which they are attached form HET according to formula (g):

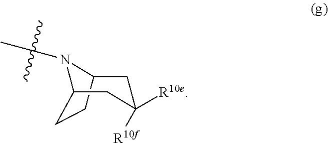

19. The compound of claim 1, or a single stereoisomer or mixture of isomers thereof and additionally optionally as a pharmaceutically acceptable salt thereof, where R² is —NR³R⁴ where R³ and R⁴ together with the nitrogen to which they are attached form HET according to formula (h):

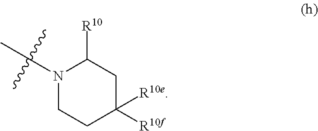

20. The compound of claim 1, which is selected from:

| Entry | |
|---|---|
| No. | Structure |
| 1 | 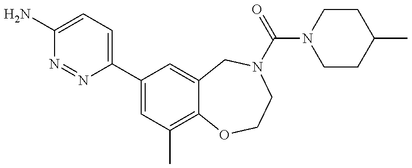 [SMILES: Cc1cc(-c2ccc(N)nn2)cc2c1OCCN(C(=O)N1CCC(C)CC1)C2] |
| 2 | 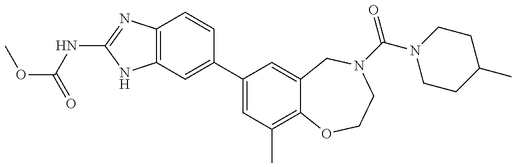 |
| 3 | 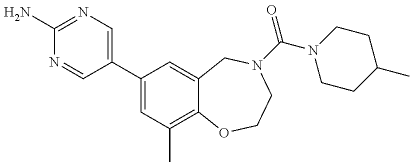 [SMILES: Cc1cc(-c2cnc(N)nc2)cc2c1OCCN(C(=O)N1CCC(C)CC1)C2] |
| 4 | 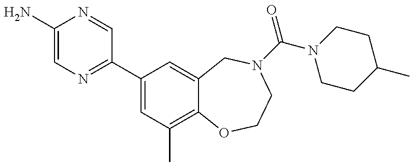 [SMILES: Cc1cc(-c2cnc(N)cn2)cc2c1OCCN(C(=O)N1CCC(C)CC1)C2] |
| 5 | 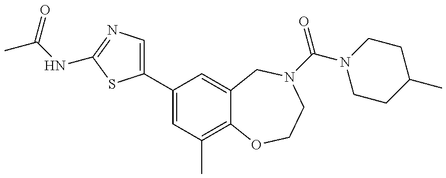 [SMILES: CC(=O)Nc1ncc(-c2cc(C)c3c(c2)CN(C(=O)N2CCC(C)CC2)CCO3)s1] |
| 6 | 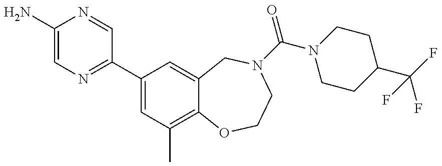 [SMILES: Cc1cc(-c2cnc(N)cn2)cc2c1OCCN(C(=O)N1CCC(C(F)(F)F)CC1)C2] |
| 7 | 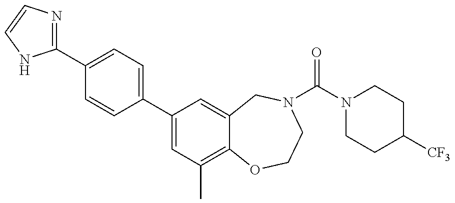 [SMILES: Cc1cc(-c2ccc(-c3ncc[nH]3)cc2)cc2c1OCCN(C(=O)N1CCC(C(F)(F)F)CC1)C2] |
| 8 | 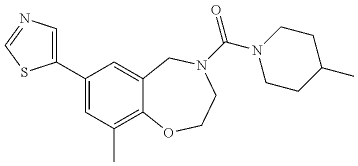 [SMILES: Cc1cc(-c2cncs2)cc2c1OCCN(C(=O)N1CCC(C)CC1)C2] |
| 9 | 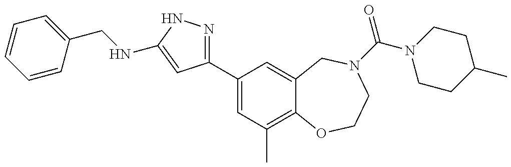 [SMILES: Cc1cc(-c2cc(NCc3ccccc3)[nH]n2)cc2c1OCCN(C(=O)N1CCC(C)CC1)C2] |
| 10 | 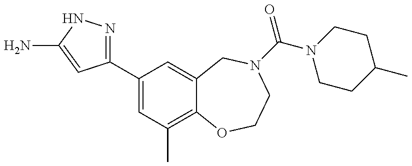 [SMILES: Cc1cc(-c2cc(N)[nH]n2)cc2c1OCCN(C(=O)N1CCC(C)CC1)C2] |
| 11 | 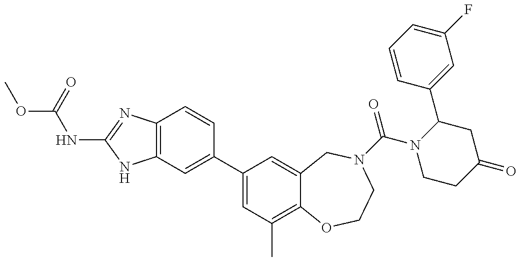 [SMILES: COC(=O)Nc1nc2ccc(-c3cc(C)c4c(c3)CN(C(=O)N3CCC(=O)CC3c3cccc(F)c3)CCO4)cc2[nH]1] |
| 12 | 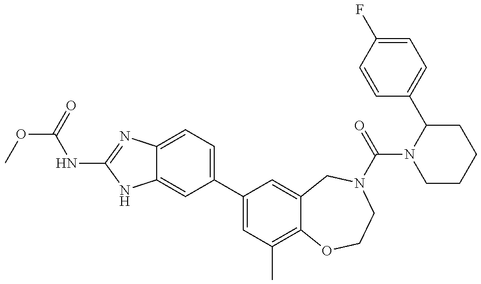 [SMILES: COC(=O)Nc1nc2ccc(-c3cc(C)c4c(c3)CN(C(=O)N3CCCCC3c3ccc(F)cc3)CCO4)cc2[nH]1] |
| 13 | 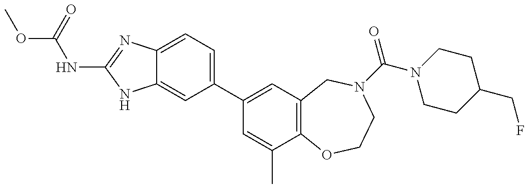 [SMILES: COC(=O)Nc1nc2ccc(-c3cc(C)c4c(c3)CN(C(=O)N3CCC(CF)CC3)CCO4)cc2[nH]1] |
| 14 | 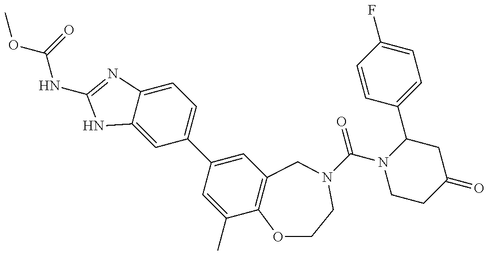 [SMILES: COC(=O)Nc1nc2ccc(-c3cc(C)c4c(c3)CN(C(=O)N3CCC(=O)CC3c3ccc(F)cc3)CCO4)cc2[nH]1] |
| 15 | 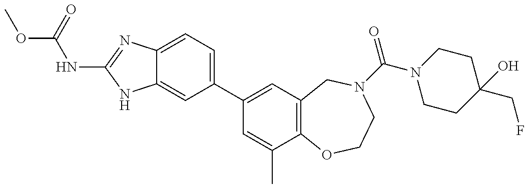 [SMILES: COC(=O)Nc1nc2ccc(-c3cc(C)c4c(c3)CN(C(=O)N3CCC(O)(CF)CC3)CCO4)cc2[nH]1] |
| 16 | 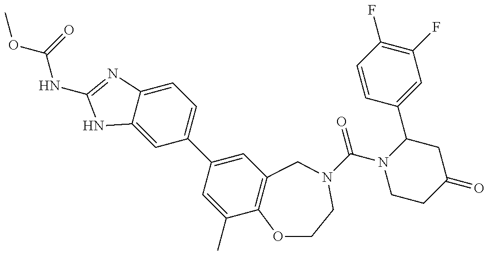 [SMILES: COC(=O)Nc1nc2ccc(-c3cc(C)c4c(c3)CN(C(=O)N3CCC(=O)CC3c3ccc(F)c(F)c3)CCO4)cc2[nH]1] |
| 17 | 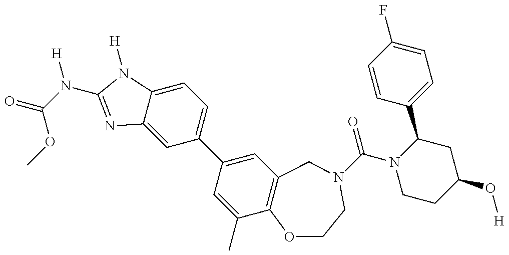 [SMILES: COC(=O)Nc1nc2cc(-c3cc(C)c4c(c3)CN(C(=O)N3CC[C@H](O)C[C@@H]3c3ccc(F)cc3)CCO4)ccc2[nH]1] |
| 18 | 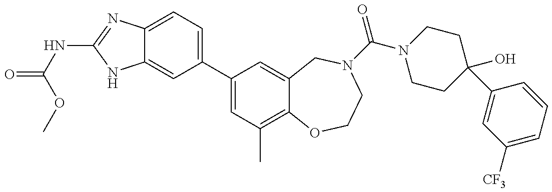 [SMILES: COC(=O)Nc1nc2ccc(-c3cc(C)c4c(c3)CN(C(=O)N3CCC(O)(c5cccc(C(F)(F)F)c5)CC3)CCO4)cc2[nH]1] |
| 19 | 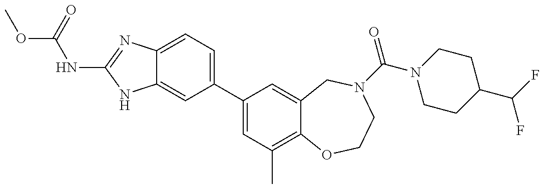 [SMILES: COC(=O)Nc1nc2ccc(-c3cc(C)c4c(c3)CN(C(=O)N3CCC(C(F)F)CC3)CCO4)cc2[nH]1] |
| 20 | 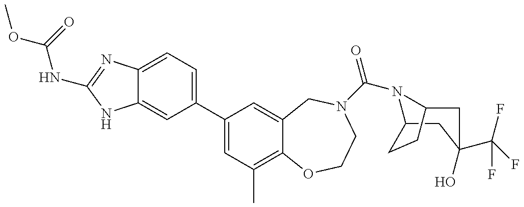 [SMILES: COC(=O)Nc1nc2ccc(-c3cc(C)c4c(c3)CN(C(=O)N3C5CCC3CC(O)(C(F)(F)F)C5)CCO4)cc2[nH]1] |
| 21 | 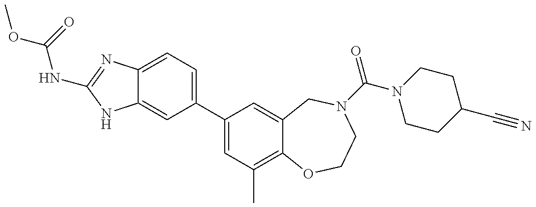 [SMILES: COC(=O)Nc1nc2ccc(-c3cc(C)c4c(c3)CN(C(=O)N3CCC(C#N)CC3)CCO4)cc2[nH]1] |
| 22 | 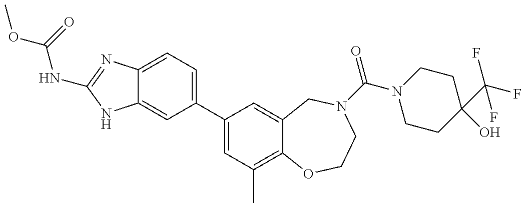 [SMILES: COC(=O)Nc1nc2ccc(-c3cc(C)c4c(c3)CN(C(=O)N3CCC(O)(C(F)(F)F)CC3)CCO4)cc2[nH]1] |
| 23 | 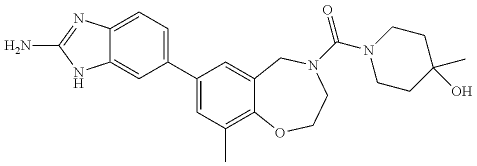 [SMILES: Cc1cc(-c2ccc3nc(N)[nH]c3c2)cc2c1OCCN(C(=O)N1CCC(C)(O)CC1)C2] |
| 24 | 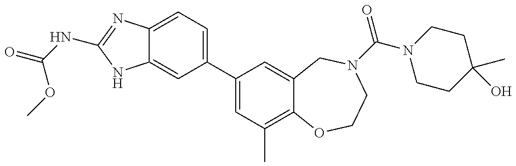 [SMILES: COC(=O)Nc1nc2ccc(-c3cc(C)c4c(c3)CN(C(=O)N3CCC(C)(O)CC3)CCO4)cc2[nH]1] |
| 25 | 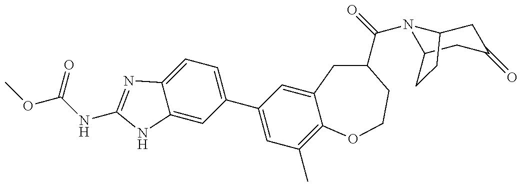 [SMILES: COC(=O)Nc1nc2ccc(-c3cc(C)c4c(c3)CC(C(=O)N3C5CCC3CC(=O)C5)CCO4)cc2[nH]1] |
| 26 | 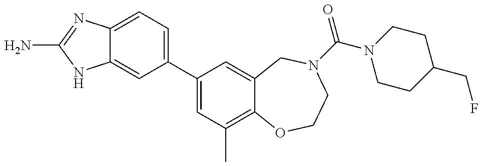 [SMILES: Cc1cc(-c2ccc3nc(N)[nH]c3c2)cc2c1OCCN(C(=O)N1CCC(CF)CC1)C2] |
| 27 | 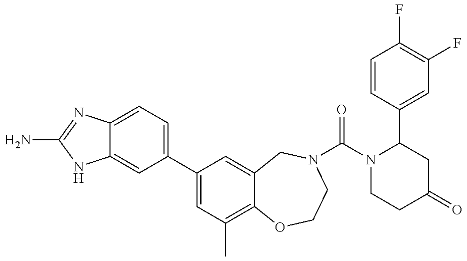 [SMILES: Cc1cc(-c2ccc3nc(N)[nH]c3c2)cc2c1OCCN(C(=O)N1CCC(=O)CC1c1ccc(F)c(F)c1)C2] |
| 28 | 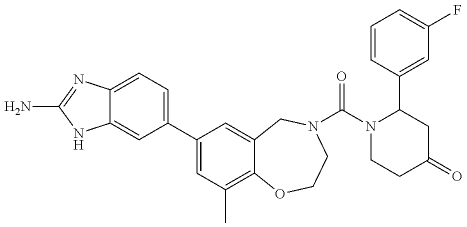 [SMILES: Cc1cc(-c2ccc3nc(N)[nH]c3c2)cc2c1OCCN(C(=O)N1CCC(=O)CC1c1cccc(F)c1)C2] |
| 29 | 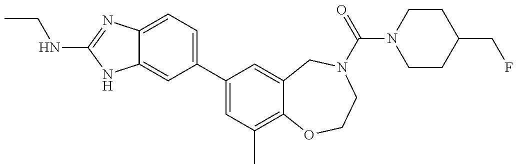 [SMILES: CCNc1nc2ccc(-c3cc(C)c4c(c3)CN(C(=O)N3CCC(CF)CC3)CCO4)cc2[nH]1] |
| 30 | 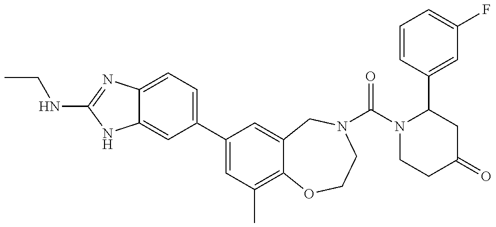 [SMILES: CCNc1nc2ccc(-c3cc(C)c4c(c3)CN(C(=O)N3CCC(=O)CC3c3cccc(F)c3)CCO4)cc2[nH]1] |
| 31 | 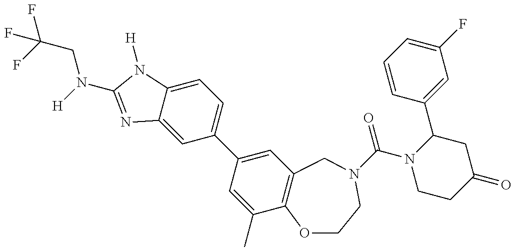 [SMILES: Cc1cc(-c2ccc3[nH]c(NCC(F)(F)F)nc3c2)cc2c1OCCN(C(=O)N1CCC(=O)CC1c1cccc(F)c1)C2] |
| 32 | 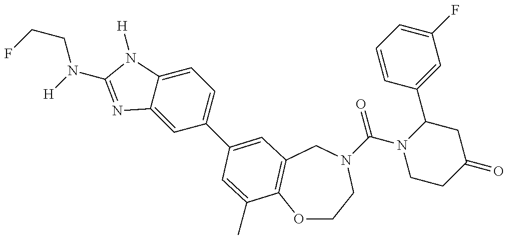 [SMILES: Cc1cc(-c2ccc3[nH]c(NCCF)nc3c2)cc2c1OCCN(C(=O)N1CCC(=O)CC1c1cccc(F)c1)C2] |
| 33 | 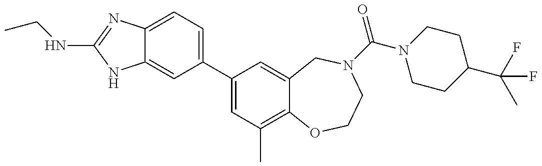 [SMILES: CCNc1nc2ccc(-c3cc(C)c4c(c3)CN(C(=O)N3CCC(C(C)(F)F)CC3)CCO4)cc2[nH]1] |
| 34 | 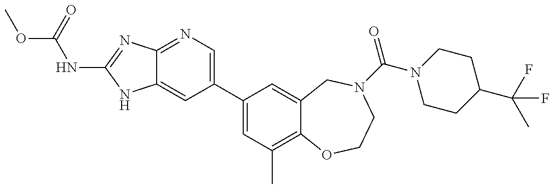 [SMILES: COC(=O)Nc1nc2ncc(-c3cc(C)c4c(c3)CN(C(=O)N3CCC(C(C)(F)F)CC3)CCO4)cc2[nH]1] |
| 35 | 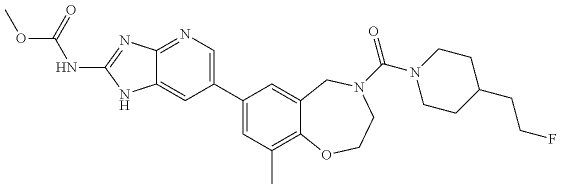 [SMILES: COC(=O)Nc1nc2ncc(-c3cc(C)c4c(c3)CN(C(=O)N3CCC(CCF)CC3)CCO4)cc2[nH]1] |
| 36 | 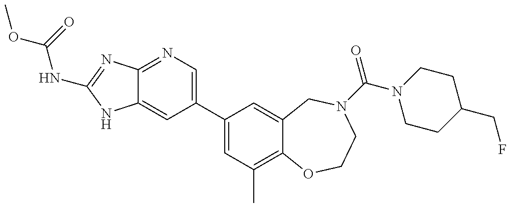 [SMILES: COC(=O)Nc1nc2ncc(-c3cc(C)c4c(c3)CN(C(=O)N3CCC(CF)CC3)CCO4)cc2[nH]1] |
| 37 | 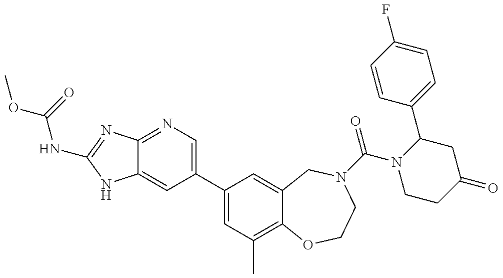 [SMILES: COC(=O)Nc1nc2ncc(-c3cc(C)c4c(c3)CN(C(=O)N3CCC(=O)CC3c3ccc(F)cc3)CCO4)cc2[nH]1] |
| 38 | 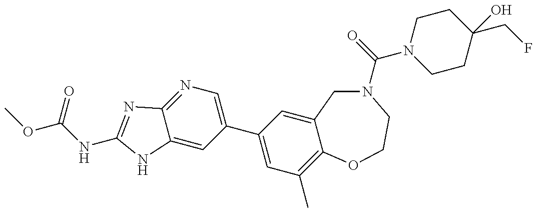 [SMILES: COC(=O)Nc1nc2ncc(-c3cc(C)c4c(c3)CN(C(=O)N3CCC(O)(CF)CC3)CCO4)cc2[nH]1] |
| 39 | 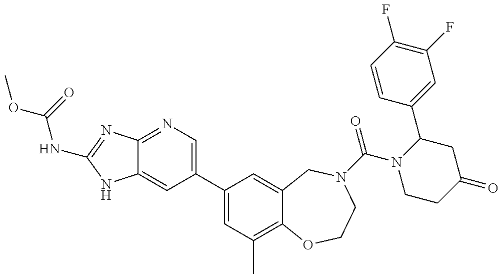 [SMILES: COC(=O)Nc1nc2ncc(-c3cc(C)c4c(c3)CN(C(=O)N3CCC(=O)CC3c3ccc(F)c(F)c3)CCO4)cc2[nH]1] |
| 40 | 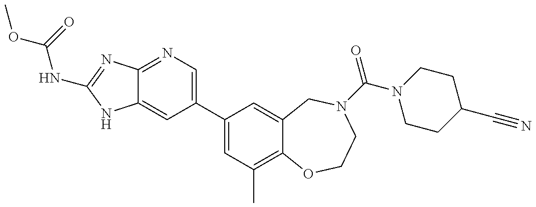 [SMILES: COC(=O)Nc1nc2ncc(-c3cc(C)c4c(c3)CN(C(=O)N3CCC(C#N)CC3)CCO4)cc2[nH]1] |
| 41 | 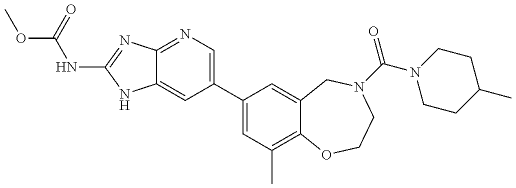 |
| 42 | 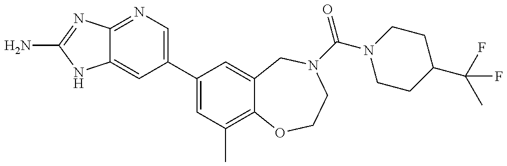 [SMILES: Cc1cc(-c2cnc3nc(N)[nH]c3c2)cc2c1OCCN(C(=O)N1CCC(C(C)(F)F)CC1)C2] |
| 43 | 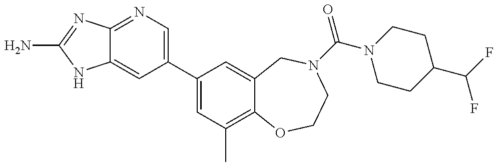 [SMILES: Cc1cc(-c2cnc3nc(N)[nH]c3c2)cc2c1OCCN(C(=O)N1CCC(C(F)F)CC1)C2] |
| 44 | 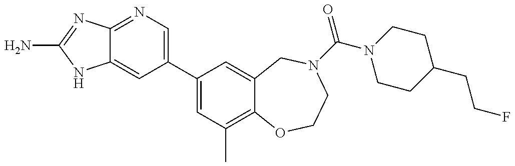 [SMILES: Cc1cc(-c2cnc3nc(N)[nH]c3c2)cc2c1OCCN(C(=O)N1CCC(CCF)CC1)C2] |
| 45 | 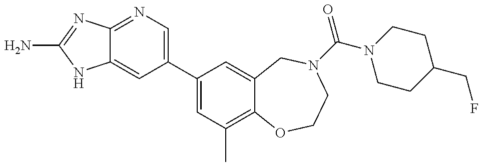 [SMILES: Cc1cc(-c2cnc3nc(N)[nH]c3c2)cc2c1OCCN(C(=O)N1CCC(CF)CC1)C2] |
| 46 | 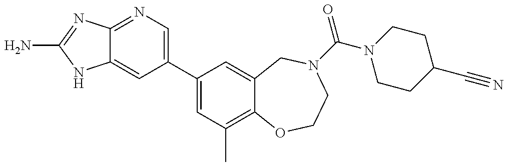 [SMILES: Cc1cc(-c2cnc3nc(N)[nH]c3c2)cc2c1OCCN(C(=O)N1CCC(C#N)CC1)C2] |
| 47 | 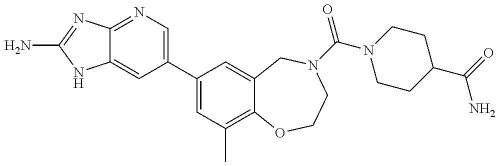 [SMILES: Cc1cc(-c2cnc3nc(N)[nH]c3c2)cc2c1OCCN(C(=O)N1CCC(C(N)=O)CC1)C2] |
| 48 | 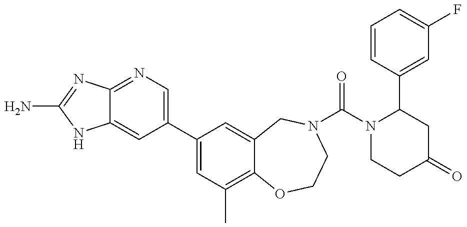 [SMILES: Cc1cc(-c2cnc3nc(N)[nH]c3c2)cc2c1OCCN(C(=O)N1CCC(=O)CC1c1cccc(F)c1)C2] |
| 49 | 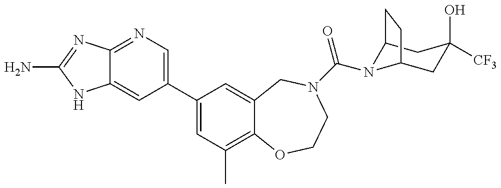 [SMILES: Cc1cc(-c2cnc3nc(N)[nH]c3c2)cc2c1OCCN(C(=O)N1C3CCC1CC(O)(C(F)(F)F)C3)C2] |
| 50 | 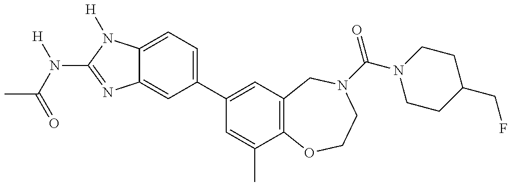 [SMILES: CC(=O)Nc1nc2cc(-c3cc(C)c4c(c3)CN(C(=O)N3CCC(CF)CC3)CCO4)ccc2[nH]1] |
| 51 | 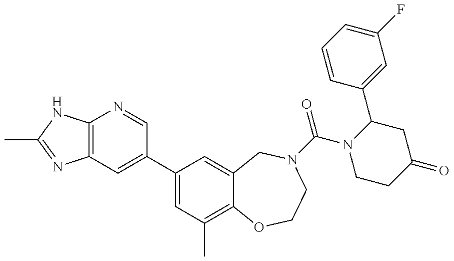 [SMILES: Cc1nc2cc(-c3cc(C)c4c(c3)CN(C(=O)N3CCC(=O)CC3c3cccc(F)c3)CCO4)cnc2[nH]1] |
| 52 | 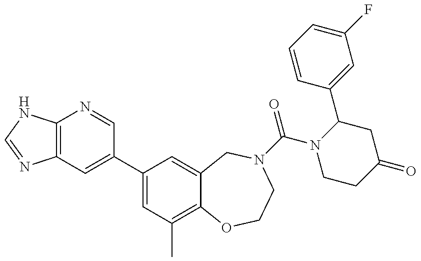 [SMILES: Cc1cc(-c2cnc3[nH]cnc3c2)cc2c1OCCN(C(=O)N1CCC(=O)CC1c1cccc(F)c1)C2] |
| 53 | 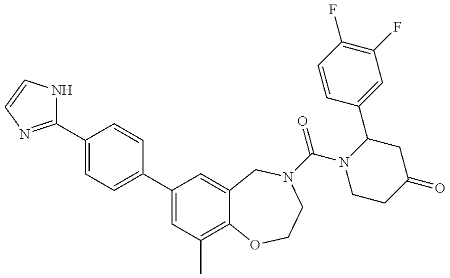 [SMILES: Cc1cc(-c2ccc(-c3ncc[nH]3)cc2)cc2c1OCCN(C(=O)N1CCC(=O)CC1c1ccc(F)c(F)c1)C2] |
| 54 | 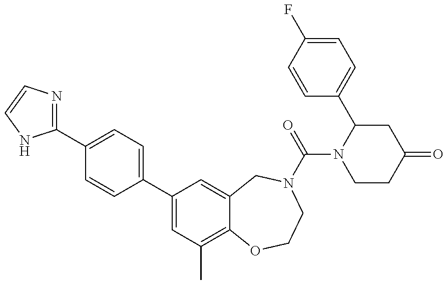 [SMILES: Cc1cc(-c2ccc(-c3ncc[nH]3)cc2)cc2c1OCCN(C(=O)N1CCC(=O)CC1c1ccc(F)cc1)C2] |
| 55 | 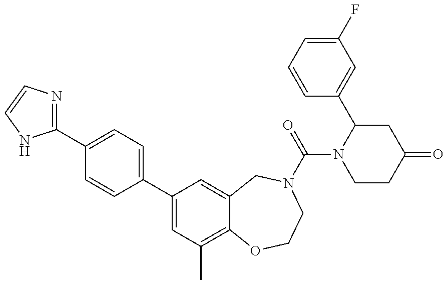 [SMILES: Cc1cc(-c2ccc(-c3ncc[nH]3)cc2)cc2c1OCCN(C(=O)N1CCC(=O)CC1c1cccc(F)c1)C2] |
| 56 | 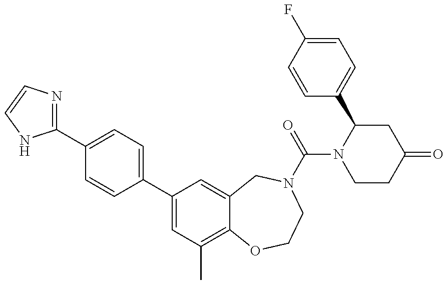 [SMILES: Cc1cc(-c2ccc(-c3ncc[nH]3)cc2)cc2c1OCCN(C(=O)N1CCC(=O)C[C@@H]1c1ccc(F)cc1)C2] |
| 57 | 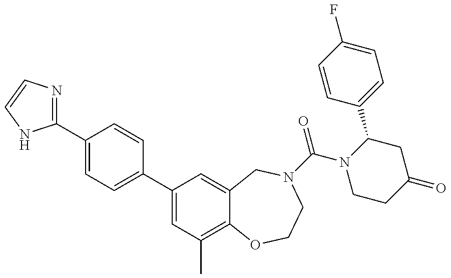 [SMILES: Cc1cc(-c2ccc(-c3ncc[nH]3)cc2)cc2c1OCCN(C(=O)N1CCC(=O)C[C@H]1c1ccc(F)cc1)C2] |
| 58 | 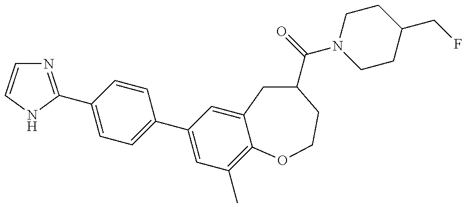 [SMILES: Cc1cc(-c2ccc(-c3ncc[nH]3)cc2)cc2c1OCCC(C(=O)N1CCC(CF)CC1)C2] |
| 59 | 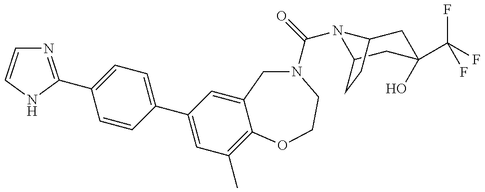 [SMILES: Cc1cc(-c2ccc(-c3ncc[nH]3)cc2)cc2c1OCCN(C(=O)N1C3CCC1CC(O)(C(F)(F)F)C3)C2] |
| 60 | 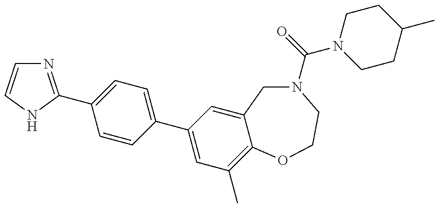 [SMILES: Cc1cc(-c2ccc(-c3ncc[nH]3)cc2)cc2c1OCCN(C(=O)N1CCC(C)CC1)C2] |
| 61 | 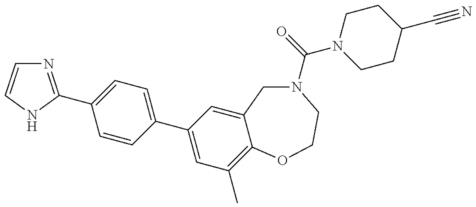 [SMILES: Cc1cc(-c2ccc(-c3ncc[nH]3)cc2)cc2c1OCCN(C(=O)N1CCC(C#N)CC1)C2] |
| 62 | 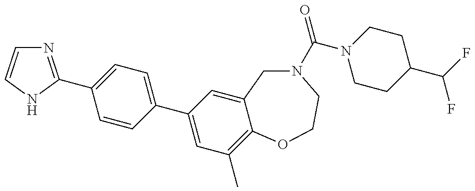 [SMILES: Cc1cc(-c2ccc(-c3ncc[nH]3)cc2)cc2c1OCCN(C(=O)N1CCC(C(F)F)CC1)C2] |
| 63 | 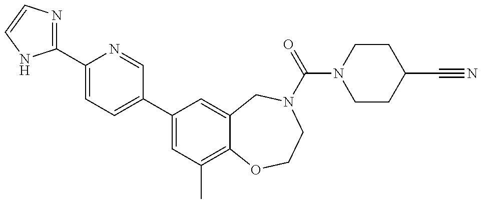 [SMILES: Cc1cc(-c2ccc(-c3ncc[nH]3)nc2)cc2c1OCCN(C(=O)N1CCC(C#N)CC1)C2] |
| 64 | 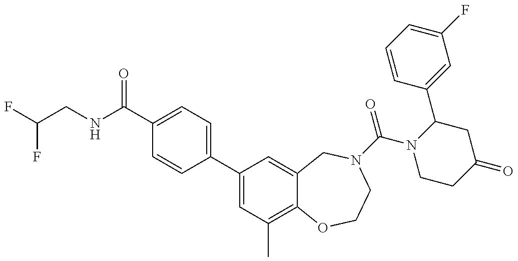 [SMILES: Cc1cc(-c2ccc(C(=O)NCC(F)F)cc2)cc2c1OCCN(C(=O)N1CCC(=O)CC1c1cccc(F)c1)C2] |
| 65 | 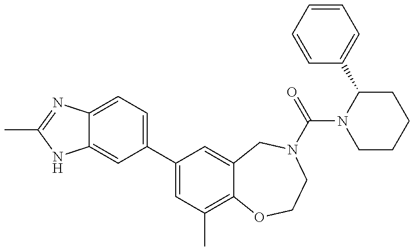 [SMILES: Cc1nc2ccc(-c3cc(C)c4c(c3)CN(C(=O)N3CCCC[C@H]3c3ccccc3)CCO4)cc2[nH]1] |
| 66 | 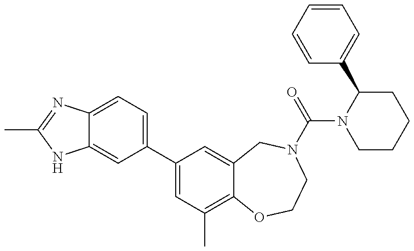 [SMILES: Cc1nc2ccc(-c3cc(C)c4c(c3)CN(C(=O)N3CCCC[C@@H]3c3ccccc3)CCO4)cc2[nH]1] |
| 67 | 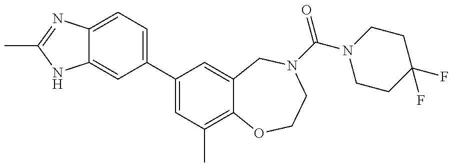 [SMILES: Cc1nc2ccc(-c3cc(C)c4c(c3)CN(C(=O)N3CCC(F)(F)CC3)CCO4)cc2[nH]1] |
| 68 | 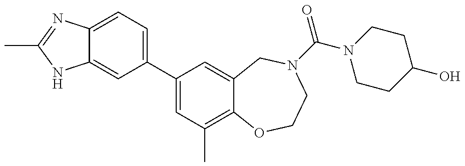 [SMILES: Cc1nc2ccc(-c3cc(C)c4c(c3)CN(C(=O)N3CCC(O)CC3)CCO4)cc2[nH]1] |
| 69 | 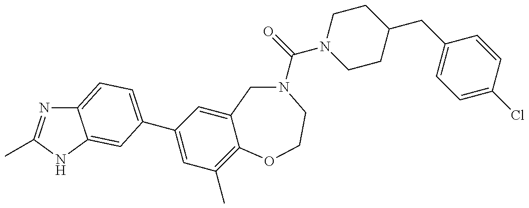 [SMILES: Cc1nc2ccc(-c3cc(C)c4c(c3)CN(C(=O)N3CCC(Cc5ccc(Cl)cc5)CC3)CCO4)cc2[nH]1] |
| 70 | 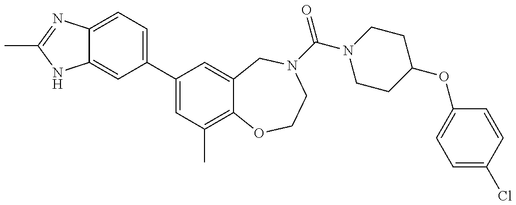 [SMILES: Cc1nc2ccc(-c3cc(C)c4c(c3)CN(C(=O)N3CCC(Oc5ccc(Cl)cc5)CC3)CCO4)cc2[nH]1] |
| 71 | 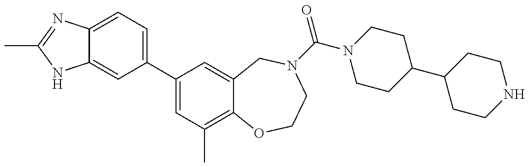 [SMILES: Cc1nc2ccc(-c3cc(C)c4c(c3)CN(C(=O)N3CCC(C5CCNCC5)CC3)CCO4)cc2[nH]1] |
| 72 | 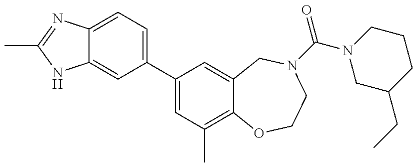 [SMILES: CCC1CCCN(C(=O)N2CCOc3c(C)cc(-c4ccc5nc(C)[nH]c5c4)cc3C2)C1] |
| 73 | 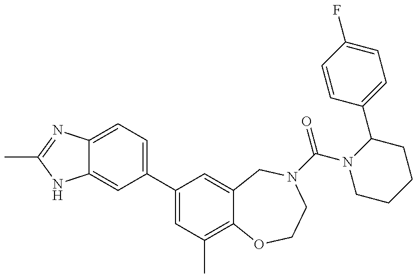 [SMILES: Cc1nc2ccc(-c3cc(C)c4c(c3)CN(C(=O)N3CCCCC3c3ccc(F)cc3)CCO4)cc2[nH]1] |
| 74 | 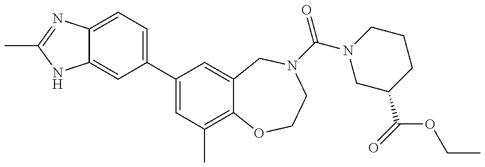 [SMILES: CCOC(=O)[C@H]1CCCN(C(=O)N2CCOc3c(C)cc(-c4ccc5nc(C)[nH]c5c4)cc3C2)C1] |
| 75 | 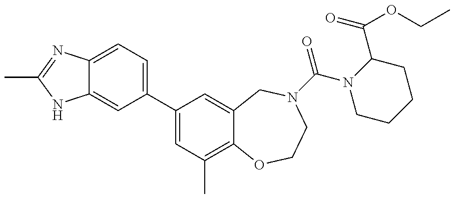 [SMILES: CCOC(=O)C1CCCCN1C(=O)N1CCOc2c(C)cc(-c3ccc4nc(C)[nH]c4c3)cc2C1] |
| 76 | 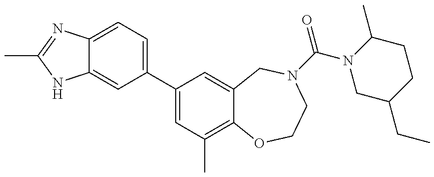 [SMILES: CCC1CCC(C)N(C(=O)N2CCOc3c(C)cc(-c4ccc5nc(C)[nH]c5c4)cc3C2)C1] |
| 77 | 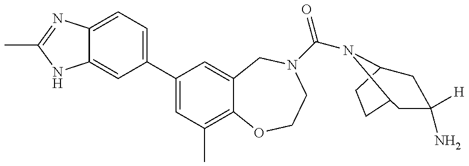 [SMILES: Cc1nc2ccc(-c3cc(C)c4c(c3)CN(C(=O)N3C5CCC3CC(N)C5)CCO4)cc2[nH]1] |
| 78 | 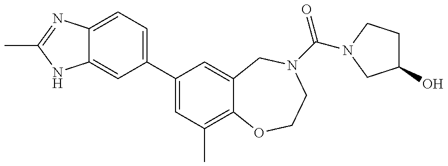 [SMILES: Cc1nc2ccc(-c3cc(C)c4c(c3)CN(C(=O)N3CC[C@@H](O)C3)CCO4)cc2[nH]1] |
| 79 | 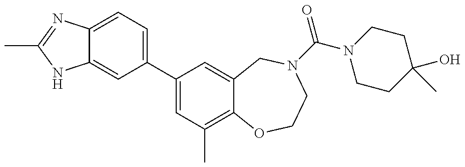 [SMILES: Cc1nc2ccc(-c3cc(C)c4c(c3)CN(C(=O)N3CCC(C)(O)CC3)CCO4)cc2[nH]1] |
| 80 | 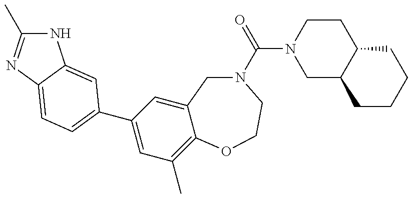 [SMILES: Cc1nc2ccc(-c3cc(C)c4c(c3)CN(C(=O)N3CC[C@@H]5CCCC[C@H]5C3)CCO4)cc2[nH]1] |
| 81 | 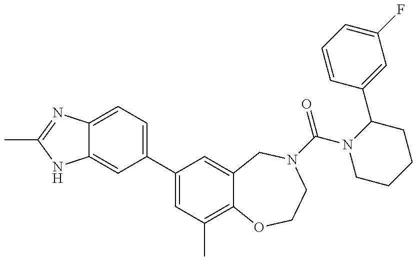 [SMILES: Cc1nc2ccc(-c3cc(C)c4c(c3)CN(C(=O)N3CCCCC3c3cccc(F)c3)CCO4)cc2[nH]1] |
| 82 | 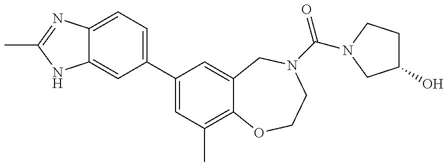 [SMILES: Cc1nc2ccc(-c3cc(C)c4c(c3)CN(C(=O)N3CC[C@H](O)C3)CCO4)cc2[nH]1] |
| 83 | 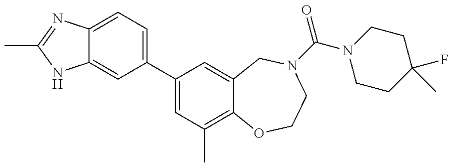 [SMILES: Cc1nc2ccc(-c3cc(C)c4c(c3)CN(C(=O)N3CCC(C)(F)CC3)CCO4)cc2[nH]1] |
| 84 | 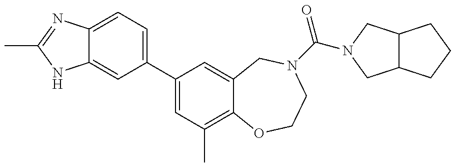 [SMILES: Cc1nc2ccc(-c3cc(C)c4c(c3)CN(C(=O)N3CC5CCCC5C3)CCO4)cc2[nH]1] |
| 85 | 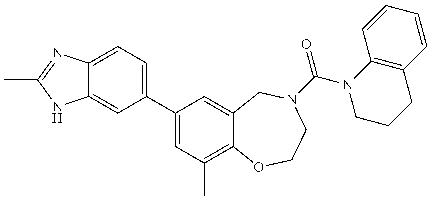 [SMILES: Cc1nc2ccc(-c3cc(C)c4c(c3)CN(C(=O)N3CCCc5ccccc53)CCO4)cc2[nH]1] |
| 86 | 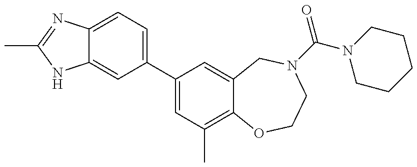 [SMILES: Cc1nc2ccc(-c3cc(C)c4c(c3)CN(C(=O)N3CCCCC3)CCO4)cc2[nH]1] |
| 87 | 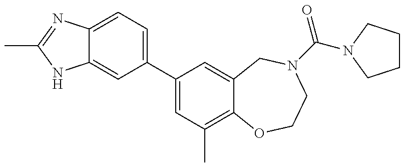 [SMILES: Cc1nc2ccc(-c3cc(C)c4c(c3)CN(C(=O)N3CCCC3)CCO4)cc2[nH]1] |
| 88 | 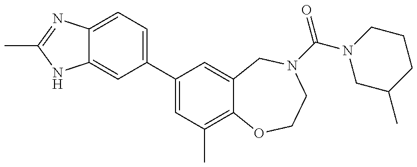 [SMILES: Cc1nc2ccc(-c3cc(C)c4c(c3)CN(C(=O)N3CCCC(C)C3)CCO4)cc2[nH]1] |
| 89 | 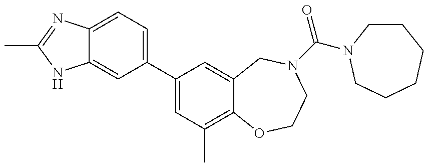 [SMILES: Cc1nc2ccc(-c3cc(C)c4c(c3)CN(C(=O)N3CCCCCC3)CCO4)cc2[nH]1] |
| 90 | 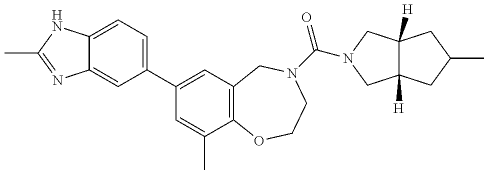 |
| 91 | 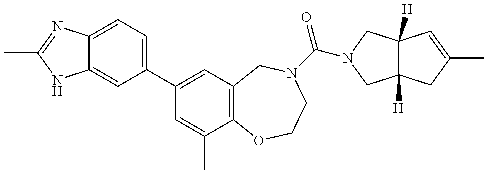 |
| 92 | 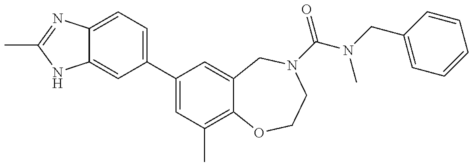 [SMILES: Cc1nc2ccc(-c3cc(C)c4c(c3)CN(C(=O)N(C)Cc3ccccc3)CCO4)cc2[nH]1] |
| 93 | 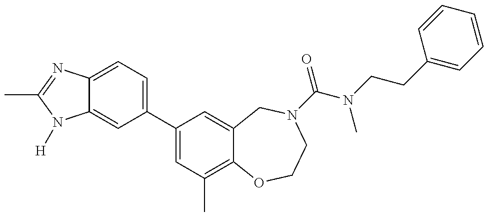 [SMILES: Cc1nc2ccc(-c3cc(C)c4c(c3)CN(C(=O)N(C)CCc3ccccc3)CCO4)cc2[nH]1] |
| 94 | 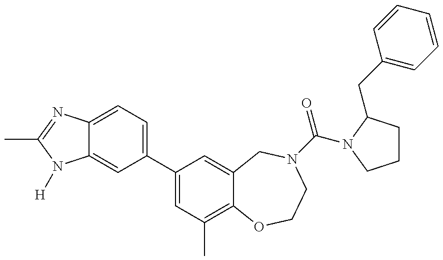 [SMILES: Cc1nc2ccc(-c3cc(C)c4c(c3)CN(C(=O)N3CCCC3Cc3ccccc3)CCO4)cc2[nH]1] |
| 95 | 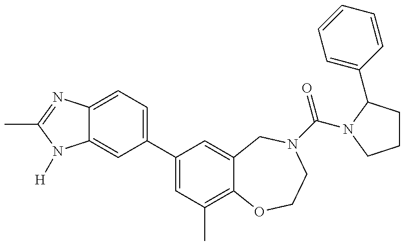 [SMILES: Cc1nc2ccc(-c3cc(C)c4c(c3)CN(C(=O)N3CCCC3c3ccccc3)CCO4)cc2[nH]1] |
| 96 | 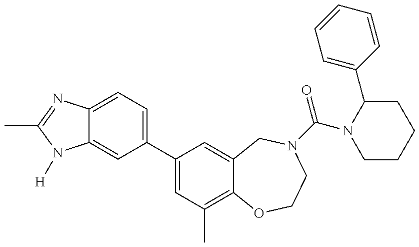 [SMILES: Cc1nc2ccc(-c3cc(C)c4c(c3)CN(C(=O)N3CCCCC3c3ccccc3)CCO4)cc2[nH]1] |
| 97 | 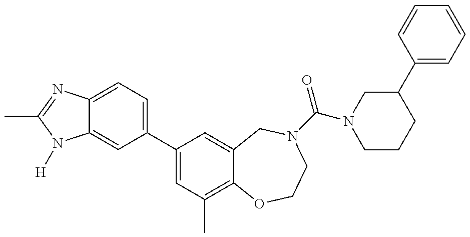 [SMILES: Cc1nc2ccc(-c3cc(C)c4c(c3)CN(C(=O)N3CCCC(c5ccccc5)C3)CCO4)cc2[nH]1] |
| 98 | 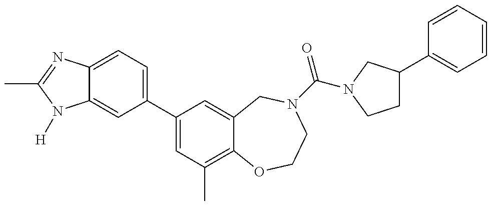 [SMILES: Cc1nc2ccc(-c3cc(C)c4c(c3)CN(C(=O)N3CCC(c5ccccc5)C3)CCO4)cc2[nH]1] |
| 99 | 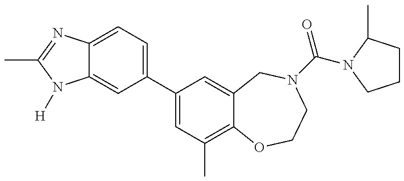 [SMILES: Cc1nc2ccc(-c3cc(C)c4c(c3)CN(C(=O)N3CCCC3C)CCO4)cc2[nH]1] |
| 100 | 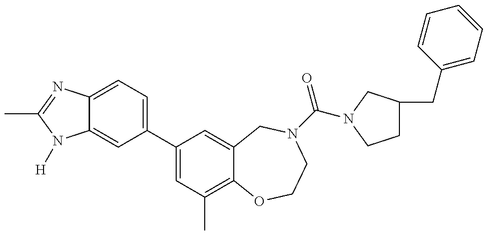 [SMILES: Cc1nc2ccc(-c3cc(C)c4c(c3)CN(C(=O)N3CCC(Cc5ccccc5)C3)CCO4)cc2[nH]1] |
| 101 | 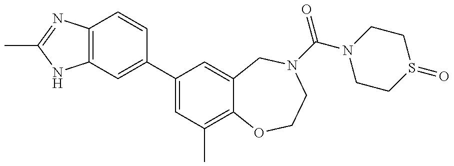 [SMILES: Cc1nc2ccc(-c3cc(C)c4c(c3)CN(C(=O)N3CCS(=O)CC3)CCO4)cc2[nH]1] |
| 102 | 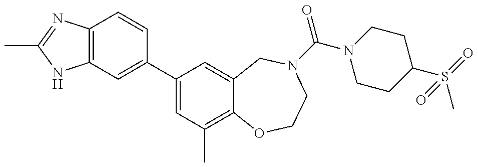 [SMILES: Cc1nc2ccc(-c3cc(C)c4c(c3)CN(C(=O)N3CCC(S(C)(=O)=O)CC3)CCO4)cc2[nH]1] |
| 103 | 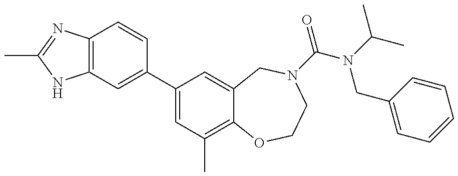 [SMILES: Cc1nc2ccc(-c3cc(C)c4c(c3)CN(C(=O)N(Cc3ccccc3)C(C)C)CCO4)cc2[nH]1] |
| 104 | 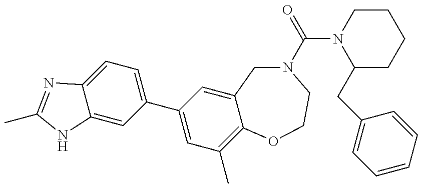 [SMILES: Cc1nc2ccc(-c3cc(C)c4c(c3)CN(C(=O)N3CCCCC3Cc3ccccc3)CCO4)cc2[nH]1] |
| 105 | 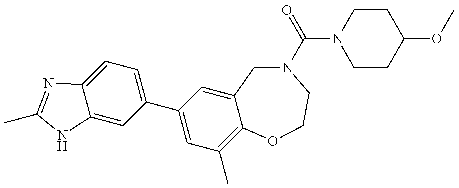 |
| 106 | 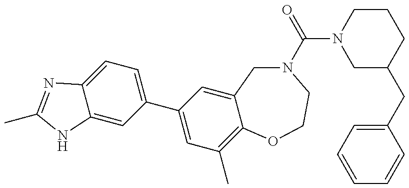 [SMILES: Cc1nc2ccc(-c3cc(C)c4c(c3)CN(C(=O)N3CCCC(Cc5ccccc5)C3)CCO4)cc2[nH]1] |
| 107 | 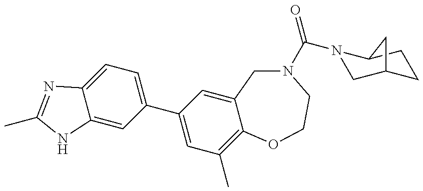 [SMILES: Cc1nc2ccc(-c3cc(C)c4c(c3)CN(C(=O)N3CC5CCC3C5)CCO4)cc2[nH]1] |
| 108 | 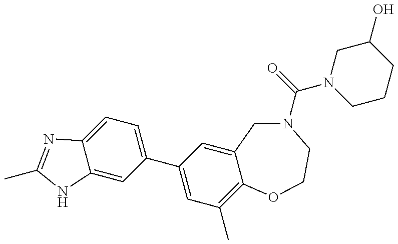 [SMILES: Cc1nc2ccc(-c3cc(C)c4c(c3)CN(C(=O)N3CCCC(O)C3)CCO4)cc2[nH]1] |
| 109 | 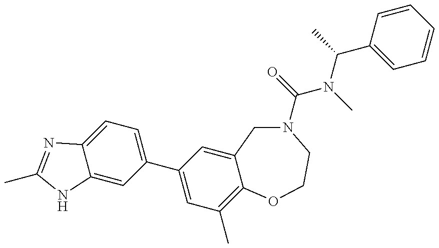 [SMILES: Cc1nc2ccc(-c3cc(C)c4c(c3)CN(C(=O)N(C)[C@H](C)c3ccccc3)CCO4)cc2[nH]1] |
| 110 | 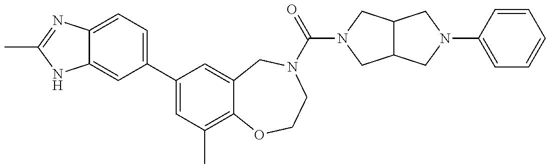 [SMILES: Cc1nc2ccc(-c3cc(C)c4c(c3)CN(C(=O)N3CC5CN(c6ccccc6)CC5C3)CCO4)cc2[nH]1] |
| 111 | 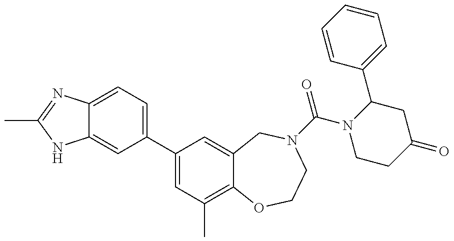 [SMILES: Cc1nc2ccc(-c3cc(C)c4c(c3)CN(C(=O)N3CCC(=O)CC3c3ccccc3)CCO4)cc2[nH]1] |
| 112 | 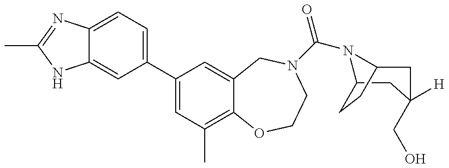 [SMILES: Cc1nc2ccc(-c3cc(C)c4c(c3)CN(C(=O)N3C5CCC3CC(CO)C5)CCO4)cc2[nH]1] |
| 113 | 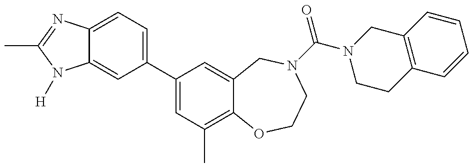 [SMILES: Cc1nc2ccc(-c3cc(C)c4c(c3)CN(C(=O)N3CCc5ccccc5C3)CCO4)cc2[nH]1] |
| 114 | 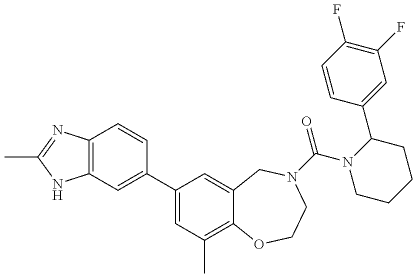 [SMILES: Cc1nc2ccc(-c3cc(C)c4c(c3)CN(C(=O)N3CCCCC3c3ccc(F)c(F)c3)CCO4)cc2[nH]1] |
| 115 | 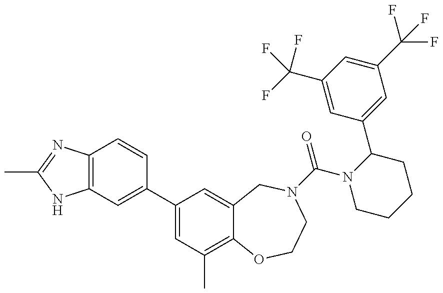 [SMILES: Cc1nc2ccc(-c3cc(C)c4c(c3)CN(C(=O)N3CCCCC3c3cc(C(F)(F)F)cc(C(F)(F)F)c3)CCO4)cc2[nH]1] |
| 116 | 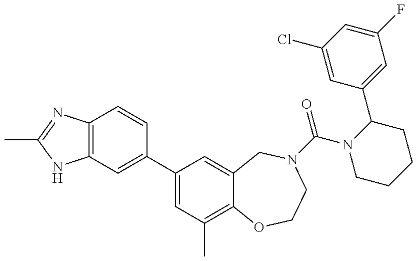 [SMILES: Cc1nc2ccc(-c3cc(C)c4c(c3)CN(C(=O)N3CCCCC3c3cc(F)cc(Cl)c3)CCO4)cc2[nH]1] |
| 117 | 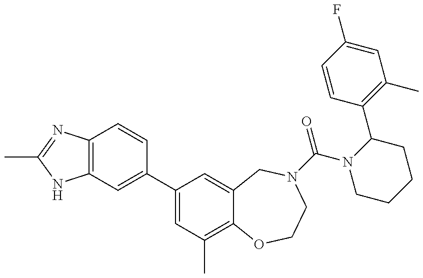 |
| 118 | 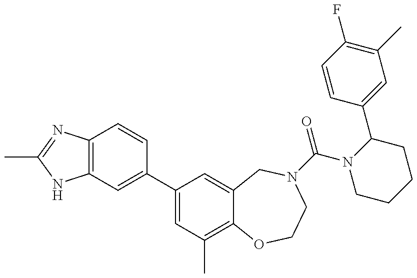 [SMILES: Cc1nc2ccc(-c3cc(C)c4c(c3)CN(C(=O)N3CCCCC3c3ccc(F)c(C)c3)CCO4)cc2[nH]1] |
| 119 | 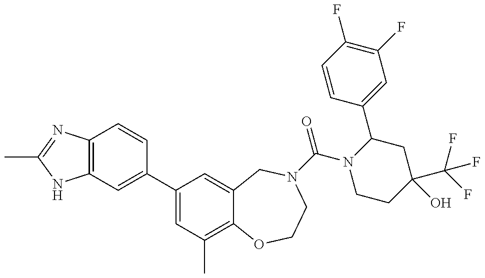 [SMILES: Cc1nc2ccc(-c3cc(C)c4c(c3)CN(C(=O)N3CCC(O)(C(F)(F)F)CC3c3ccc(F)c(F)c3)CCO4)cc2[nH]1] |
| 120 | 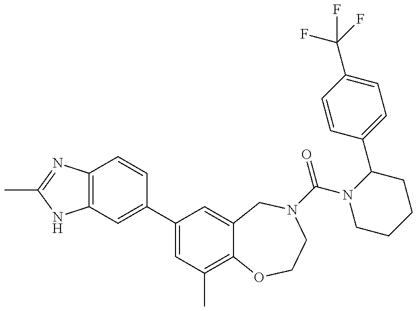 [SMILES: Cc1nc2ccc(-c3cc(C)c4c(c3)CN(C(=O)N3CCCCC3c3ccc(C(F)(F)F)cc3)CCO4)cc2[nH]1] |
| 121 | 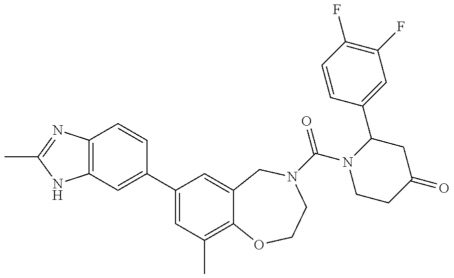 [SMILES: Cc1nc2ccc(-c3cc(C)c4c(c3)CN(C(=O)N3CCC(=O)CC3c3ccc(F)c(F)c3)CCO4)cc2[nH]1] |
| 122 | 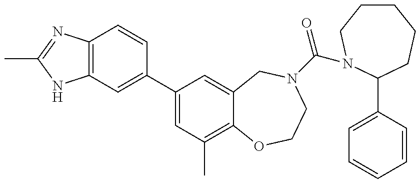 [SMILES: Cc1nc2ccc(-c3cc(C)c4c(c3)CN(C(=O)N3CCCCCC3c3ccccc3)CCO4)cc2[nH]1] |
| 123 | 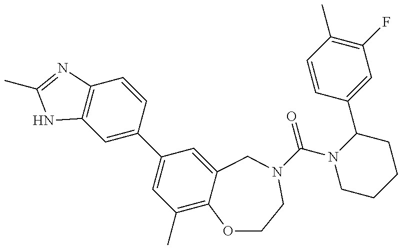 [SMILES: Cc1nc2ccc(-c3cc(C)c4c(c3)CN(C(=O)N3CCCCC3c3ccc(C)c(F)c3)CCO4)cc2[nH]1] |
| 124 | 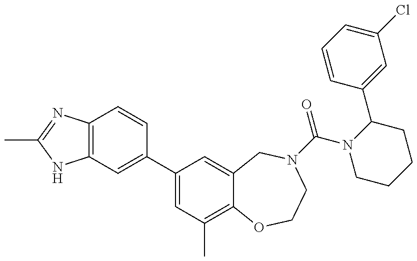 [SMILES: Cc1nc2ccc(-c3cc(C)c4c(c3)CN(C(=O)N3CCCCC3c3cccc(Cl)c3)CCO4)cc2[nH]1] |
| 125 | 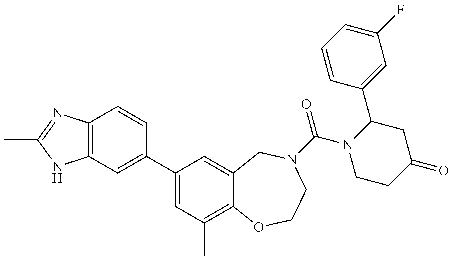 [SMILES: Cc1nc2ccc(-c3cc(C)c4c(c3)CN(C(=O)N3CCC(=O)CC3c3cccc(F)c3)CCO4)cc2[nH]1] |
| 126 | 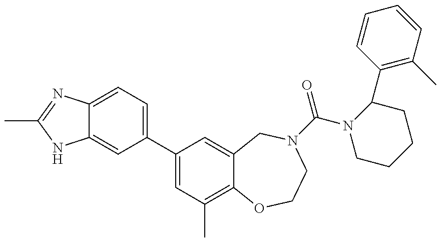 |
| 127 | 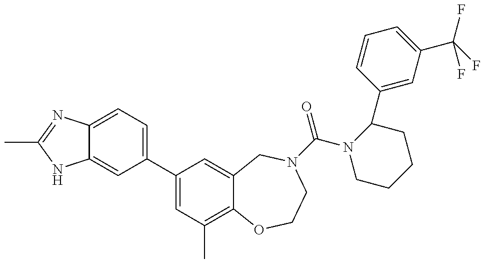 [SMILES: Cc1nc2ccc(-c3cc(C)c4c(c3)CN(C(=O)N3CCCCC3c3cccc(C(F)(F)F)c3)CCO4)cc2[nH]1] |
| 128 | 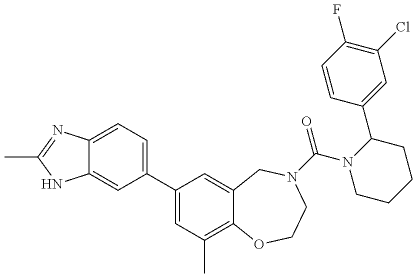 [SMILES: Cc1nc2ccc(-c3cc(C)c4c(c3)CN(C(=O)N3CCCCC3c3ccc(F)c(Cl)c3)CCO4)cc2[nH]1] |
| 129 | 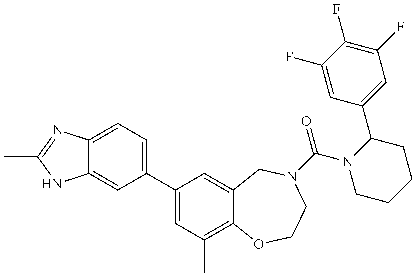 [SMILES: Cc1nc2ccc(-c3cc(C)c4c(c3)CN(C(=O)N3CCCCC3c3cc(F)c(F)c(F)c3)CCO4)cc2[nH]1] |
| 130 | 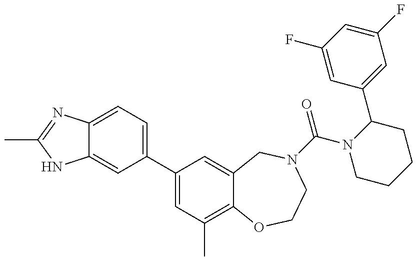 [SMILES: Cc1nc2ccc(-c3cc(C)c4c(c3)CN(C(=O)N3CCCCC3c3cc(F)cc(F)c3)CCO4)cc2[nH]1] |
| 131 | 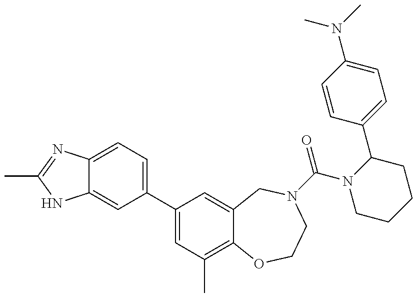 [SMILES: Cc1nc2ccc(-c3cc(C)c4c(c3)CN(C(=O)N3CCCCC3c3ccc(N(C)C)cc3)CCO4)cc2[nH]1] |
| 132 | 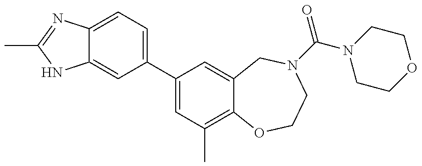 [SMILES: Cc1nc2ccc(-c3cc(C)c4c(c3)CN(C(=O)N3CCOCC3)CCO4)cc2[nH]1] |
| 133 | 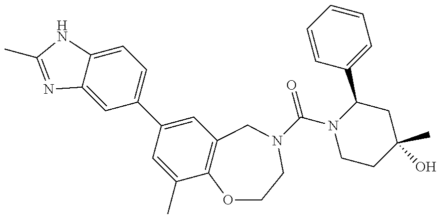 |
| 134 | 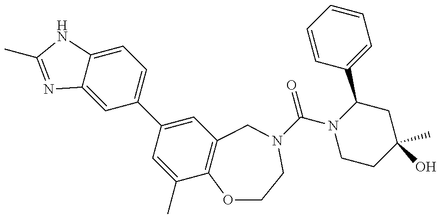 |
| 135 | 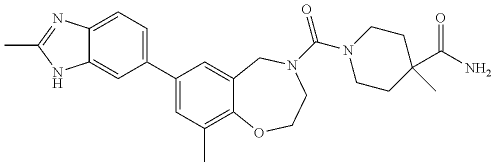 [SMILES: Cc1nc2ccc(-c3cc(C)c4c(c3)CN(C(=O)N3CCC(C)(C(N)=O)CC3)CCO4)cc2[nH]1] |
| 136 | 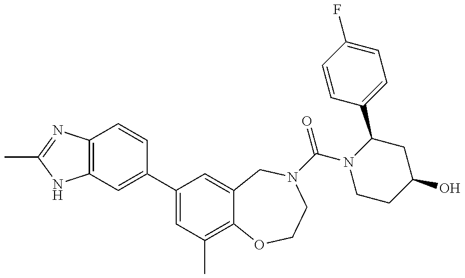 [SMILES: Cc1nc2ccc(-c3cc(C)c4c(c3)CN(C(=O)N3CC[C@H](O)C[C@@H]3c3ccc(F)cc3)CCO4)cc2[nH]1] |
| 137 | 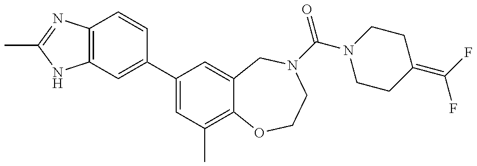 [SMILES: Cc1nc2ccc(-c3cc(C)c4c(c3)CN(C(=O)N3CCC(=C(F)F)CC3)CCO4)cc2[nH]1] |
| 138 | 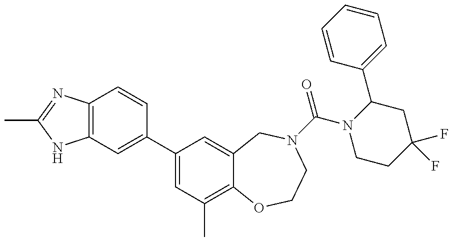 [SMILES: Cc1nc2ccc(-c3cc(C)c4c(c3)CN(C(=O)N3CCC(F)(F)CC3c3ccccc3)CCO4)cc2[nH]1] |
| 139 | 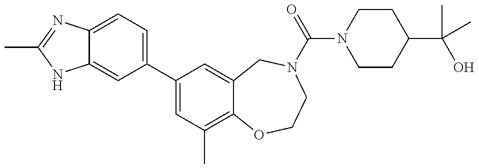 [SMILES: Cc1nc2ccc(-c3cc(C)c4c(c3)CN(C(=O)N3CCC(C(C)(C)O)CC3)CCO4)cc2[nH]1] |
| 140 | 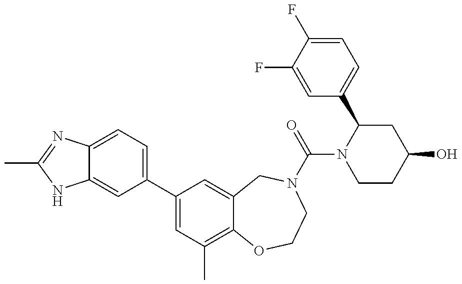 [SMILES: Cc1nc2ccc(-c3cc(C)c4c(c3)CN(C(=O)N3CC[C@H](O)C[C@@H]3c3ccc(F)c(F)c3)CCO4)cc2[nH]1] |
| 141 | 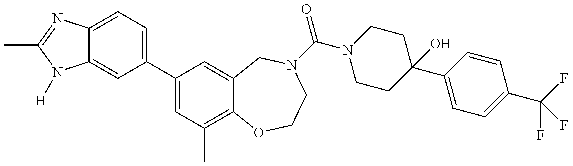 [SMILES: Cc1nc2ccc(-c3cc(C)c4c(c3)CN(C(=O)N3CCC(O)(c5ccc(C(F)(F)F)cc5)CC3)CCO4)cc2[nH]1] |
| 142 | 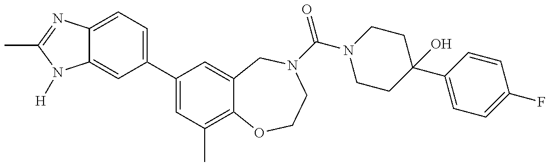 [SMILES: Cc1nc2ccc(-c3cc(C)c4c(c3)CN(C(=O)N3CCC(O)(c5ccc(F)cc5)CC3)CCO4)cc2[nH]1] |
| 143 | 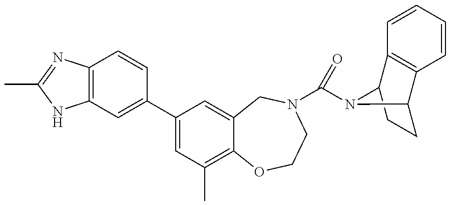 [SMILES: Cc1nc2ccc(-c3cc(C)c4c(c3)CN(C(=O)N3C5CCC3c3ccccc35)CCO4)cc2[nH]1] |
| 144 | 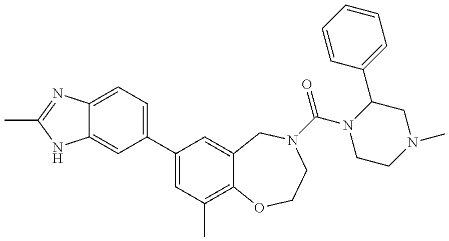 |
| 145 | 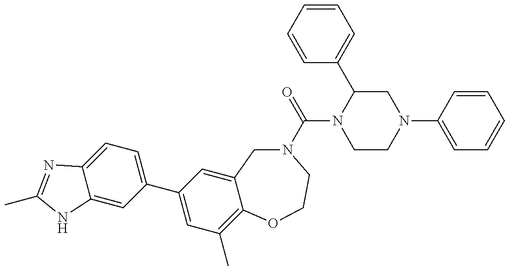 [SMILES: Cc1nc2ccc(-c3cc(C)c4c(c3)CN(C(=O)N3CCN(c5ccccc5)CC3c3ccccc3)CCO4)cc2[nH]1] |
| 146 | 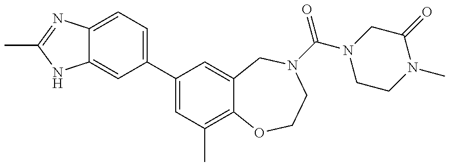 |
| 147 | 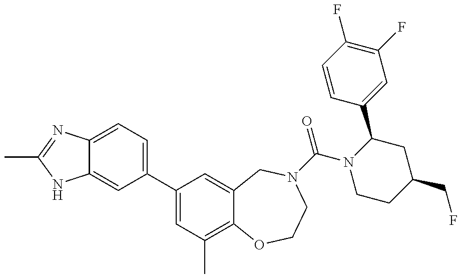 [SMILES: Cc1nc2ccc(-c3cc(C)c4c(c3)CN(C(=O)N3CC[C@H](CF)C[C@@H]3c3ccc(F)c(F)c3)CCO4)cc2[nH]1] |
| 148 | 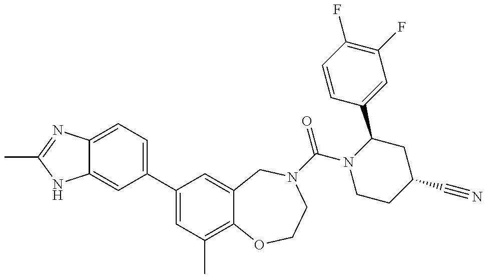 [SMILES: Cc1nc2ccc(-c3cc(C)c4c(c3)CN(C(=O)N3CC[C@@H](C#N)C[C@@H]3c3ccc(F)c(F)c3)CCO4)cc2[nH]1] |
| 149 | 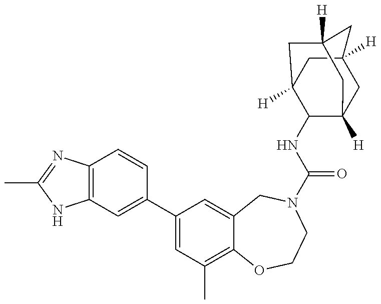 [SMILES: Cc1nc2ccc(-c3cc(C)c4c(c3)CN(C(=O)NC3[C@H]5C[C@@H]6C[C@@H](C[C@H]3C6)C5)CCO4)cc2[nH]1] |
| 150 | 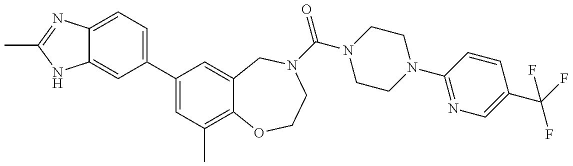 [SMILES: Cc1nc2ccc(-c3cc(C)c4c(c3)CN(C(=O)N3CCN(c5ccc(C(F)(F)F)cn5)CC3)CCO4)cc2[nH]1] |
| 151 | 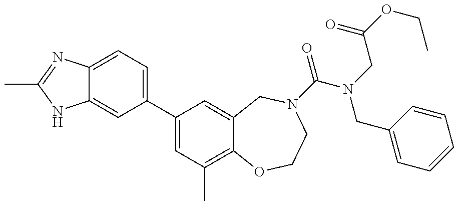 [SMILES: CCOC(=O)CN(Cc1ccccc1)C(=O)N1CCOc2c(C)cc(-c3ccc4nc(C)[nH]c4c3)cc2C1] |
| 152 | 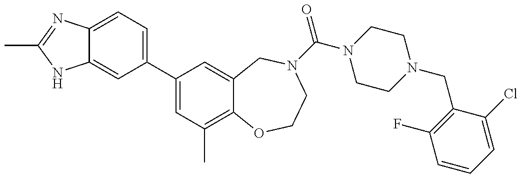 [SMILES: Cc1nc2ccc(-c3cc(C)c4c(c3)CN(C(=O)N3CCN(Cc5c(F)cccc5Cl)CC3)CCO4)cc2[nH]1] |
| 153 | 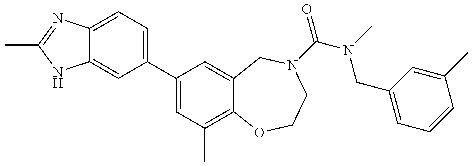 |
| 154 | 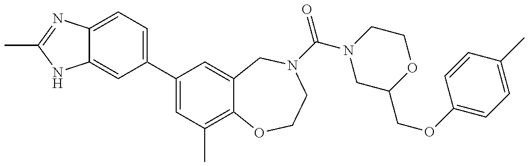 |
| 155 | 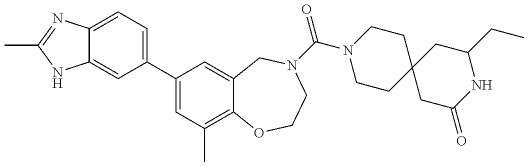 [SMILES: CCC1CC2(CCN(C(=O)N3CCOc4c(C)cc(-c5ccc6nc(C)[nH]c6c5)cc4C3)CC2)CC(=O)N1] |
| 156 | 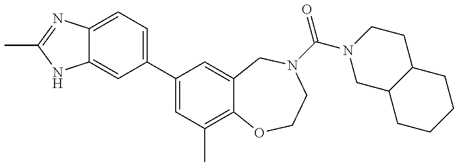 [SMILES: Cc1nc2ccc(-c3cc(C)c4c(c3)CN(C(=O)N3CCC5CCCCC5C3)CCO4)cc2[nH]1] |
| 157 | 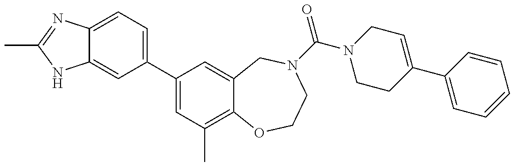 [SMILES: Cc1nc2ccc(-c3cc(C)c4c(c3)CN(C(=O)N3CC=C(c5ccccc5)CC3)CCO4)cc2[nH]1] |
| 158 | 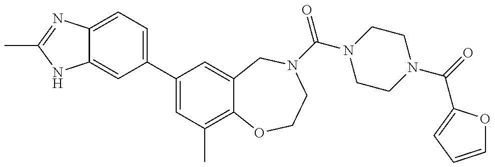 [SMILES: Cc1nc2ccc(-c3cc(C)c4c(c3)CN(C(=O)N3CCN(C(=O)c5ccco5)CC3)CCO4)cc2[nH]1] |
| 159 | 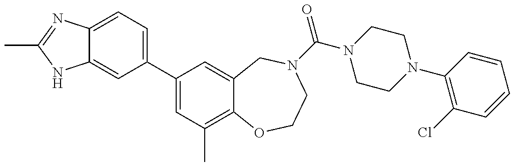 [SMILES: Cc1nc2ccc(-c3cc(C)c4c(c3)CN(C(=O)N3CCN(c5ccccc5Cl)CC3)CCO4)cc2[nH]1] |
| 160 | 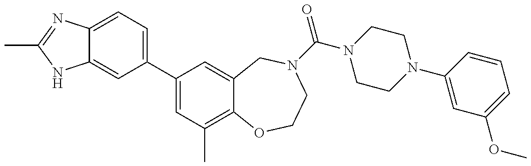 |
| 161 | 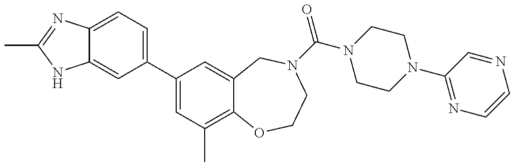 [SMILES: Cc1nc2ccc(-c3cc(C)c4c(c3)CN(C(=O)N3CCN(c5cnccn5)CC3)CCO4)cc2[nH]1] |
| 162 | 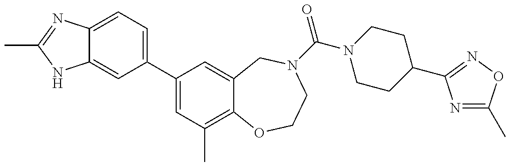 |
| 163 | 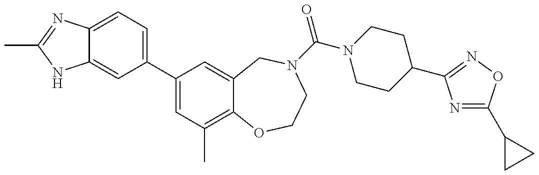 [SMILES: Cc1nc2ccc(-c3cc(C)c4c(c3)CN(C(=O)N3CCC(c5noc(C6CC6)n5)CC3)CCO4)cc2[nH]1] |
| 164 | 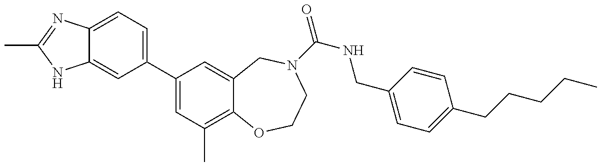 [SMILES: CCCCCc1ccc(CNC(=O)N2CCOc3c(C)cc(-c4ccc5nc(C)[nH]c5c4)cc3C2)cc1] |
| 165 | 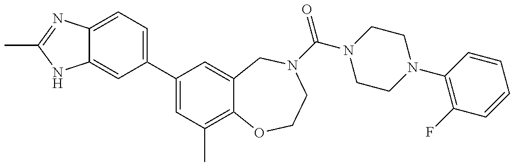 [SMILES: Cc1nc2ccc(-c3cc(C)c4c(c3)CN(C(=O)N3CCN(c5ccccc5F)CC3)CCO4)cc2[nH]1] |
| 166 | 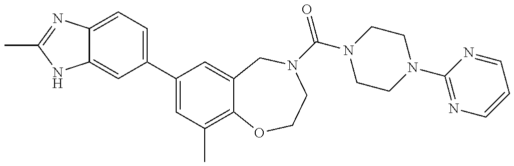 [SMILES: Cc1nc2ccc(-c3cc(C)c4c(c3)CN(C(=O)N3CCN(c5ncccn5)CC3)CCO4)cc2[nH]1] |
| 167 | 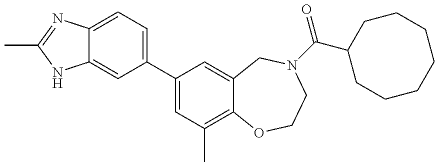 [SMILES: Cc1nc2ccc(-c3cc(C)c4c(c3)CN(C(=O)C3CCCCCCC3)CCO4)cc2[nH]1] |
| 168 | 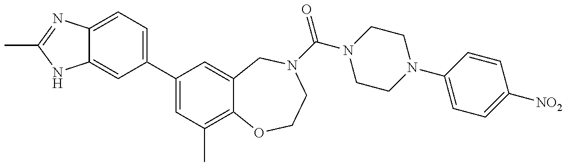 [SMILES: Cc1nc2ccc(-c3cc(C)c4c(c3)CN(C(=O)N3CCN(c5ccc([N+](=O)[O-])cc5)CC3)CCO4)cc2[nH]1] |
| 169 | 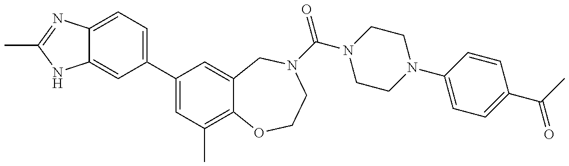 [SMILES: CC(=O)c1ccc(N2CCN(C(=O)N3CCOc4c(C)cc(-c5ccc6nc(C)[nH]c6c5)cc4C3)CC2)cc1] |
| 170 | 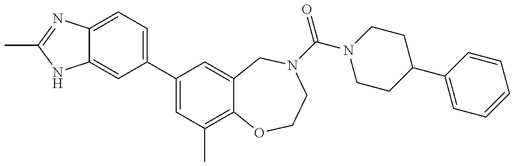 [SMILES: Cc1nc2ccc(-c3cc(C)c4c(c3)CN(C(=O)N3CCC(c5ccccc5)CC3)CCO4)cc2[nH]1] |
| 171 | 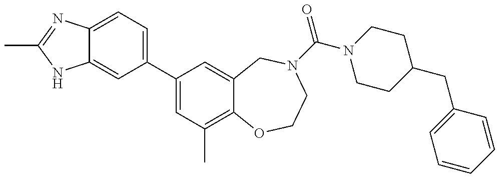 [SMILES: Cc1nc2ccc(-c3cc(C)c4c(c3)CN(C(=O)N3CCC(Cc5ccccc5)CC3)CCO4)cc2[nH]1] |
| 172 | 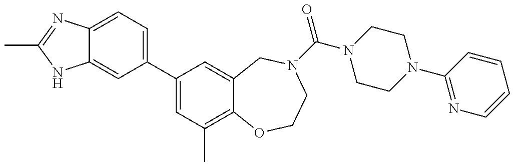 [SMILES: Cc1nc2ccc(-c3cc(C)c4c(c3)CN(C(=O)N3CCN(c5ccccn5)CC3)CCO4)cc2[nH]1] |
| 173 | 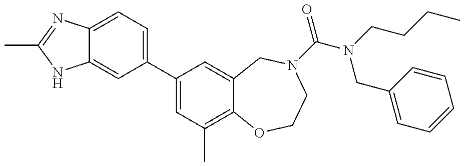 [SMILES: CCCCN(Cc1ccccc1)C(=O)N1CCOc2c(C)cc(-c3ccc4nc(C)[nH]c4c3)cc2C1] |
| 174 | 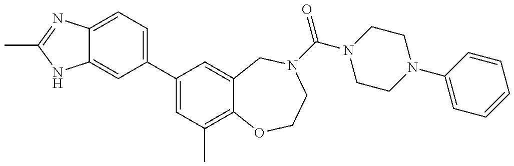 [SMILES: Cc1nc2ccc(-c3cc(C)c4c(c3)CN(C(=O)N3CCN(c5ccccc5)CC3)CCO4)cc2[nH]1] |
| 175 | 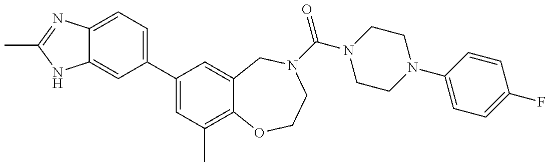 [SMILES: Cc1nc2ccc(-c3cc(C)c4c(c3)CN(C(=O)N3CCN(c5ccc(F)cc5)CC3)CCO4)cc2[nH]1] |
| 176 | 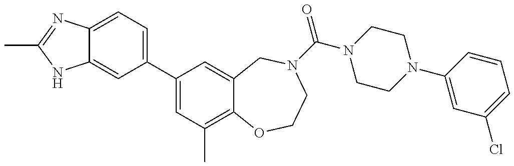 [SMILES: Cc1nc2ccc(-c3cc(C)c4c(c3)CN(C(=O)N3CCN(c5cccc(Cl)c5)CC3)CCO4)cc2[nH]1] |
| 177 | 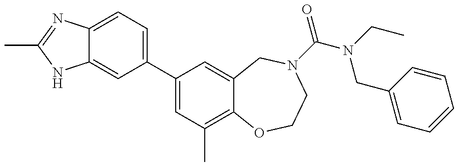 [SMILES: CCN(Cc1ccccc1)C(=O)N1CCOc2c(C)cc(-c3ccc4nc(C)[nH]c4c3)cc2C1] |
| 178 | 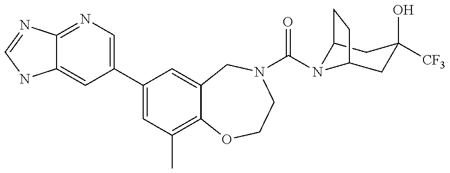 [SMILES: Cc1cc(-c2cnc3nc[nH]c3c2)cc2c1OCCN(C(=O)N1C3CCC1CC(O)(C(F)(F)F)C3)C2] |
| 179 | 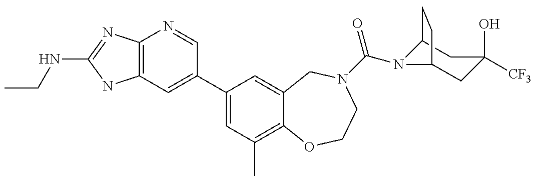 [SMILES: CCNc1nc2ncc(-c3cc(C)c4c(c3)CN(C(=O)N3C5CCC3CC(O)(C(F)(F)F)C5)CCO4)cc2[nH]1] |
| 180 | 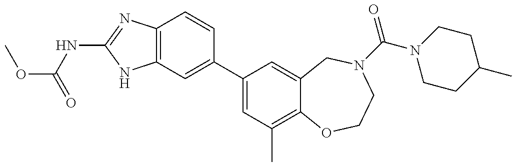 |
| 181 | 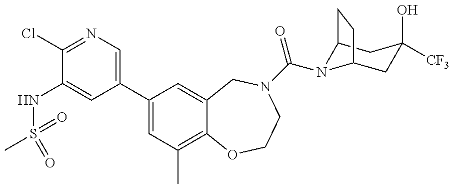 [SMILES: Cc1cc(-c2cnc(Cl)c(NS(C)(=O)=O)c2)cc2c1OCCN(C(=O)N1C3CCC1CC(O)(C(F)(F)F)C3)C2] |

optionally as a pharmaceutically acceptable salt thereof.

21. A pharmaceutical composition which comprises a compound, optionally as pharmaceutically acceptable salt thereof, of claim 1 and a pharmaceutically acceptable carrier, excipient, or diluent.

22. A method of making a Compound of Formula I, according to claim 1 which method comprises
(a) reacting the following, or a salt thereof: 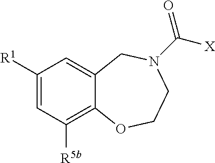
where X is halo and R¹ is as defined in claim 1; with an intermediate of formula R²H where R² is as defined in claim 1 to yield a compound of Formula I; and optionally separating individual isomers; and optionally modifying any of the R¹ and R² groups; and optionally forming a pharmaceutically acceptable salt thereof; or
(b) reacting the following, or a salt thereof: 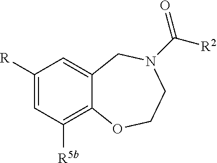
where R is halo or —B(OR′)₂ (where both R¹ are hydrogen or the two R¹ together form a boronic ester), and R² is as defined in claim 1; with an intermediate of formula R¹Y where Y is halo when R is —B(OR′)₂ and Y is —B(OR′)₂ when R is halo, and R² is as defined in claim 1, to yield a compound of Formula I; and optionally separating individual isomers; and optionally modifying any of the R¹ and R² groups; and optionally forming a pharmaceutically acceptable salt, hydrate, solvate or combination thereof.

23. A method for treating a disease, disorder, or syndrome which method comprises administering to a patient a therapeutically effective amount of a compound of claim 1, optionally as a pharmaceutically acceptable salt thereof, or a pharmaceutical composition comprising a compound of claim 1 and a pharmaceutically acceptable carrier, excipient, or diluent.

24. The method of claim 23 where the disease is cancer.

25. The method of claim 23 where the cancer is breast cancer, mantle cell lymphoma, renal cell carcinoma, acute myelogenous leukemia, chronic myelogenous leukemia, NPM/ALK-transformed anaplastic large cell lymphoma, diffuse large B cell lymphoma, rhabdomyosarcoma, ovarian cancer, endometrial cancer, cervical cancer, non small cell lung carcinoma, small cell lung carcinoma, adenocarcinoma, colon cancer, rectal cancer, gastric carcinoma, hepatocellular carcinoma, melanoma, pancreatic cancer, prostate carcinoma, thyroid carcinoma, anaplastic large cell lymphoma, hemangioma, glioblastoma, or head and neck cancer.
